(19) United States
(12) Patent Application Publication
(10) Pub. No.: US 2009/0325964 A1
(43) Pub. Date: Dec. 31, 2009

(54) Piperazine Metabotropic Glutamate Receptor 5 (MGLUR5) Negative Allosteric Modulators For Anxiety/Depression

(75) Inventors: Matthew Gregory Bursavich, Salt Lake City, UT (US); Adam Matthew Gilbert, Congers, NY (US); Joseph Raymond Stock, Monroe, NY (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/470,814

(22) Filed: May 22, 2009

Related U.S. Application Data
(60) Provisional application No. 61/055,671, filed on May 23, 2008.

(51) Int. Cl. A61K 31/53; C07D 401/14; A61K 31/497; C07D 251/30

(52) U.S. Cl. 514/241; 544/364; 514/253.13; 544/357; 514/252.18; 544/219

(57) ABSTRACT

The present teachings relate to piperazine metabotropic glutamate receptor 5 (mGluR5) negative allosteric modulators having Formula I:

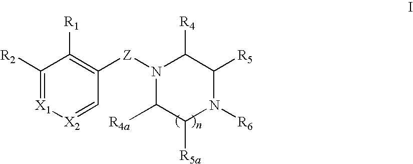

wherein the constituent variables are as defined herein. The present teachings further relate to methods for the preparation of the compounds, and to methods for using the compounds for treatment of diseases and disorders including schizophrenia, paranoia, depression, manic-depressive illness and anxiety.

### CROSS-REFERENCE TO RELATED APPLICATIONS

This application claims the benefit under 35 USC 119(e) of U.S. provisional application 61/055,671 filed on May 23, 2008, which is incorporated herein by reference in their entirety.

### FIELD OF THE INVENTION

In one aspect, this invention relates to piperazine metabotropic glutamate receptor 5 (mGluR5) negative allosteric modulators, and methods for their preparation. In a further aspect, the invention provides methods for using the mGluR5 negative allosteric modulators for treatment of diseases and disorders including schizophrenia, paranoia, depression, manic-depressive illness, anxiety (including panic disorders, social anxiety, obsessive compulsive disorders, generalized anxiety disorders, phobias), post-traumatic stress disorder, bipolar disorder, Asperger's syndrome, pervasive developmental disorders, gastrointestinal disorders such as gastroesophageal reflux disease, dyspepsia, irritable bowel syndrome, functional bloating, functional diarrhea, chronic constipation, functional disturbances of the biliary tract, migraine, chronic pain, fibromyalgia, neuropathic pain, post-herpatic neuropathic pain, addiction, Parkinson's disease, senile dementia, levadopa-induced dyskinesia, Alzheimer's disease, Huntington's chorea, amyotrophic lateral sclerosis, multiple sclerosis, Down Syndrome, fragile-X syndrome, autistic spectrum disorders, attention deficit hyperactivity disorder, stroke, ischemic injury, epilepsy, hypoglycemia and obesity.

### BACKGROUND OF THE INVENTION

The metabotropic glutamate 5 receptor (mGluR5) is a G-protein-coupled metabolic glutamate receptor that plays a role as a modulator of synaptic plasticity, ion channel activity, and excitotoxicity (Bach et al., Metabotropic Glutamate Receptor 5 Modulators and their Potential Therapeutic Applications, Department of Med. Chemistry, AstraZeneca R and D Moelndal, Moelndal, Sweden, Expert Opinion on Therapeutic Patents 2007, 17(4), 371-384 and references therein).

Recent evidence indicates that current mGluR5 negative allosteric modulators are not sufficiently selective, and cause off-target effects, such as inhibition of NMDA receptors. Thus, there exists an ongoing need for compounds that more selectively bind to mGluR5, and that are useful in repressing and/or treating disorders such as schizophrenia, paranoia, depression, manic-depressive illness and anxiety. This invention is directed to these, as well as other, important ends.

### SUMMARY OF THE INVENTION

In one aspect, the invention provides compounds of Formula I:

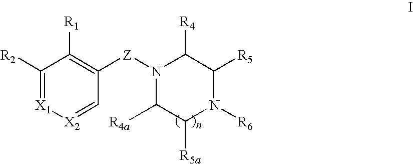

wherein the constituent variables are as defined herein.

In another aspect, the invention provides pharmaceutical compositions containing a compound of the invention, and a pharmaceutically acceptable carrier.

In a further aspect, the invention provides methods for the treatment of a patient suffering from a chronic condition such as, schizophrenia, paranoia, manic-depressive illness, depression, or anxiety (including panic disorders, social anxiety, obsessive compulsive disorders, generalized anxiety disorders, phobias), post-traumatic stress disorder, bipolar disorder, Asperger's syndrome, pervasive developmental disorders, gastrointestinal disorders such as gastroesophageal reflux disease, dyspepsia, irritable bowel syndrome, functional bloating, functional diarrhea, chronic constipation, functional disturbances of the biliary tract, migraine, chronic pain, fibromyalgia, neuropathic pain, post-herpatic neuropathic pain, addiction, Parkinson's disease, senile dementia, levadopa-induced dyskinesia, Alzheimer's disease, Huntington's chorea, amyotrophic lateral sclerosis, multiple sclerosis, Down Syndrome, fragile-X syndrome, autistic spectrum disorders, attention deficit hyperactivity disorder, stroke, ischemic injury, epilepsy, hypoglycemia and obesity.

In yet another aspect, the invention provides methods for producing compounds of Formula I.

Other aspects of the present teachings are described further in the following detailed description.

### DETAILED DESCRIPTION

In accordance with the invention, there are provided A compound of Formula I:

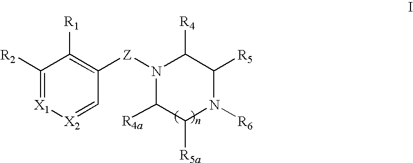

wherein:

R₁ is each independently selected from H, C₁₋₆ alkyl, halogen, OH, and OC₁₋₆ alkyl;

R₂ is selected from -(L₁)ₐ-(Y)_{c}-(L₂)_{b}-Q₃, -L₃-Q₄ and -L₄-Q₅;

L₃ is C₂₋₁₂ alkynyl optionally substituted with 1-3 substituents selected from OH and halogen;

L₁ and L₂ are each independently C₁₋₃ alkyl;

L₄ is C₂₋₁₂ alkenyl optionally substituted with 1-3 substituents selected from OH and halogen;

n is 1 or 2

R₄, R₄ₐ, R₅, and R₅ₐ are each independently selected from H, (═O) and C₁₋₆ alkyl; or R₄ and one of R₅ₐ together can form a bridging methylene; or R₅ can be together with the carbon to which it is attached —C(═O)

R₆ is selected from H, CH₃, -(L₅)-(3- to 14-membered heterocycle), -(L₅)-(5 to 14 membered heteroaromatic), (L₅)-(3- to 10-membered cycloalkyl), (L₅)-(C₆₋₁₄ aryl) and -(L₅)-C₁₋₆ alkyl each of which except H can be optionally substituted with 1 to 3 substituents independently selected from H, C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl, CN, (5- to 14-membered heteroaromatic), NR₁R₁, SO₂C₁₋₆ alkyl, SO₂, SO₂NR₁R₁, C₁₋₆alkylaryl, COC₁₋₆ alkyl, and (3- to 14-membered heterocycle) optionally substituted with NO₂.

L₅ is selected from a bond, C₁₋₃ alkyl, —C(═O)—, SO₂, (3- to 6-membered heterocycle) and (5- to 14-membered heteroaromatic).

X₁, X₂ are independently CR₃ or N;

each R₃ is independently H, C₁₋₆ alkyl, halogen, OH, OC₁₋₆ alkyl, SO₂, 3- to 14-membered heterocycle or 5- to 14-membered heteroaromatic, wherein each of C₁₋₁₆ alkyl or OC₁₋₆ alkyl can be optionally substituted with 1 to 3 substituents independently selected from halogen, OH, OC₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl), cycloalkyl, NR₁R₁, or CN;

Z is CO;

Y is CR₇R₈, NR₉, O, or S;

R₇, R₈, R₉ are independently H, C₁₋₆ alkyl, halogen, OH, or OC₁₋₆ alkyl

a, b, c are independently 0 or 1; and

Q₃ is C₆₋₁₄ aryl, 5 to 14 membered heterocyclic, 5 to 14 membered heteroaromatic, or 4 to 9 membered carbocyclic; each of which can be optionally substituted with 1 to 3 substituents independently selected from C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl), OC₁₋₆ haloalkyl, OC₁₋₆ alkylaryl and CN;

Q₄ is H, C₆₋₁₄ aryl, 5 to 14 membered heterocyclic, 5 to 14 membered heteroaromatic, or 4 to 9 membered carbocyclic; each of which except H can be optionally substituted with 1 to 3 substituents independently selected from C₁₋₆ alkyl, halogen, OH, OC₁₋₆ alkyl, —C(═O)O—(C₁₋₆ alkyl), —C(═O)C₁₋₁₆ alkyl, NO₂, C₁₋₃ haloalkyl, —S—C₁₋₆ alkyl —NH₂, —NH—(C₁₋₆ alkyl), —N(C₁₋₆ alkyl)(C₁₋₆ alkyl), CO₁₋₃haloalkyl, CO₁₋₆alkylaryl and CN;

Q₅ is C₆₋₁₄ aryl, 5 to 14 membered heterocyclic, 5 to 14 membered heteroaromatic, or 4 to 9 membered carbocyclic; each of which can be optionally substituted with 1 to 3 substituents independently selected from C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl), CO₁₋₃haloalkyl, CO₁₋₆alkylaryl and CN.

In some embodiments of formula I, n is 1.

In some embodiments, R₂ is -L₃-Q₄. In some embodiments, Z is CO. In some embodiments, R₁, R₄, R₄ₐ, R₅, R₅ₐ, and R₆ are each H. In some embodiments, R₃ is H, methyl, methoxy or halogen.

In some embodiments, R₂ is -L₃-Q₄, and Z is CO. In some such embodiments, R₁, R₄, R₄ₐ, R₅, and R₅ₐ, are each H. In some further such embodiments, R₁, R₄, R₄ₐ, R₅, and R₅ₐ, are each H; and R₃ is H, methyl, methoxy or halogen. In some further such embodiments, Q₄ is H. In some further such embodiments, Q₄ is phenyl optionally substituted with 1 to 3 substituents independently selected from H, C₁₋₆ alkyl, halogen, OH, and OC₁₋₆ alkyl. In some further such embodiments, Q₄ is 5 to 14 membered heterocyclic optionally substituted with 1 to 3 substituents independently selected from H, C₁₋₆ alkyl, halogen, OH, and OC₁₋₆ alkyl. In some further such embodiments, Q₄ is 5 to 14 membered heteroaromatic optionally substituted with 1 to 3 substituents independently selected from H, C₁₋₆ alkyl, halogen, OH, and OC₁₋₆ alkyl.

In some embodiments R₂ is -L₃-Q₄, Z is CO, and R₆ is -(L₅)-2-pyridyl, -(L₅)-4-pyridyl, -(L₅)-pyrazinyl, -(L₅)-phenyl, -(L₅)-(tetrazole-5-yl), pyrimidin-2-yl, -(4-phenyl)-pyrimidin-2-yl or -(L₅)-1,2,5-diathiazole-3-yl, each of which can be optionally substituted with 1 to 3 substituents independently selected from C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl and CN. In some such embodiments, L₅ is a bond.

In some embodiments of the compounds of Formula I, X₁ and X₂ are each independently CR₃ or N.

In some embodiments of the compounds of Formula I, one of X₁ and X₂ is CR₃, and the other of X₁ and X₂ is N. In some such embodiments, Z is CO. In some further such embodiments, Z is CO; R₂ is -L₃-Q₄, and L₃ is C₂ alkynyl. In some further such embodiments, Z is CO; R₂ is -L₃-Q₄, L₃ is C₂ alkynyl, and Q₄ is phenyl optionally substituted with 1 to 3 substituents independently selected from C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl) and CN. In some such embodiments, R₄, R₄ₐ, R₅, and R₅ₐ, are each H. In some such embodiments, R₆ is 5 to 14 membered heteroaromatic, each of which is optionally substituted with 1 to 3 substituents independently selected from C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl and CN.

In some embodiments of the compounds of Formula I, X₁ and X₂ are each independently CR₃. In some such embodiments, R₆ is H.

In some embodiments of the compounds of Formula I, X₁ is CR₃, X₂ is CH, and R₆ is H. In some such embodiments, Z is CO.

In some embodiments of the compounds of Formula I, X₁ is CR₃, X₂ is CH, R₆ is H, Z is CO and R₁, R₄, R₄ₐ, R₅, and R₅ₐ, are each H.

In some embodiments of the compounds of formula I, X₁ is CR₃, X₂ is CH, R₆ is -(L₅)-phenyl optionally substituted with halogen or C₁₋₆ alkyl, wherein L₅ is a bond, Z is CO and R₄ₐ and R₅ form a bridging methylene, R₂ is -L₃-Q₄, L₃ is C₂ alkynyl, and Q₄ is 2-pyridyl or phenyl optionally substituted with 1 to 3 substituents independently selected from C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl) and CN. In some such further embodiments R₃ is OC₁₋₆ alkyl.

In some other embodiments of the compounds of formula I, R₆ is H, CH₃, -(L₅)-2-pyridyl, -(L₅)-4-pyridyl, -(L₅)-pyrazinyl, -(L₅)-phenyl, -(L₅)-(3-14-membered heterocycle), -(L₅)-(5- to 14-membered heteroaromatic), (L₅)-cycloalkyl, (L₅)-(3- to 10-membered cycloalkyl), (L₅)-(C₆₋₁₄ aryl) or -(L₅)-C₁₋₆ alkyl each of which except H can be optionally substituted with 1 to 3 substituents independently selected from H, C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl, CN, a 3- to 14-membered heterocycle or 5- to 14-membered heteroaromatic, NR₁, SO₂, SO₂NR₁R₁ or C₁₋₆ alkylaryl.

In other embodiments of the compounds of formula I, R₆ is -(L₅)-(3- to 14-membered heterocycle), -(L₅)-(5 to 14 membered heteroaromatic) or (L₅)-(C₆₋₁₄ aryl), wherein L₅ can be a bond, SO₂

### or —C(═O)—.

In some embodiments of the compounds of Formula I, X₁ is CR₃, X₂ is CH, R₆ is H, Z is CO, R₁, R₄, R₄ₐ, R₅, and R₅ₐ, are each H, and R₂ is -(L₁)ₐ-(Y)_{c}-(L₂)_{b}-Q₃ or -L₄-Q₅. In some such embodiments, Y is O. In some further such embodiments, Y is O, and Q₃ and Q₅ are each phenyl optionally substituted with 1 to 3 substituents independently selected from C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl) and CN. In some further such embodiments, R₂ is —CH═CH—, —CH₂—O— or —O—CH₂—; Y is Q; and Q₃ and Q₅ are each phenyl optionally substituted with 1 to 3 substituents independently selected from C₁₋₆ alkyl, halogen, OH, OC₁₋₆ alkyl, —C(═O)O—(C₁₋₁₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl) and CN.

In some embodiments of the compounds of Formula I, Z is CH₂. In some such embodiments, X₁ and X₂ are each CH.

In some embodiments of the compounds of Formula I, Z is CH₂, X₁ and X₂ are each CH, and R₆ is -(L₅)-(5 to 14 membered heteroaromatic), optionally substituted with 1 to 3 substituents independently selected from H, C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl and CN.

In some embodiments of the compounds of Formula I, Z is CH₂, X₁ and X₂ are each CH, and R₂ is -L₃-Q₄; wherein Q₄ is phenyl or 4 to 9 membered carbocyclic, each of which is optionally substituted with 1 to 3 substituents independently selected from C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl) and CN.

In some embodiments of the compounds of Formula I, Z is CH₂, X₁ and X₂ are each CH, and R₂ is -L₃-Q₄; wherein Q₄ is phenyl or 4 to 9 membered carbocyclic, each of which is optionally substituted with 1 to 3 substituents independently selected from C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl) and CN; and R₆ is -(L₅)-(5 to 14 membered heteroaromatic), optionally substituted with 1 to 3 substituents independently selected from H, C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl and CN.

In some embodiments of the compounds of Formula I, Z is CH₂, X₁ and X₂ are each CH, and R₂ is -L₃-Q₄; wherein Q₄ is phenyl or 4 to 9 membered carbocyclic, each of which is optionally substituted with 1 to 3 substituents independently selected from C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl) and CN; and R₆ is (L₅)-(C₆₋₁₄ aryl), optionally substituted with 1 to 3 substituents independently selected from H, C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl and CN.

In some embodiments of the compounds of Formula I, Z is CH₂, X₁ and X₂ are each CH, and R₂ is -L₃-Q₄; wherein Q₄ is phenyl, cyclopentyl, cyclohexyl, cyclopentenyl or cyclohexenyl, each of which is optionally substituted with 1 or 2 substituents independently selected from C₁₋₆ alkyl, halogen, OH, OC₁₋₆ alkyl and —NH₂; and R₆ is pyrid-2-yl. In some such embodiments, R₁, R₄, R₄ₐ, R₅, and R₅ₐ, are each H, and L₃ is C₂₋₃ alkynyl.

In some embodiments of the compounds of Formula I, Z is SO₂. In some such embodiments, X₁ and X₂ are each CH.

In some embodiments of the compounds of Formula I, Z is SO₂, X₁ and X₂ are each CH, and R₆ is -(L₅)-(5 to 14 membered heteroaromatic), optionally substituted with 1 to 3 substituents independently selected from H, C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl and CN.

In some embodiments of the compounds of Formula I, Z is SO₂, X₁ and X₂ are each CH, and R₂ is -L₃-Q₄; wherein Q₄ is phenyl or 4 to 9 membered carbocyclic, each of which is optionally substituted with 1 to 3 substituents independently selected from C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl and CN.

In some embodiments of the compounds of Formula I, Z is SO₂, X₁ and X₂ are each CH, and R₂ is -L₃-Q₄; wherein Q₄ is phenyl or 4 to 9 membered carbocyclic, each of which is optionally substituted with 1 to 3 substituents independently selected from C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl and CN; and R₆ is -(L₅)-(5 to 14 membered heteroaromatic), optionally substituted with 1 to 3 substituents independently selected from H, C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl and CN.

In some embodiments of the compounds of Formula I, Z is SO₂, X₁ and X₂ are each CH, and R₂ is -L₃-Q₄; wherein Q₄ is phenyl, cyclopentyl, cyclohexyl, cyclopentenyl or cyclohexenyl, each of which is optionally substituted with 1 or 2 substituents independently selected from C₁₋₆ alkyl, halogen, OH, and OC₁₋₆ alkyl; and R₆ is pyrid-2-yl. In some such embodiments, R₁, R₄, R₄ₐ, R₅, and R₅ₐ, are each H, and L₃ is C₂₋₃ alkynyl.

In some embodiments of the compounds of Formula I, R₂ is -L₃-Q₄; Q₄ is 5 to 14 membered heteroaromatic optionally substituted with 1 to 3 substituents independently selected from C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl) and CN; and R₆ is -(L₅)-(5 to 14 membered heteroaromatic) optionally substituted with 1 to 3 substituents independently selected from H, C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl and CN.

In some embodiments of the compounds of Formula I, R₂ is -L₃-Q₄; Q₄ is 5 to 14 membered heteroaromatic optionally substituted with 1 to 3 substituents independently selected from C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl) and CN; and R₆ is -(L₅)-(5 to 14 membered heteroaromatic) optionally substituted with 1 to 3 substituents independently selected from H, C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl and CN.

In some such embodiments, Q₄ is pyridyl, preferably pyrid-2-yl, optionally substituted with 1 to 3 substituents independently selected from C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl) and CN.

In some further such embodiments, R₆ is -(L₅)-(pyridyl), preferably -(L₅)-(pyrid-2-yl), optionally substituted with 1 to 3 substituents independently selected from H, C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl and CN.

In some further such embodiments, Z is CO. In some further such embodiments, X₁ is CR₃ and X₂ is CH. In some further such embodiments, R₁ is H. In some further such embodiments, R₄, R₄ₐ, R₅, and R₅ₐ are each H, and in some further such embodiments, R₁ is H.

In some embodiments of the compounds of Formula I, one or more of the following conditions a-g exist:

(a) if R₂ is -L₃-Q₄, L₃ is C₂ alkynyl, Q₄ is cyclohexanol-1-yl, Z is CO, R₁, R₄, R₄ₐ, R₅, and R₅ₐ, are each H, and X₁ and X₂ are each CH, then R₆ is not 2-methoxyphenyl;

(b) if R₂ is -L₃-Q₄, L₃ is C₂ alkynyl, Q₄ is phenyl, Z is CO, R₁, R₄, R₄ₐ, R₅, and R₅ₐ, are each H, and X₁ and X₂ are each CH, then R₆ is not pyrimidin-2-yl;

(c) if R₂ is -L₃-Q₄, L₃ is C₂ alkynyl, Q₄ is phenyl, Z is CO, R₁, R₄, R₄ₐ, R₅, and R₅ₐ, are each H, and X₁ and X₂ are each CH, then R₆ is not 3-trifluoromethylphenyl;

(d) if R₂ is -L₃-Q₄, L₃ is C₂ alkynyl, Q₄ is phenyl, Z is CO, R₁, R₄, R₄ₐ, R₅, and R₅ₐ, are each H, and X₁ and X₂ are each CH, then R₆ is not 2-methoxyphenyl;

(e) if R₂ is -L₃-Q₄, L₃ is C₂ alkynyl, Q₄ is phenyl, Z is CO, R₁, R₄, R₄ₐ, R₅, and R₅ₐ, are each H, and X₁ and X₂ are each CH, then R₆ is not pyrid-2-yl;

(f) if R₂ is -L₃-Q₄, L₃ is C₂ alkynyl, Q₄ is phenyl, Z is CO, R₁, R₄, R₄ₐ, R₅, and R₅ₐ, are each H, and X₁ and X₂ are each CH, then R₆ is not 2-fluorophenyl;

(g) if R₂ is -L₃-Q₄, L₃ is C₂ alkynyl, Q₄ is cyclohexanol-1-yl, Z is CO, R₁, R₄, R₄ₐ, R₅, and R₅ₐ, are each H, and X₁ and X₂ are each CH, then R₆ is not 4-nitrophenyl.

In some embodiments of the compounds of Formula I, all of the foregoing conditions a-g exist. In some embodiments of the compounds of Formula I, none of the foregoing conditions a-g exist. In some embodiments of the compounds of Formula I, one or more, but less than all of the foregoing conditions a-g exist.

Prodrugs of the compounds of Formula I are also embraced by the present invention. The term “prodrug”, as used herein, means a compound which is convertible in vivo by metabolic means (e.g. by hydrolysis) to a compound of formula I. Various forms of prodrugs are known in the art, for example, as discussed in, for example, Bundgaard, (ed.), Design of Prodrugs, Elsevier (1985); Widder, et al. (ed.), Methods in Enzymology, vol. 4, Academic Press (1985); Krogsgaard-Larsen, et al. (ed.), “Design and Application of Prodrugs”, Textbook of Drug Design and Development, Chapter 5, 113-191 (1991), Bundgaard, et al., Journal of Drug Deliver reviews, 8:1-38 (1992), Bundgaard, J. of Pharmaceutical Sciences, 77:285 et seq. (1988); and Higuchi and Stella (eds.) Prodrugs as Novel Drug Delivery Systems, American Chemical Society (1975), each of which is incorporated by reference in its entirety.

The mGluR5 negative allosteric modulators disclosed herein are useful for treating diseases and disorders including schizophrenia, paranoia, depression, including manic-depressive illness, anxiety (including panic disorders, social anxiety, obsessive compulsive disorders, generalized anxiety disorders, phobias), post-traumatic stress disorder, bipolar disorder, Asperger's syndrome, pervasive developmental disorders, gastrointestinal disorders such as gastroesophageal reflux disease, dyspepsia, irritable bowel syndrome, functional bloating, functional diarrhea, chronic constipation, functional disturbances of the biliary tract, migraine, chronic pain, fibromyalgia, neuropathic pain, post-herpatic neuropathic pain, addiction, Parkinson's disease, senile dementia, levadopa-induced dyskinesia, Alzheimer's disease, Huntington's chorea, amyotrophic lateral sclerosis, multiple sclerosis, Down Syndrome, fragile-X syndrome, autistic spectrum disorders, attention deficit hyperactivity disorder, stroke, ischemic injury, epilepsy, hypoglycemia and obesity. Accordingly, in some embodiments, the invention provides pharmaceutical compositions comprising a pharmaceutically acceptable carrier and a compound of Formula I, or a pharmaceutically acceptable salt, hydrate or prodrug thereof. In further embodiments, the invention provides methods of treating a patient suffering from a chronic condition such as schizophrenia, paranoia, manic-depressive illness or anxiety, comprising providing a therapeutically effective amount of compound of Formula I, or a pharmaceutically acceptable salt, hydrate, solvate or prodrug thereof.

Some compounds of the present invention can contain an asymmetric atom (also referred as a chiral center), and some of the compounds can contain one or more asymmetric atoms or centers, which can thus give rise to optical isomers (enantiomers) and diastereomers (geometric isomers). The present invention includes such optical isomers and diastereomers, as well as, the racemic and resolved, enantiomerically pure R and S stereoisomers, as well as, other mixtures of the R and S stereoisomers and pharmaceutically acceptable salts, hydrates, solvates, metabolites and prodrugs thereof. Optical isomers can be obtained in pure form by standard procedures known to those skilled in the art, and include, but are not limited to, chiral chromatography, diastereomeric salt formation, kinetic resolution, and asymmetric synthesis. The present teachings also encompass cis and trans or E/Z isomers of compounds containing alkenyl moieties (e.g., alkenes and imines). It is also understood that this invention encompasses all possible regioisomers, and mixtures thereof, which can be obtained in pure form by standard separation procedures known to those skilled in the art, and include, but are not limited to, column chromatography, thin-layer chromatography, and high-performance liquid chromatography.

Compounds of the invention can also include all isotopes of atoms occurring in the intermediates or final compounds. Isotopes include those atoms having the same atomic number but different mass numbers. For example, isotopes of hydrogen include tritium and deuterium.

Compounds of the invention can also include tautomeric forms, such as keto-enol tautomers. Tautomeric forms can be in equilibrium or sterically locked into one form by appropriate substitution.

As used herein, the term “alkyl” as a group or part of a group is intended to denote hydrocarbon groups including straight chain, branched and cyclic saturated hydrocarbons. Alkyl groups can contain 1-20, or 1-12, or 1-6 carbon atoms. The term “lower alkyl” is intended to mean an alkyl group having up to 6 carbon atoms. Nonlimiting examples of straight chain and branched alkyl groups include methyl (Me), ethyl (Et), propyl (e.g., n-propyl and isopropyl), butyl (e.g., n-butyl, isobutyl, s-butyl, and t-butyl), pentyl groups (e.g., n-pentyl, isopentyl, and neopentyl), hexyl groups, and the like.

The term “cycloalkyl” is intended to mean a monocyclic or bicyclic saturated hydrocarbon group having the indicated number of carbon atoms. For example, a C₃-C₈ cycloalkyl group would include cyclopropyl, cyclobutyl, cyclopentyl, cyclohexyl, cycloheptyl and cyclooctyl groups, as well as polycyclic systems (e.g., containing fused, bridged, and/or spiro ring systems). Any suitable ring position of a cyclic alkyl group can be covalently linked to the defined chemical structure. Unless otherwise indicated, alkyl groups are unsubstituted. However, where indicated, alkyl groups may be substituted with one or more independently selected substituents as described herein.

As used herein, the term “alkenyl” as a group or part of a group is intended to denote an alkyl group that contains at least one carbon-carbon double bond. Alkenyl groups can contain 2-20, or 2-12, or 2-6 carbon atoms. The term “lower alkenyl” is intended to mean an alkenyl group having up to 6 carbon atoms. Nonlimiting examples of straight chain and branched alkenyl groups include ethenyl, propenyl, butenyl, pentenyl, hexenyl, butadienyl, pentadienyl, hexadienyl, vinyl, allyl, 2-methyl-allyl, 4-but-3-enyl, 4-hex-5-enyl, 3-methyl-but-2-enyl, cyclohex-2-enyl, and the like. The one or more carbon-carbon double bonds can be internal (such as in 2-butene) or terminal (such as in 1-butene). Additionally, hydrocarbon alkenyl moieties may be mono or polyunsaturated, and may exist in the E or Z configurations. The compounds of this invention are meant to include all possible E and Z configurations. Alkenyl groups may be substituted with one or more independently selected substituents as described herein.

The term “cycloalkenyl” is intended to mean a cycloalkyl group that contains at least one carbon-carbon double bond. Examples of cycloalkenyl groups include, but are not limited to, cyclopentenyl, cyclohexenyl, cyclohexadienyl, cycloheptatrienyl, and the like. Alkenyl groups may be substituted with one or more independently selected substituents as described herein. Any suitable ring position of a cycloalkenyl group can be covalently linked to the defined chemical structure. Unless otherwise indicated, alkenyl groups are unsubstituted. However, where indicted, alkenyl groups may be substituted with one or more independently selected substituents as described herein.

As used herein, the term “alkynyl” is intended to denote an alkyl group that contains at least one carbon-carbon triple bond. Alkynyl groups can contain 2-20, or 2-12, or 2-6, or 2-3 carbon atoms. Examples of alkynyl groups include, but are not limited to, ethynyl, propynyl, butynyl, pentynyl, pent-2-yne, ethynyl-cyclohexyl, and the like. The one or more carbon-carbon triple bonds can be internal (such as in 2-butyne) or terminal (such as in 1-butyne). Alkynyl groups may be substituted with one or more independently selected substituents as described herein.

As used herein, the term “aryl” as a group or part of a group refers to an aromatic monocyclic hydrocarbon ring system or a polycyclic ring system (e.g., bicyclic or tricyclic), e.g., of 6-14 carbon atoms where at least one of the rings present in the ring system is an aromatic hydrocarbon ring and any other aromatic rings present in the ring system include only hydrocarbons. Any suitable ring position of the aryl group can be covalently linked to the defined chemical structure. In some embodiments, an aryl group can have only aromatic carbocyclic rings e.g., phenyl, 1-naphthyl, 2-naphthyl, anthracenyl, phenanthrenyl groups, and the like. In other embodiments, an aryl group can be a polycyclic ring system in which at least one aromatic carbocyclic ring is fused (i.e., having a bond in common with) to one or more cyclic alkyl or heterocyclic alkyl rings, provided that the group is attached to the remainder of the molecule through the aromatic portion thereof. Examples of such aryl groups include, among others, benzo derivatives of cyclopentane (i.e., an indanyl group, which is a 5,6-bicyclic cyclic alkyl/aromatic ring system), cyclohexane (i.e., a tetrahydronaphthyl group, which is a 6,6-bicyclic cyclic alkyl/aromatic ring system), imidazoline (i.e., a benzimidazolinyl group, which is a 5,6-bicyclic heterocyclic alkyl/aromatic ring system), and pyran (i.e., a chromenyl group, which is a 6,6-bicyclic heterocyclic alkyl/aromatic ring system). Other examples of aryl groups include, but are not limited to, benzodioxanyl, benzodioxolyl, chromanyl, indolinyl groups, and the like.

In some embodiments, an aryl group can be substituted with one or more (e.g., up to 4) independently selected substituents as described herein.

As used herein, the terms, “carbocyclyl”, “carbocycle” or “carbocyclic” refer to (1) a non-aromatic cyclic hydrocarbon group having from 3 to 10 ring carbon atoms. In some embodiments (“C₃₋₈ carbocyclyl”), a carbocyclyl group can have from 3 to 8 ring carbon atoms. In some embodiments (“C₃₋₆ carbocyclyl”), a carbocyclyl group can have from 3 to 6 ring carbon atoms. Examples of such C₃₋₆ carbocyclyl groups include cyclopropyl, cyclobutyl, cyclopentyl, cyclopentenyl, cyclohexyl, cyclohexenyl, cyclohexadienyl and the like. Examples of such C₃₋₈ carbocyclyl groups include the aforementioned C₃₋₆ carbocyclyl groups as well as cycloheptyl, cycloheptadienyl, cycloheptatrienyl, cyclooctyl, bicyclo[2.2.1]heptanyl, bicyclo[2.2.2]octanyl and the like. Examples of such C₃₋₁₀ carbocyclyl groups include the aforementioned C₃₋₈ carbocyclyl groups as well as octahydro-1H-indenyl, decahydronaphthalenyl, spiro[4.5]decanyl and the like. As the foregoing examples illustrate, in some embodiments a carbocyclyl group can be monocyclic (“monocyclic carbocyclyl”) or bicyclic (e.g., containing a fused, bridged or spiro ring system), and can be saturated or can contain one or more carbon-carbon double or triple bonds. “Carbocyclyl” also refers to (2) a phenyl group; (3) an aryl group (as defined herein); and (4) a 5- or 6-membered heteroaryl group (as defined herein) fused to a monocyclic carbocyclyl group, where the point of attachment is on the carbocyclyl portion of the group. Examples of such carbocyclyl groups include 1,2,3,4-tetrahydronaphthalen-1-yl, 1,2,3,4-tetrahydronaphthalen-2-yl, 2,3-dihydro-1H-inden-1-yl, 2,3-dihydro-1H-inden-2-yl, 1H-inden-1-yl, 5,6,7,8-tetrahydroquinolin-5-yl, 5,6,7,8-tetrahydroquinolin-7-yl, 4,5,6,7-tetrahydro-1H-indol-4-yl, 4,5,6,7-tetrahydro-1H-indol-6-yl, 4,5,6,7-tetrahydrobenzofuran-7-yl and the like.

The term “heterocyclic” or “heterocyclic group” or “heterocycle” is used herein to describe a 3-14 membered monocyclic or polycyclic, ring system having at least 1, and up to 4, ring heteroatoms independently selected from N, O and S. Heterocyclic groups can be saturated, partially unsaturated, or wholly unsaturated, but cannot be aromatic. When the heterocyclic ring contains nitrogen or sulfur atoms in the backbone of the ring, the nitrogen or sulfur atoms can be oxidized, for example, N-oxides, SO or SO₂. Heterocyclic groups include, without limitation, oxygen-containing rings, nitrogen-containing rings, sulfur-containing rings, and mixed heteroatom-containing rings. Nonlimiting examples of heterocyclic groups include aziridinyl, azetidinyl, 1,4-dioxanyl, hexahydroazepinyl, piperazinyl, piperidinyl, piperazinyl, pyrrolidinyl, morpholinyl, thiomorpholinyl, dihydrobenzimidazolyl, dihydrobenzofuranyl, dihydrobenzothienyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, dihydro-1,4-dioxanyl, tetrahydrofuranyl, tetrahydrothienyl, tetrahydroquinolinyl, and tetrahydroisoquinolinyl.

The term “heteroaromatic” as used herein is intended to denote 3-14 membered monocyclic or polycyclic ring systems having at least one aromatic ring that contains at least 1, and up to 4, ring heteroatoms independently selected from N, O and S. Heteroaromatic groups can contain one or more non-aromatic rings fused to (i.e., sharing a bound in common with) the monocyclic or polycyclic heteroatom-containing ring described above, provided that the group is attached to the remainder of the molecule through the aromatic portion thereof. Thus, the term “heteroaromatic” includes groups such as 5,6,7,8-tetrahydroquinolin-2-yl groups. Further examples of heteroaromatic groups include furyl, thienyl, pyridyl, pyrrolyl, oxazolyl, thiazolyl, isothiazolyl, imidazolyl, pyrazolyl, isoxazolyl, triazolyl, oxadiazolyl, pyrimidinyl, pyrazinyl, indolyl, benzimidazolyl, benzothiophenyl, quinolinyl, isoquinolinyl, quinoxalinyl, and benzothiazolyl.

The term “optionally substituted” is used herein to refer to the optional substitution of one or more protons with a named substituent or substituents.

The term “alkoxy” as used herein refers to a group of formula —O-alkyl. Examples of alkoxy groups include but are not limited to methoxy, ethoxy, propoxy, isopropoxy, butoxy, isobutoxy, tertiary butoxy, pentoxy, isopentoxy, neopentoxy, tertiary pentoxy, hexoxy, isohexoxy, heptoxy, octoxy, prop-2-oxy, but-2-oxy and methylprop-2-oxy.

The term “halogen” refers to Cl, Br, F, and I.

The term “haloalkyl” is intended to include both branched and straight-chain saturated aliphatic hydrocarbon groups having the specified number of carbon atoms, substituted with 1 or more halogen atom. Haloalkyl groups include perhaloalkyl groups, wherein all hydrogens of an alkyl group have been replaced with halogens (e.g., —CF₃, —CF₂CF₃). The halogens can be the same (e.g., CHF₂, —CF₃) or different (e.g., CF₂Cl). Haloalkyl groups can optionally be substituted with one or more substituents in addition to halogen. Examples of haloalkyl groups include, but are not limited to, fluoromethyl, dichloroethyl, trifluoromethyl, trichloromethyl, pentafluoroethyl, and pentachloroethyl groups.

Methods of treating the diseases and syndromes listed herein are understood to involve administering to an individual in need of such treatment a therapeutically effective amount of a compound of the invention, or a salt, hydrate or solvate thereof, or a composition comprising one or more of the same. Accordingly, methods are provided in accordance with the invention for treating disorders involving the mGluR5 receptor, such as anxiety and depression diseases and/or disorders, including those specifically listed above, comprising the administration to a patient in need thereof a compound of the invention, or a pharmaceutically acceptable salt, hydrate or solvate thereof. Such methods comprise administering to the patient in need of such treatment a pharmaceutically or therapeutically effective amount of a compound of this invention. In the instances of combination therapies described herein, it will be understood the administration further includes a pharmaceutically or therapeutically effective amount of the second pharmaceutical agent in question. The second or additional pharmacological agents described herein may be administered in the doses and regimens known in the art.

As used herein, the phrase “therapeutically effective amount” refers to the amount of active compound or pharmaceutical agent that is effective to treat the condition of interest—i.e., the amount of active compound or pharmaceutical agent that is effective to elicit a biological or medicinal response in a tissue, system, animal, individual or human that is being sought by a researcher, veterinarian, medical doctor or other clinician, which includes one or more of the following:

(1) preventing the disease; for example, preventing a disease, condition or disorder in an individual that may be predisposed to the disease, condition or disorder but does not yet experience or display the pathology or symptomotology of the disease;

(2) inhibiting the disease; for example, inhibiting a disease, condition or disorder in an individual that is experiencing or displaying the pathology or symptomotology of the disease, condition or disorder (i.e., arresting or slowing further development of the pathology and/or symptomotology); and

(3) ameliorating the disease; for example, ameliorating a disease, condition or disorder in an individual that is experiencing or displaying the pathology or symptomotology of the disease, condition or disorder (i.e., reversing the pathology and/or symptomotology).

When administered for the treatment or inhibition of a particular disease state or disorder, it is understood that the effective dosage may vary depending upon the particular compound utilized, the mode of administration, the condition, and severity thereof, of the condition being treated, as well as the various physical factors related to the individual being treated. Effective administration of the compounds (including the salts) and the compositions of the present invention may be given at an oral dose of from about 0.1 mg/day to about 1,000 mg/day. Preferably, administration will be from about 10 mg/day to about 600 mg/day, more preferably from about 50 mg/day to about 600 mg/day. The dosing regimen can be adjusted to provide the optimal therapeutic response, and the projected daily dosages are expected to vary with route of administration. Several divided doses can be delivered daily or a single daily dosage can be delivered. The dose can be proportionally reduced or increased as indicated by the exigencies of the therapeutic situation.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

Therapeutic doses of compounds or compositions of the invention can be administered in any manner useful in directing the active compounds herein to the recipient's bloodstream. For example, compounds and compositions of the invention can be delivered by a route such as oral, via implants, dermal, transdermal, intrabronchial, intranasal, parental (including intravenous, intraperitoneal, intraarticularly and subcutaneous injections), intraperitoneal, sublingual, intracranial, epidural, intratracheal, vaginal, rectal, topical, ocular (via eye drops) or by sustained release. Optionally, one or more of the compounds of Formula I can be mixed with other active agents.

When the compound is delivered orally, it can be sub-divided in a dose containing appropriate quantities of the active ingredient. The unit dosage forms can be packaged compositions, for example, packeted powders, vials, ampoules, prefilled syringes or sachets containing liquids. The unit dosage form can be, for example, a capsule or tablet itself, or it can be the appropriate number of any such compositions in package form. The powders and tablets can contain up to 99% of the active ingredient.

The compounds of Formula I can be combined with one or more pharmaceutically acceptable carriers or excipients including, without limitation, solid and liquid carriers, which are compatible with the compounds of Formula I. Oral formulations containing the active compounds (including the salts, hydrates and solvates thereof) and the compositions of the present invention may comprise any conventionally used oral forms, including tablets, capsules, buccal forms, troches, lozenges and oral liquids, suspensions or solutions. Such carriers can include adjuvants, syrups, elixirs, diluents, binders, lubricants, surfactants, granulating agents, disintegrating agents, emollients, solubilizers, suspending agents, fillers, glidants, compression aids, encapsulating materials, emulsifiers, buffers, preservatives, thickening agents, colors, viscosity regulators, stabilizers, osmoregulators, and combinations thereof. Optionally, one or more of the compounds of Formula I can be mixed with other active agents.

Adjuvants can include, without limitation, flavoring agents, sweeteners, coloring agents, preservatives, and supplemental antioxidants, which can include vitamin E, ascorbic acid, butylated hydroxytoluene (BHT) and butylated hydroxyanisole (NHA).

Elixirs and syrups can be prepared from acceptable sweeteners such as sugar, saccharine or a biological sweetener, a flavoring agent, and/or solvent.

Capsules and tablets may contain mixtures of the active compound(s) with inert fillers, diluents, binders, lubricants, granulating agents, disintegrating agents, emollients, surface modifying agents (including surfactants), suspending or stabilizing agents, and the like. Nonlimiting examples of diluents and fillers include materials in which the compound can be dispersed, dissolved, or incorporated, such as water, lower monovalent alcohols, polyhydric alcohols, and low molecular weight glycols and polyols, including, for example, propylene glycol, glycerol, butylenes glycol, 1,2,4-butanetriol, sorbitol esters, 1,2,6-hexanetriol, ethanol, isopropanol, butanediol, ethyl oleate, isopropyl myristate, ether propanol, ethoxylated ethers, propoxylated ethers, oils such as corn, peanut, fractionated coconut, arachis, sesame oils, dimethylsulfoxide (DMSO), dimethylformamide (DMF), waxes, dextrin, and combinations thereof. Examples of binders include, without limitation, cellulose, methylcellulose, hydroxymethylcellulose, polypropylpyrrolidone, polyvinylpyrrolidone, polyvinylpyrrolidine, gelatin, gum Arabic, polyethylene glycol, starch, sugars such as, for example, sucrose kaolin, cellulose kaolin, and lactose. Nonlimiting examples of surface modifying agents include, but are not limited to, poloxamer 188, benzalkonium chloride, calcium stearate, cetostearl alcohol, sorbitan esters, colloidal, silicon dioxide, phosphates, sodium dodecylsulfate, magnesium aluminum silicate, lauryl sulfates, and triethanolamine. Examples of lubricants include, without limitation, magnesium stearate, light anhydrous silicic acid, talc and sodium lauryl sulfate. Examples of granulating agents include, without limitation, silicon dioxide, microcrystalline cellulose, starch, calcium carbonate, pectin, crospovidone, and polyplasdone. Examples of disintegrating agents include, without limitation, pharmaceutically acceptable starches (e.g. corn, potato or tapioca starch), carboxymethylcellulose, hydroxypropylstarch, substituted hydroxypropylcellulose, sodium bicarbonate, calcium phosphate, and calcium citrate. Examples of emollients include, without limitation, stearyl alcohol, mink oil, cetyl alcohol, oleyl alcohol, isopropyl laurate, polyethylene glycol, olive oil, petroleum jelly, palmitic acid, oleic acid, and myristyl myristate.

Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents as described above.

Oral formulations herein may utilize standard delay or time-release formulations to alter the absorption of the active compound(s). The oral formulation may also consist of administering the active ingredient in water or a fruit juice, containing appropriate solubilizers or emulsifiers as needed.

In some cases it may be desirable to administer the compounds (including the salts) and the compositions of the present invention directly to the airways in the form of an aerosol.

The compounds (including salts, hydrates and solvates) and the compositions of the present invention may also be administered parenterally or intraperitoneally. Solutions or suspensions of these active compounds (including the salts) and the compositions of the present invention can be prepared in water optionally mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to inhibit the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

For the purposes of this disclosure, transdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Transdermal administration may be accomplished through the use of a transdermal patch containing the active compound and a carrier that is inert to the active compound, is non-toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream such as a semi-permeable membrane covering a reservoir containing the active ingredient with or without a carrier, or a matrix containing the active ingredient. Other occlusive devices are known in the literature.

In some embodiments, sustained delivery devices can be used, in order to avoid the necessity to take medications on a daily basis. The term “sustained delivery” is used herein to refer to delaying the release of an active agent, i.e., a compound of Formula I, until after placement in a delivery environment, followed by a sustained release of the agent at a later time. A number of sustained delivery devices are known in the art and include, for example, hydrogels (U.S. Pat. Nos. 5,266,325; 4,959,217; 5,292,515), osmotic pumps (U.S. Pat. Nos. 4,295,987 and 5,273,752 and European Pat. No. 314,206, among others; hydrophobic membrane materials, such as ethylenemethacrylate (EMA) and ethylenevinylacetate (EVA); bioresorbable polymer systems (International Patent Publication No. WO 98/44964 and U.S. Pat. Nos. 5,756,127 and 5,854,388); and other bioresorbable implant devises composed of, for example, polyesters, polyanhydrides, or lactic acid/glycolic acid copolymers (U.S. Pat. No. 5,817,343). For use in such sustained delivery devices, the compounds of the invention can be formulated as described herein.

Suppository formulations may be made from traditional materials, including cocoa butter, with or without the addition of waxes to alter the suppository's melting point, and glycerin. Water-soluble suppository bases, such as polyethylene glycols of various molecular weights, may also be used.

Additional numerous various excipients, dosage forms, dispersing agents and the like that are suitable for use in connection with the salt forms of the invention are known in the art and described in, for example, Remington's Pharmaceutical Sciences, 17th ed., Mack Publishing Company, Easton, Pa., 1985, which is incorporated herein by reference in its entirety.

The compounds of Formula I have utility for the repression and/or treatment of disorders involving the mGluR5 receptor, such as anxiety and depression disorders. Examples of disorders or conditions which can be treated by the compounds, compositions and methods of this invention include anxiety and depression disorders. Anxiety disorders can include, for example, generalized anxiety disorder, panic disorder, PTSD, and social anxiety disorder. Depression disorders can include, for example, depression in cancer patients, depression in Parkinson's patients, post-myocardial infarction depression, depression in patients with human immunodeficiency virus (HIV), Subsyndromal Symptomatic depression, depression in infertile women, pediatric depression, major depression, single episode depression, recurrent depression, child abuse induced depression, post partum depression, DSM-IV major depression, treatment-refractory major depression, severe depression, psychotic depression, post-stroke depression, neuropathic pain, manic depressive illness, including manic depressive illness with mixed episodes and manic depressive illness with depressive episodes, seasonal affective disorder, bipolar depression BP 1, bipolar depression BP II, or major depression with dysthymia.

### Preparation of Compounds of the Invention

### General Preparative Schemes

Compounds of the invention can be prepared using the six general schemes outlined below, together with synthetic methods known in the synthetic organic arts or variations of these methods by one skilled in the art. See, Comprehensive Organic Synthesis, “Selectivity, Strategy & Efficiency in Modern Organic Chemistry”, ed., I. Fleming, Pergamon Press, New York (1991); Comprehensive Organic Chemistry, “The Synthesis and Reactions of Organic Compounds”, ed. J.F. Stoddard, Pergamon Press, New York (1979).

In some embodiments, compounds of the invention are produced in accordance with Scheme 1 below. Unless otherwise indicated, the constituent variables of the following Schemes are as defined above.

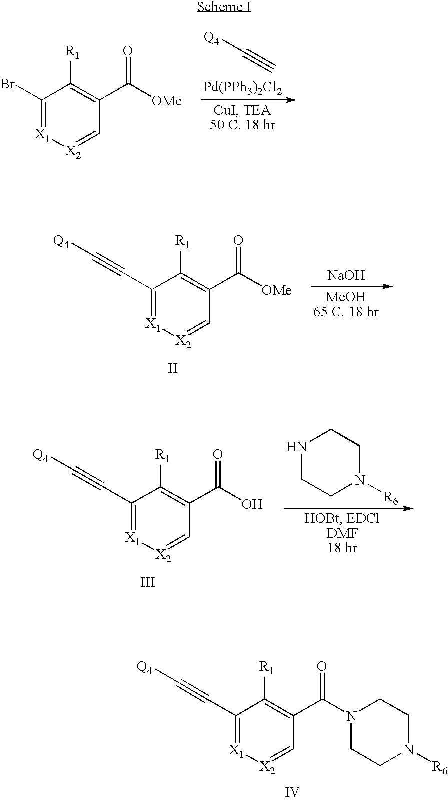

In accordance with Scheme 1, Sonagashira coupling of bromoaromatics with alkenes using Pd and catalytic CuI in TEA is used to produce the desired acetylenes (II) (Matsunaga, N. et al. Bioorg. Med. Chem. 2004, 12, 2251). Basic hydrolysis using NaOH in aqueous methanol produces acid (III). Reaction of the acid (III) with N-substituted piperazines using EDCl peptide coupling conditions (Rich, D. H. et al., Peptides (New York, 1979-1987) 1979, 1, 241-261) produced the target compounds (IV).

Accordingly, in some embodiments, the invention provides a method for preparing compound a compound of Formula IV:

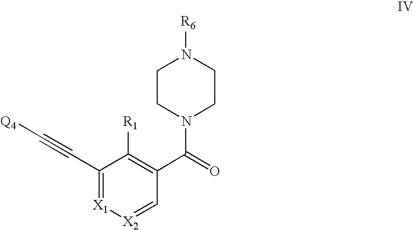

comprising reacting a compound of Formula III:

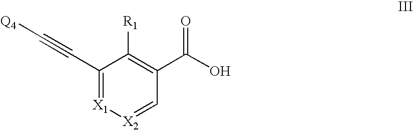

with an N-substituted piperazine of Formula IIIa:

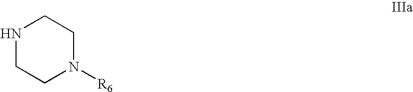

for a time and under conditions effective to form the compound of Formula IV;
wherein X₁, X₂, R₆, R₁ and Q₄ are as defined above.

In some embodiments, compounds of the invention are produced in accordance with Scheme 2 below.

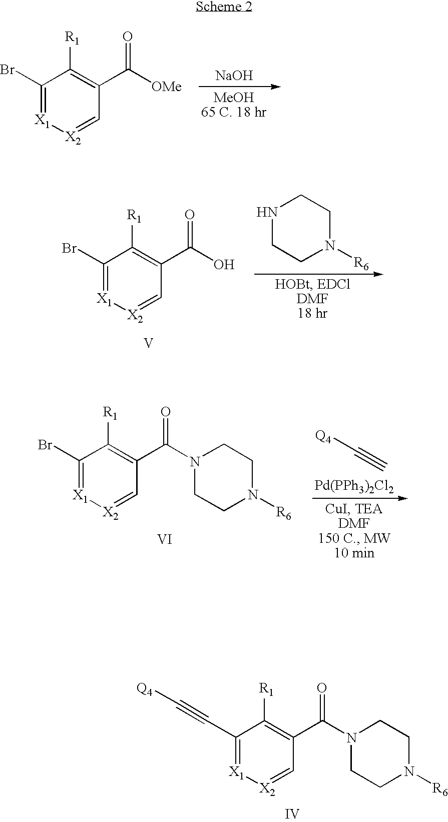

In this procedure, basic hydrolysis using NaOH in aqueous methanol produces an acid (V). The acid (V) is reacted with N-substituted piperazines using EDCl peptide coupling conditions (Rich, D. H. et al., Peptides (New York, 1979-1987) 1979, 1, 241-261) producing amides (VI). Sonagashira coupling of Bromoaromatics (VI) with Acetylenes using Pd(PPh₃)₂Cl₂ in the presence of CuI and TEA under microwave conditions produced the desired target compounds (IV) (see WO 2005/123713). Accordingly, in some embodiments, processes are provided for preparing a compound of Formula IV comprising reacting a compound of Formula VI:

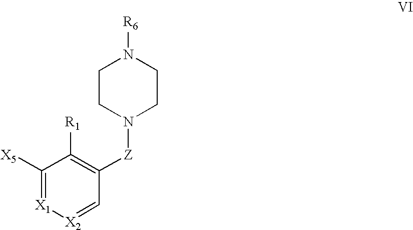

where the Rᵣ, R₆, X₁, X₂ and Z variables are as described above and X₅ is halogen or bromine, with an acetylene of Formula Q₄-CCH, in the presence of a palladium triphenylphosphine-containing catalyst for a time and under conditions effective to form a compound of Formula IV. In some embodiments, the palladium triphenylphosphine-containing catalyst is Pd(PPh₃)₂Cl₂.

In further embodiments, compounds of the invention having the general Formula IX are produced in accordance with Scheme 3 below.

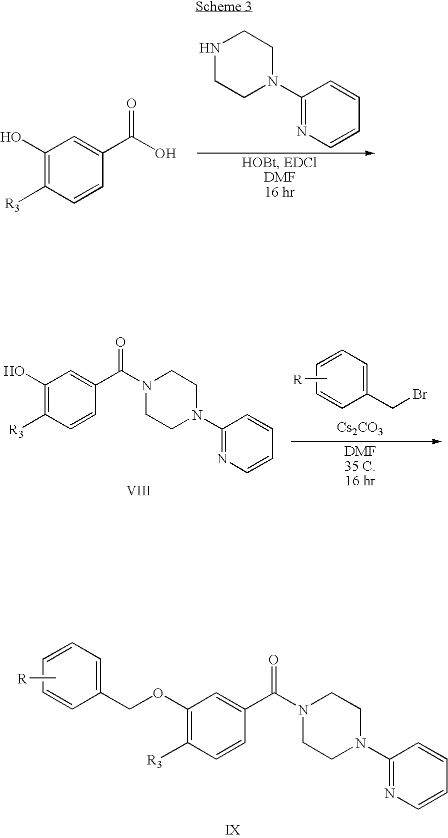

In accordance with Scheme 3, reaction of benzoic acids with N-substituted piperazines using EDCl peptide coupling conditions (Rich, D. H. et al., Peptides (New York, 1979-1987) 1979, 1, 241-261) produced amides (VIII). Subsequent alkylation of the phenol (VIII) with Cs₂CO₃ and the benzyl bromide derivatives produced the desired target compounds (IX). Accordingly, in some embodiments, processes are provided for preparing compounds of Formula IX:

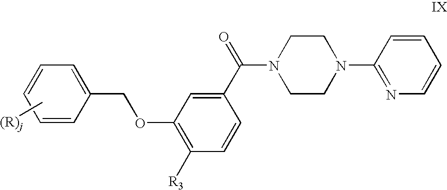

wherein R₃ is as defined above, R is C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl) or CN; and j is 0, 1, 2, or 3;
comprising reacting a compound of Formula VIII:

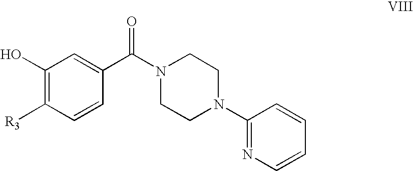

with a benzyl halide derivative of Formula VIIIa:

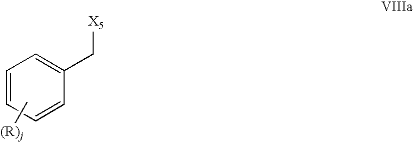

where X₅ is halogen, for a time and under conditions effective to form the compound of Formula IX. In some embodiments, X₅ is bromine.

In further embodiments, compounds of the invention having the general Formula XI are produced in accordance with Scheme 4 below.

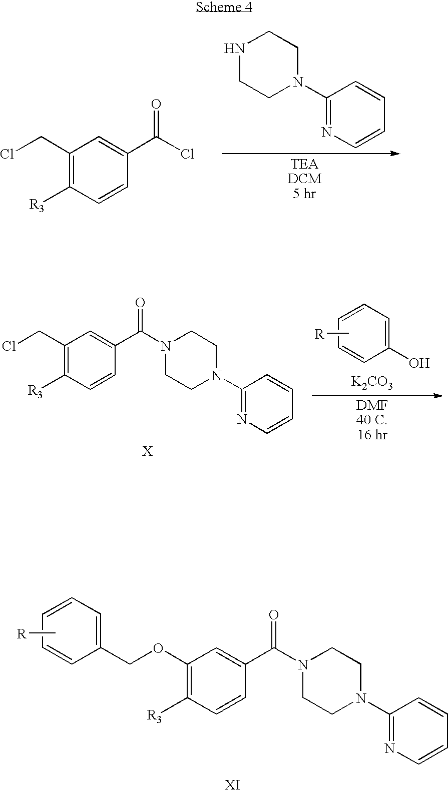

In accordance with Scheme 4, acid chlorides are reacted with N-substituted piperazines using TEA in DCM producing an amide (X). Alkylation of the resulting benzyl chloride (X) with phenol derivatives and K₂CO₃ produced the desired target compounds (XI).

Accordingly, in some embodiments, processes are provided for preparing compounds of Formula XI:

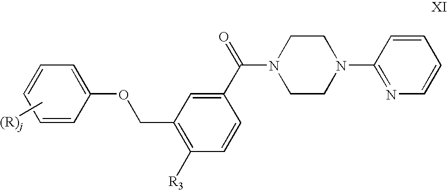

comprising reacting a compound of Formula X:

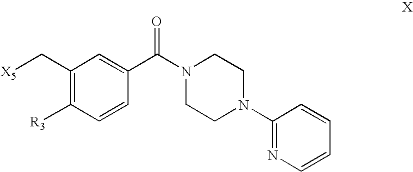

with a phenol derivative of Formula Xa:

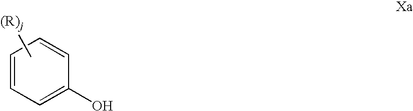

for a time and under conditions effective to form the compound of Formula XI; wherein the constituent variables are as defined above.

In further embodiments, compounds of the invention having the general Formula XII are produced in accordance with Scheme 5 below.

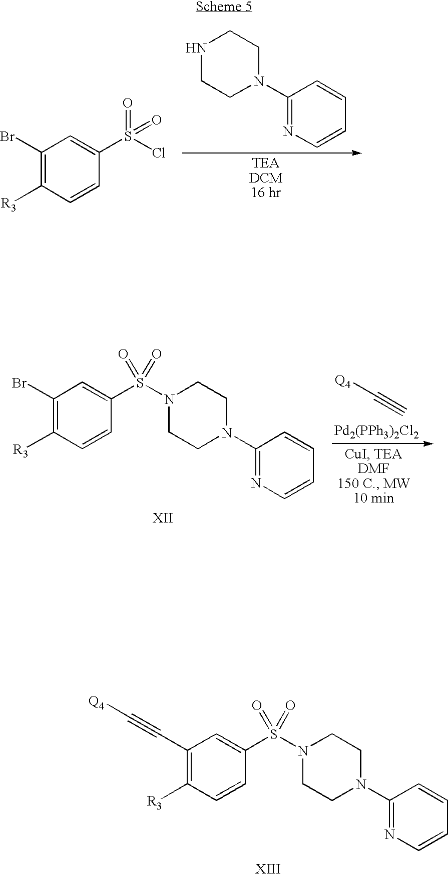

In accordance with Scheme 5, reaction of sulfonyl chlorides with N-substituted piperazines using TEA in DCM produced sulfonamides (XII). Sonagashira coupling of bromoaromatics (XII) with acetylenes using Pd(PPh₃)₂Cl₂ in the presence of CuI and TEA under microwave conditions produced the desired target compounds (XIII) (see WO 2005/123713).

Accordingly, in some embodiments, processes are provided for preparing a compound of Formula XIII:

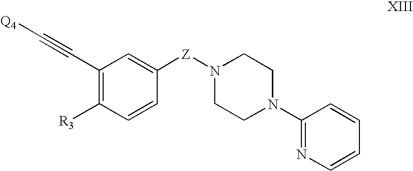

comprising reacting a compound of Formula XII:

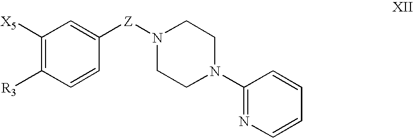

wherein the constituent variables are as defined above, and X₅ is halogen, with an acetylene of Formula Q₄-CCH; in the presence of a palladium triphenylphosphine-containing catalyst for a time and under conditions effective to form the compounds of Formula XII. In some embodiments, the palladium triphenylphosphine-containing catalyst is Pd(PPh₃)₂Cl₂.

In further embodiments, compounds of the invention having the general Formula XV are produced in accordance with Scheme 6 below.

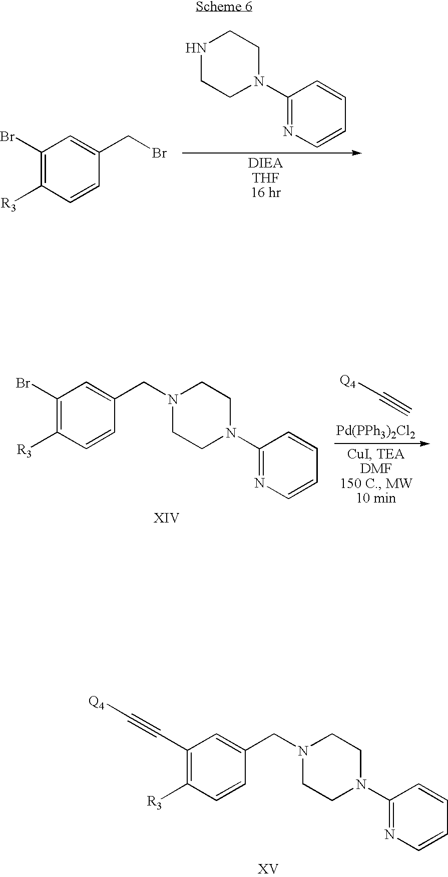

In accordance with Scheme 6, reaction of benzyl bromides with N-substituted piperazines using DIEA in THF produced benzyl piperazines (XIV). Sonagashira coupling of bromoaromatics (XIV) with acetylenes using Pd (PPh₃)₂Cl₂ in the presence of CuI and TEA under microwave conditions produced the desired product (XV) (see WO 2005/123713). Accordingly, in some embodiments, processes are provided for preparing compounds of Formula XV, wherein the constituent variables are as defined above, comprising reacting a compound of Formula XIV with an acetylene as shown in Scheme 6, in the presence of a palladium triphenylphosphine-containing catalyst, for example Pd(PPh₃)₂Cl₂, for a time and under conditions effective to form the compound of Formula XV.

### Analytical Methods

The following methods were used for the characterization of compounds appearing in the Examples below.

Standard LCMS Conditions for Compound Characterization:

### HPLC Conditions: Instrument—Agilent 1100

Column: Thermo Aquasil C18, 50×2.1 mm, 5 μm

Mobile Phase A: 0.1% Formic Acid in water
B: 0.1% Formic Acid in ACN

Flow Rate: 0.800 mL/min

Column Temperature: 40° C.

Injection Volume: 5 mL

UV: monitor 215, 230, 254, 280, and 300 nm

Purity is reported at 254 nm unless otherwise noted.

Gradient Table:

| Time (min) | % B |
|---|---|
| 0 | 0 |
| 2.5 | 100 |
| 4.0 | 100 |
| 4.1 | 0 |
| 5.5 | 0 |

MS Conditions: Instrument: Agilent MSD; Ionization Mode: API-ES; Gas Temperature: 350° C.; Drying Gas: 11.0 L/min.; Nebulizer Pressure: 55 psig; Polarity: 50% positive, 50% negative; VCap: 3000 V (positive), 2500 V (negative); Fragmentor: 80 (positive), 120 (negative); Mass Range: 100-1000 m/z; Threshold: 150; Step size: 0.15; Gain: 1; Peak width: 0.15 minutes.
Preparative reverse-phase HPLC(RP-HPLC): Compounds were in dissolved in 2 mL of 1:1 DMSO:MeCN, filtered through a 0.45 μm GMF, and purified on a Gilson HPLC, using a Phenomenex LUNA C₁₈ column: 60 mm×21.2 mm I.D., 5 um particle size: with ACN/H₂O (containing 0.2% TFA) gradient elution (95:5 H₂O:MeCN to 10:90 H₂O:MeCN; 8 minute run.

### Determination of Activity of Compounds

Compounds of the invention were prepared and analyzed to identify affinity at the rat mGluR5 receptor, based on their ability to displace [³H] labeled 2-methyl-6-(phenylethyl)-pyridine (“MPEP”; a mGluR5 selective negative allosteric modulator) from Hek-293 cell membranes expressing a rat mGluR5 receptor.

MGluR5 expressing HEK-293 cells were scraped off a plate, transferred to centrifuge tubes and washed twice by centrifugation (2000 rpm for 10 minutes, at 4° C.) in buffer (50 mM Tris pH 7.5). The resulting pellets were aliquoted and stored at minus 80° C. On the day of assay, the cells were thawed on ice and re-suspended in buffer. The binding assay was performed in a 96 well microtiter plate in a total volume of 250 μm. Non-specific binding was determined in the presence of 10 μM MPEP. The binding reaction included a final radioligand [³H]-MPEP concentration of 4 nM and 12-25 μg membrane protein per well. Following a 60 minute incubation at room temperature, the reaction was terminated by the addition of ice cold buffer and rapid filtration through a GF/B filter that had been presoaked for 30 minutes in 0.5% PEI. Compounds were initially tested in a single point assay to determine percent inhibition at 10 μM. Subsequently, Kᵢ values were determined for compounds considered to be active.

Percent inhibition and Kᵢ values were generated by GraphPad Prism and Excel Fit. IC₅₀ values were calculated using GraphPad by fitting to a 1 or 2 site-binding model. Kᵢ values were calculated from the apparent IC₅₀ values using the Cheng-Prussof Equation (Biochem. Pharmacol. 22:3099-3108, 1973):

Kᵢ=IC₅₀/1+([L]/K_{d})

where [L] is the concentration of free radioligand and K_{d} is the dissociation constant of radioligand for the receptor.

### Preparation of Exemplary Compounds

The following examples are provided to illustrate the production and activity of representative compounds of the present teachings and to illustrate their performance in a screening assay. One skilled in the art will appreciate that although specific reagents and conditions are outlined in the following examples, these reagents and conditions are not a limitation on the present teachings. In the following examples, chemical structures and names were produced using Chemdraw v 7.0.3. In any conflict between chemical nomenclature and structure, the structure should prevail.

### EXAMPLES

### Example 1

### 1-{4-methoxy-3-[(3-methoxyphenyl)ethynyl]benzoyl}-4-pyridin-2-ylpiperazin (Compound 17)

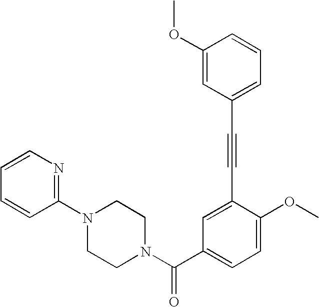

### Step 1: (3-bromo-4-methoxyphenyl)(4-(pyridine-2-yl)piperazin-1-yl)methanone

1-(pyridin-2-yl)piperazine (13 mmol) was added to a solution of 3-bromo-4-methoxybenzoic acid (8.7 mmol) in DMF (100 mL) and DIEA (17.4 mmol). The solution was allowed to stir at room temperature for 10 minutes, and then HOBt (13 mmol) and 1-(3-(dimethylamino)propyl)-3-ethyl-carbodiimide hydrochloride (WSCDI) (13 mmol) were added. The reaction was allowed to stir at room temperature for 16 hours, at which time Liquid Chromatography—Mass Spectrophotometry (LCMS) analysis indicated the reaction was complete. The solution was diluted with 100 mL ethyl acetate (EtOAc) and washed with 100 mL water. The organic layer was dried over MgSO₄, and concentrated in vacuo. Purification via silica column chromatography (Hex:EtOAc as eluent) produced the intermediate compound (3-bromo-4-methoxyphenyl)(4-(pyridin-2-yl)piperazin-1-yl)methanone.

### Step 2: 1-{4-methoxy-3-[(3-methoxyphenyl)ethynyl]benzoyl}-4-pyridin-2-ylpiperazin (Compound 17)

To a solution of (3-bromo-4-methoxyphenyl)(4-(pyridin-2-yl)piperazin-1-yl)methanone (0.15 mmol) and 3-ethynylanisole (0.23 mmol) in DMF (2 mL) in a microwave vial was added copper iodide (0.03 mmol) and TEA (0.45 mmol). Pd(PPh₃)₂Cl₂ (0.03 mmol) was added to the resulting suspension, and the vial was purged with N₂, capped, and microwaved for 10 minutes at 150° C. The solution was concentrated on a speedvac and purified via preparative HPLC (Gilson with NH₄OH additive) to produce the title compound. LCMS Rt=1.84 min (MS=370).

Compounds 1-68, shown in Tables 1 and 1A below, were prepared using the procedure of Example 1 described above.

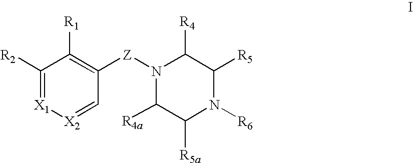

### THE FOLLOWING VALUES REFER TO FORMULA I

### WHEREIN R₁, R₄, R₄ₐ, R₅, R₅ₐ═H; X₂═CH; Z=CO

TABLE 1

| Compound | Name | R₂ | X₁ | R₆ |
|---|---|---|---|---|
| 1 | 1-{3-[(4- methylphenyl)ethynyl]benzoyl}- 4-pyridin-2-ylpiperazine | 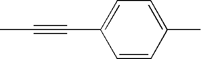 | CH | 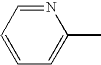 |
| 2 | 1-{3-[(4- methoxyphenyl)ethynyl]benzoyl}- 4-pyridin-2-ylpiperazine | 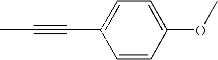 | CH | 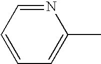 |
| 3 | 1-{3-[(4- chlorophenyl)ethynyl]benzoyl}- 4-pyridin-2-ylpiperazine | 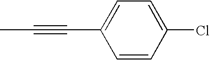 | CH | 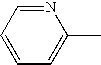 |
| 4 | 1-{3-[(2- methylphenyl)ethynyl]benzoyl}- 4-pyridin-2-ylpiperazine | 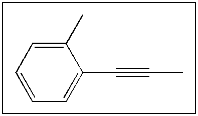 | CH | 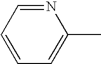 |
| 5 | 1-pyridin-2-yl-4-(3-{[2- (trifluoromethyl)phenyl]ethynyl} benzoyl)piperazine | 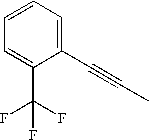 | CH | 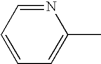 |
| 6 | 3-({3-[(4-pyridin-2-ylpiperazin-1- yl)carbonyl]phenyl}ethynyl)phenol | 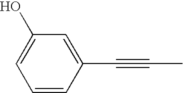 | CH | 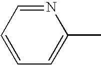 |
| 7 | 1-{3-[(1-methyl-1H-imidazol-5- yl)ethynyl]benzoyl}-4-pyridin-2- ylpiperazine | 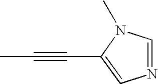 | CH | 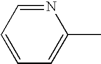 |
| 8 | 1-[3-(cyclohex-1-en-1- ylethynyl)benzoyl]-4-pyridin-2- ylpiperazine | 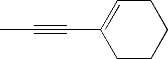 | CH | 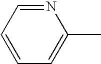 |
| 9 | 1-({3-[(4-pyridin-2-ylpiperazin-1- yl)carbonyl]phenyl}ethynyl) cyclopentanol | 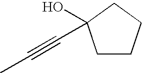 | CH | 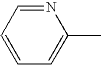 |
| 10 | 1-[3-(3-phenylprop-1-yn-1- yl)benzoyl]-4-pyridin-2- ylpiperazine | 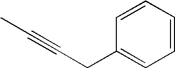 | CH | 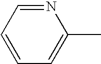 |
| 11 | 3-({3-[(4-pyridin-2-ylpiperazin-1- yl)carbonyl]phenyl}ethynyl) aniline | 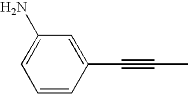 | CH | 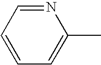 |
| 12 | 1-({3-[(4-pyridin-2-ylpiperazin-1- yl)carbonyl]phenyl}ethynyl) cyclohexanol | 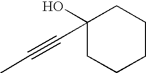 | CH | 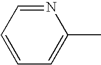 |
| 13 | 1-phenyl-3-{3-[(4-pyridin-2- ylpiperazin-1- yl)carbonyl]phenyl}prop-2-yn-1-ol | 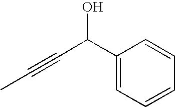 | CH | 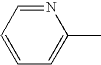 |
| 14 | 1-{3-[(3- methoxyphenyl)ethynyl]benzoyl}- 4-pyridin-2-ylpiperazine | 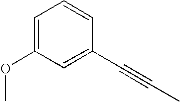 | CH | 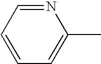 |
| 15 | 1-[3-(cyclohex-1-en-1- ylethynyl)-4-methoxybenzoyl]-4- pyridin-2-ylpiperazine | 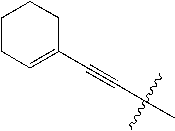 | COMe | 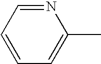 |
| 16 | 1-[4-methoxy-3-(3-phenylprop- 1-yn-1-yl)benzoyl]-4-pyridin-2- ylpiperazine | 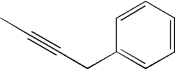 | COMe | 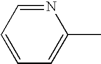 |
| 17 | 1-{4-methoxy-3-[(3- methoxyphenyl)ethynyl]benzoyl}- 4-pyridin-2-ylpiperazine | 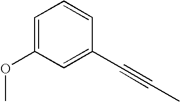 | COMe | 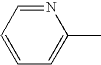 |
| 18 | 1-{4-methoxy-3-[(3- methylphenyl)ethynyl]benzoyl}-4- pyridin-2-ylpiperazine | 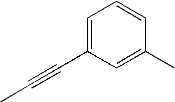 | COMe | 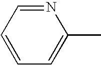 |
| 19 | 1-{3-[(3-chlorophenyl)ethynyl]- 4-methoxybenzoyl}-4-pyridin- 2-ylpiperazine | 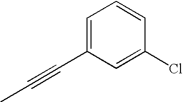 | COMe | 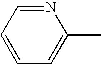 |
| 20 | 1-{3-[(3,5- dimethoxyphenyl)ethynyl]-4- methoxybenzoyl}-4-pyridin-2- ylpiperazine | 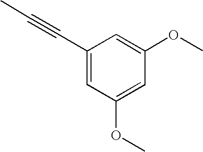 | COMe | 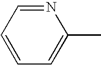 |
| 21 | 1-{3-[(3,5-difluorophenyl)ethynyl]- 4-methoxybenzoyl}-4-pyridin-2- ylpiperazine | 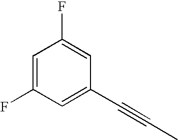 | COMe | 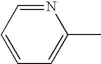 |
| 22 | 1-{3-[(2,5- dimethylphenyl)ethynyl]-4- methoxybenzoyl}-4-pyridin-2- ylpiperazine | 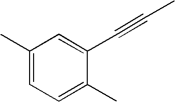 | COMe | 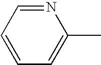 |
| 23 | 1-{4-methoxy-3-[(2,4,5- trimethylphenyl)ethynyl]benzoyl}- 4-pyridin-2-ylpiperazine | 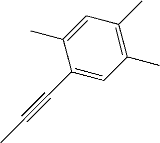 | COMe | 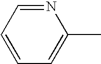 |
| 24 | 1-[3-(cyclohex-1-en-1- ylethynyl)-4-methylbenzoyl]-4- pyridin-2-ylpiperazine | 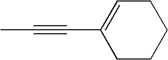 | CCH₃ | 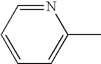 |
| 25 | 1-[4-methyl-3-(3-phenylprop-1- yn-1-yl)benzoyl]-4-pyridin-2- ylpiperazine | 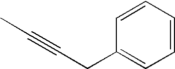 | CCH₃ | 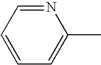 |
| 26 | 1-{3-[(3-methoxyphenyl)ethynyl]- 4-methylbenzoyl}-4-pyridin-2- ylpiperazine | 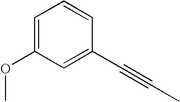 | CCH₃ | 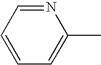 |
| 27 | 1-(4-methyl-3-{[3- (trifluoromethyl) phenyl]ethynyl}benzoyl)-4- pyridin-2-ylpiperazine | 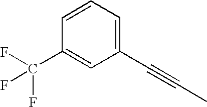 | CCH₃ | 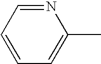 |
| 28 | 1-{3-[(3-chlorophenyl)ethynyl]- 4-methylbenzoyl}-4-pyridin-2- ylpiperazine | 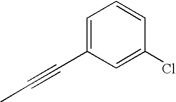 | CCH₃ | 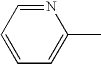 |
| 29 | 1-{3-[(3,5-difluorophenyl)ethynyl]- 4-methylbenzoyl}-4-pyridin-2- ylpiperazine | 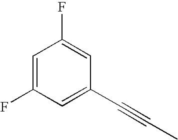 | CCH₃ | 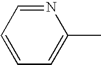 |
| 30 | 1-{3-[(2,5-dimethylphenyl)ethynyl]- 4-methylbenzoyl}-4-pyridin-2- ylpiperazine | 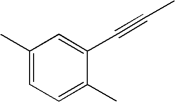 | CCH₃ | 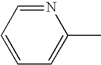 |
| 31 | 1-{3-[(4-fluoro-3- methylphenyl)ethynyl]-4- methylbenzoyl}-4-pyridin-2- ylpiperazine | 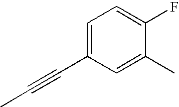 | CCH₃ | 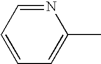 |
| 32 | 1-[4-methyl-3-(pyridin-3- ylethynyl)benzoyl]-4-pyridin-2- ylpiperazine | 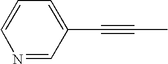 | CCH₃ | 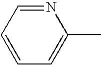 |
| 33 | 3-({2-methoxy-5-[(4-pyridin-2- ylpiperazin-1- yl)carbonyl]phenyl}ethynyl)phenol | 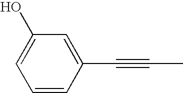 | COMe | 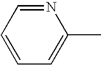 |
| 34 | 3-({2-methoxy-5-[(4-pyridin-2- ylpiperazin-1- yl)carbonyl]phenyl}ethynyl)aniline | 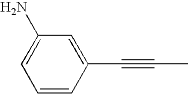 | COMe | 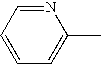 |
| 35 | 1-[4-methoxy-3-(pyridin-3- ylethynyl)benzoyl]-4-pyridin-2- ylpiperazine | 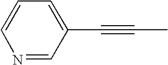 | COMe | 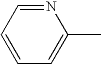 |
| 36 | 3-({2-methyl-5-[(4-pyridin-2- ylpiperazin-1- yl)carbonyl]phenyl}ethynyl)aniline | 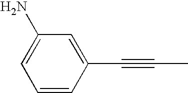 | CCH₃ | 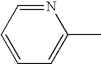 |
| 37 | 1-{3-[(4-fluoro-3- methylphenyl)ethynyl]-4- methoxybenzoyl}-4-pyridin-2- ylpiperazine | 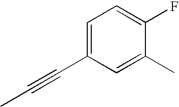 | COMe | 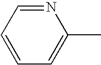 |
| 38 | 1-[4-methoxy-3-(pyridin-2- ylethynyl)benzoyl]-4-pyridin-2- ylpiperazine | 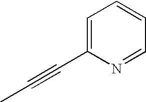 | COMe | 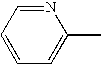 |
| 39 | 1-{4-methyl-3-[(3- methylphenyl)ethynyl]benzoyl}-4- pyridin-2-ylpiperazine | 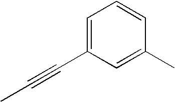 | CCH₃ | 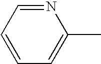 |
| 40 | 1-{3-[(3,5- dimethoxyphenyl)ethynyl]-4- methylbenzoyl}-4-pyridin-2- ylpiperazine | 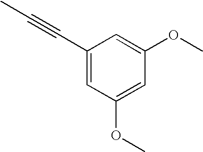 | CCH₃ | 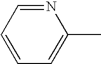 |
| 41 | 1-{4-methyl-3-[(2,4,5- trimethylphenyl)ethynyl]benzoyl}- 4-pyridin-2-ylpiperazine | 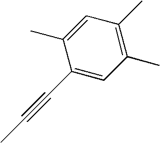 | CCH₃ | 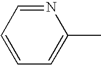 |
| 42 | 1-[4-methyl-3-(pyridin-2- ylethynyl)benzoyl]-4-pyridin-2- ylpiperazine | 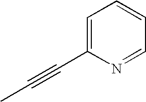 | CCH₃ | 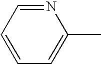 |
| 43 | 1-(4-methoxy-3-{[3- (trifluoromethyl)phenyl]ethynyl} benzoyl)-4-pyridin-2-ylpiperazine | 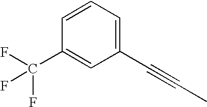 | COMe | 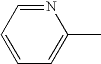 |
| 44 | 1-(3-{[3,5- bis(trifluoromethyl)phenyl]ethynyl}- 4-methoxybenzoyl)-4-pyridin-2- ylpiperazine | 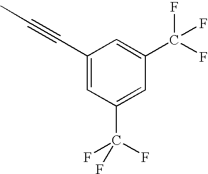 | COMe | 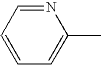 |
| 45 | 1-[4-methoxy-3-(pyridin-4- ylethynyl)benzoyl]-4-pyridin-2- ylpiperazine | 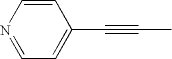 | COMe | 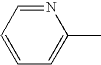 |
| 46 | 3-({2-methyl-5-[(4-pyridin-2- ylpiperazin-1- yl)carbonyl]phenyl}ethynyl)phenol | 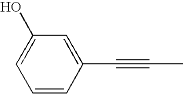 | CCH₃ | 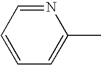 |
| 47 | 1-(3-{[3,5- bis(trifluoromethyl)phenyl]ethynyl}- 4-methylbenzoyl)-4-pyridin-2- ylpiperazine | 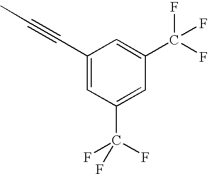 | CCH₃ | 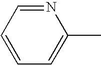 |
| 48 | 1-[4-methyl-3-(pyridin-4- ylethynyl)benzoyl]-4-pyridin-2- ylpiperazine | 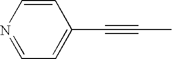 | CCH₃ | 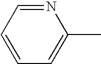 |
| 49 | 2-{4-[4-methoxy-3- (phenylethynyl)benzoyl]piperazin- 1-yl}pyrazine | 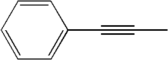 | COMe | 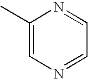 |
| 50 | 2-{4-[3-(cyclohex-1-en-1- ylethynyl)-4- methoxybenzoyl]piperazin-1- yl}pyrazine | 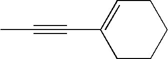 | COMe | 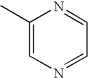 |
| 51 | 2-{4-[4-methoxy-3-(3- phenylprop-1-yn-1- yl)benzoyl]piperazin-1-yl}pyrazine | 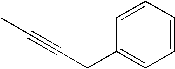 | COMe | 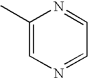 |
| 52 | 2-(4-{4-methoxy-3-[(3- methoxyphenyl)ethynyl]benzoyl} piperazin-1-yl)pyrazine | 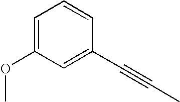 | COMe | 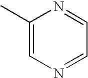 |
| 53 | 2-(4-{4-methoxy-3-[(3- methylphenyl)ethynyl]benzoyl} piperazin-1-yl)pyrazine | 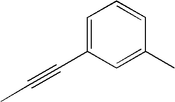 | COMe | 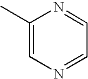 |
| 54 | 2-[4-(4-methoxy-3-{[3- (trifluoromethyl)phenyl]ethynyl} benzoyl)piperazin-1-yl]pyrazine | 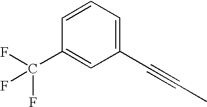 | COMe | 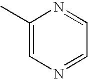 |
| 55 | 2-{4-[4-methyl-3- (phenylethynyl)benzoyl]piperazin- 1-yl}pyrazine | 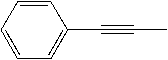 | CCH₃ | 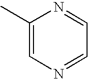 |
| 56 | 2-{4-[3-(cyclohex-1-en-1- ylethynyl)-4- methylbenzoyl]piperazin-1- yl}pyrazine | 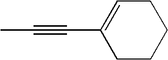 | CCH₃ | 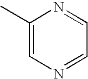 |
| 57 | 2-{4-[4-methyl-3-(3- phenylprop-1-yn-1- yl)benzoyl]piperazin-1 - yl}pyrazine | 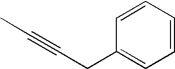 | CCH₃ | 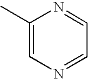 |
| 58 | 2-(4-{3-[(3- methoxyphenyl)ethynyl]-4- methylbenzoyl}piperazin-1- yl)pyrazine | 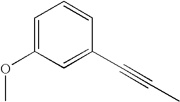 | CCH₃ | 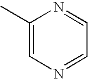 |
| 59 | 2-(4-{4-methyl-3-[(3- methylphenyl)ethynyl]benzoyl} piperazin-1-yl)pyrazine | 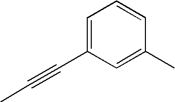 | CCH₃ | 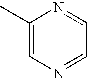 |
| 60 | 2-[4-(4-methyl-3-{[3- (trifluoromethyl)phenyl]ethynyl} benzoyl)piperazin-1-yl]pyrazine | 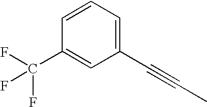 | CCH₃ | 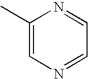 |
| 61 | 2-(4-{3-[(3-fluorophenyl)ethynyl]- 4-methoxybenzoyl} piperazin-1-yl)pyrazine | 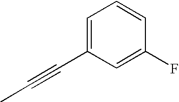 | COMe | 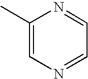 |
| 62 | 2-(4-{3-[(3-fluorophenyl)ethynyl]- 4-methylbenzoyl}piperazin-1- yl)pyrazine | 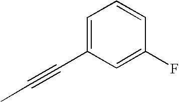 | CCH₃ | 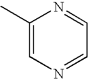 |
| 63 | 1-{3-[(3-fluorophenyl)ethynyl]- 4-methoxybenzoyl}-4-pyridin- 2-ylpiperazine | 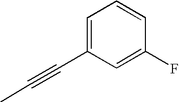 | COMe | 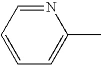 |
| 64 | 1-{3-[(3-fluorophenyl)ethynyl]- 4-methylbenzoyl}-4-pyridin-2- ylpiperazine | 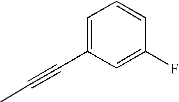 | CCH₃ | 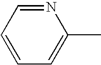 |
| 65 | 2-(4-{3-[(3-chlorophenyl)ethynyl]- 4-methoxybenzoyl}piperazin-1- yl)pyrazine | 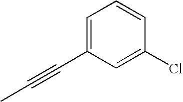 | COMe | 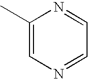 |
| 66 | 2-{4-[4-methoxy-3-(pyridin-2- ylethynyl)benzoyl]piperazin-1- yl}pyrazine | 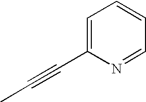 | COMe | 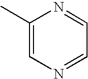 |
| 67 | 2-(4-{3-[(3-chlorophenyl)ethynyl]- 4-methylbenzoyl}piperazin-1- yl)pyrazine | 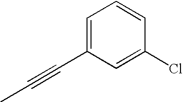 | CCH₃ | 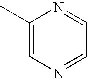 |
| 68 | 2-{4-[4-methyl-3-(pyridin-2- ylethynyl)benzoyl]piperazin-1- yl}pyrazine | 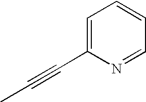 | CCH₃ | 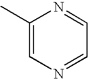 |

TABLE 1A

| | | | | | Biological Activity | |
|---|---|---|---|---|---|---|
| | | LCMS data | | | | PCT |
| | | Time | | | Median | INHIB |
| Cmpd | Name | (min.) | Mass | Ion | Ki (μM) | (%) @ 10 μm |
| 1 | 1-{3-[(4-methylphenyl)ethynyl]benzoyl}- | 2.24 | 382.2 | M + H | 1.906 | 55 |
| | 4-pyridin-2- | | | | | |
| | ylpiperazine | | | | | |
| 2 | 1-{3-[(4-methoxyphenyl)ethynyl]benzoyl}- | 2.14 | 398.2 | M + H | 2.356 | 58 |
| | 4-pyridin-2- | | | | | |
| | ylpiperazine | | | | | |
| 3 | 1-{3-[(4-chlorophenyl)ethynyl]benzoyl}- | 2.3 | 402.1 | M + H | | 45 |
| | 4-pyridin-2- | | | | | |
| | ylpiperazine | | | | | |
| 4 | 1-{3-[(2-methylphenyl)ethynyl]benzoyl}- | 2.23 | 382.2 | M + H | | 0 |
| | 4-pyridin-2- | | | | | |
| | ylpiperazine | | | | | |
| 5 | 1-pyridin-2-yl-4-(3-{[2- | 2.25 | 436.2 | M + H | | 0 |
| | (trifluoromethyl)phenyl]ethynyl}benzoyl)piperazine | | | | | |
| 6 | 3-({3-[(4-pyridin-2- | 1.87 | 384.2 | M + H | 1.11554 | 75 |
| | ylpiperazin-1-yl)carbonyl]phenyl}ethynyl)phenol | | | | | |
| 7 | 1-{3-[(1-methyl-1H-imidazol- | 1.44 | 372.2 | M + H | | 0 |
| | 5-yl)ethynyl]benzoyl}-4- | | | | | |
| | pyridin-2-ylpiperazine | | | | | |
| 8 | 1-[3-(cyclohex-1-en-1-ylethynyl)benzoyl]- | 2.18 | 372.2 | M + H | 1.21615 | 78 |
| | 4-pyridin-2- | | | | | |
| | yl piperazine | | | | | |
| 9 | 1-({3-[(4-pyridin-2-ylpiperazin- | 1.76 | 376.2 | M + H | | 0 |
| | 1-yl)carbonyl]phenyl}ethynyl)cyclopentanol | | | | | |
| 10 | 1-[3-(3-phenylprop-1-yn-1- | 2.08 | 382.2 | M + H | 0.13881 | 89 |
| | yl)benzoyl]-4-pyridin-2-yl | | | | | |
| | piperazine | | | | | |
| 11 | 3-({3-[(4-pyridin-2- | 1.75 | 383.2 | M + H | 0.42992 | 85 |
| | ylpiperazin-1-yl)carbonyl]phenyl}ethynyl)aniline | | | | | |
| 12 | 1-({3-[(4-pyridin-2-ylpiperazin- | 1.83 | 390.2 | M + H | | 0 |
| | 1-yl)carbonyl]phenyl}ethynyl)cyclohexanol | | | | | |
| 13 | 1-phenyl-3-{3-[(4-pyridin-2- | 1.8 | 398.2 | M + H | | 15 |
| | ylpiperazin-1-yl)carbonyl]phenyl}prop- | | | | | |
| | 2-yn-1-ol | | | | | |
| 14 | 1-{3-[(3-methoxyphenyl)ethynyl]benzoyl}- | 2.08 | 398.2 | M + H | 0.21238 | 87 |
| | 4-pyridin-2- | | | | | |
| | ylpiperazine | | | | | |
| 15 | 1-[3-(cyclohex-1-en-1- | 2.18 | 402.2 | M + H | 0.236 | 76 |
| | ylethynyl)-4-methoxybenzoyl]- | | | | | |
| | 4-pyridin-2-ylpiperazine | | | | | |
| 16 | 1-[4-methoxy-3-(3- | 1.95 | 412.2 | M + H | 0.043 | 84 |
| | phenylprop-1-yn-1-yl)benzoyl]- | | | | | |
| | 4-pyridin-2-yl | | | | | |
| | piperazine | | | | | |
| 17 | 1-{4-methoxy-3-[(3-methoxyphenyl)ethynyl]benzoyl}- | 2.07 | 428.2 | M + H | 0.051 | 91 |
| | 4- | | | | | |
| | pyridin-2-ylpiperazine | | | | | |
| 18 | 1-{4-methoxy-3-[(3-methylphenyl)ethynyl]benzoyl}- | 2.02 | 412.2 | M + H | 0.006 | 99 |
| | 4- | | | | | |
| | pyridin-2-ylpiperazine | | | | | |
| 19 | 1-{3-[(3-chlorophenyl)ethynyl]- | 2.22 | 432.1 | M + H | 0.006 | 97 |
| | 4-methoxybenzoyl}- | | | | | |
| | 4-pyridin-2-ylpiperazine | | | | | |
| 20 | 1-{3-[(3,5-dimethoxyphenyl)ethynyl]- | 2.11 | 458.2 | M + H | | 0 |
| | 4-methoxybenzoyl}- | | | | | |
| | 4-pyridin-2-ylpiperazine | | | | | |
| 21 | 1-{3-[(3,5-difluorophenyl)ethynyl]- | 2.16 | 434.2 | M + H | 1.942 | 79 |
| | 4-methoxybenzoyl}- | | | | | |
| | 4-pyridin-2-ylpiperazine | | | | | |
| 22 | 1-{3-[(2,5-dimethylphenyl)ethynyl]- | 2.28 | 426.2 | M + H | | 43 |
| | 4-methoxybenzoyl}- | | | | | |
| | 4-pyridin-2-ylpiperazine | | | | | |
| 23 | 1-{4-methoxy-3-[(2,4,5- | 2.36 | 440.2 | M + H | | 0 |
| | trimethylphenyl)ethynyl]benzoyl}- | | | | | |
| | 4-pyridin-2-ylpiperazine | | | | | |
| 24 | 1-[3-(cyclohex-1-en-1- | 2.34 | 386.2 | M + H | 1.287 | 71 |
| | ylethynyl)-4-methylbenzoyl]- | | | | | |
| | 4-pyridin-2-ylpiperazine | | | | | |
| 25 | 1-[4-methyl-3-(3-phenylprop- | 2.06 | 396.2 | M + H | 0.452 | 63 |
| | 1-yn-1-yl)benzoyl]-4-pyridin- | | | | | |
| | 2-ylpiperazine | | | | | |
| 26 | 1-{3-[(3-methoxyphenyl)ethynyl]- | 2.21 | 412.2 | M + H | 0.322 | 86 |
| | 4-methylbenzoyl}-4- | | | | | |
| | pyridin-2-ylpiperazine | | | | | |
| 27 | 1-(4-methyl-3-{[3-(trifluoromethyl)phenyl]ethynyl}benzoyl)- | 2.38 | 450.2 | M + H | 0.469 | 81 |
| | 4-pyridin-2-ylpiperazine | | | | | |
| 28 | 1-{3-[(3-chlorophenyl)ethynyl]- | 2.36 | 416.1 | M + H | 0.076 | 79 |
| | 4-methylbenzoyl}-4-pyridin- | | | | | |
| | 2-ylpiperazine | | | | | |
| 29 | 1-{3-[(3,5-difluorophenyl)ethynyl]- | 2.29 | 418.2 | M + H | | 41 |
| | 4-methylbenzoyl}-4- | | | | | |
| | pyridin-2-ylpiperazine | | | | | |
| 30 | 1-{3-[(2,5-dimethylphenyl)ethynyl]- | 2.414 | 410.2 | M + H | 3.81 | 55 |
| | 4-methylbenzoyl}-4- | | | | | |
| | pyridin-2-ylpiperazine | | | | | |
| 31 | 1-{3-[(4-fluoro-3-methylphenyl)ethynyl]- | 2.35 | 414.2 | M + H | 0.472 | 68 |
| | 4-methylbenzoyl}- | | | | | |
| | 4-pyridin-2- | | | | | |
| | ylpiperazine | | | | | |
| 32 | 1-[4-methyl-3-(pyridin-3- | 1.88 | 383.2 | M + H | 3.107 | 68 |
| | ylethynyl)benzoyl]-4-pyridin- | | | | | |
| | 2-ylpiperazine | | | | | |
| 33 | 3-({2-methoxy-5-[(4-pyridin-2- | 1.88 | 414.2 | M + H | 0.54077 | 82 |
| | ylpiperazin-1-yl)carbonyl]phenyl}ethynyl)phenol | | | | | |
| 34 | 3-({2-methoxy-5-[(4-pyridin-2- | 1.76 | 413.2 | M + H | 0.29887 | 62 |
| | ylpiperazin-1-yl)carbonyl]phenyl}ethynyl)aniline | | | | | |
| 35 | 1-[4-methoxy-3-(pyridin-3- | 1.75 | 399.2 | M + H | 0.89806 | 67 |
| | ylethynyl)benzoyl]-4-pyridin- | | | | | |
| | 2-ylpiperazine | | | | | |
| 36 | 3-({2-methyl-5-[(4-pyridin-2- | 1.89 | 397.2 | M + H | 0.47427 | 79 |
| | ylpiperazin-1-yl)carbonyl]phenyl}ethynyl)aniline | | | | | |
| 37 | 1-{3-[(4-fluoro-3-methylphenyl)ethynyl]- | 2.19 | 430.2 | M + H | 0.05471 | 74 |
| | 4-methoxybenzoyl}- | | | | | |
| | 4-pyridin-2-yl | | | | | |
| | piperazine | | | | | |
| 38 | 1-[4-methoxy-3-(pyridin-2- | 1.77 | 399.2 | M + H | 0.02236 | 81 |
| | ylethynyl)benzoyl]-4-pyridin- | | | | | |
| | 2-ylpiperazine | | | | | |
| 39 | 1-{4-methyl-3-[(3-methylphenyl)ethynyl]benzoyl}- | 2.29 | 396.2 | M + H | 0.05706 | 83 |
| | 4- | | | | | |
| | pyridin-2-ylpiperazine | | | | | |
| 40 | 1-{3-[(3,5-dimethoxyphenyl)ethynyl]- | 2.22 | 442.2 | M + H | | 0 |
| | 4- | | | | | |
| | methylbenzoyl}-4-pyridin-2- | | | | | |
| | ylpiperazine | | | | | |
| 41 | 1-{4-methyl-3-[(2,4,5- | 2.51 | 424.2 | M + H | | 0 |
| | trimethylphenyl)ethynyl]benzoyl}- | | | | | |
| | 4-pyridin-2-ylpiperazine | | | | | |
| 42 | 1-[4-methyl-3-(pyridin-2- | 1.89 | 383.2 | M + H | 0.0059 | 96 |
| | ylethynyl)benzoyl]-4-pyridin- | | | | | |
| | 2-ylpiperazine | | | | | |
| 43 | 1-(4-methoxy-3-{[3-(trifluoromethyl)phenyl]ethynyl}benzoyl)- | 2.26 | 466.2 | M + H | 0.27753 | 82 |
| | 4-pyridin-2-ylpiperazine | | | | | |
| 44 | 1-(3-{[3,5-bis(trifluoromethyl)phenyl]ethynyl}- | 2.44 | 534.2 | M + H | | 0 |
| | 4- | | | | | |
| | methoxybenzoyl)-4-pyridin-2- | | | | | |
| | ylpiperazine | | | | | |
| 45 | 1-[4-methoxy-3-(pyridin-4- | 1.61 | 399.2 | M + H | 0.99013 | 68 |
| | ylethynyl)benzoyl]-4-pyridin- | | | | | |
| | 2-ylpiperazine | | | | | |
| 46 | 3-({2-methyl-5-[(4-pyridin-2- | 1.97 | 398.2 | M + H | 1.306 | 71 |
| | ylpiperazin-1-yl)carbonyl]phenyl}ethynyl)phenol | | | | | |
| 47 | 1-(3-{[3,5-bis(trifluoromethyl)phenyl]ethynyl}- | 2.52 | 518.2 | M + H | | 0 |
| | 4-methylbenzoyl)- | | | | | |
| | 4-pyridin-2-yl | | | | | |
| | piperazine | | | | | |
| 48 | 1-[4-methyl-3-(pyridin-4- | 1.71 | 383.2 | M + H | | 49 |
| | ylethynyl)benzoyl]-4-pyridin- | | | | | |
| | 2-ylpiperazine | | | | | |
| 49 | 2-{4-[4-methoxy-3-(phenylethynyl)benzoyl]piperazin- | 2.45 | 399.2 | M + H | 0.02343 | 95 |
| | 1- | | | | | |
| | yl}pyrazine | | | | | |
| 50 | 2-{4-[3-(cyclohex-1-en-1- | 2.57 | 403.2 | M + H | 0.17084 | 90 |
| | ylethynyl)-4-methoxybenzoyl]piperazin- | | | | | |
| | 1-yl}pyrazine | | | | | |
| 51 | 2-{4-[4-methoxy-3-(3- | 2.45 | 413.2 | M + H | 0.0339 | 92 |
| | phenylprop-1-yn-1-yl)benzoyl]piperazin- | | | | | |
| | 1-yl}pyrazine | | | | | |
| 52 | 2-(4-{4-methoxy-3-[(3- | 2.49 | 429.2 | M + H | 0.06008 | 81 |
| | methoxyphenyl)ethynyl]benzoyl}piperazin- | | | | | |
| | 1-yl)pyrazine | | | | | |
| 53 | 2-(4-{4-methoxy-3-[(3-methylphenyl)ethynyl]benzoyl}piperazin- | 2.56 | 413.2 | M + H | 0.00863 | 95 |
| | 1-yl)pyrazine | | | | | |
| 54 | 2-[4-(4-methoxy-3-{[3- | 2.65 | 467.2 | M + H | 0.10893 | 96 |
| | (trifluoromethyl)phenyl]ethynyl}benzoyl)piperazin- | | | | | |
| | 1-yl]pyrazine | | | | | |
| 55 | 2-{4-[4-methyl-3-(phenylethynyl)benzoyl]piperazin- | 2.63 | 383.2 | M + H | 0.14539 | 88 |
| | 1- | | | | | |
| | yl}pyrazine | | | | | |
| 56 | 2-{4-[3-(cyclohex-1-en-1- | 2.79 | 387.2 | M + H | 1.88662 | 70 |
| | ylethynyl)-4-methylbenzoyl]piperazin- | | | | | |
| | 1-yl}pyrazine | | | | | |
| 57 | 2-{4-[4-methyl-3-(3-phenylprop- | 2.61 | 397.2 | M + H | 0.36268 | 87 |
| | 1-yn-1-yl)benzoyl]piperazin- | | | | | |
| | 1-yl}pyrazine | | | | | |
| 58 | 2-(4-{3-[(3-methoxyphenyl)ethynyl]- | 2.61 | 413.2 | M + H | 0.3222 | 93 |
| | 4-methylbenzoyl}piperazin- | | | | | |
| | 1-yl)pyrazine | | | | | |
| 59 | 2-(4-{4-methyl-3-[(3-methylphenyl)ethynyl]benzoyl}piperazin- | 2.74 | 397.2 | M + H | 0.06254 | 100 |
| | 1-yl)pyrazine | | | | | |
| 60 | 2-[4-(4-methyl-3-{[3-(trifluoromethyl)phenyl]ethynyl}benzoyl)piperazin- | 2.75 | 451.2 | M + H | 0.74903 | 85 |
| | 1-yl]pyrazine | | | | | |
| 61 | 2-(4-{3-[(3-fluorophenyl)ethynyl]- | 2.52 | 417.2 | M + H | 0.02172 | |
| | 4-methoxybenzoyl}piperazin- | | | | | |
| | 1-yl)pyrazine | | | | | |
| 62 | 2-(4-{3-[(3-fluorophenyl)ethynyl]- | 2.7 | 401.2 | M + H | 0.12052 | |
| | 4-methylbenzoyl}piperazin- | | | | | |
| | 1-yl)pyrazine | | | | | |
| 63 | 1-{3-[(3-fluorophenyl)ethynyl]- | 2.02 | 416.2 | M + H | 0.00734 | |
| | 4-methoxybenzoyl}-4-pyridin- | | | | | |
| | 2-ylpiperazine | | | | | |
| 64 | 1-{3-[(3-fluorophenyl)ethynyl]- | 2.17 | 400.2 | M + H | 0.13084 | |
| | 4-methylbenzoyl}-4-pyridin-2- | | | | | |
| | ylpiperazine | | | | | |
| 65 | 2-(4-{3-[(3-chlorophenyl)ethynyl]- | 2.67 | 433.1 | M + H | 0.00211 | |
| | 4-methoxybenzoyl}piperazin- | | | | | |
| | 1-yl)pyrazine | | | | | |
| 66 | 2-{4-[4-methoxy-3-(pyridin-2- | 2.15 | 400.2 | M + H | 0.02116 | |
| | ylethynyl)benzoyl]piperazin-1- | | | | | |
| | yl}pyrazine | | | | | |
| 67 | 2-(4-{3-[(3-chlorophenyl)ethynyl]- | 2.86 | 417.1 | M + H | 0.04907 | |
| | 4-methylbenzoyl}piperazin- | | | | | |
| | 1-yl)pyrazine | | | | | |
| 68 | 2-{4-[4-methyl-3-(pyridin-2- | 2.32 | 384.2 | M + H | 0.00884 | |
| | ylethynyl)benzoyl]piperazin-1- | | | | | |
| | yl}pyrazine | | | | | |

### Example 2

### 1-[3-(phenylethynyl)benzoyl]-4-pyridin-2-ylpiperazin (Compound 69)

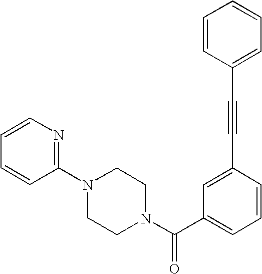

### Step 1: Ethyl 3-(phenylethynyl)benzoate

To ethyl 3-bromobenzoate (12.49 mmol), phenylacetylene (13.74 mmol), and bis(triphenylphosphine)palladium(II) dichloride (0.350 mmol) in TEA (40 ml) was added to copper(I) iodide (0.300 mmol). The reaction was flushed with N₂, capped and stirred at 50° C. overnight. The reaction was cooled to room temperature, filtered through Celite, and the filtrate evaporated. The resultant residue was passed through short silica gel filtration in a fritted funnel (3:1 Hexanes:EtOAc) affording crude ethyl 3-(phenylethynyl)benzoate.

### Step 2: 3-(phenylethynyl)benzoic acid

To the crude ethyl 3-(phenylethynyl)benzoate was added 10% aqueous NaOH (60 ml) and MeOH (30 ml). This reaction mixture was heated to 65° C. and stirred overnight. After the reaction was determined to be complete via Liquid Chromatography/Mass Spectrophotometer (LCMS), the organic solvent was evaporated. To the remaining solution was added water and EtOAC and then the phases were separated. The aqueous layer was acidified to pH 2 and extracted with EtOAc. The organic layer was dried, filtered and evaporated to afford 1.16 grams of 3-(phenylethynyl)benzoic acid, 42% over two steps.

### Step 3: 2-Chloro-N-[3-(morpholin-4-ylcarbonyl)-5,6,7,8-tetrahydro-4H-cyclohepta[b]thien-2-yl]benzamide

1-(pyridin-2-yl)piperazine (0.051 ml, 0.337 mmol) was added to 3-(phenylethynyl)benzoic acid (50 mg, 0.225 mmol) in DMF (1 ml). This solution was stirred for 15 minutes at which time HOBt (51.7 mg, 0.337 mmol) and EDCl (64.7 mg, 0.337 mmol) were added, and the reaction was allowed to stir overnight. The reaction was then concentrated on a speedvac and purified via prep HPLC (Gilson with TFA additive) to afford 53.4 mg of 2-chloro-N-[3-(morpholin-4-ylcarbonyl)-5,6,7,8-tetrahydro-4H-cyclohepta[b]thien-2-yl]benzamide as a white TFA salt. LCMS Rt=1.99 min (MS=368.2)

Compounds 69-149, shown in Tables 2 and 2A below, were prepared using the procedure of Example 2 described above.

### UNLESS NOTED OTHERWISE THE FOLLOWING VALUES IN TABLE 2 REFER TO FORMULA I

### WHEREIN R₁, R₄, R₄ₐ, R₅, R₅ₐ═H; X₁ AND X₂═CH; AND Z=CO

TABLE 2

| Cmpd | Name | R₂ | Noted Values | R₆ |
|---|---|---|---|---|
| 69 | 1-[3-(phenylethynyl) benzoyl]-4-pyridin-2-yl piperazine | 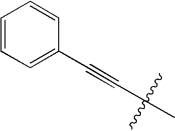 | | 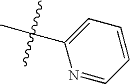 |
| 70 | 1-methyl-4-[3- (phenylethynyl)benzoyl] piperazine | 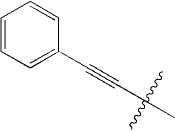 | | CH₃ |
| 71 | 1-(4-methoxyphenyl)-4-[3- (phenylethynyl)benzoyl] piperazine | 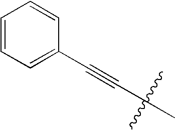 | | 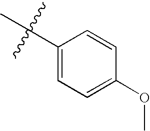 |
| 72 | 1-(4-chlorophenyl)-4-[3- (phenylethynyl)benzoyl] piperazine | 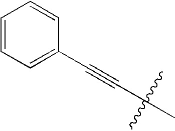 | | 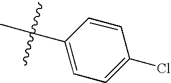 |
| 73 | 1-(4-methylphenyl)-4-[3- (phenylethynyl)benzoyl] piperazine | 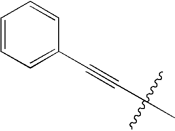 | | 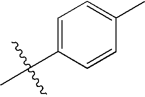 |
| 74 | 1-(4-{-[3- (phenylethynyl)benzoyl] piperazine-1-yl}phenyl) ethanone | 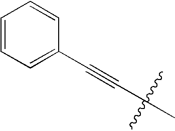 | | 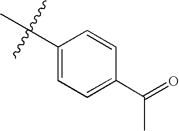 |
| 75 | 1-(4-nitrophenyl)-4-[3- (phenylethynyl)benzoyl] piperazine | 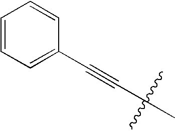 | | 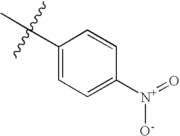 |
| 76 | 1-phenyl-4-[3- (phenylethynyl)benzoyl] piperazine | 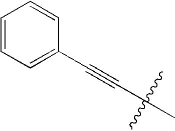 | | 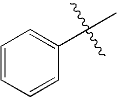 |
| 77 | 1-[3- (phenylethynyl)benzoyl]-4- [4- (trifluoromethyl)phenyl] piperazine | 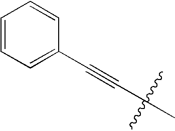 | | 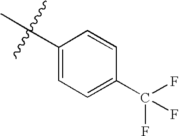 |
| 78 | 1-(2,6-dimethylphenyl)-4-[3- (phenylethynyl)benzoyl] piperazine | 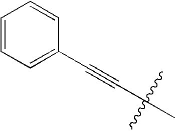 | | 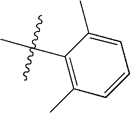 |
| 79 | 1-(4-fluorophenyl)-4-[3- (phenylethynyl)benzoyl] piperazine | 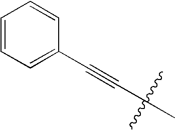 | | 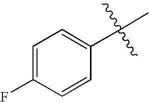 |
| 80 | 1-[2-(methylthio)phenyl]-4- [3- (phenylethynyl)benzoyl] piperazine | 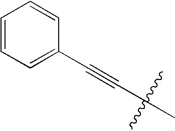 | | 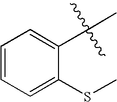 |
| 81 | 1-(3-methoxyphenyl)-4-[3- (phenylethynyl)benzoyl] piperazine | 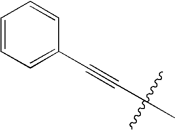 | | 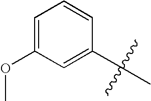 |
| 82 | 1-(3-chlorophenyl)-4-[3- (phenylethynyl)benzoyl] piperazine | 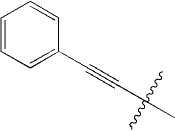 | | 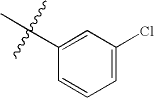 |
| 83 | 4-{4-[3- (phenylethynyl)benzoyl] piperazin-1-yl}phenol | 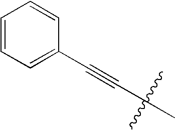 | | 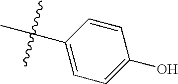 |
| 84 | 1-(3,5-dichlorophenyl)-4-[3- (phenylethynyl)benzoyl] piperazine | 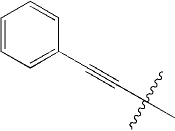 | | 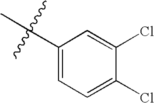 |
| 85 | 1-[3- (phenylethynyl)benzoyl]-4- [3- (trifluoromethyl)phenyl] piperazine | 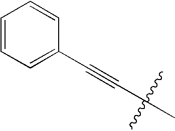 | | 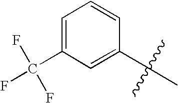 |
| 86 | 2-{4-[3- (phenylethynyl)benzoyl] piperazin-1-yl}pyrazine | 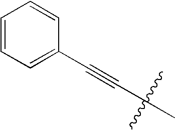 | | 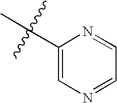 |
| 87 | 1-{[5-(phenylethynyl)pyridin- 3-yl]carbonyl}-4-pyridin-2- ylpiperazine | 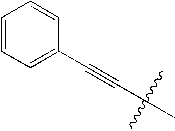 | X₂ = N | 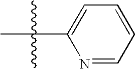 |
| 88 | 1-[4-methyl-3- (phenylethynyl)benzoyl]-4- pyridin-2-ylpiperazine | 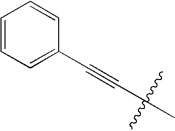 | R₁ = F | 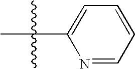 |
| 89 | 1-[4-fluoro-3- (phenylethynyl)benzoyl]-4- pyridin-2-ylpiperazine | 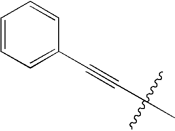 | R₁ = OCH₃ | 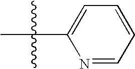 |
| 90 | 1-[4-methoxy-3- (phenylethynyl)benzoyl]-4- pyridin-2-ylpiperazine | 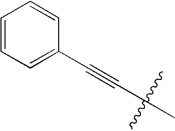 | R₁ = OCH₃ | 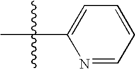 |
| 91 | 1-[2-methyl-3- (phenylethynyl)benzoyl]-4- pyridin-2-ylpiperazine | 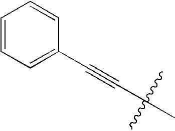 | R₁ = CH₃ | 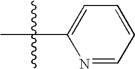 |
| 92 | 1-pyridin-2-yl-4-[3-(pyridin-2- ylethynyl)benzoyl]piperazine | 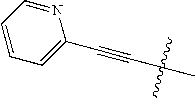 | | 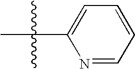 |
| 93 | 1-pyridin-2-yl-4-[3-(pyridin-3- ylethynyl)benzoyl]piperazine | 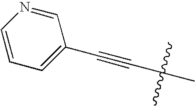 | | 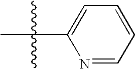 |
| 94 | 2-{4-[3-(pyridin-2- ylethynyl)benzoyl]piperazin- 1-yl}pyrazine | 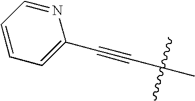 | | 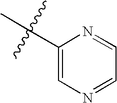 |
| 95 | 1-pyridin-2-yl-4-[3-(pyridin-2- ylethynyl)benzoyl]piperazine | 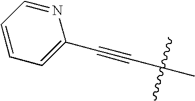 | | 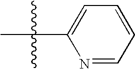 |
| 96 | 6-{4-[3-(pyridin-2- ylethynyl)benzoyl]piperazin- 1-yl}nicotinonitrile | 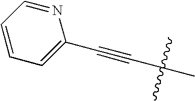 | | 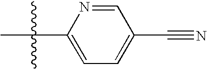 |
| 97 | 1-[3-(pyridin-2- ylethynyl)benzoyl]-4-(1H- tetrazol-5-yl)piperazine | 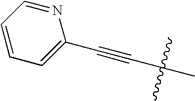 | | 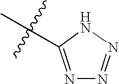 |
| 98 | 2-methyl-4-pyridin-2-yl-1-[3- (pyridin-2- ylethynyl)benzoyl]piperazine | 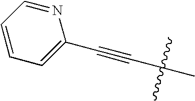 | R₄ = CH₃ | 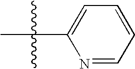 |
| 99 | 2-{4-[3-(pyridin-2- ylethynyl)benzoyl]piperazin- 1-yl}pyrimidin-5-ol | 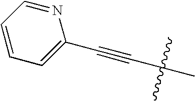 | | 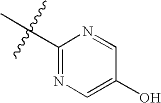 |
| 100 | 4,6-dimethyl-2-{4-[3-(pyridin- 2- ethynyl)benzoyl]piperazin- 1-yl}pyrimidine | 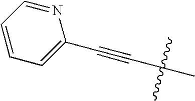 | | 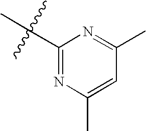 |
| 101 | 1-(3-methoxypyridin-2-yl)-4- [3-(pyridin-2- ylethynyl)benzoyl]piperazine | 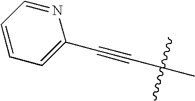 | | 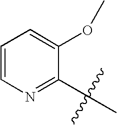 |
| 102 | 1-(4-methoxy-1,2,5- thiadiazol-3-yl)-4-[3-(pyridin- 2- ylethynyl)benzoyl]piperazine | 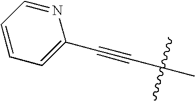 | | 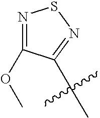 |
| 103 | 2,5-dimethyl-3-{4-[3-(pyridin- 2- ylethynyl)benzoyl]piperazin- 1-yl}pyrazine | 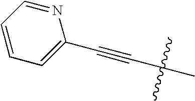 | | 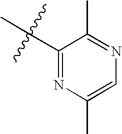 |
| 104 | 2-chloro-6-{4-[3-(pyridin-2- ylethynyl)benzoyl]piperazin- 1-yl}pyrazine | 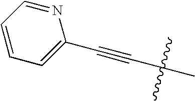 | | 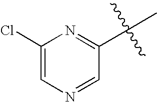 |
| 105 | 2-methyl-1-pyridin-2-yl-4-[3- (pyridin-2-ylethynyl)benzoyl] piperazine (R₅ = CH₃) | 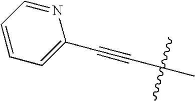 | | 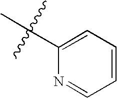 |
| 106 | 4-phenyl-2-{4-[3-(pyridin-2- ylethynyl)benzoyl]piperazin- 1-yl}pyrimidine | 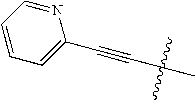 | | 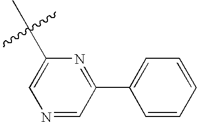 |
| 107 | 2-{4-[3-(phenylethynyl) benzoyl]piperazin-1-yl} pyrimidine | 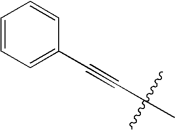 | | 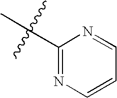 |
| 108 | 1-[3-(phenylethynyl) benzoyl]-4-[5-(trifluoro methyl)pyridin-2-yl] piperazine | 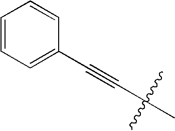 | | 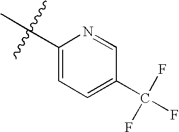 |
| 109 | 1-(4-methylpyridin-2-yl)-4-[3- (phenylethynyl)benzoyl] piperazine | 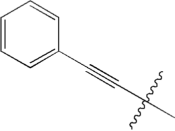 | | 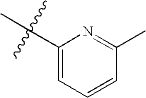 |
| 110 | 1-(6-methylpyridin-2-yl)-4-[3- (phenylethynyl)benzoyl] piperazine | 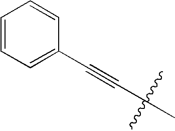 | | 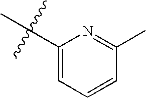 |
| 111 | 4,6-dimethyl-2-{4-[3- (phenylethynyl)benzoyl] piperazin-1-yl}pyrimidine | 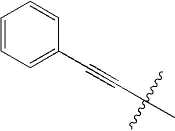 | | 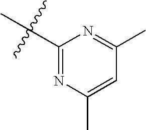 |
| 112 | 2-{4-[4-methoxy-3- (phenylethynyl)benzoyl] piperazin-1-yl}pyrimidine | 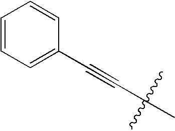 | X₁ = COMe | 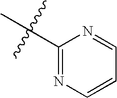 |
| 113 | 6-{4-[4-methoxy-3- (phenylethynyl)benzoyl] piperazin-1-yl}nicotinonitrile | 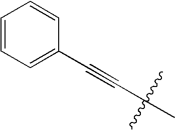 | X₁ = COMe | 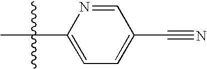 |
| 114 | 1-[4-methoxy-3- (phenylethynyl)benzoyl]-4- [5-(trifluoromethyl)pyridin-2- yl]piperazine | 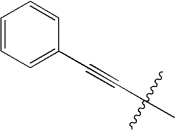 | X₁ = COMe | 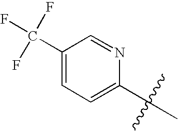 |
| 115 | 1-[4-methoxy-3-(phenyl ethynyl)benzoyl]-4-(4- methylpyridin-2- yl]piperazine | 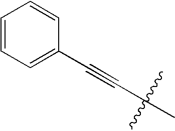 | X₁ = COMe | 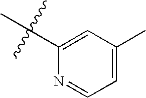 |
| 116 | 1-[4-methoxy-3-(phenyl ethynyl)benzoyl]-4-(6- methylpyridin-2- yl]piperazine | 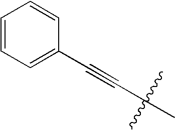 | X₁ = COMe | 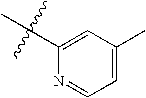 |
| 117 | 1-[4-methoxy-3-(phenyl ethynyl)benzoyl]-4-[6- (trifluoromethyl)pyridin-2- yl]piperazine | 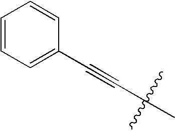 | X₁ = COMe | 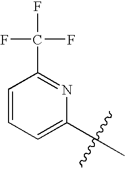 |
| 118 | 2-{4-[4-methoxy-3-(phenyl ethynyl)benzoyl]piperazin-1- yl}nicotinonitrile | 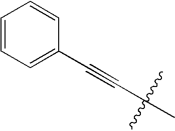 | X₁ = COMe | 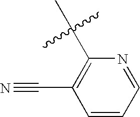 |
| 119 | 2-{4-[4-methoxy-3-(phenyl ethynyl)benzoyl]piperazin-1- yl}-4,6-dimethylpyrimidine | 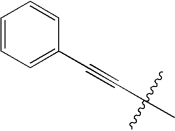 | X₁ = COMe | 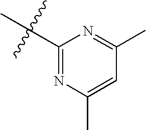 |
| 120 | 2-{4-[2-(phenylethynyl) isonicotinoyl]piperazin-1- yl}pyrimidine | 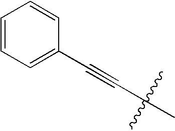 | X₂ = N | 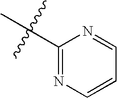 |
| 121 | 1-[2-(phenylethynyl) isonicotinoyl]-4-pyridin-2- ylpiperazine | 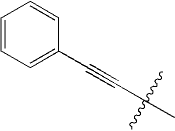 | X₂ = N | 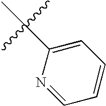 |
| 122 | 6-{4-[2-(phenylethynyl) isonicotinoyl]piperazin-1- yl}nicotinonitrile | 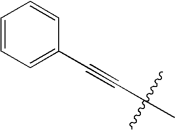 | X₂ = N | 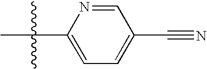 |
| 123 | 1-[2-(phenylethynyl) isonicotinoyl]-4-[5- (trifluoromethyl)pyridin-2- yl]piperazine | 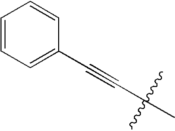 | X₂ = N | 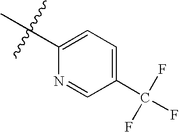 |
| 124 | 1-(4-methylpyridin-2-yl)-4-[2- (phenylethynyl)isonicotinoyl] piperazine | 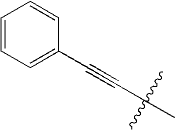 | X₂ = N | 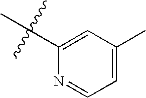 |
| 125 | 1-(6-methylpyridin-2-yl)-4-[2- (phenylethynyl)isonicotinoyl] piperazine | 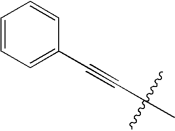 | X₂ = N | 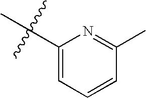 |
| 126 | 1-[2-(phenylethynyl) isonicotinoyl]-4-[6- (trifluoromethyl)pyridin-2- yl]piperazine | 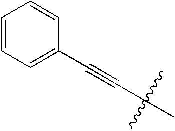 | X₂ = N | 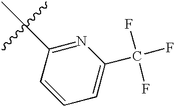 |
| 127 | 2-{4-[2-(phenylethynyl) isonicotinoyl]piperazin-1- yl}pyrazine | 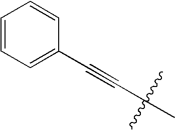 | X₂ = N | 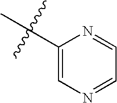 |
| 128 | 6-{4-[3-(phenylethynyl) benzoyl]piperazin-1- yl}nicotinonitrile | 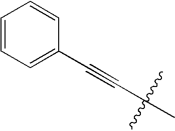 | | 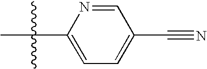 |
| 129 | 1-[3- (phenylethynyl)benzoyl]-4- [3-(trifluoromethyl)pyridin-2- yl]piperazine | 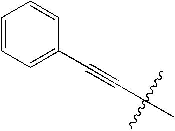 | | 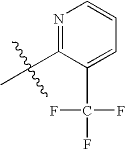 |
| 130 | 2-(4-{[5- (phenylethynyl)pyridin-3- yl]carbonyl}piperazin-1- yl)pyrimidine | 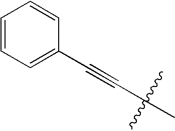 | | 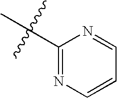 |
| 131 | 6-(4-{[5- (phenylethynyl)pyridin-3- yl]carbonyl}piperazin-1- yl)nicotinonitrile | 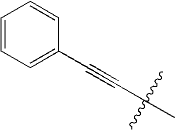 | | 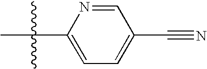 |
| 132 | 1-{[5-(phenylethynyl)pyridin- 3-yl]carbonyl}-4-[5- (trifluoromethyl)pyridin-2- yl]piperazine | 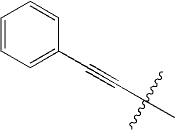 | | 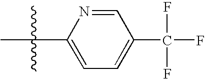 |
| 133 | 1-(4-methylpyridin-2-yl)-4- {[5-(phenylethynyl)pyridin-3- yl]carbonyl}piperazine | 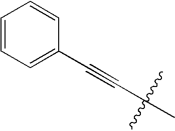 | | 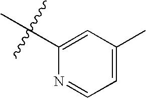 |
| 134 | 1-{[5-(phenylethynyl)pyridin- 3-yl]carbonyl}-4-[3- (trifluoromethyl)pyridin-2- yl]piperazine | 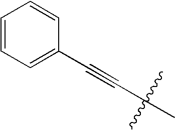 | | 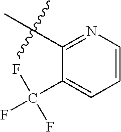 |
| 135 | 2-(4-{[5-(phenyl ethynyl)pyridin-3- yl]carbonyl}piperazin-1- yl)nicotinonitrile | 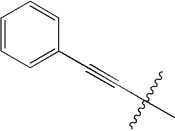 | | 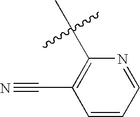 |
| 136 | 4,6-dimethyl-2-(4-{[5- (phenylethynyl)pyridin-3- yl]carbonyl}piperazin-1- yl)pyrimidine | 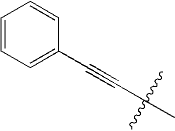 | | 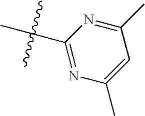 |
| 137 | 2-(4-{[5- (phenylethynyl)pyridin-3- yl]carbonyl}piperazin-1- yl)pyrazine | 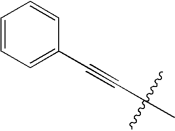 | | 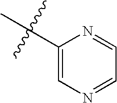 |
| 138 | 1-{[5-(phenylethynyl)pyridin- 3-yl]carbonyl}-4-pyridin-4- ylpiperazine | 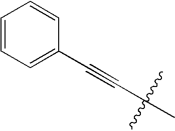 | | 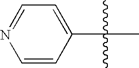 |
| 139 | 2-{4-[2- (phenylethynyl)isonicotinoyl] piperazin-1-yl}nicotinonitrile | 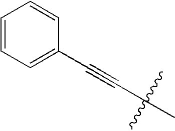 | | 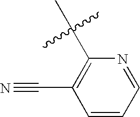 |
| 140 | 4,6-dimethyl-2-{4-[2- (phenylethynyl)isonicotinoyl] piperazin-1-yl}pyrimidine | 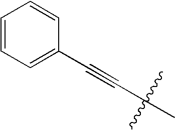 | X₁ = N | 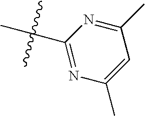 |
| 141 | 2-{4-[3- (phenylethynyl)benzoyl] piperazin-1-yl}nicotinonitrile | 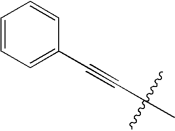 | | 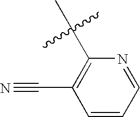 |
| 142 | 1-[3- (phenylethynyl)benzoyl]-4- pyridin-4-ylpiperazine | 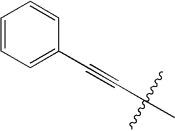 | | 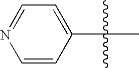 |
| 143 | 1-(6-methylpyridin-2-yl)-4- {(5-(phenylethynyl)pyridin-3- yl]carbonyl}piperazine | 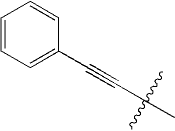 | X₂ = N | 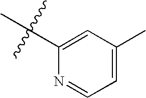 |
| 144 | 1-[2- (phenylethynyl)isonicotinoyl]- 4-pyridin-4-ylpiperazine | 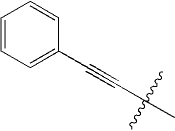 | X₁ = N | 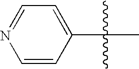 |
| 145 | 1-[4-methoxy-3- (phenylethynyl)benzoyl]- 4-pyridin-4-ylpiperazine | 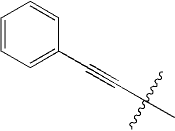 | | 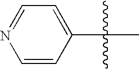 |
| 146 | 1-[3-(pyridin-2- ylethynyl)benzoyl]-4-[3- (trifluoromethyl)pyridin-2- yl]piperzine | 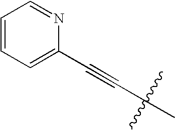 | | 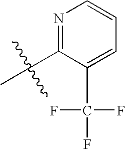 |
| 147 | 1-(6-methylpyridin-2-yl)-4-[3- (pyridin-2- ylethynyl)benzoyl]piperazine | 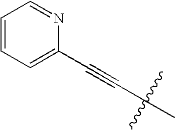 | | 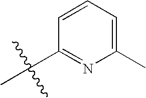 |
| 148 | 2-{4-[3-pyridin-2- ylethynyl)beznoyl]piperazin- 1-yl}nicotinonitrile | 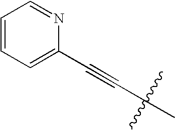 | | 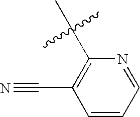 |
| 149 | 1-[3-chloro-5- (trifluoromethyl)pyridin-2-yl]- 4-[3-(pyridin-2- ylethynyl)benzoyl]piperazine | 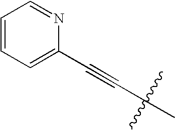 | | 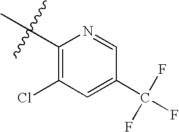 |

TABLE 2A

| | | LCMS Data | | | Biological Activity | |
|---|---|---|---|---|---|---|
| | | Time | | | Median Ki | PCT INHIB (%) |
| Cmpd | Name | (min.) | Mass | Ion | (μM) | @ 10 μM |
| 69 | 1-[3-(phenylethynyl)benzoyl]- | 1.99 | 368.2 | M + H | 0.15473 | 95 |
| | 4-pyridin-2-yl | | | | | |
| | piperazine | | | | | |
| 70 | 1-methyl-4-[3-(phenylethynyl)benzoyl]piperazine | 1.82 | 305.2 | M + H | | 0 |
| 71 | 1-(4-methoxyphenyl)-4-[3- | 2.43 | 397.2 | M + H | | 40 |
| | (phenylethynyl)benzoyl]piperazine | | | | | |
| 72 | 1-(4-chlorophenyl)-4-[3- | 2.59 | 401.1 | M + H | | 30 |
| | (phenylethynyl)benzoyl]piperazine | | | | | |
| 73 | 1-(4-methylphenyl)-4-[3- | 2.56 | 381.2 | M + H | | 0 |
| | (phenylethynyl)benzoyl]piperazine | | | | | |
| 74 | 1-(4-{4-[3-(phenylethynyl)benzoyl]piperazin- | 2.4 | 409.2 | M + H | | 20 |
| | 1-yl}phenyl)ethanone | | | | | |
| 75 | 1-(4-nitrophenyl)-4-[3- | 2.45 | 412.2 | M + H | | 20 |
| | (phenylethynyl)benzoyl]piperazine | | | | | |
| 76 | 1-phenyl-4-[3-(phenylethynyl)benzoyl]piperazine | 2.5 | 367.2 | M + H | 1.287 | 86 |
| 77 | 1-[3-(phenylethynyl)benzoyl]- | 2.6 | 435.2 | M + H | | 0 |
| | 4-[4-(trifluoromethyl)phenyl]piperazine | | | | | |
| 78 | 1-(2,6-dimethylphenyl)-4- | 2.7 | 395.2 | M + H | >10.000 | 53 |
| | [3-(phenylethynyl)benzoyl]piperazine | | | | | |
| 79 | 1-(4-fluorophenyl)-4-[3- | 2.5 | 385.2 | M + H | | 28 |
| | (phenylethynyl)benzoyl]piperazine | | | | | |
| 80 | 1-[2-(methylthio)phenyl]-4- | 2.61 | 413.2 | M + H | | 24 |
| | [3-(phenylethynyl)benzoyl]piperazine | | | | | |
| 81 | 1-(3-methoxyphenyl)-4-[3- | 2.48 | 397.2 | M + H | 3.389 | 78 |
| | (phenylethynyl)benzoyl]piperazine | | | | | |
| 82 | 1-(3-chlorophenyl)-4-[3- | 2.59 | 401.1 | M + H | 2.774 | 58 |
| | (phenylethynyl)benzoyl]piperazine | | | | | |
| 83 | 4-{4-[3-(phenylethynyl)benzoyl]piperazin- | 2.18 | 383.2 | M + H | >10.000 | 74 |
| | 1-yl}phenol | | | | | |
| 84 | 1-(3,4-dichlorophenyl)-4- | 2.67 | 435.1 | M + H | | 0 |
| | [3-(phenylethynyl)benzoyl]piperazine | | | | | |
| 85 | 1-[3-(phenylethynyl)benzoyl]- | 2.6 | 435.2 | M + H | | 32 |
| | 4-[3-(trifluoromethyl)phenyl]piperazine | | | | | |
| 86 | 2-{4-[3-(phenylethynyl)benzoyl]piperazin- | 2.37 | 369.2 | M + H | 0.444 | 85 |
| | 1-yl}pyrazine | | | | | |
| 87 | 1-{[5-(phenylethynyl)pyridin- | 1.95 | 369.2 | M + H | 3.312 | 56 |
| | 3-yl]carbonyl}-4- | | | | | |
| | pyridin-2-ylpiperazine | | | | | |
| 88 | 1-[4-methyl-3-(phenylethynyl)benzoyl]- | 2.22 | 382.2 | M + H | 0.059 | 84 |
| | 4-pyridin- | | | | | |
| | 2-ylpiperazine | | | | | |
| 89 | 1-[4-fluoro-3-(phenylethynyl)benzoyl]- | 2.15 | 386.2 | M + H | 0.05 | 51 |
| | 4-pyridin- | | | | | |
| | 2-ylpiperazine | | | | | |
| 90 | 1-[4-methoxy-3-(phenylethynyl)benzoyl]- | 2.06 | 398.2 | M + H | 0.025 | 83 |
| | 4-pyridin- | | | | | |
| | 2-ylpiperazine | | | | | |
| 91 | 1-[2-methyl-3-(phenylethynyl)benzoyl]- | 2.2 | 382.2 | M + H | | 18 |
| | 4-pyridin- | | | | | |
| | 2-ylpiperazine | | | | | |
| 92 | 1-pyridin-2-yl-4-[3-(pyridin- | 1.8 | 369.2 | M + H | 0.04852 | 98 |
| | 2-ylethynyl)benzoyl]piperazine | | | | | |
| 93 | 1-pyridin-2-yl-4-[3-(pyridin- | 1.83 | 369.2 | M + H | | 26 |
| | 3-ylethynyl)benzoyl]piperazine | | | | | |
| 94 | 2-{4-[3-(pyridin-2-yl | 2.23 | 370.2 | M + H | 0.39535 | 79 |
| | ethynyl)benzoyl]piperazin- | | | | | |
| | 1-yl}pyrazine | | | | | |
| 95 | 1-pyridin-4-yl-4-[3-(pyridin- | 1.74 | 369.2 | M + H | | 30 |
| | 2-ylethynyl)benzoyl]piperazine | | | | | |
| 96 | 6-{4-[3-(pyridin-2-yl | 2.29 | 394.2 | M + H | 3.23792 | 50 |
| | ethynyl)benzoyl]piperazin- | | | | | |
| | 1-yl}nicotinonitrile | | | | | |
| 97 | 1-[3-(pyridin-2-ylethynyl)benzoyl]- | 1.84 | 360.1 | M + H | | 0 |
| | 4-(1H-tetrazol-5- | | | | | |
| | yl)piperazine | | | | | |
| 98 | 2-methyl-4-pyridin-2-yl-1- | 1.84 | 383.2 | M + H | 0.67926 | 72 |
| | [3-(pyridin-2-ylethynyl)benzoyl]piperazine | | | | | |
| 99 | 2-{4-[3-(pyridin-2-yl | 1.89 | 386.2 | M + H | 1.45242 | 64 |
| | ethynyl)benzoyl]piperazin- | | | | | |
| | 1-yl}pyrimidin-5-ol | | | | | |
| 100 | 4,6-dimethyl-2-{4-[3- | 2.31 | 398.2 | M + H | 2.16349 | 55 |
| | (pyridin-2-ylethynyl)benzoyl]piperazine- | | | | | |
| | 1-yl}pyrimidine | | | | | |
| 101 | 1-(3-methoxypyridin-2-yl)- | 2.08 | 399.2 | M + H | 2.34784 | 52 |
| | 4-[3-(pyridin-2-ylethynyl)benzoyl]piperazine | | | | | |
| 102 | 1-(4-methoxy-1,2,5- | 2.41 | 406.1 | M + H | 0.08 | 92 |
| | thiadiazol-3-yl)-4-[3- | | | | | |
| | (pyridin-2-ylethynyl)benzoyl]piperazine | | | | | |
| 103 | 2,5-dimethyl-3-{4-[3- | 2.41 | 398.2 | M + H | | 16 |
| | (pyridin-2-ylethynyl)benzoyl]piperazin- | | | | | |
| | 1- | | | | | |
| | yl}pyrazine | | | | | |
| 104 | 2-chloro-6-{4-[3-(pyridin-2- | 2.26 | 404.1 | M + H | 1.46122 | 71 |
| | ylethynyl)benzoyl]piperazin- | | | | | |
| | 1-yl}pyrazine | | | | | |
| 105 | 2-methyl-1-pyridin-2-yl-4- | 1.83 | 383.2 | M + H | 0.8261 | 58 |
| | [3-(pyridin-2-ylethynyl)benzoyl]piperazine | | | | | |
| 106 | 4-phenyl-2-{4-[3-(pyridin- | 2.64 | 446.2 | M + H | 0.00219 | 86 |
| | 2-ylethynyl)benzoyl]piperazin- | | | | | |
| | 1-yl}pyrimidine | | | | | |
| 107 | 2-{4-[3-(phenylethynyl)benzoyl]piperazin- | 369.3 | 2.8 | M + H | 0.45101 | 90 |
| | 1-yl}pyrimidine | | | | | |
| 108 | 1-[3-(phenylethynyl)benzoyl]- | 436.4 | 3 | M + H | | 0 |
| | 4-[5-(trifluoromethyl)pyridin- | | | | | |
| | 2-yl]iperazine | | | | | |
| 109 | 1-(4-methylpyridin-2-yl)-4- | 382.3 | 3 | M + H | 0.06506 | 91 |
| | [3-(phenylethynyl)benzoyl]piperazine | | | | | |
| 110 | 1-(6-methylpyridin-2-yl)-4- | 382.3 | 3 | M + H | 3.41435 | 68 |
| | [3-(phenylethynyl)benzoyl]piperazine | | | | | |
| 111 | 4,6-dimethyl-2-{4-[3- | 397.4 | 3.1 | M + H | | 0 |
| | (phenylethynyl)benzoyl]piperazin- | | | | | |
| | 1-yl}pyrimidine | | | | | |
| 112 | 2-{4-[4-methoxy-3- | 399.3 | 2.8 | M + H | 0.06247 | 90 |
| | (phenylethynyl)benzoyl]piperazin- | | | | | |
| | 1-yl}pyrimidine | | | | | |
| 113 | 6-{4-[4-methoxy-3- | 423.4 | 2.7 | M + H | 0.35656 | 81 |
| | (phenylethynyl)benzoyl]piperazin- | | | | | |
| | 1-yl}nicotinonitrile | | | | | |
| 114 | 1-[4-methoxy-3- | 466.4 | 3 | M + H | | 26 |
| | (phenylethynyl)benzoyl]-4- | | | | | |
| | [5-(trifluoromethyl)pyridin- | | | | | |
| | 2-yl]piperazine | | | | | |
| 115 | 1-[4-methoxy-3-(phenylethynyl)benzoyl]- | 412.4 | 2.9 | M + H | 0.00315 | 93 |
| | 4-(4- | | | | | |
| | methylpyridin-2-yl)piperazine | | | | | |
| 116 | 1-[4-methoxy-3-(phenylethynyl)benzoyl]- | 412.4 | 2.9 | M + H | 0.85296 | 77 |
| | 4-(6- | | | | | |
| | methylpyridin-2-yl)piperazine | | | | | |
| 117 | 1-[4-methoxy-3-(phenylethynyl)benzoyl]- | 466.4 | 3 | M + H | | 35 |
| | 4-[6- | | | | | |
| | (trifluoromethyl)pyridin-2- | | | | | |
| | yl]piperazine | | | | | |
| 118 | 2-{4-[4-methoxy-3- | 423.4 | 2.8 | M + H | 1.75877 | 78 |
| | (phenylethynyl)benzoyl]piperazin- | | | | | |
| | 1-yl}nicotinonitrile | | | | | |
| 119 | 2-{4-[4-methoxy-3-(phenylethynyl)benzoyl]piperazin- | 427.4 | 3 | M + H | | 35 |
| | 1-yl}-4,6-dimethyl | | | | | |
| | pyrimidine | | | | | |
| 120 | 2-{4-[2-(phenylethynyl)isonicotinoyl]piperazin- | 370.4 | 2.6 | M + H | 2.88919 | 49 |
| | 1- | | | | | |
| | yl}pyrimidine | | | | | |
| 121 | 1-[2-(phenylethynyl)isonicotinoyl]- | 369.4 | 2.7 | M + H | 1.12941 | 72 |
| | 4-pyridin-2- | | | | | |
| | ylpiperazine | | | | | |
| 122 | 6-{4-[2-(phenylethynyl)isonicotinoyl]piperazin- | 394.4 | 2.6 | M + H | | 0 |
| | 1- | | | | | |
| | yl}nicotinonitrile | | | | | |
| 123 | 1-[2-(phenylethynyl)isonicotinoyl]- | 437.4 | 2.8 | M + H | | 0 |
| | 4-[5- | | | | | |
| | (trifluoromethyl)pyridin-2- | | | | | |
| | yl]piperazine | | | | | |
| 124 | 1-(4-methylpyridin-2-yl)-4- | 383.4 | 2.8 | M + H | 0.33843 | 84 |
| | [2-(phenylethynyl)isonicotinoyl]piperazine | | | | | |
| 125 | 1-(6-methylpyridin-2-yl)-4- | 383.4 | 2.8 | M + H | | 30 |
| | [2-(phenylethynyl)isonicotinoyl]piperazine | | | | | |
| 126 | 1-[2-(phenylethynyl)isonicotinoyl]- | 437.4 | 2.9 | M + H | | 54 |
| | 4-[6- | | | | | |
| | (trifluoromethyl)pyridin-2- | | | | | |
| | yl]piperazine | | | | | |
| 127 | 2-{4-[2-(phenylethynyl)isonicotinoyl]piperazin- | 370.4 | 2.5 | M + H | | 36 |
| | 1- | | | | | |
| | yl}pyrazine | | | | | |
| 128 | 6-{4-[3-(phenylethynyl)benzoyl]piperazin- | 393.3 | 2.8 | M + H | | 48 |
| | 1-yl}nicotinonitrile | | | | | |
| 129 | 1-[3-(phenylethynyl)benzoyl]- | 436.4 | 3.1 | M + H | | 45 |
| | 4-[3-(trifluoromethyl)pyridin- | | | | | |
| | 2-yl]piperazine | | | | | |
| 130 | 2-(4-{[5-(phenylethynyl)pyridin- | 370.4 | 2.7 | M + H | | 47 |
| | 3- | | | | | |
| | yl]carbonyl}piperazin-1- | | | | | |
| | yl)pyrimidine | | | | | |
| 131 | 6-(4-{[5-(phenylethynyl)pyridin- | 394.4 | 2.6 | M + H | | 0 |
| | 3- | | | | | |
| | yl]carbonyl}piperazin-1- | | | | | |
| | yl)nicotinonitrile | | | | | |
| 132 | 1-{[5-(phenylethynyl)pyridin- | | | | | 0 |
| | 3-yl]carbonyl}-4-[5- | | | | | |
| | (trifluoromethyl)pyridin-2- | | | | | |
| | yl]piperazine | | | | | |
| 133 | 1-(4-methylpyridin-2-yl)-4- | 383.4 | 2.8 | M + H | 0.65119 | 52 |
| | {[5-(phenylethynyl)pyridin- | | | | | |
| | 3-yl]carbonyl}piperazine | | | | | |
| 134 | 1-{[5-(phenylethynyl)pyridin- | 437.4 | 2.9 | M + H | | 0 |
| | 3-yl]carbonyl}-4-[3- | | | | | |
| | (trifluoromethyl)pyridin-2- | | | | | |
| | yl]piperazine | | | | | |
| 135 | 2-(4-{[5-(phenylethynyl)pyridin- | 394.4 | 2.7 | M + H | | 28 |
| | 3-yl]carbonyl}piperazin- | | | | | |
| | 1-yl)nicotinonitrile | | | | | |
| 136 | 4,6-dimethyl-2-(4-{[5- | 398.4 | 2.9 | M + H | | 0 |
| | (phenylethynyl)pyridin-3- | | | | | |
| | yl]carbonyl}piperazin-1- | | | | | |
| | yl)pyrimidine | | | | | |
| 137 | 2-(4-{[5-(phenylethynyl)pyridin- | 370.3 | 2.6 | M + H | | 32 |
| | 3-yl]carbonyl}piperazin- | | | | | |
| | 1-yl)pyrazine | | | | | |
| 138 | 1-{[5-(phenylethynyl)pyridin- | 369.4 | 3.4 | M + H | | 0 |
| | 3-yl]carbonyl}-4- | | | | | |
| | pyridin-4-ylpiperazine | | | | | |
| 139 | 2-{4-[2-(phenylethynyl)isonicotinoyl]piperazin- | 394.4 | 2.6 | M + H | | 40 |
| | 1- | | | | | |
| | yl}nicotinonitrile | | | | | |
| 140 | 4,6-dimethyl-2-{4-[2- | 398.4 | 2.9 | M + H | | 37 |
| | (phenylethynyl)isonicotino | | | | | |
| | yl]piperazin-1-yl}pyrimidine | | | | | |
| 141 | 2-{4-[3-(phenylethynyl)benzoyl]piperazin- | 393.3 | 2.8 | M + H | 1.08866 | 78 |
| | 1-yl}nicotinonitrile | | | | | |
| 142 | 1-[3-(phenylethynyl)benzoyl]- | 368.4 | 3.5 | M + H | | 53 |
| | 4-pyridin-4-yl | | | | | |
| | piperazine | | | | | |
| 143 | 1-(6-methylpyridin-2-yl)-4- | 383.4 | 2.9 | M + H | | 49 |
| | {[5-(phenylethynyl)pyridin- | | | | | |
| | 3-yl]carbonyl}piperazine | | | | | |
| 144 | 1-[2-(phenylethynyl)isonicotinoyl]- | 369.4 | 3.1 | M + H | | 10 |
| | 4-pyridin-4- | | | | | |
| | ylpiperazine | | | | | |
| 145 | 1-[4-methoxy-3-(phenylethynyl)benzoyl]- | 398.4 | 3.4 | M + H | 1.29949 | 91 |
| | 4-pyridin- | | | | | |
| | 4-ylpiperazine | | | | | |
| 146 | 1-[3-(pyridin-2-ylethynyl)benzoyl]- | 2.5 | 437.2 | M + H | | 48 |
| | 4-[3-(trifluoromethyl)pyridin- | | | | | |
| | 2-yl]piperazine | | | | | |
| 147 | 1-(6-methylpyridin-2-yl)-4- | 1.83 | 383.2 | M + H | 3.01907 | 80 |
| | [3-(pyridin-2-ylethynyl)benzoyl]piperazine | | | | | |
| 148 | 2-{4-[3-(pyridin-2-yl | 2.27 | 394.2 | M + H | 1.62992 | 74 |
| | ethynyl)benzoyl]piperazin- | | | | | |
| | 1-yl}nicotinonitrile | | | | | |
| 149 | 1-[3-chloro-5-(trifluoromethyl)pyridin- | 2.64 | 471.1 | M + H | 2.76451 | 72 |
| | 2-yl]-4-[3- | | | | | |
| | (pyridin-2-ylethynyl)benzoyl]piperazine | | | | | |

### Example 3

### 1-[3-(benzyloxy)benzoyl]-4-pyridin-2-ylpiperazin (Compound 153)

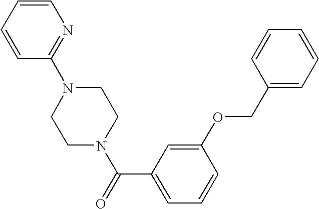

### Step 1: (3-hydroxyphenyl)(4-(pyridin-2-yl)piperazin-1-yl)methanone

To 1-(pyridin-2-yl)piperazine (5.18 mmol) and 3-hydroxybenzoic acid (5.18 mmol) was added 50 ml DMF. To this mixture was added HOBT (6.48 mmol), 1-(3-(dimethylamino)propyl)-3-ethyl-carbodiimide hydrochloride (WSCDI) (6.48 mmol), followed by DIEA (10.37 mmol). The solution was stirred for 16 hours at which time LCMS indicated the reaction was complete. 200 mL Water and 150 mL EtOAc were added to the solution. The organic layer was collected, dried with Na₂SO₄, and concentrated in vacuo giving 0.83 g of (3-hydroxyphenyl)(4-(pyridin-2-yl)piperazin-1-yl)methanone compound as an off white solid that was triturated with Et₂O. LCMS Rt=0.29 min (MS=284)

### Step 2: 1-[3-(benzyloxy)benzoyl]-4-pyridin-2-ylpiperazin

2 ml DMF was added to cesium carbonate (0.265 mmol) and (3-hydroxyphenyl)(4-(pyridin-2-yl)piperazin-1-yl)methanone (0.176 mmol). This mixture was heated to 35° C. for 30 minutes and (bromomethyl)benzene (0.194 mmol) was added to the mixture. The mixture was stirred for 16 hours at 35° C. The mixture was concentrated on a speedvac and purified via prep HPLC (Gilson with NH₄OH additive) giving 30 mg of 1-[3-(benzyloxy)benzoyl]-4-pyridin-2-ylpiperazin. LCMS Rt=1.95 min (MS=374).

Compounds 150-163, shown in Tables 3 and 3A below, were prepared using the procedure of Example 3 described above.

### THE FOLLOWING VALUES REFER TO FORMULA I

### WHEREIN R₁, R₄, R₄ₐ, R₅, R₅ₐ═H; X₂═CH; Z=CO

TABLE 3

| Cmpd | Name | R₂ | X₁ | R₆ |
|---|---|---|---|---|
| 150 | 1-{3-[(3- methylbenzyl)oxy]benzoyl}-4- pyridin-2-ylpiperazine | 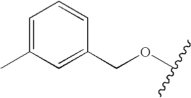 | CH | 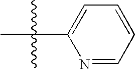 |
| 151 | 1-{4-methoxy-3-[(3- methylbenzyl)oxy]benzoyl}-4- pyridin-2-ylpiperazine | 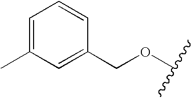 | COMe | 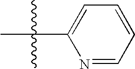 |
| 152 | 1-[3-(benzyloxy)benzoyl]-4- pyridin-2-ylpiperazine | 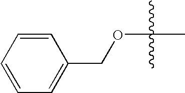 | CH | 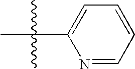 |
| 153 | 1-{3-[(3- bromobenzyl)oxy]benzoyl}-4- pyridin-2-ylpiperazine | 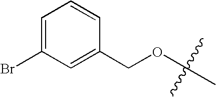 | CH | 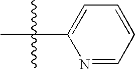 |
| 154 | 1-{3-[(3- chlorobenzyl)oxy]benzoyl}-4- pyridin-2-ylpiperazine | 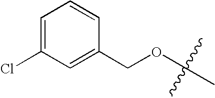 | CH | 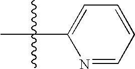 |
| 155 | 1-{3-[(3- methoxybenzyl)oxy]benzoyl}- 4-pyridin-2-ylpiperazine | 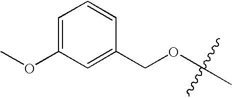 | CH | 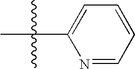 |
| 156 | 3-({3-[(4-pyridin-2- ylpiperazin-1- yl)carbonyl]phenoxy}methyl) benzonitrile | 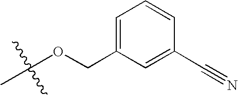 | CH | 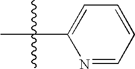 |
| 157 | 1-{3-[(3- fluorobenzyl)oxy]benzoyl}-4- pyridin-2-ylpiperazine | 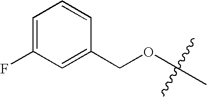 | CH | 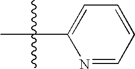 |
| 158 | 1-[3-(benzyloxy)-4- methoxybenzoyl]-4-pyridin-2- ylpiperazine | 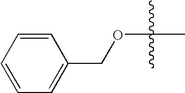 | COMe | 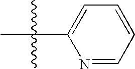 |
| 159 | 1-{3-[(3-bromobenzyl)oxy]-4- methoxybenzoyl}-4-pyridin-2- ylpiperazine | 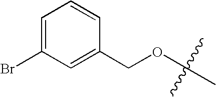 | COMe | 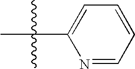 |
| 160 | 1-{3-[(3-chlorobenzyl)oxy]-4- methoxybenzoyl}-4-pyridin-2- ylpiperazine | 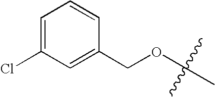 | COMe | 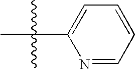 |
| 161 | 1-{4-methoxy-3-[(3- methoxybenzyl)oxy]benzoyl}- 4-pyridin-2-ylpiperazine | 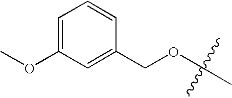 | COMe | 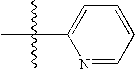 |
| 162 | 3-({2-methoxy-5-[(4-pyridin- 2-ylpiperazin-1- yl)carbonyl]phenoxy}methyl) benzonitrile | 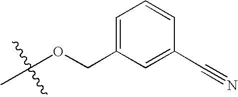 | COMe | 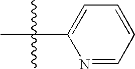 |
| 163 | 1-{3-[(3-fluorobenzyl)oxy]-4- methoxybenzoyl}-4-pyridin-2- ylpiperazine | 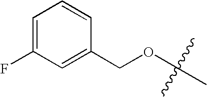 | COMe | 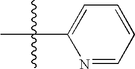 |

TABLE 3A

| | | LCMS Data | | | Biological Activity |
|---|---|---|---|---|---|
| | | Time | | | PCT INHIB |
| Cmpd | Name | (min.) | Mass | Ion | (%) @ 10 μM |
| 150 | 1-{3-[(3- | 2.04 | 388.2 | M + H | 0 |
| | methylbenzyl)oxy]benzoyl}-4- | | | | |
| | pyridin-2-ylpiperazine | | | | |
| 151 | 1-{4-methoxy-3-[(3- | 1.99 | 418.2 | M + H | 0 |
| | methylbenzyl)oxy]benzoyl}-4- | | | | |
| | pyridin-2-ylpiperazine | | | | |
| 152 | 1-[3-(benzyloxy)benzoyl]-4- | 1.95 | 374.2 | M + H | 0 |
| | pyridin-2-ylpiperazine | | | | |
| 153 | 1-{3-[(3- | 2.11 | 452.1 | M + H | 23 |
| | bromobenzyl)oxy]benzoyl}-4- | | | | |
| | pyridin-2-ylpiperazine | | | | |
| 154 | 1-{3-[(3- | 2.07 | 408.1 | M + H | 36 |
| | chlorobenzyl)oxy]benzoyl}-4- | | | | |
| | pyridin-2-ylpiperazine | | | | |
| 155 | 1-{3-[(3- | 1.96 | 404.2 | M + H | 0 |
| | methoxybenzyl)oxy]benzoyl}-4- | | | | |
| | pyridin-2-ylpiperazine | | | | |
| 156 | 3-({3-[(4-pyridin-2-ylpiperazin-1- | 1.9 | 399.2 | M + H | 0 |
| | yl)carbonyl]phenoxy}methyl)benzonitrile | | | | |
| 157 | 1-{3-[(3- | 1.98 | 392.2 | M + H | 18 |
| | fluorobenzyl)oxy]benzoyl}-4- | | | | |
| | pyridin-2-ylpiperazine | | | | |
| 158 | 1-[3-(benzyloxy)-4- | 1.89 | 404.2 | M + H | 0 |
| | methoxybenzoyl]-4-pyridin-2- | | | | |
| | ylpiperazine | | | | |
| 159 | 1-{3-[(3-bromobenzyl)oxy]-4- | 2.05 | 482.1 | M + H | 26 |
| | methoxybenzoyl}-4-pyridin-2- | | | | |
| | ylpiperazine | | | | |
| 160 | 1-{3-[(3-chlorobenzyl)oxy]-4- | 2.02 | 438.2 | M + H | 11 |
| | methoxybenzoyl}-4-pyridin-2- | | | | |
| | ylpiperazine | | | | |
| 161 | 1-{4-methoxy-3-[(3- | 1.91 | 434.2 | M + H | 0 |
| | methoxybenzyl)oxy]benzoyl}-4- | | | | |
| | pyridin-2-ylpiperazine | | | | |
| 162 | 3-({2-methoxy-5-[(4-pyridin-2- | 1.86 | 429.2 | M + H | 0 |
| | ylpiperazin-1- | | | | |
| | yl)carbonyl]phenoxy}methyl)benzonitrile | | | | |
| 163 | 1-{3-[(3-fluorobenzyl)oxy]-4- | 1.94 | 422.2 | M + H | 11 |
| | methoxybenzoyl}-4-pyridin-2- | | | | |
| | ylpiperazine | | | | |

### Example 4

### 1-[3-(phenoxymethyl)benzoyl]-4-pyridin-2-ylpiperazin (Compound 171)

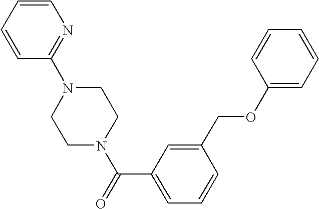

### Step 1: 3-(chloromethyl)phenyl)(4-(pyridin-2-yl)piperazin-1-yl)methanone

3-(chloromethyl)benzoyl chloride (5.29 mmol) was added to a solution of 1-(pyridin-2-yl)piperazine (5.29 mmol) and TEA (5.29 mmol) in 50 mL DCM cooled to 0° C. The reaction was stirred at room temperature for 5 hours at which time LCMS indicated the reaction was complete. The reaction was washed with 100 mL water, 100 mL saturated NaHCO₃, and 100 mL dilute HCl. The organic layer was dried with Na₂SO₄ and concentrated in vacuo producing 0.98 g (3-(chloromethyl)phenyl)(4-(pyridin-2-yl)piperazin-1-yl)methanone as a slightly yellow oily solid. LCMS Rt=0.61 min (MS=316).

### Step 2: 1-[3-(phenoxymethyl)benzoyl]-4-pyridin-2-ylpiperazin

A solution of potassium carbonate (0.277 mmol) and phenol (0.277 mmol) in DMF (1.5 ml) was prepared and stirred for 25 minutes. To this was added a solution of (3-(chloromethyl)phenyl)(4-(pyridin-2-yl)piperazin-1-yl)methanone (0.222 mmol) in DMF (1.5 ml). After stirring for 30 minutes at room temperature, the reaction was heated to 40° C. and stirred for 16 hours. The reaction was concentrated on a speedvac and purified via prep HPLC (Gilson with TFA additive) producing 37 mg of 1-[3-(phenoxymethyl)benzoyl]-4-pyridin-2-ylpiperazin. LCMS Rt=1.86 min (MS=374)

Compounds 164-171, shown in Tables 4 and 4A below, were prepared using the procedure of Example 4 described above.

### THE FOLLOWING VALUES REFER TO FORMULA I

### WHEREIN R₁, R₄, R₄ₐ, R₅, R₅ₐ═H; X₁, X₂═CH; Z=CO

TABLE 4

| Cmpd | Name | R₂ | R₆ |
|---|---|---|---|
| 164 | 1-{3-[(3-methylphenoxy)methyl] benzoyl}-4-pyridin-2-yl piperazine | 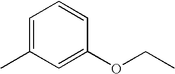 | 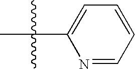 |
| 165 | 3-({3-[(4-pyridin-2-ylpiperazin- 1-yl)carbonyl]benzyl}oxy) benzonitrile | 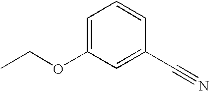 | 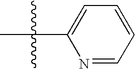 |
| 166 | 1-{3-[(3- ethylphenoxy)methyl]benzoyl}- 4-pyridin-2-ylpiperazine | 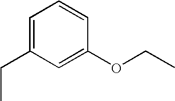 | 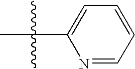 |
| 167 | 1-{3-[(3- methoxyphenoxy)methyl]benzoyl}- 4-pyridin-2-ylpiperazine | 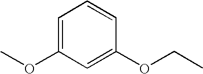 | 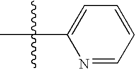 |
| 168 | 1-{3-[(3- chlorophenoxy)methyl]benzoyl}- 4-pyridin-2-ylpiperazine | 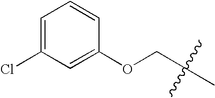 | 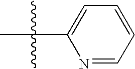 |
| 169 | 1-{3-[(3- bromophenoxy)methyl]benzoyl}- 4-pyridin-2-ylpiperazine | 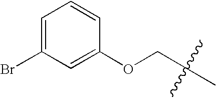 | 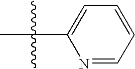 |
| 170 | 1-pyridin-2-yl-4-{3-[(pyridin-2- yloxy)methyl]benzoyl} piperazine | 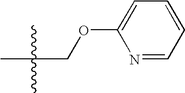 | 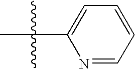 |
| 171 | 1-[3-(phenoxymethyl)benzoyl]- 4-pyridin-2-ylpiperazine | 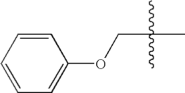 | 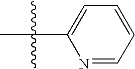 |

TABLE 4A

| | | LCMS Data | | | |
|---|---|---|---|---|---|
| | | Time | | | Biological Activity |
| Cmpd | Name | (min.) | Mass | Ion | Median Ki (μM) |
| 164 | 1-{3-[(3-methylphenoxy)methyl]benzoyl}- | 1.98 | 388.2 | M + H | 1.747 |
| | 4-pyridin-2-ylpiperazine | | | | |
| 165 | 3-({3-[(4-pyridin-2-ylpiperazin-1- | 1.83 | 399.2 | M + H | 2.966 |
| | yl)carbonyl]benzyl}oxy)benzonitrile | | | | |
| 166 | 1-{3-[(3-ethylphenoxy)methyl]benzoyl}- | 2.07 | 402.2 | M + H | >10.00 |
| | 4-pyridin-2-ylpiperazine | | | | |
| 167 | 1-{3-[(3-methoxyphenoxy)methyl]benzoyl}- | 1.88 | 404.2 | M + H | >10.00 |
| | 4-pyridin-2-ylpiperazine | | | | |
| 168 | 1-{3-[(3-chlorophenoxy)methyl]benzoyl}- | 2.02 | 408.1 | M + H | 0.813 |
| | 4-pyridin-2-ylpiperazine | | | | |
| 169 | 1-{3-[(3-bromophenoxy)methyl]benzoyl}- | 2.05 | 452.1 | M + H | 0.862 |
| | 4-pyridin-2-ylpiperazine | | | | |
| 170 | 1-pyridin-2-yl-4-{3-[(pyridin-2- | 1.46 | 375.2 | M + H | >10.00 |
| | yloxy)methyl]benzoyl}piperazine | | | | |
| 171 | 1-[3-(phenoxymethyl)benzoyl]-4- | 1.86 | 374.2 | M + H | >10.00 |
| | pyridin-2-ylpiperazine | | | | |

### Example 5

### 3-({3-[(4-pyridin-2-ylpiperazin-1-yl)sulfonyl]phenyl}ethynyl)phenol (Compound 172)

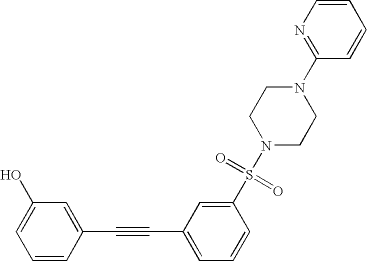

### Step 1: 3-bromo-N-(2-(ethyl(pyridin-2-yl)amino)ethyl)-N-methylbenzenesulfonamide

To a solution of 1-(pyridin-2-yl)piperazine (6.21 mmol) and TEA (6.71 mmol) in 30 mL DCM was added dropwise a solution of 3-bromobenzene-1-sulfonyl chloride (6.21 mmol) in 10 mL DCM. The reaction was stirred at room temperature for 16 hours. The reaction was diluted with 20 mL DCM, washed with 30 mL water, 20 mL 5 percent (%) aq. K₂CO₃ solution, and brine. The organic layer was dried with MgSO4 and concentrated in vacuo producing 3-bromo-N-(2-(ethyl(pyridin-2-yl)amino)ethyl)-N-methylbenzenesulfonamide as a white solid used without further purification.

### Step 2: 3-({3-[(4-pyridin-2-ylpiperazin-1-yl)sulfonyl]phenyl}ethynyl)phenol

To a solution of 3-bromo-N-(2-(ethyl(pyridin-2-yl)amino)ethyl)-N-methylbenzenesulfonamide (0.13 mmol) and 3-hydroxyphenylacetylene (0.19 mmol) in DMF (2 mL) in a microwave vial was added copper iodide (0.026 mmol) and TEA (0.39 mmol). To the suspension was added Pd(PPh₃)₂Cl₂ (0.026 mmol). The vial was purged with N₂, capped, and microwaved for 10 minutes at 150° C. The product was concentrated on a speedvac and purified via prep HPLC (Gilson with TFA additive) to produce 3-({3-[(4-pyridin-2-ylpiperazin-1-yl)sulfonyl]phenyl}ethynyl)phenol. LCMS Rt=2.07 min (MS=420).

Compounds 172-176, shown in Table 5 below, were prepared using the procedure of Example 5 described above.

### THE FOLLOWING VALUES REFER TO FORMULA I

### WHEREIN R₁, R₄, R₄ₐ, R₅, R₅ₐ═H; X₁, X₂═CH; Z=SO₂

TABLE 5

| | | | | | | | Biological |
|---|---|---|---|---|---|---|---|
| | | | | LCMS Data | | | Activity |
| | | | | Time | | | PCT INHIB |
| Cmpd | Name | R₂ | R₆ | (min.) | Mass | Ion | (%) @ 10 μM |
| 172 | 3-({3-[(4-pyridin-2- ylpiperazin-1-yl)sulfonyl] phenyl}ethynyl)phenol | 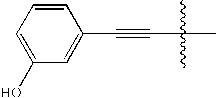 | 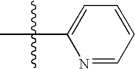 | 2.07 | 420.1 | M + H | 0 |
| 173 | 1-{[3-(cyclohex-1-en-1- ylethynyl)phenyl]sulfonyl}- 4-pyridin-2-ylpiperazine | 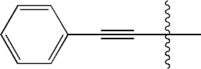 | 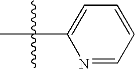 | 2.46 | 408.2 | M + H | 0 |
| 174 | 1-{[3-(3-phenylprop-1-yn- 1-yl)phenyl]sulfonyl}-4- pyridin-2-ylpiperazine | 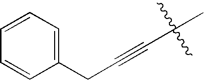 | 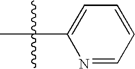 | 2.02 | 418.2 | M + H | 0 |
| 175 | 1-({3-[(3-methoxyphenyl) ethynyl]phenyl}sulfonyl)- 4-pyridin-2-ylpiperazine | 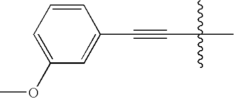 | 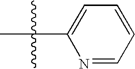 | 2.34 | 434.1 | M + H | 0 |
| 176 | 1-{[3-(phenylethynyl) phenyl]sulfonyl}-4-pyridin- 2-ylpiperazine | 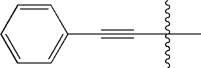 | 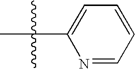 | 2.32 | 404.1 | M + H | 0 |

### Example 6

### 3-({3-[(4-pyridin-2-ylpiperazin-1-yl)methyl]phenyl}ethynyl)phenol (Compound 177)

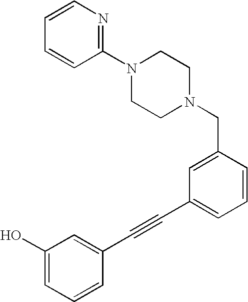

### Step 1: 1-(3-bromobenzyl)-4-(pyridin-2-yl)piperazine

To a solution of 1-(pyridin-2-yl)piperazine (6.1 mmol) and DIEA (18.4 mmol) in 20 mL THF was added 1-bromo-3-(bromomethyl)benzene (7.4 mmol). The reaction was stirred at room temperature for 16 hours at which time LCMS indicated the reaction was complete. The reaction was diluted with 50 mL EtOAc and washed with 10 mL saturated NH₄Cl, 10 mL water, and 50 mL brine. The organic layer was dried over MgSO₄ and concentrated in vacuo. Purification via silica column chromatography (Hex:EtOac as eluent) produced 1-(3-bromobenzyl)-4-(pyridin-2-yl)piperazine.

### Step 2: 3-({3-[(4-pyridin-2-ylpiperazin-1-yl)methyl]phenyl}ethynyl)phenol

To a solution of 1-(3-bromobenzyl)-4-(pyridin-2-yl)piperazine (0.15 mmol) and 3-hydroxyphenylacetylene (0.23 mmol) in DMF (2 mL) was added copper iodide (0.03 mmol) and TEA (0.45 mmol). To the suspension was added Pd(PPh₃)₂Cl₂ (0.03 mmol). The vial was purged with N₂, capped, and microwaved for 10 minutes at 150° C. The reaction was concentrated on a speedvac and purified via prep HPLC (Gilson with TFA additive) producing 3-({3-[(4-pyridin-2-ylpiperazin-1-yl)methyl]phenyl}ethynyl)phenol. LCMS Rt=1.84 min (MS=370).

Compounds 177-181, shown in Table 6 below, were prepared using the procedure of Example 6 described above.

### THE FOLLOWING VALUES REFER TO FORMULA I

### WHEREIN R₁, R₄, R₄ₐ, R₅, R₅ₐ═H; X₁, X₂═CH; Z=CH₂

TABLE 6

| | | | | | | | Biological |
|---|---|---|---|---|---|---|---|
| | | | | LCMS Data | | | Activity |
| | | | | Time | | | PCT INHIB |
| Cmpd | Name | R₂ | R₆ | (min.) | Mass | Ion | (%) @ 10 μM |
| 177 | 3-({3-[(4-pyridin-2- ylpiperazin-1-yl)methyl] phenyl}ethynyl)phenol | 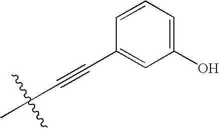 | 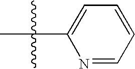 | 1.84 | 370.2 | M + H | 13 |
| 178 | 1-[3-(cyclohex-1-en-1- ylethynyl)benzyl]-4- pyridin-2-ylpiperazine | 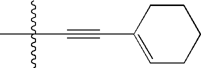 | 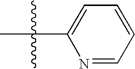 | 2.15 | 358.2 | M + H | 0 |
| 179 | 1-[3-(3-phenylprop-1- yn-1-yl)benzyl]-4- pyridin-2-ylpiperazine | 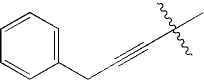 | 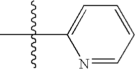 | 2.14 | 368.2 | M + H | 0 |
| 180 | 3-({3-[(4-pyridin-2- ylpiperazin-1-yl)methyl] phenyl}ethynyl)aniline | 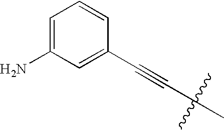 | 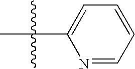 | 1.74 | 369.2 | M + H | 0 |
| 181 | 1-{3-[(3-methoxy phenyl)ethynyl]benzyl}- 4-pyridin-2-ylpiperazine | 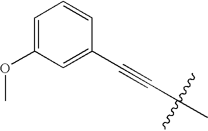 | 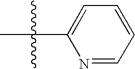 | 1.98 | 384.2 | M + H | 0 |

### Example 7

### 1-{4-methoxy-3-[(E)-2-phenylvinyl]benzoyl}-4-pyridin-2-ylpiperazin (Compound 182)

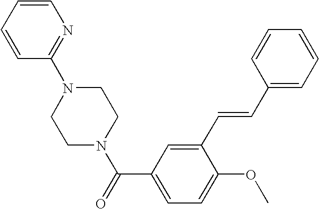

To a solution of (3-bromo-4-methoxyphenyl)(4-(pyridin-2-yl)piperazin-1-yl)methanone (0.2 mmol; as synthesized in Example 1) in NMP (1 mL) was added N,N dimethyl glycine (0.02 mmol), K₂CO₃ (0.4 mmol), styrene (0.3 mmol), and Pd(OAc)₂ (0.02 mmol). The vial was purged with N₂, capped, and heated to 130° C. for 18 hours. The reaction was concentrated on a speedvac and purified via prep HPLC (Gilson with TFA additive) to produce 1-{4-methoxy-3-[(E)-2-phenylvinyl]benzoyl}-4-pyridin-2-ylpiperazin. LCMS Rt=2.15 min (MS=400.2).

The properties of Compound 182 are shown in Tables 7 and 7A below.

### THE FOLLOWING VALUES REFER TO FORMULA I

### R₁, R₄, R₄ₐ, R₅, R₅ₐ═H; X₂═H; Z=CO

TABLE 7

| Cmpd | Name | R₂ | X₁ | R₆ |
|---|---|---|---|---|
| 182 | 1-{4-methoxy-3-[(E)-2- phenylvinyl]benzoyl}-4- pyridin-2-ylpiperazine | 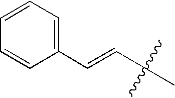 | COMe | 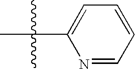 |

TABLE 7A

| | | | | | Biological | |
|---|---|---|---|---|---|---|
| | | | | | Activity | |
| | | | | | | PCT |
| | | LCMS Data | | | Median | INHIB |
| | | Time | | | Kᵢ | (%) @ |
| Cmpd | Name | (min.) | Mass | Ion | (μM) | 10 μM |
| 182 | 1-{4-methoxy- | 2.15 | 400.2 | M + H | 0.82716 | 86 |
| | 3-[(E)-2- | | | | | |
| | phenylvinyl]- | | | | | |
| | benzoyl}-4- | | | | | |
| | pyridin-2- | | | | | |
| | ylpiperazine | | | | | |

Compounds 183-291, shown in Table 8 and 8A below, were prepared using the procedure of Example 2 described above.

### UNLESS NOTED OTHERWISE THE FOLLOWING VALUES REFER TO FORMULA I

### WHEREIN R₁, R₄, R₄ₐ, R₅, R₅ₐ═H; X₁═COMe, X₂═CH; Z=CO

TABLE 8

| Cmpd | Name | R₂ | Noted Values | R₆ |
|---|---|---|---|---|
| 183 | 1-[4-methoxy-3- (pyridin-2-ylethynyl) benzoyl]-4-[5- (trifluoromethyl)pyridin- 2-yl]piperazine | 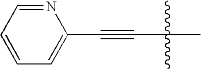 | | 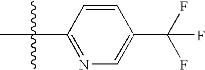 |
| 184 | 1-[4-methoxy-3- (pyridin-2-ylethynyl) benzoyl]-4-[3- (trifluoromethyl)pyridin- 2-yl]piperazine | 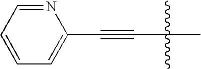 | | 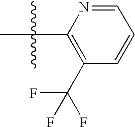 |
| 185 | 1-(3,5-dichloro pyridin-2-yl)-4-[4- methoxy-3-(pyridin-2- ylethynyl) benzoyl]piperazine | 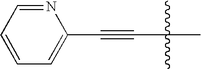 | | 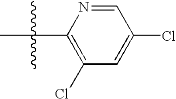 |
| 186 | 1-(3-chloropyridin-2- yl)-4-[4-methoxy-3- (pyridin-2-yl ethynyl) benzoyl]piperazine | 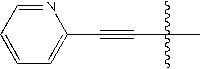 | | 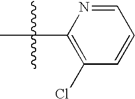 |
| 187 | 1-[4-methoxy-3- (pyridin-2-ylethynyl) benzoyl]-4-[3- (trifluoromethyl) phenyl]piperazine | 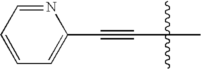 | | 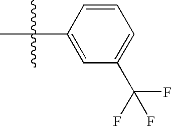 |
| 188 | 1-(5-chloropyridin-2- yl)-4-[4-methoxy-3- (pyridin-2-yl_ethynyl) benzoyl]piperazine | 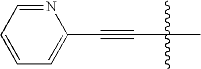 | | 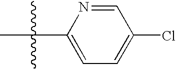 |
| 189 | 1-(3-chlorophenyl)-4- [4-methoxy-3- (pyridin-2-ylethynyl) benzoyl]piperazine | 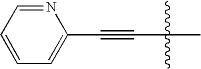 | | 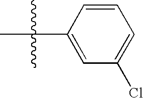 |
| 190 | 1-[3-chloro-5- (trifluoromethyl)pyridin- 2-yl]-4-[4-methoxy- 3-(pyridin-2-ylethynyl) benzoyl]piperazine | 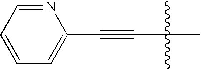 | | 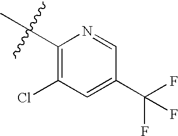 |
| 191 | 1-[4-methoxy-3- (pyridin-2-ylethynyl) benzoyl]-4-(4-methyl pyridin-2-yl)piperazine | 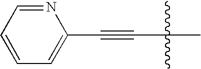 | | 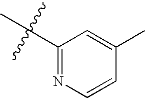 |
| 192 | 2-{4-[4-methoxy-3- (pyridin-2-ylethynyl) benzoyl]piperazin-1- yl}-4,6-dimethyl pyrimidine | 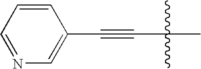 | | 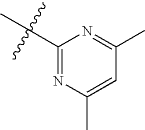 |
| 193 | 3-{4-[4-methoxy-3- (pyridin-2-ylethynyl) benzoyl]piperazin-1- yl}pyrazine-2- carbonitrile | 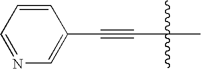 | | 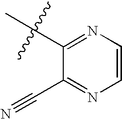 |
| 194 | 2-{4-[4-methoxy-3- (pyridin-2-ylethynyl) benzoyl]piperazin-1- yl}-4-(trifluoro methyl)pyrimidine | 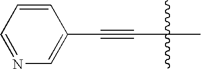 | | 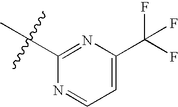 |
| 195 | 3-{4-[4-methoxy-3- (pyridin-2-ylethynyl) benzoyl]piperazin-1- yl}phenol | 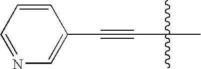 | | 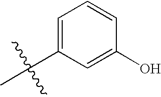 |
| 196 | 1-[4-methoxy-3- (pyridin-2-ylethynyl) benzoyl]-4-(3-methyl- phenyl)piperazine | 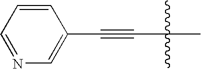 | | 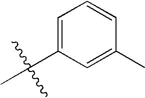 |
| 197 | 5-bromo-4-methoxy_- 2-{4-[4-methoxy-3- (pyridin-2-ylethynyl) benzoyl]piperazin-1- yl}pyrimidine | 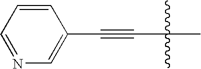 | | 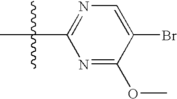 |
| 198 | 1-[4-methoxy-3- (pyridin-2-ylethynyl) benzoyl]-4-(6- methylpyridin-2- yl)piperazine | 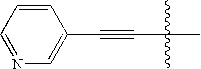 | | 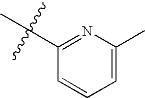 |
| 199 | (1R,4S)-2-(4- chlorophenyl)-5-{4- methoxy-3-(pyridin-2- ylethynyl)_benzoyl]- 2,5-diazabicyclo [2.2.1]_heptane | 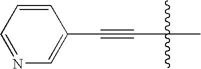 | R₄ₐ/R₅ = Bridging Methylene | 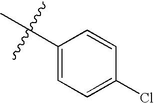 |
| 200 | 4-methoxy-2-{4-[4- methoxy-3-(pyridin-2- ylethynyl)benzoyl] piperazin-1-yl} pyrimidine | 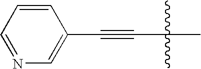 | | 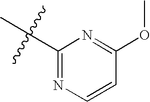 |
| 201 | 3-{4-[4-methoxy-3- (pyridin-2-ylethynyl) benzoyl]piperazin-1- yl}-1,2-benziso thiazole | 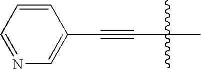 | | 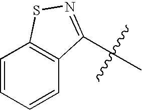 |
| 202 | 6-{4-[4-methoxy-3- (pyridin-2-ylethynyl) benzoyl]-1,4- diazepan-1-yl} nicotinonitrile | 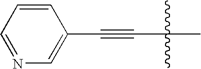 | | 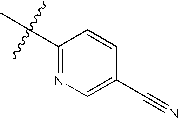 |
| 203 | 1-[4-methoxy-3- (pyridin-2-ylethynyl) benzoyl]-4-(5- methylpyridin-2-yl) piperazine | 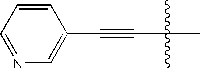 | | 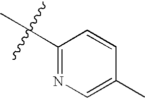 |
| 204 | 2-{4-[4-methoxy-3- (pyridin-2-ylethynyl) benzoyl]piperazin-1- yl}-6-methyl_pyrazine | 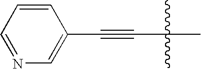 | | 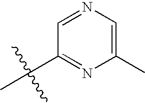 |
| 205 | 1-[4-methoxy-3- (pyridin-2-ylethynyl) benzoyl]-4-pyridin-2- yl-1,4-diazepane [n = 2] | 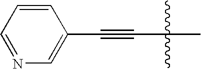 | | 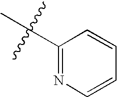 |
| 206 | 1-[4-methoxy-3- (pyridin-2-ylethynyl) benzoyl]-4-[5- (trifluoromethyl)- 1,3,4-thiadiazol-2- yl]piperazine | 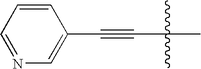 | | 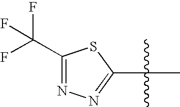 |
| 207 | (1R,4S)-2-(3- fluorophenyl)-5-[4- methoxyl-3-(pyridin-2- ylethynyl)_benzoyl]- 2,5-diazabicyclo [2.2.1]heptane | 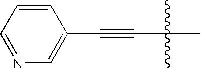 | R₄ₐ/R₅ = Bridging Methylene | 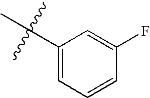 |
| 208 | 1-[4-methoxy-3- (pyridin-2-ylethynyl) benzoyl]-4-(3-methyl pyridin-2-yl) piperazine | 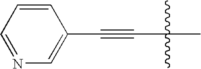 | | 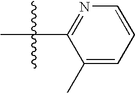 |
| 209 | 1-[4-methoxy-3- (pyridin-2-ylethynyl) benzoyl]-4-[3- (trifluoromethyl)pyridin- 2-yl]-1,4-diazepane | 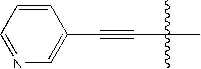 | | 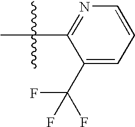 |
| 210 | 1-[4-methoxy-3- (pyridin-2-ylethynyl) benzoyl]-4-[5- n-2-yl]-1,4-diazepane [n = 2] | 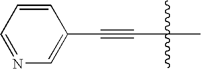 | | 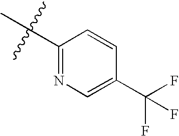 |
| 211 | 1-[4-methoxy-3- (pyridin-2-ylethynyl) benzoyl]-4-(6- methylpyridin-2-yl)- 1,4-diazepane | 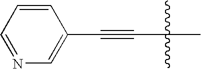 | | 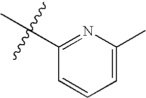 |
| 212 | 1-[3-chloro-5- (trifluoromethyl)pyridin- 2-yl]-4-[4-methoxy- 3-(pyridin-2-ylethynyl) benzoyl]-1,4-diazepane [n = 2] | 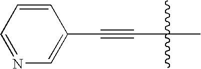 | | 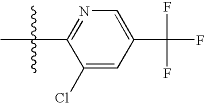 |
| 213 | 1-(6-methoxy_pyridin- 2-yl)-4-[4-methoxy-3- (pyridin-2-ylethynyl) benzoyl]_piperazine | 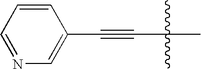 | | 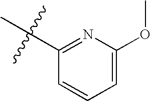 |
| 214 | 2-{4-[4-methoxy-3- (pyridin-2-ylethynyl) benzoyl]-1,4-diazepan- 1-yl} nicotinonitrile [n = 2] | 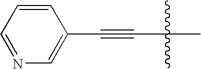 | | 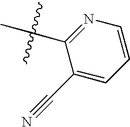 |
| 215 | 1-[4-methoxy-3- (pyridin-2-ylethynyl) benzoyl]-4-(5-nitro- pyridin-2-yl)-1,4- diazepane [n = 2] | 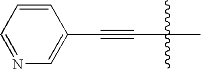 | | 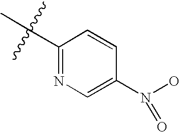 |
| 216 | 1-(2-chlorophenyl)-4- [4-methoxy-3- (pyridin-2-ylethynyl) benzoyl]piperazine | 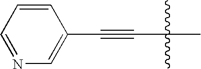 | | 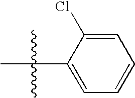 |
| 217 | 1-(4-chlorophenyl)-4- [4-methoxy-3- (pyridin-2-ylethynyl) benzoyl]piperazine | 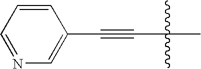 | | 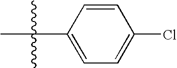 |
| 218 | 1-(3,4-dichloro phenyl)-4-[4-methoxy- 3-(pyridin-2-yl ethynyl)benzoyl] piperazine | 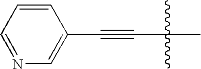 | | 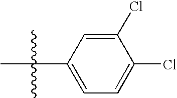 |
| 219 | 1-(2,3-dimethyl phenyl)-4-[4- methoxy-3-(pyridin-2- ylethynyl)benzoyl] piperazine | 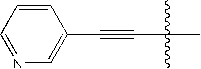 | | 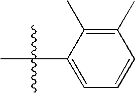 |
| 220 | 2-isopropyl-4-{4-[4- methoxy-3-(pyridin-2- ylethynyl)benzoyl] piperazin-1-yl}-6- methylpyrimidine | 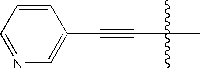 | | 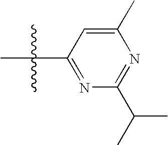 |
| 221 | 1-[4-methoxy-3- (pyridin-2-ylethynyl) benzoyl]-4-(2- methylphenyl) piperazine | 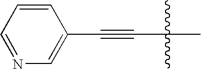 | | 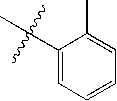 |
| 222 | 1-[4-methoxy-3- (pyridin-2-ylethynyl) benzoyl]-4-(4-methyl phenyl) piperazine | 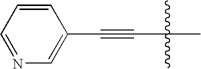 | | 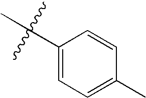 |
| 223 | 1-(3-fluorophenyl)-4- [4-methoxy-3- (pyridin-2-ylethynyl) benzoyl]piperazine | 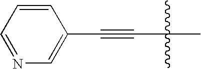 | | 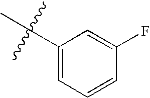 |
| 224 | 1-[4-methoxy-3- (pyridin-2-ylethynyl) benzoyl]-4-(phenyl sulfonyl)piperazine | 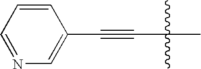 | | 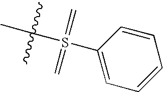 |
| 225 | 1-[(5-chloro-2- thienyl)sulfonyl]-4-[4- methoxy-3-(pyridin-2- ylethynyl)_benzoyl] piperazine | 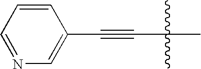 | | 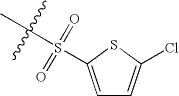 |
| 226 | (1R,4S)-2-[4- methoxy-3-(pyridin-2- ylethynyl)benzoyl]-5- (4-methylphenyl)-2,5- diazabicyclo_[2.2.1] heptane | 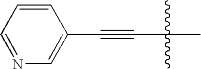 | R₄ₐ/R₅ = Bridging Methylene | 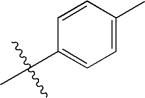 |
| 227 | (1S,4R)-2-(4- fluorophenyl)-5-[4- methoxy-3-(pyridin-2- ylethynyl)benzoyl]_- 2,5-diazabicyclo [2.2.1]heptane | 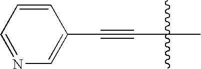 | R₄ₐ/R₅ = Bridging Methylene | 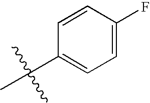 |
| 228 | 1-[4-methoxy-3- (pyridin-2-ylethynyl) benzoyl]-4-[4- (trifluoromethyl)phenyl] piperazine | 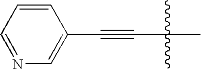 | | 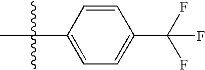 |
| 229 | 1-[4-methoxy-3- (pyridin-2-ylethynyl) benzoyl]-4-(5-nitro- pyridin-2-yl)piperazine | 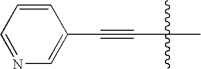 | | 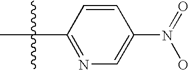 |
| 230 | 1-(2-methoxy phenyl)-4- [4-methoxy-3-(pyridin-2- ylethynyl)benzoyl] piperazine | 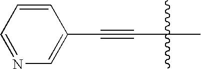 | | 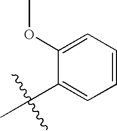 |
| 231 | 1-(4-fluorophenyl)-4- [4-methoxy-3- (pyridin-2-ylethynyl) benzoyl]piperazine | 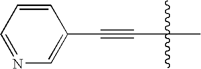 | | 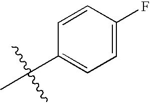 |
| 232 | 1-[4-methoxy-3- (pyridin-2-ylethynyl) benzoyl]-4-(4-nitro phenyl)piperazine | 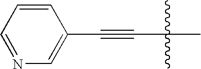 | | 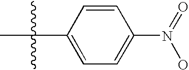 |
| 233 | 1-(4-methoxy phenyl)-4- [4-methoxy-3-(pyridin-2- ylethynyl)benzoyl] piperazine | 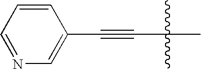 | | 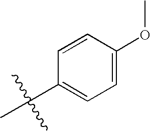 |
| 234 | 1-(benzylsulfonyl)-4- [4-methoxy-3- (pyridin-2-ylethynyl) benzoyl]piperazine | 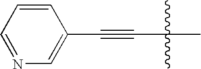 | | 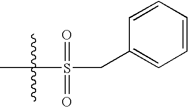 |
| 235 | 1-(2,3-dihydro-1,4- benzodioxin-6- ylsulfonyl)-4-[4- methoxy-3-(pyridin-2- ylethynyl)benzoyl] piperazine | 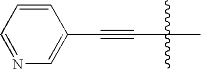 | | 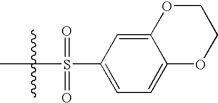 |
| 236 | 1-[4-methoxy-3- (pyridin-2-ylethynyl) benzoyl]-4-pyridin-4- ylpiperazine | 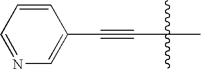 | | 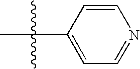 |
| 237 | 1-4-{4-[4-methoxy-3- (pyridin-2-ylethynyl) benzoyl]piperazin-1- yl}phenyl)ethanone | 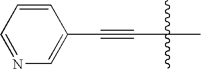 | | 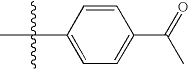 |
| 238 | 1-[4-methoxy-3- (pyridin-2-ylethynyl) benzoyl]-4-[4-(methyl sulfonyl)phenyl] piperazine | 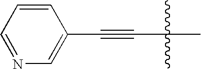 | | 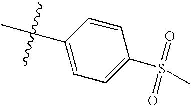 |
| 239 | 1-[(3,4-dichloro phenyl)sulfonyl]-4-[4- methoxy-3-(pyridin-2- ylethynyl)benzoyl] piperazine | 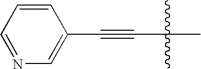 | | 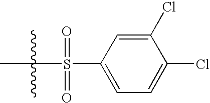 |
| 240 | 1-[4-fluoro-2- (methylsulfonyl)phenyl]- 4-[4-methoxy-3- (pyridin-2-ylethynyl) benzoyl]piperazine | 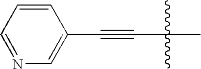 | | 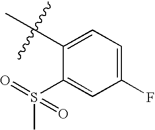 |
| 241 | 1-(3-methoxy phenyl)-4- [4-methoxy-3-(pyridin-2- yl ethynyl)benzoyl] piperazine | 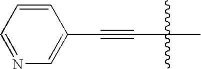 | | 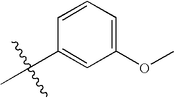 |
| 242 | 1-(2,5-dimethyl phenyl)- 4-[4-methoxy-3-(pyridin- 2-yl ethynyl)benzoyl] piperazine | 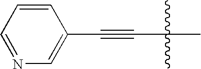 | | 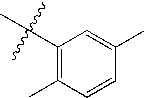 |
| 243 | 1-[(4-chlorophenyl) sulfonyl]-4-[4- methoxy-3-(pyridin-2- ylethynyl)benzoyl] piperazine | 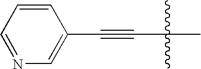 | | 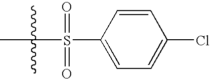 |
| 244 | 1-benzoyl-4-[4- methoxy-3-(pyridin-2- ylethynyl)benzoyl] piperazine | 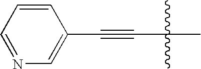 | | 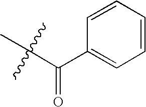 |
| 245 | 1-(ethylsulfonyl)-4-[4- methoxy-3-(pyridin-2- ylethynyl) benzoyl] piperazine | 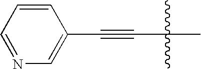 | | 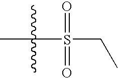 |
| 246 | 1-[4-methoxy-3- (pyridin-2-ylethynyl) benzoyl]-4-[2- (trifluoromethyl)phenyl] piperazine | 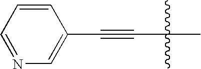 | | 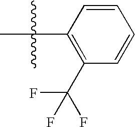 |
| 247 | 1-[4-methoxy-3- (pyridin-2-yl ethynyl) benzoyl]-4-(1,3- thiazol-2-yl) piperazine | 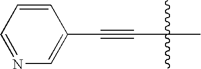 | | 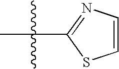 |
| 248 | 1-(cyclopropyl carbonyl)-4-[4-methoxy- 3-(pyridin-2-ylethynyl) benzoyl] piperazine | 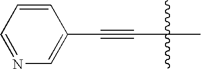 | | 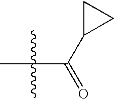 |
| 249 | 1-[4-methoxy-3- (pyridin-2-yl ethynyl) benzoyl]-4-(tetrahydro- furan-2-ylcarbonyl) piperazine | 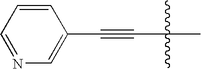 | | 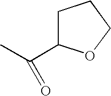 |
| 250 | 1-[4-methoxy-3- (pyridin-2-ylethynyl) benzoyl]-2-methyl-4- phenylpiperazine [R₄ = CH₃] | 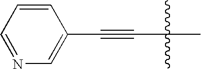 | | 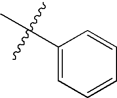 |
| 251 | 3-{4-[4-methoxy-3- (pyridin-2-ylethynyl) benzoyl]piperazin-1- yl}-1,2-benzisoxazole | 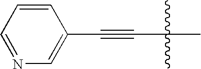 | | 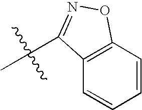 |
| 252 | 6-{4-[4-methoxy-3- (pyridin-2-ylethynyl) benzoyl]piperazin-1- yl}nicotinonitrile | 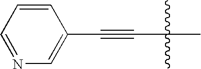 | | 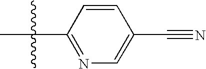 |
| 253 | 1-[4-methoxy-3- (pyridin-2-ylethynyl) benzoyl]-4-[(4- methylphenyl)sulfonyl] piperazine | 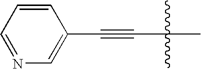 | | 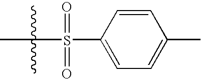 |
| 254 | 5-{4-[4-methoxy-3- (pyridin-2-yl ethynyl) benzoyl]piperazin-1- yl}-4-nitrothiophene- 2-sulfonamide | 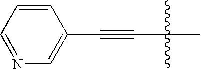 | | 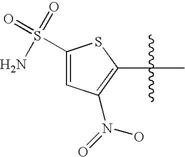 |
| 255 | 1-(6-chloropyridin-2- yl)-4-[4-methoxy-3- (pyridin-2-yl ethynyl) benzoyl] piperazine | 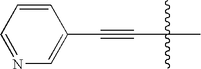 | | 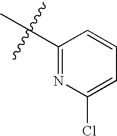 |
| 256 | 2-{4-[4-methoxy-3- (pyridin-2-ylethynyl) benzoyl]piperazin-1- yl}-1,3-benzothiazole | 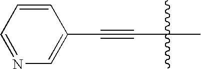 | | 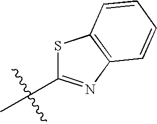 |
| 257 | 2-{4-[4-methoxy-3- (pyridin-2-ylethynyl) benzoyl]piperazin-1- yl}-1,3-benzoxazole | 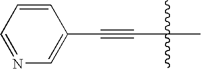 | | 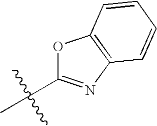 |
| 258 | 1-(2-furoyl)-4-[4- methoxy-3-(pyridin-2- ylethynyl)benzoyl] piperazine | 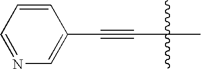 | | 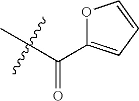 |
| 259 | 1-(1,3-benzodioxol-5- ylmethyl)-4-[4-methoxy- 3-(pyridin-2-ylethynyl) benzoyl] piperazine | 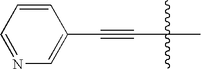 | | 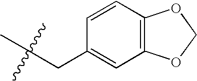 |
| 260 | 7-chloro-3-{4-[4- methoxy-3-(pyridin-2- ylethynyl)benzoyl] piperazin-1- yl}isoquinoline | 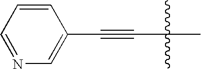 | | 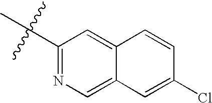 |
| 261 | 7-bromo-3-{4-[4- methoxy-3-(pyridin-2- ylethynyl)benzoyl] piperazin-1-yl} Isoquinoline | 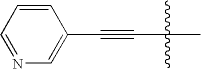 | | 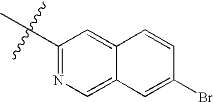 |
| 262 | 5-bromo-2-{4-[4- methoxy-3-(pyridin-2- ylethynyl)benzoyl] piperazin-1- yl}pyrimidine | 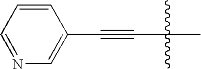 | | 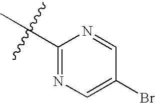 |
| 263 | 1-(2-methoxy benzoyl)- 4-[4-methoxy-3-(pyridin- 2-ylethynyl)benzoyl] piperazine | 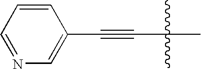 | | 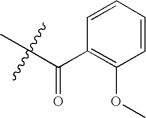 |
| 264 | 1-(3-methoxy benzoyl)- 4-[4-methoxy-3-(pyridin- 2-ylethynyl)benzoyl] piperazine | 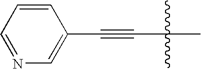 | | 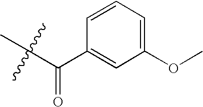 |
| 265 | 1-(4-methoxy benzoyl)- 4-[4-methoxy-3-(pyridin- 2-ylethynyl)benzoyl] piperazine | 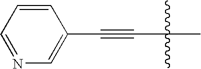 | | 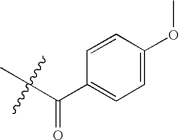 |
| 266 | 1-(2-fluorobenzyl)-4- [4-methoxy-3- (pyridin-2-ylethynyl) benzoyl]piperazine | 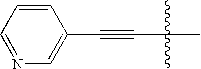 | | 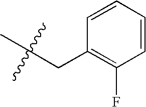 |
| 267 | 1-(3-fluorobenzyl)-4- [4-methoxy-3-(pyridin-2- ylethynyl)benzoyl] piperazine | 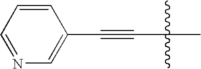 | | 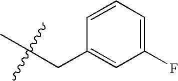 |
| 268 | 1-(4-fluorobenzyl)-4- [4-methoxy-3-(pyridin-2- ylethynyl)benzoyl] piperazine | 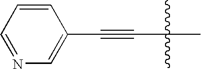 | | 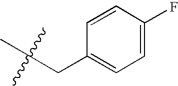 |
| 269 | 1-[(5-bromo-2- thienyl)sulfonyl]-4-[4- methoxy-3-(pyridin-2- ylethynyl)benzoyl] piperazine | 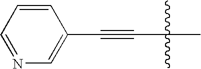 | | 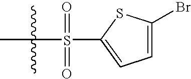 |
| 270 | 1-[(3,5-dimethyl- isoxazol-4-yl)sulfonyl]- 4-[4-methoxy-3-(pyridin- 2-ylethynyl)benzoyl] piperazine | 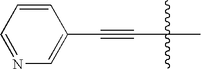 | | 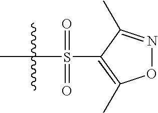 |
| 271 | 1-(3,5-dichlorophenyl)- 4-[4-methoxy-3- (pyridin-2-ylethynyl) benzoyl]piperazine | 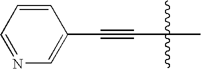 | | 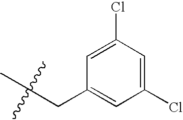 |
| 272 | 1-[4-methoxy-3- (pyridin-2-ylethynyl) benzoyl]-4-{[3- methoxy-4-(1H- tetrazol-1-yl)phenyl] sulfonyl}piperazine | 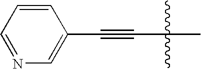 | | 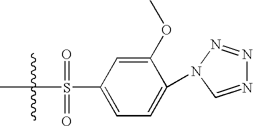 |
| 273 | 5-({4-[4-methoxy-3- (pyridin-2-ylethynyl) benzoyl]piperazin-1- yl}sulfonyl)-N,N- dimethylnaphthalen- 1-amine | 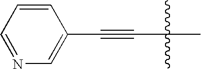 | | 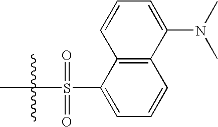 |
| 274 | 1-(3-chlorobenzyl)-4- [4-methoxy-3- (pyridin-2-ylethynyl) benzoyl]piperazine | 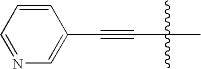 | | 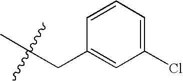 |
| 275 | 1-(4-chlorobenzyl)-4- [4-methoxy-3-(pyridin- 2-yl ethynyl)benzoyl] piperazine | 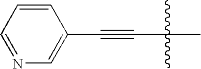 | | 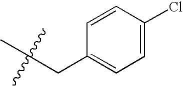 |
| 276 | 1-[4-methoxy-3- (pyridin-2-yl ethynyl) benzoyl]-4-(5-nitro- 1,3,4-thiadiazol-2- yl)piperazine | 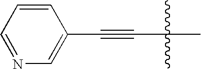 | | 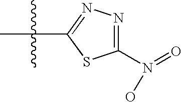 |
| 277 | 1-(2,6-dichlorobenzyl)- 4-[4-methoxy-3- (pyridin-2-ylethynyl) benzoyl]piperazine | 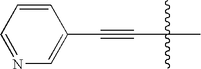 | | 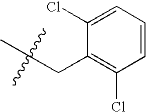 |
| 278 | 1-[(2-chlorophenyl) sulfonyl]-4-[4- methoxy-3-(pyridin-2- ylethynyl)benzoyl] piperazine | 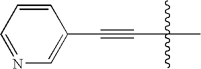 | | 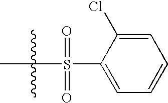 |
| 279 | 1-[(3-chlorophenyl) sulfonyl]-4-[4- methoxy-3-(pyridin-2- ylethynyl)benzoyl] piperazine | 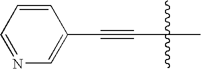 | | 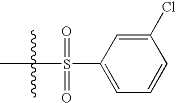 |
| 280 | 1-(2,4-dichlorobenzyl)- 4-[4-methoxy-3- (pyridin-2-ylethynyl) benzoyl]piperazine | 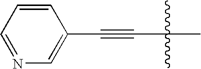 | | 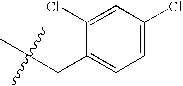 |
| 281 | 1-[4-methoxy-3- (pyridin-2-ylethynyl) benzoyl]-4-(3-phenyl- 1,2,4-thiadiazol-5-yl) piperazine | 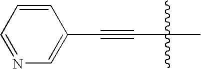 | | 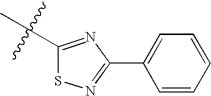 |
| 282 | 2-{4-[4-methoxy-3- (pyridin-2-ylethynyl) benzoyl]piperazin-1- yl}-6-nitro-1,3- benzothiazole | 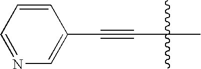 | | 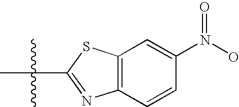 |
| 283 | 1-[4-methoxy-3- (pyridin-2-ylethynyl) benzoyl]-4-[5-(1- methyl-5-nitro-1H- imidazol-2-yl)-1,3,4- thiadiazol-2-yl] piperazine | 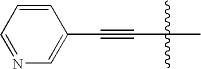 | | 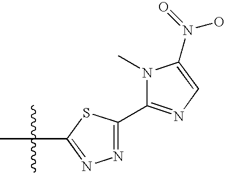 |
| 284 | 1-[4-methoxy-3- (pyridin-2-ylethynyl) benzoyl]-4-{[4-(1H- tetrazol-1-yl)phenyl] sulfonyl}piperazine | 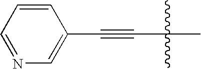 | | 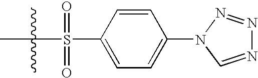 |
| 285 | 1-(4-bromo-2-fluoro- benzyl)-4-[4-methoxy-3- (pyridin-2-ylethynyl) benzoyl]piperazine | 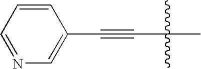 | | 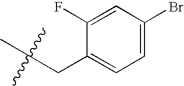 |
| 286 | tert-butyl 4-[4-methoxy- 3-(pyridin-2-ylethynyl) benzoyl] piperazine-1- carboxylate | 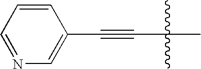 | | 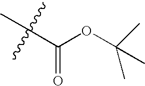 |
| 287 | 1-[4-methoxy-3- (pyridin-2-ylethynyl) benzoyl]-4-(2- naphthylsulfonyl) piperazine | 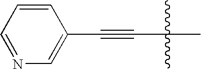 | | 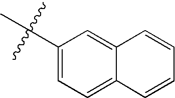 |
| 288 | 1-(3,4-dichloro benzyl)-4-[4-methoxy- 3-(pyridin-2-ylethynyl) benzoyl]piperazine | 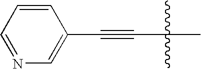 | | 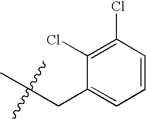 |
| 289 | 1-(2-chloro-6- fluorobenzyl)-4-[4- methoxy-3-(pyridin-2- ylethynyl)benzoyl] piperazine | 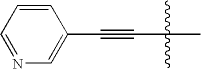 | | 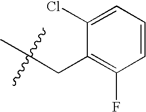 |
| 290 | 1-(1-benzothiophen- 2-yl)-4-[4-methoxy-3- (pyridin-2-ylethynyl) benzoyl]piperazine | 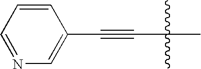 | | 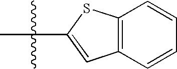 |
| 291 | 1-[4-methoxy-3- (pyridin-2-ylethynyl) benzoyl]-4-(5-phenyl- 4H-1,2,4-triazol-3-yl) piperazine | 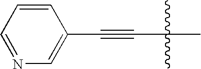 | | 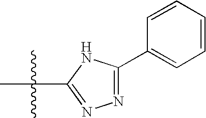 |

TABLE 8A

| | | | | Biological Activity | |
|---|---|---|---|---|---|
| | | LCMS data | | Median Ki | |
| Cmpd | Name | Mass | Ion | (μM) | IC50 (uM) |
| 183 | 1-[4-methoxy-3-(pyridin-2-ylethynyl)benzoyl]- | 467.2 | M + H | 0.041 | 0.066 |
| | 4-[5-(trifluoromethyl)pyridin-2- | | | | |
| | yl]piperazine | | | | |
| 184 | 1-[4-methoxy-3-(pyridin-2-ylethynyl)benzoyl]- | 467.2 | M + H | 0.078 | 0.117 |
| | 4-[3-(trifluoromethyl)pyridin-2- | | | | |
| | yl]piperazine | | | | |
| 185 | 1-(3,5-dichloropyridin-2-yl)-4-[4- | 467.1 | M + H | 0.047 | 0.043 |
| | methoxy-3-(pyridin-2-ylethynyl)benzoyl]piperazine | | | | |
| 186 | 1-(3-chloropyridin-2-yl)-4-[4-methoxy-3- | 433.1 | M + H | 0.019 | 0.037 |
| | (pyridin-2-ylethynyl) benzoyl]piperazine | | | | |
| 187 | 1-[4-methoxy-3-(pyridin-2- | 466.2 | M + H | 0.005 | 0.834 |
| | ylethynyl)benzoyl]-4-[3-(trifluoromethyl)phenyl]piperazine | | | | |
| 188 | 1-(5-chloropyridin-2-yl)-4-[4-methoxy-3- | 433.2 | M + H | 0.007 | 0.086 |
| | (pyridin-2-ylethynyl) benzoyl]piperazine | | | | |
| 189 | 1-(3-chlorophenyl)-4-[4-methoxy-3- | 432.2 | M + H | 0.004 | 0.148 |
| | (pyridin-2-ylethynyl)benzoyl] piperazine | | | | |
| 190 | 1-[3-chloro-5-(trifluoromethyl)pyridin-2- | 501.1 | M + H | 0.413 | 0.408 |
| | yl]-4-[4-methoxy-3-(pyridin-2- | | | | |
| | ylethynyl)benzoyl]piperazine | | | | |
| 191 | 1-[4-methoxy-3-(pyridin-2- | 413.2 | M + H | 0.019 | 0.031 |
| | ylethynyl)benzoyl]-4-(4-methylpyridin-2- | | | | |
| | yl)piperazine | | | | |
| 192 | 2-{4-[4-methoxy-3-(pyridin-2- | 428.3 | M + H | 0.045 | 0.109 |
| | ylethynyl)benzoyl]piperazin-1-yl}-4,6- | | | | |
| | dimethylpyrimidine | | | | |
| 193 | 3-{4-[4-methoxy-3-(pyridin-2- | 425.2 | M + H | 0.034 | 0.046 |
| | ylethynyl)benzoyl]piperazin-1- | | | | |
| | yl}pyrazine-2-carbonitrile | | | | |
| 194 | 2-{4-[4-methoxy-3-(pyridin-2- | 468.2 | M + H | 0.004 | 0.078 |
| | ylethynyl)benzoyl]piperazin-1-yl}-4- | | | | |
| | (trifluoromethyl)pyrimidine | | | | |
| 195 | 3-{4-[4-methoxy-3-(pyridin-2- | 414.2 | M + H | 0.026 | 0.702 |
| | ylethynyl)benzoyl]piperazin-1-yl}phenol | | | | |
| 196 | 1-[4-methoxy-3-(pyridin-2- | 412.2 | M + H | 0.016 | 0.065 |
| | ylethynyl)benzoyl]-4-(3- | | | | |
| | methylphenyl)piperazine | | | | |
| 197 | 5-bromo-4-methoxy-2-{4-[4-methoxy-3- | 508.1 | M + H | 0.012 | 0.056 |
| | (pyridin-2-ylethynyl) benzoyl]piperazin- | | | | |
| | 1-yl}pyrimidine | | | | |
| 198 | 1-[4-methoxy-3-(pyridin-2- | 413.2 | M + H | 0.030 | 0.034 |
| | ylethynyl)benzoyl]-4-(6-methyl pyridin-2- | | | | |
| | yl)piperazine | | | | |
| 199 | (1R,4S)-2-(4-chlorophenyl)-5-[4- | 444.2 | M + H | 0.076 | >1.000 |
| | methoxy-3-(pyridin-2-ylethynyl)benzoyl]- | | | | |
| | 2,5-diazabicyclo[2.2.1] heptane | | | | |
| 200 | 4-methoxy-2-{4-[4-methoxy-3-(pyridin-2- | 430.2 | M + H | 0.021 | 0.024 |
| | ylethynyl)benzoyl] piperazin-1- | | | | |
| | yl}pyrimidine | | | | |
| 201 | 3-{4-[4-methoxy-3-(pyridin-2- | 455.2 | M + H | 0.003 | 0.024 |
| | ylethynyl)benzoyl]piperazin-1-yl}-1,2- | | | | |
| | benzisothiazole | | | | |
| 202 | 6-{4-[4-methoxy-3-(pyridin-2- | 438.2 | M + H | 1.041 | |
| | ylethynyl)benzoyl]-1,4-diazepan-1- | | | | |
| | yl}nicotinonitrile | | | | |
| 203 | 1-[4-methoxy-3-(pyridin-2- | 413.2 | M + H | 0.029 | 0.032 |
| | ylethynyl)benzoyl]-4-(5-methylpyridin-2- | | | | |
| | yl)piperazine | | | | |
| 204 | 2-{4-[4-methoxy-3-(pyridin-2- | 414.2 | M + H | 0.029 | 0.132 |
| | ylethynyl)benzoyl]piperazin-1-yl}-6- | | | | |
| | methylpyrazine | | | | |
| 205 | 1-[4-methoxy-3-(pyridin-2- | 413.2 | M + H | 0.131 | >1.000 |
| | ylethynyl)benzoyl]-4-pyridin-2-yl-1,4- | | | | |
| | diazepane | | | | |
| 206 | 1-[4-methoxy-3-(pyridin-2- | 474.2 | M + H | 0.066 | 0.295 |
| | ylethynyl)benzoyl]-4-[5-(trifluoromethyl)- | | | | |
| | 1,3,4-thiadiazol-2-yl]piperazine | | | | |
| 207 | (1R,4S)-2-(3-fluorophenyl)-5-[4- | 428.2 | M + H | 0.019 | 0.306 |
| | methoxy-3-(pyridin-2-ylethynyl)benzoyl]- | | | | |
| | 2,5-diazabicyclo[2.2.1]heptane | | | | |
| 208 | 1-[4-methoxy-3-(pyridin-2- | 413.2 | M + H | 0.021 | 0.141 |
| | ylethynyl)benzoyl]-4-(3-methylpyridin-2- | | | | |
| | yl)piperazine | | | | |
| 209 | 1-[4-methoxy-3-(pyridin-2- | 481.2 | M + H | 0.674 | |
| | ylethynyl)benzoyl]-4-[3- | | | | |
| | (trifluoromethyl)pyridin-2-yl]-1,4- | | | | |
| | diazepane | | | | |
| 210 | 1-[4-methoxy-3-(pyridin-2- | 481.2 | M + H | 0.851 | |
| | ylethynyl)benzoyl]-4-[5- | | | | |
| | (trifluoromethyl)pyridin-2-yl]-1,4- | | | | |
| | diazepane | | | | |
| 211 | 1-[4-methoxy-3-(pyridin-2- | 427.2 | M + H | 0.619 | |
| | ylethynyl)benzoyl]-4-(6-methylpyridin-2- | | | | |
| | yl)-1,4-diazepane | | | | |
| 212 | 1-[3-chloro-5-(trifluoromethyl)pyridin-2- | 515.1 | M + H | 0.704 | |
| | yl]-4-[4-methoxy-3-(pyridin-2- | | | | |
| | ylethynyl)benzoyl]-1,4-diazepane | | | | |
| 213 | 1-(6-methoxypyridin-2-yl)-4-[4-methoxy- | 429.2 | M + H | 0.011 | 0.086 |
| | 3-(pyridin-2-ylethynyl)benzoyl]piperazine | | | | |
| 214 | 2-{4-[4-methoxy-3-(pyridin-2- | 438.2 | M + H | 0.458 | |
| | ylethynyl)benzoyl]-1,4-diazepan-1- | | | | |
| | yl}nicotinonitrile | | | | |
| 215 | 1-[4-methoxy-3-(pyridin-2- | 458.2 | M + H | 2.193 | |
| | ylethynyl)benzoyl]-4-(5-nitropyridin-2-yl)- | | | | |
| | 1,4-diazepane | | | | |
| 216 | 1-(2-chlorophenyl)-4-[4-methoxy-3- | 432.1 | M + H | 0.107 | >1.000 |
| | (pyridin-2-ylethynyl)benzoyl] piperazine | | | | |
| 217 | 1-(4-chlorophenyl)-4-[4-methoxy-3- | 432.1 | M + H | 0.027 | 0.336 |
| | (pyridin-2-ylethynyl)benzoyl] piperazine | | | | |
| 218 | 1-(3,4-dichlorophenyl)-4-[4-methoxy-3- | 466 | M + H | 0.048 | >1.000 |
| | (pyridin-2-ylethynyl)benzoyl] piperazine | | | | |
| 219 | 1-(2,3-dimethylphenyl)-4-[4-methoxy-3- | 426.1 | M + H | 0.168 | >1.000 |
| | (pyridin-2-ylethynyl)benzoyl] piperazine | | | | |
| 220 | 2-isopropyl-4-{4-[4-methoxy-3-(pyridin- | 456.2 | M + H | 0.089 | >1.000 |
| | 2-ylethynyl)benzoyl]piperazin-1-yl}-6- | | | | |
| | methylpyrimidine | | | | |
| 221 | 1-[4-methoxy-3-(pyridin-2- | 412.1 | M + H | 0.100 | >1.000 |
| | ylethynyl)benzoyl]-4-(2- | | | | |
| | methylphenyl)piperazine | | | | |
| 222 | 1-[4-methoxy-3-(pyridin-2- | 412.1 | M + H | 0.074 | >1.000 |
| | ylethynyl)benzoyl]-4-(4- | | | | |
| | methylphenyl)piperazine | | | | |
| 223 | 1-(3-fluorophenyl)-4-[4-methoxy-3- | 416.1 | M + H | 0.010 | 0.103 |
| | (pyridin-2-ylethynyl)benzoyl]piperazine | | | | |
| 224 | 1-[4-methoxy-3-(pyridin-2- | 462.1 | M + H | 0.115 | |
| | ylethynyl)benzoyl]-4- | | | | |
| | (phenylsulfonyl)piperazine | | | | |
| 225 | 1-[(5-chloro-2-thienyl)sulfonyl]-4-[4- | 502 | M + H | 0.051 | >1.000 |
| | methoxy-3-(pyridin-2- | | | | |
| | ylethynyl)benzoyl]piperazine | | | | |
| 226 | (1R,4S)-2-[4-methoxy-3-(pyridin-2- | 424.1 | M + H | 0.277 | >1.000 |
| | ylethynyl)benzoyl]-5-(4-methylphenyl)- | | | | |
| | 2,5-diazabicyclo[2.2.1]heptane | | | | |
| 227 | (1S,4R)-2-(4-fluorophenyl)-5-[4- | 428.1 | M + H | 0.071 | >1.000 |
| | methoxy-3-(pyridin-2-ylethynyl)benzoyl]- | | | | |
| | 2,5-diazabicyclo[2.2.1]heptane | | | | |
| 228 | 1-[4-methoxy-3-(pyridin-2- | 466.1 | M + H | 0.674 | |
| | ylethynyl)benzoyl]-4-[4- | | | | |
| | (trifluoromethyl)phenyl]piperazine | | | | |
| 229 | 1-[4-methoxy-3-(pyridin-2- | 444.1 | M + H | 0.056 | >1.000 |
| | ylethynyl)benzoyl]-4-(5-nitropyridin-2- | | | | |
| | yl)piperazine | | | | |
| 230 | 1-(2-methoxyphenyl)-4-[4-methoxy-3- | 428.1 | M + H | 0.734 | |
| | (pyridin-2-ylethynyl)benzoyl]piperazine | | | | |
| 231 | 1-(4-fluorophenyl)-4-[4-methoxy-3- | 416.1 | M + H | 0.023 | 0.244 |
| | (pyridin-2-ylethynyl)benzoyl]piperazine | | | | |
| 232 | 1-[4-methoxy-3-(pyridin-2- | 443.1 | M + H | 0.096 | >1.000 |
| | ylethynyl)benzoyl]-4-(4- | | | | |
| | nitrophenyl)piperazine | | | | |
| 233 | 1-(4-methoxyphenyl)-4-[4-methoxy-3- | 428.1 | M + H | 0.066 | >1.000 |
| | (pyridin-2-ylethynyl)benzoyl]piperazine | | | | |
| 234 | 1-(benzylsulfonyl)-4-[4-methoxy-3- | 476.1 | M + H | IC50 > 10 | |
| | (pyridin-2-ylethynyl)benzoyl]piperazine | | | uM | |
| 235 | 1-(2,3-dihydro-1,4-benzodioxin-6- | 520 | M + H | 0.075 | >1.000 |
| | ylsulfonyl)-4-[4-methoxy-3-(pyridin-2- | | | | |
| | ylethynyl)benzoyl]piperazine | | | | |
| 236 | 1-[4-methoxy-3-(pyridin-2- | 399.1 | M + H | 0.078 | >1.000 |
| | ylethynyl)benzoyl]-4-pyridin-4- | | | | |
| | ylpiperazine | | | | |
| 237 | 1-(4-{4-[4-methoxy-3-(pyridin-2- | 440.12 | M + H | 0.021 | 0.119 |
| | ylethynyl)benzoyl]piperazin-1- | | | | |
| | yl}phenyl)ethanone | | | | |
| 238 | 1-[4-methoxy-3-(pyridin-2- | 476.1 | M + H | 0.521 | |
| | ylethynyl)benzoyl]-4-[4- | | | | |
| | (methylsulfonyl)phenyl]piperazine | | | | |
| 239 | 1-[(3,4-dichlorophenyl)sulfonyl]-4-[4- | 530 | M + H | 1.412 | |
| | methoxy-3-(pyridin-2- | | | | |
| | ylethynyl)benzoyl]piperazine | | | | |
| 240 | 1-[4-fluoro-2-(methylsulfonyl)phenyl]-4- | 494 | M + H | IC50 > 10 | |
| | [4-methoxy-3-(pyridin-2- | | | uM | |
| | ylethynyl)benzoyl]piperazine | | | | |
| 241 | 1-(3-methoxyphenyl)-4-[4-methoxy-3- | 428.1 | M + H | 0.015 | 0.081 |
| | (pyridin-2-ylethynyl)benzoyl]piperazine | | | | |
| 242 | 1-(2,5-dimethylphenyl)-4-[4-methoxy-3- | 426.2 | M + H | 1.005 | |
| | (pyridin-2-ylethynyl)benzoyl]piperazine | | | | |
| 243 | 1-[(4-chlorophenyl)sulfonyl]-4-[4- | 496 | M + H | 0.014 | 0.154 |
| | methoxy-3-(pyridin-2- | | | | |
| | ylethynyl)benzoyl]piperazine | | | | |
| 244 | 1-benzoyl-4-[4-methoxy-3-(pyridin-2- | 426.1 | M + H | 0.054 | 0.237 |
| | ylethynyl)benzoyl]piperazine | | | | |
| 245 | 1-(ethylsulfonyl)-4-[4-methoxy-3- | 414.1 | M + H | 0.231 | 0.344 |
| | (pyridin-2-ylethynyl)benzoyl]piperazine | | | | |
| 246 | 1-[4-methoxy-3-(pyridin-2- | 466.1 | M + H | 2.842 | |
| | ylethynyl)benzoyl]-4-[2- | | | | |
| | (trifluoromethyl)phenyl]piperazine | | | | |
| 247 | 1-[4-methoxy-3-(pyridin-2- | 405 | M + H | 0.017 | 0.085 |
| | ylethynyl)benzoyl]-4-(1,3-thiazol-2- | | | | |
| | yl)piperazine | | | | |
| 248 | 1-(cyclopropylcarbonyl)-4-[4-methoxy-3- | 390.1 | M + H | 0.083 | >1.000 |
| | (pyridin-2-ylethynyl)benzoyl]piperazine | | | | |
| 249 | 1-[4-methoxy-3-(pyridin-2- | 420.1 | M + H | 0.181 | |
| | ylethynyl)benzoyl]-4-(tetrahydrofuran-2- | | | | |
| | ylcarbonyl)piperazine | | | | |
| 250 | 1-[4-methoxy-3-(pyridin-2- | 412.1 | M + H | 0.024 | 0.096 |
| | ylethynyl)benzoyl]-2-methyl-4- | | | | |
| | phenylpiperazine | | | | |
| 251 | 3-{4-[4-methoxy-3-(pyridin-2- | 439.1 | M + H | 0.006 | 0.025 |
| | ylethynyl)benzoyl]piperazin-1-yl}-1,2- | | | | |
| | benzisoxazole | | | | |
| 252 | 6-{4-[4-methoxy-3-(pyridin-2- | 424.1 | M + H | 0.033 | 0.192 |
| | ylethynyl)benzoyl]piperazin-1- | | | | |
| | yl}nicotinonitrile | | | | |
| 253 | 1-[4-methoxy-3-(pyridin-2- | 476.1 | M + H | 0.021 | 0.180 |
| | ylethynyl)benzoyl]-4-[(4- | | | | |
| | methylphenyl)sulfonyl]piperazine | | | | |
| 254 | 5-{4-[4-methoxy-3-(pyridin-2- | 528 | M + H | 0.108 | |
| | ylethynyl)benzoyl]piperazin-1-yl}-4- | | | | |
| | nitrothiophene-2-sulfonamide | | | | |
| 255 | 1-(6-chloropyridin-2-yl)-4-[4-methoxy-3- | 433.1 | M + H | 0.009 | 0.129 |
| | (pyridin-2-ylethynyl)benzoyl]piperazine | | | | |
| 256 | 2-{4-[4-methoxy-3-(pyridin-2- | 445.1 | M + H | 0.043 | 0.100 |
| | ylethynyl)benzoyl]piperazin-1-yl}-1,3- | | | | |
| | benzothiazole | | | | |
| 257 | 2-{4-[4-methoxy-3-(pyridin-2- | 439.1 | M + H | 0.038 | 0.110 |
| | ylethynyl)benzoyl]piperazin-1-yl}-1,3- | | | | |
| | benzoxazole | | | | |
| 258 | 1-(2-furoyl)-4-[4-methoxy-3-(pyridin-2- | 416.1 | M + H | 0.041 | 0.095 |
| | ylethynyl)benzoyl]piperazine | | | | |
| 259 | 1-(1,3-benzodioxol-5-ylmethyl)-4-[4- | 456.1 | M + H | 0.038 | 0.157 |
| | methoxy-3-(pyridin-2-ylethynyl)benzoyl]piperazine | | | | |
| 260 | 7-chloro-3-{4-[4-methoxy-3-(pyridin-2- | 483.1 | M + H | 0.951 | |
| | ylethynyl)benzoyl]piperazin-1- | | | | |
| | yl}isoquinoline | | | | |
| 261 | 7-bromo-3-{4-[4-methoxy-3-(pyridin-2- | 527 | M + H | 1.377 | |
| | ylethynyl)benzoyl]piperazin-1- | | | | |
| | yl}isoquinoline | | | | |
| 262 | 5-bromo-2-{4-[4-methoxy-3-(pyridin-2- | 478 | M + H | 0.017 | 0.148 |
| | ylethynyl)benzoyl]piperazin-1- | | | | |
| | yl}pyrimidine | | | | |
| 263 | 1-(2-methoxybenzoyl)-4-[4-methoxy-3- | 456.1 | M + H | 0.169 | |
| | (pyridin-2-ylethynyl)benzoyl] piperazine | | | | |
| 264 | 1-(3-methoxybenzoyl)-4-[4-methoxy-3- | 456.1 | M + H | 0.106 | |
| | (pyridin-2-ylethynyl)benzoyl] piperazine | | | | |
| 265 | 1-(4-methoxybenzoyl)-4-[4-methoxy-3- | 456.1 | M + H | 0.054 | >1.000 |
| | (pyridin-2-ylethynyl)benzoyl] piperazine | | | | |
| 266 | 1-(2-fluorobenzyl)-4-[4-methoxy-3- | 430.3 | M + H | 0.033 | >1.000 |
| | (pyridin-2-ylethynyl)benzoyl] piperazine | | | | |
| 267 | 1-(3-fluorobenzyl)-4-[4-methoxy-3- | 430.3 | M + H | 0.057 | 0.167 |
| | (pyridin-2-ylethynyl)benzoyl] piperazine | | | | |
| 268 | 1-(4-fluorobenzyl)-4-[4-methoxy-3- | 430.3 | M + H | 0.057 | 0.219 |
| | (pyridin-2-ylethynyl)benzoyl] piperazine | | | | |
| 269 | 1-[(5-bromo-2-thienyl)sulfonyl]-4-[4- | 546 | M + H | 0.049 | 0.000 |
| | methoxy-3-(pyridin-2-ylethynyl)benzoyl]piperazine | | | | |
| 270 | 1-[(3,5-dimethylisoxazol-4-yl)sulfonyl]-4- | 481.2 | M + H | 1.539 | |
| | [4-methoxy-3-(pyridin-2- | | | | |
| | ylethynyl)benzoyl]piperazine | | | | |
| 271 | 1-(3,5-dichlorophenyl)-4-[4-methoxy-3- | 466.1 | M + H | 0.018 | |
| | (pyridin-2-ylethynyl)benzoyl] piperazine | | | | |
| 272 | 1-[4-methoxy-3-(pyridin-2- | 560.1 | M + H | IC50 > 10 | |
| | ylethynyl)benzoyl]-4-{[3-methoxy-4-(1H- | | | uM | |
| | tetrazol-1-yl)phenyl]sulfonyl} piperazine | | | | |
| 273 | 5-({4-[4-methoxy-3-(pyridin-2- | 555.1 | M + H | 1.740 | |
| | ylethynyl)benzoyl]piperazin-1- | | | | |
| | yl}sulfonyl)-N,N-dimethylnaphthalen-1- | | | | |
| | amine | | | | |
| 274 | 1-(3-chlorobenzyl)-4-[4-methoxy-3- | 446.1 | M + H | 0.089 | 0.316 |
| | (pyridin-2-ylethynyl)benzoyl]piperazine | | | | |
| 275 | 1-(4-chlorobenzyl)-4-[4-methoxy-3- | 446.1 | M + H | 0.017 | 0.144 |
| | (pyridin-2-ylethynyl)benzoyl]piperazine | | | | |
| 276 | 1-[4-methoxy-3-(pyridin-2- | 451 | M + H | 0.139 | |
| | ylethynyl)benzoyl]-4-(5-nitro-1,3,4- | | | | |
| | thiadiazol-2-yl)piperazine | | | | |
| 277 | 1-(2,6-dichlorobenzyl)-4-[4-methoxy-3- | 480.1 | M + H | 0.272 | |
| | (pyridin-2-ylethynyl)benzoyl]piperazine | | | | |
| 278 | 1-[(2-chlorophenyl)sulfonyl]-4-[4- | 496.1 | M + H | 0.876 | |
| | methoxy-3-(pyridin-2- | | | | |
| | ylethynyl)benzoyl]piperazine | | | | |
| 279 | 1-[(3-chlorophenyl)sulfonyl]-4-[4- | 496.1 | M + H | 0.832 | |
| | methoxy-3-(pyridin-2- | | | | |
| | ylethynyl)benzoyl]piperazine | | | | |
| 280 | 1-(2,4-dichlorobenzyl)-4-[4-methoxy-3- | 480.1 | M + H | 0.043 | 0.317 |
| | (pyridin-2-ylethynyl)benzoyl]piperazine | | | | |
| 281 | 1-[4-methoxy-3-(pyridin-2- | 482.1 | M + H | 0.197 | |
| | ylethynyl)benzoyl]-4-(3-phenyl-1,2,4- | | | | |
| | thiadiazol-5-yl)piperazine | | | | |
| 282 | 2-{4-[4-methoxy-3-(pyridin-2- | 500 | M + H | IC50 > 10 | |
| | ylethynyl)benzoyl]piperazin-1-yl}-6-nitro- | | | uM | |
| | 1,3-benzothiazole | | | | |
| 283 | 1-[4-methoxy-3-(pyridin-2- | 531.1 | M + H | 2.308 | |
| | ylethynyl)benzoyl]-4-[5-(1-methyl-5- | | | | |
| | nitro-1H-imidazol-2-yl)-1,3,4-thiadiazol- | | | | |
| | 2-yl]piperazine | | | | |
| 284 | 1-[4-methoxy-3-(pyridin-2- | 530.1 | M + H | 0.376 | |
| | ylethynyl)benzoyl]-4-{[4-(1H-tetrazol-1- | | | | |
| | yl)phenyl]sulfonyl}piperazine | | | | |
| 285 | 1-(4-bromo-2-fluorobenzyl)-4-[4- | 508 | M + H | 0.007 | 0.326 |
| | methoxy-3-(pyridin-2- | | | | |
| | ylethynyl)benzoyl]piperazine | | | | |
| 286 | tert-butyl 4-[4-methoxy-3-(pyridin-2- | 422.1 | M + H | | |
| | ylethynyl)benzoyl]piperazine-1- | | | | |
| | carboxylate | | | | |
| 287 | 1-[4-methoxy-3-(pyridin-2- | 512.1 | M + H | 0.070 | >1000 |
| | ylethynyl)benzoyl]-4-(2- | | | | |
| | naphthylsulfonyl)piperazine | | | | |
| 288 | 1-(3,4-dichlorobenzyl)-4-[4-methoxy-3- | 480 | M + H | 0.170 | |
| | (pyridin-2-ylethynyl)benzoyl]piperazine | | | | |
| 289 | 1-(2-chloro-6-fluorobenzyl)-4-[4- | 464 | M + H | 0.269 | |
| | methoxy-3-(pyridin-2- | | | | |
| | ylethynyl)benzoyl]piperazine | | | | |
| 290 | 1-(1-benzothiophen-2-yl)-4-[4-methoxy- | 454.1 | M + H | IC50 > 10 | |
| | 3-(pyridin-2-ylethynyl)benzoyl]piperazine | | | uM | |
| 291 | 1-[4-methoxy-3-(pyridin-2- | 465.2 | M + H | 0.199 | |
| | ylethynyl)benzoyl]-4-(5-phenyl-4H-1,2,4- | | | | |
| | triazol-3-yl)piperazine | | | | |

### Example 9

### 4-Amino-2-(4-(4-methoxy-3-(pyridin-2-ylethynyl)benzoyl)piperazin-1-yl)pyrimidine-5-carbonitrile (Compound 293)

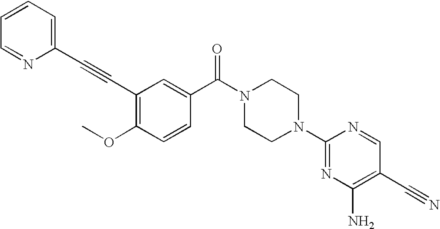

### Step 1: Methyl 4-methoxy-3-(pyridin-2-ylethynyl)benzoate

To a solution of methyl 3-bromo-4-methoxybenzoate (5.0 g, 20.4 mmol) and 2-ethylnylpyridine (3.14 mL, 31.1 mmol) in toluene (100 mL) was added CuI (0.78 g, 3.9 mmol) and TEA (6.2 mL, 44.7 mmol). Pd(Ph₃P)₂Cl₂ (2.9 g, 4.1 mmol) was then added to the resulting suspension. The vessel was purged with nitrogen and the reaction was stirred at 110° C. for 10 hours. The contents of the flask were then washed through a plug of silica gel with EtOAc and the resulting solution was concentrated at reduced pressure and purified by flash chromatography on silica (5% MeOH in DCM) to yield 4.1 g (75%) of product as a brown solid.

### Step 2: 4-Methoxy-3-(pyridin-2-ylethynyl)benzoic acid

To a solution of methyl 4-methoxy-3-(pyridin-2-ylethynyl)benzoate (4.1 g, 15.3 mmol) in THF (150 mL), MeOH (20 mL), and H₂O (40 mL) was added lithium hydroxide monohydrate (1.68 g, 40 mmol). The reaction was stirred at room temperature overnight and then concentrated at reduced pressure to an approximate volume of 40 mL. The remaining solution was diluted with an additional 50 mL of H₂O, washed with Et₂O (X₂), and acidified to pH 4.0. The resulting precipitate was collected by suction filtration. The filtrate was saturated with solid NaCl and extracted with EtOAc (2×100 mL). The organic extracts were concentrated to yield a solid residue that was added to the collected precipitate and the combined solids were dried in a vacuum oven at 50° C. for 3 hours to yield 3.44 g (84%) of the carboxylic acid as a tan solid. No additional purification of the carboxylic acid was required.

### Step 3: tert-Butyl 4-(4-methoxy-3-(pyridin-2-ylethynyl)benzoyl)piperazine-1-carboxylate

To a stirred solution of 4-methoxy-3-(pyridin-2-ylethynyl)benzoic acid (1.50 g, 5.92 mmol) in DCM (45 mL) was added HOBT (1.45 g, 9.47 mmol) and EDC (1.70 g, 8.88 mmol). The resulting solution was stirred for 15 min, at which time tert-butyl piperazine-1-carboxylate (1.43 g, 7.70 mmol) and TEA (2.46 mL, 17.76 mmol) were added and the solution was stirred for 5 h. Upon completion, the solvent was removed under reduced pressure and the residue was purified via flash chromatography on silica gel (20:1 CH₂Cl₂/MeOH) to afford 2.02 g (81%) of the boc-piperazine as a light brown solid.

### Step 4: (4-methoxy-3-(pyridin-2-ylethynyl)phenyl)(piperazin-1-yl)methanone hydrochloric acid salt

Acetyl chloride (186 mg, 2.38 mmol) was added in a dropwise fashion to a solution of tert-Butyl 4-(4-methoxy-3-(pyridin-2-ylethynyl)benzoyl)piperazine-1-carboxylate (1.00 g, 2.38 mmol) in MeOH (5 mL) cooled to 0° C. After 45 min, additional acetyl chloride (186 mg, 2.38 mmol) was added to the solution. The reaction solution solidified with quantitative formation of the piperazine hydrochloric acid salt as shown by LCMS. The product was filtered, washed with hexanes and was used without further purification or modification.

### Step 5: 4-amino-2-(4-(4-methoxy-3-(pyridin-2-ylethynyl)benzoyl)piperazin-1-yl)pyrimidine-5-carbonitrile (Compound 293)

To a solution of (4-methoxy-3-(pyridin-2-ylethynyl)phenyl)(piperazin-1-yl)methanone hydrochloric acid salt (50 mg, 0.156 mmol) in isopropyl alcohol (0.40 mL) was added 4-amino-2-chloropyrimidine-5-carbonitrile (48 mg, 0.312 mmol) and TEA (0.065 mL). The vial was purged with nitrogen and the reaction solution was heated to 85° C. The reaction was stirred for 24 h, at which point the solvent was removed under reduced pressure and the residue was purified via flash chromatography on silica gel (5% MeOH in DCM) to afford 51 mg (74%) of the title compound as an off-white solid.

Compounds 292-306, shown in Table 9 and 9A below, were prepared using the procedure of Example 9 described above.

### UNLESS NOTED OTHERWISE THE FOLLOWING VALUES REFER TO FORMULA I

### WHEREIN R₁, R₄, R₄ₐ, R₅, R₅ₐ═H; X₁═COMe, X₂═CH; Z=CO

TABLE 9

| Cmpd | Name | R₂ | Noted Values | R₆ |
|---|---|---|---|---|
| 292 | 1-[4-methoxy-3- (pyridin-2-ylethynyl) benzoyl]-4-phenyl piperazine | 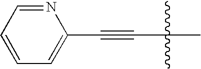 | | 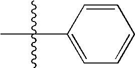 |
| 293 | 4-amino-2-{4-[4- methoxy-3-(pyridin-2- ylethynyl)benzoyl] piperazin-1-yl} pyrimidine-5-carbonitrile | 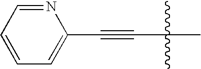 | | 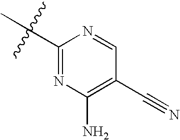 |
| 294 | 4-chloro-6-{4-[4- methoxy-3-(pyridin-2- ylethynyl)benzoyl] piperazin-1-yl}-2- (methylthio) pyrimidine | 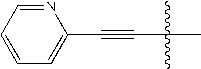 | | 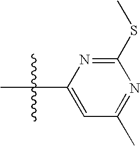 |
| 295 | 2-chloro-5-fluoro-4- {4-[4-methoxy-3- (pyridin-2-ylethynyl) benzoyl]piperazin-1- yl}pyrimidine | 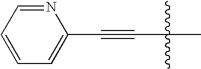 | | 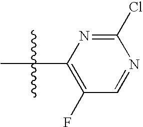 |
| 296 | 4-{4-[4-methoxy-3- (pyridin-2-ylethynyl) benzoyl]piperazin-1- yl}-2-(methylthio) pyrimidine | 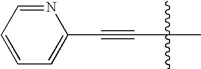 | | 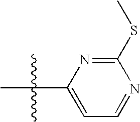 |
| 297 | 4-chloro-6-{4-[4- methoxy-3-(pyridin-2- ylethynyl)benzoyl] piperazin-1-yl}-2- methylpyrimidine | 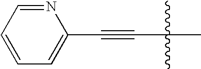 | | 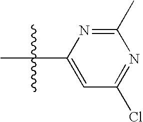 |
| 298 | 5-fluoro-2-{4-[4- methoxy-3-(pyridin-2- ylethynyl)benzoyl] piperazin-1-yl} pyrimidine | 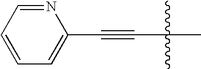 | | 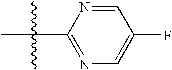 |
| 299 | 5-methoxy-2-{4-[4- methoxy-3-(pyridin-2- ylethynyl)benzoyl] piperazin-1-yl} pyrimidine | 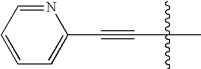 | | 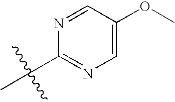 |
| 300 | 5-fluoro-2-{4-[4- methoxy-3-(pyridin-2- ylethynyl)benzoyl] piperazin-1-yl} pyrimidin-4-amine | 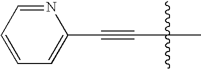 | | 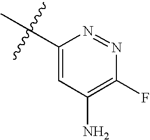 |
| 301 | 3-methoxy-6-{4-[4- methoxy-3-(pyridin-2- ylethynyl)benzoyl] piperazin-1-yl} pyridizine | 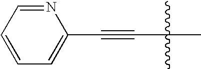 | | 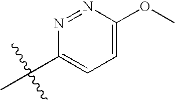 |
| 302 | 6-chloro-3-{4-[4- methoxy-3-(pyridin-2- ylethynyl)benzoyl] piperazin-1-yl}-4- methylpyridazine | 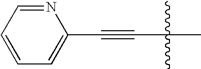 | | 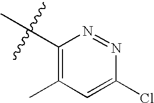 |
| 303 | 3-chloro-6-{4-[4- methoxy-3-(pyridin-2- ylethynyl)benzoyl] piperazin-1-yl} pyridizine | 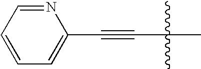 | | 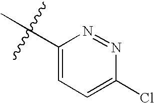 |
| 304 | 2-chloro-3-{4-[4- methoxy-3-(pyridin-2- yl ethynyl)benzoyl] piperazin-1-yl} pyrazine | 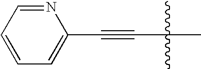 | | 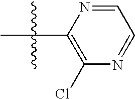 |
| 305 | 2,4-dimethoxy-6-{4- [4-methoxy-3- (pyridin-2-ylethynyl) benzoyl]piperazin-1- yl}-1,3,5-triazine | 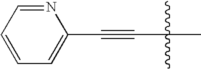 | | 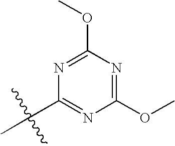 |
| 306 | 1-chloro-4-{4-[4- methoxy-3-(pyridin-2- yl ethynyl)benzoyl] piperazin-1-yl} phthalazine | 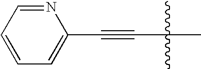 | | 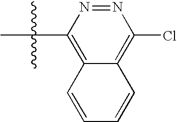 |

TABLE 9A

| | | | | Biological Activity | |
|---|---|---|---|---|---|
| | | LCMS data | | Median Ki | IC50 |
| Cmpd | Name | Mass | Ion | (μM) | (uM) |
| 292 | 1-[4-methoxy-3-(pyridin-2-ylethynyl)benzoyl]- | 398.2 | M + H | 0.014 | 0.208 |
| | 4-phenyl piperazine | | | | |
| 293 | 4-amino-2-{4-[4-methoxy-3-(pyridin-2- | 440.2 | M + H | 0.058 | >1.000 |
| | ylethynyl)benzoyl]piperazin-1-yl}pyrimidine-5- | | | | |
| | carbonitrile | | | | |
| 294 | 4-chloro-6-{4-[4-methoxy-3-(pyridin-2- | 480.1 | M + H | 0.099 | |
| | ylethynyl)benzoyl]piperazin-1-yl}-2- | | | | |
| | (methylthio)pyrimidine | | | | |
| 295 | 2-chloro-5-fluoro-4-{4-[4-methoxy-3-(pyridin-2- | 452.1 | M + H | 0.102 | |
| | ylethynyl)benzoyl]piperazin-1-yl}pyrimidine | | | | |
| 296 | 4-{4-[4-methoxy-3-(pyridin-2- | 446.2 | M + H | 0.022 | >1.000 |
| | ylethynyl)benzoyl]piperazin-1-yl}-2- | | | | |
| | (methylthio)pyrimidine | | | | |
| 297 | 4-chloro-6-{4-[4-methoxy-3-(pyridin-2- | 448 | M + H | 0.159 | |
| | ylethynyl)benzoyl]piperazin-1-yl}-2- | | | | |
| | methylpyrimidine | | | | |
| 298 | 5-fluoro-2-{4-[4-methoxy-3-(pyridin-2- | 418 | M + H | 0.033 | 0.102 |
| | ylethynyl)benzoyl] piperazin-1-yl}pyrimidine | | | | |
| 299 | 5-methoxy-2-{4-[4-methoxy-3-(pyridin-2- | 430.2 | M + H | 0.025 | 0.119 |
| | ylethynyl)benzoyl] piperazin-1-yl}pyrimidine | | | | |
| 300 | 5-fluoro-2-{4-[4-methoxy-3-(pyridin-2- | 433.2 | M + H | 0.030 | 0.198 |
| | ylethynyl)benzoyl] piperazin-1-yl}pyrimidin-4- | | | | |
| | amine | | | | |
| 301 | 3-methoxy-6-{4[4-methoxy-3-(pyridin-2- | 430.2 | M + H | 0.083 | 0.204 |
| | ylethynyl)benzoyl] piperazin-1-yl}pyridazine | | | | |
| 302 | 6-chloro-3-{4-[4-methoxy-3-(pyridin-2- | 448.2 | M + H | 0.039 | >1.000 |
| | ylethynyl)benzoyl] piperazin-1-yl}-4-methyl | | | | |
| | pyridazine | | | | |
| 303 | 3-chloro-6-{4-[4-methoxy-3-(pyridin-2- | 434.1 | M + H | 0.031 | >1.000 |
| | ylethynyl)benzoyl] piperazin-1-yl}pyridazine | | | | |
| 304 | 2-chloro-3-{4-[4-methoxy-3-(pyridin-2- | 434.1 | M + H | 0.052 | 0.408 |
| | ylethynyl)benzoyl] piperazin-1-yl}pyrazine | | | | |
| 305 | 2,4-dimethoxy-6-{4-[4-methoxy-3-(pyridin-2- | 461.1 | M + H | 0.229 | |
| | ylethynyl)benzoyl]piperazin-1-yl}-1,3,5-triazine | | | | |
| 306 | 1-chloro-4-{4-[4-methoxy-3-(pyridin-2- | 484.1 | M + H | 0.072 | 0.356 |
| | ylethynyl)benzoyl] piperazin-1-yl}phthalazine | | | | |

### Example 10

### 3-{4-[4-Methyl-3-(pyridin-2-ylethynyl)benzoyl]piperazin-1-yl}-1,2-benzisoxazole (Compound 307)

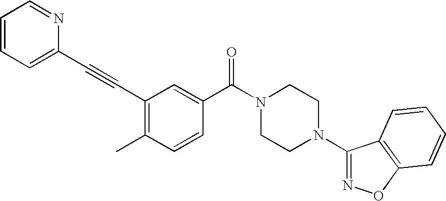

### Step 1: Methyl 4-methyl-3-(pyridin-2-ylethynyl)benzoate

Methyl 3-iodo-4-methylbenzoate (5.52 g, 20 mmol), 2-ethylnylpyridine (3.2 mL, 31 mmol), and triethylamine (6.2 mL, 44.7 mmol) were dissolved in 100 mL of toluene and purged with nitrogen. Then CuI (0.78 g, 3.9 mmol) and Pd(Ph₃P)₂Cl₂ (2.9 g, 4.1 mmol) were added and the resulting suspension was stirred at 100° C. for 6 hours. The reaction was concentrated at reduced pressure and purified by flash chromatography on silica (40:1 CH₂Cl₂/EtOAc) to yield 2.63 g (52%) of the product as a greenish solid.

### Step 2: 4-Methyl-3-(pyridin-2-ylethynyl)benzoic acid

Methyl 4-methyl-3-(pyridin-2-ylethynyl)benzoate (2.2 g, 8.7 mmol) was dissolved in a mixture of THF (75 mL), MeOH (25 mL), and H₂O (25 mL) and treated with lithium hydroxide monohydrate (420 mg, 10 mmol). The reaction was stirred at room temperature overnight and then concentrated at reduced pressure. The remaining residue was diluted with 50 mL of H₂O and acidified to pH 4.0 with 1N HCl. The resulting precipitate was collected by suction filtration. The collected precipitate was dried in a vacuum oven at 50° C. for 3 hours to yield 1.57 g (76%) of the carboxylic acid as a gray solid. No additional purification of the carboxylic acid was required.

### Step 3: (4-(Benzo[d]isoxazol-3-yl)piperazin-1-yl)(4-methyl-3-(pyridin-2-ylethynyl)phenyl)methanone (Compound 307)

4-Methyl-3-(pyridin-2-ylethynyl)benzoic acid (593 mg, 2.5 mmol), 3-(piperazin-1-yl)benzo[d]isoxazole (570 mg, 2.8 mmol), and triethylamine (1.05 mL, 7.5 mmol) are dissolved in 25 mL of CH₂Cl₂ and treated with EDCl (528 mg, 2.75 mmol) and HOBT (371 mg, 2.75 mmol). The reaction is stirred at room temperature overnight. The crude mixture is diluted EtOAc and washed with water and brine. The organic layer is dried over MgSO₄, filtered, concentrated, and purified by flash chromatography on silica gel (CH₂Cl₂/EtOAc) to yield 887 mg (84%) of the product as an off white solid.

### UNLESS NOTED OTHERWISE THE FOLLOWING VALUES REFER TO FORMULA I

### WHEREIN R₁, R₄, R₄ₐ, R₅, R₅ₐ═H; X₂═CH; Z=CO

TABLE 10

| Cmpd | Name | R₂ | X₁ | R₆ |
|---|---|---|---|---|
| 307 | 3-{4-[4-methyl-3- (pyridin-2-ylethynyl) benzoyl]piperazin-1- yl}-1,2-benzisoxazole | 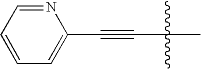 | CCH₃ | 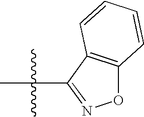 |

TABLE 10A

| | | LCMS data | | | Biological Activity | |
|---|---|---|---|---|---|---|
| | | Time | | | Median | |
| Cmpd | Name | (min.) | Mass | Ion | Kᵢ (μM) | IC50 (uM) |
| 307 | 3-{4-[4-methyl-3-(pyridin-2- | | 423.2 | M + H | 0.001 | 0.050 |
| | ylethynyl)benzoyl]piperazin-1- | | | | | |
| | yl}-1,2-benzisoxazole | | | | | |

### Example 11

### 4-Methoxy-2-{4-[4-methyl-3-(pyridin-2-ylethynyl)benzoyl]piperazin-1-yl}pyrimidine (Compound 308)

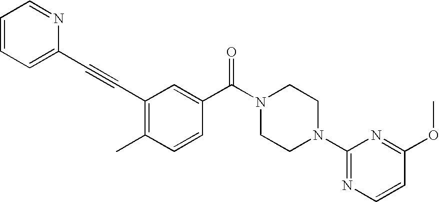

4-Methyl-3-(pyridin-2-y ethynyl)benzoic acid (47 mg, 0.2 mmol), 4-methoxy-2-(piperazin-1-yl)pyrimidine (49 mg, 0.25 mmol), and triethylamine (139 uL, 1.0 mmol) were dissolved in 3 mL of CH₂Cl₂ and treated with PyBOP (130 mg, 0.25 mmol). The reaction was stirred at room temperature overnight and directly purified by flash chromatography on silica gel (CH₂Cl₂/EtOAc) to yield 47 mg (57%) of the product as a white solid.

Compounds 308-311, shown in Table 11 and Table 11A below, were prepared using the procedure of Example 11 described above.

### UNLESS NOTED OTHERWISE THE FOLLOWING VALUES REFER TO FORMULA I

### WHEREIN R₁, R₄, R₄ₐ, R₅, R₅ₐ═H; X₂═CH; Z=CO

TABLE 11

| Cmpd | Name | R₂ | X₁ | R₆ |
|---|---|---|---|---|
| 308 | 4-methoxy-2-{4-[4- methyl-3-(pyridin-2-yl ethynyl)benzoyl]piperazin- 1-yl} pyrimidine | 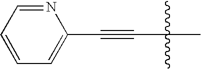 | CCH₃ | 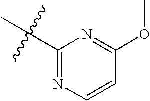 |
| 309 | 2-{4-[4-methyl-3-(pyridin- 2-yl ethynyl)benzoyl]-1- yl}pyrimidine | 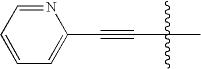 | CCH₃ | 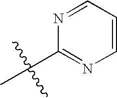 |
| 310 | 1-(3,5-dichloro pyridin-2- yl)-4-[4-methyl-3-(pyridin- 2-ylethynyl) benzoyl] piperazine | 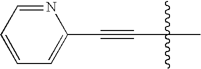 | CCH₃ | 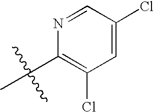 |
| 311 | 1-(3-chloropyridin-2-yl)-4- [4-methyl-3-(pyridin-2-yl ethynyl)benzoyl] piperazine | 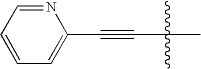 | CCH₃ | 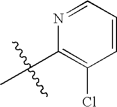 |

TABLE 11A

| | | LCMS data | | | Biological Activity | |
|---|---|---|---|---|---|---|
| | | Time | | | Median | |
| Cmpd | Name | (min.) | Mass | Ion | Ki (μM) | IC50 (uM) |
| 308 | 4-methoxy-2-{4-[4-methyl-3- | | 414.2 | M + H | 0.008 | 0.107 |
| | (pyridin-2-ylethynyl)benzoyl]piperazin- | | | | | |
| | 1-yl}pyrimidine | | | | | |
| 309 | 2-{4-[4-methyl-3-(pyridin-2- | | 384.2 | M + H | 0.034 | 0.193 |
| | ylethynyl)benzoyl]piperazin-1- | | | | | |
| | yl}pyrimidine | | | | | |
| 310 | 1-(3,5-dichloropyridin-2-yl)-4- | | 451.1 | M + H | 0.745 | |
| | [4-methyl-3-(pyridin-2-yl | | | | | |
| | ethynyl)benzoyl]piperazine | | | | | |
| 311 | 1-(3-chloropyridin-2-yl)-4-[4- | | 417.2 | M + H | 0.140 | |
| | methyl-3-(pyridin-2-yl | | | | | |
| | ethynyl)benzoyl]piperazine | | | | | |

### Example 12

### 2-{4-[4-Fluoro-3-(pyridin-2-ylethynyl)benzoyl]piperazin-1-yl}pyrimidine (Compound 312)

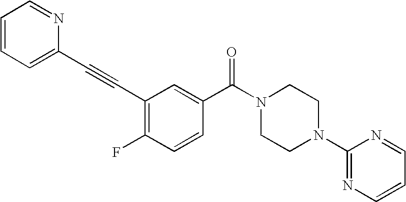

### Step 1: Methyl 4-fluoro-3-(pyridin-2-ylethynyl)benzoate

Methyl 3-bromo-4-fluorobenzoate (4.66 g, 20 mmol), 2-ethylnylpyridine (3.2 mL, 31 mmol), and triethylamine (6.2 mL, 44.7 mmol) were dissolved in 100 mL of toluene and purged with nitrogen. Then CuI (0.78 g, 3.9 mmol) and Pd(Ph₃P)₂Cl₂ (2.9 g, 4.1 mmol) were added and the resulting suspension was stirred at 100° C. for 6 hours. The reaction was concentrated at reduced pressure and purified by flash chromatography on silica (40:1 CH₂Cl₂/EtOAc) to yield 2.0 g (39%) of the product as a brown solid.

### Step 2: 4-fluoro-3-(pyridin-2-ylethynyl)benzoic acid

Methyl 4-fluoro-3-(pyridin-2-ylethynyl)benzoate (1.7 g, 6.6 mmol) was dissolved in a mixture of THF (75 mL), MeOH (25 mL), and H₂O (25 mL) and treated with lithium hydroxide monohydrate (420 mg, 10 mmol). The reaction was stirred at room temperature overnight and then concentrated at reduced pressure. The remaining residue was diluted with 50 mL of H₂O and acidified to pH 4.0 with 1N HCl. The resulting precipitate was collected by suction filtration. The collected precipitate was dried in a vacuum oven at 50° C. for 3 hours to yield 1.24 g (78%) of the carboxylic acid as a tan solid. No additional purification of the carboxylic acid was required.

### Step 3: 2-{4-[4-Fluoro-3-(pyridin-2-ylethynyl)benzoyl]piperazin-1-yl}pyrimidine (Compound 312)

4-Fluoro-3-(pyridin-2-ylethynyl)benzoic acid (48 mg, 0.2 mmol), 2-(piperazin-1-yl)pyrimidine (38 uL, 0.25 mmol), and triethylamine (139 uL, 1.0 mmol) were dissolved in 3 mL of CH₂Cl₂ and treated with PyBOP (130 mg, 0.25 mmol). The reaction was stirred at room temperature overnight and directly purified by flash chromatography on silica gel (CH₂Cl₂/EtOAc) to yield 54 mg (70%) of the product as a pink solid.

Compounds 312-317, shown in Table 12 below, were prepared using the procedure of Example 12 described above.

### UNLESS NOTED OTHERWISE THE FOLLOWING VALUES REFER TO FORMULA i

### WHEREIN R₁, R₄, R₄ₐ, R₅, R₅ₐ═H; X₂═CH; Z=CO

TABLE 12

| Cmpd | Name | R₂ | X₁ | R₆ |
|---|---|---|---|---|
| 312 | 2-{4-[4-fluoro-3- (pyridin-2-yl ethynyl)benzoyl]piperazin- 1-yl}pyrimidine | 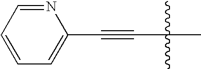 | CF | 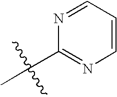 |
| 313 | 1-[4-fluoro-3-(pyridin-2- yl ethynyl)benzoyl]-4- pyridin-2-yl piperazine | 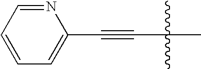 | CF | 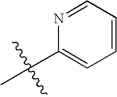 |
| 314 | 1-(3,5-dichloro pyridin- 2-yl)-4-[4-fluoro-3- (pyridin-2- ylethynyl)benzoyl] piperazine | 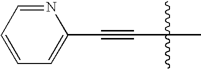 | CF | 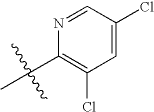 |
| 315 | 1-(3-chloropyridin-2-yl)- 4-[4-fluoro-3-(pyridin-2- yl ethynyl)benzoyl] piperazine | 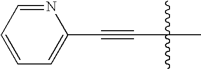 | CF | 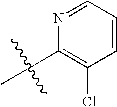 |
| 316 | 3-{4-[4-fluoro-3- (pyridin-2-yl ethynyl)benzoyl] piperazin-1-yl}-1,2- benzisoxazole | 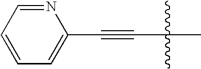 | CF | 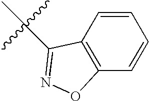 |
| 317 | 3-{4-[4-fluoro-3- (pyridin-2-yl ethynyl)benzoyl]piperazin- 1-yl}-4-methoxy pyrimidine | 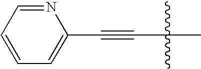 | CF | 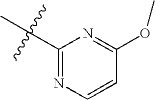 |

TABLE 12A

| | | | | Biological Activity | |
|---|---|---|---|---|---|
| | | LCMS data | | Median Ki | |
| Cmpd | Name | Mass | Ion | (μM) | IC50 (uM) |
| 312 | 2-{4-[4-fluoro-3-(pyridin-2- | 388.2 | M + H | 0.084 | |
| | ylethynyl)benzoyl]piperazin-1- | | | | |
| | yl}pyrimidine | | | | |
| 313 | 1-[4-fluoro-3-(pyridin-2-ylethynyl)benzoyl]- | 387.1 | M + H | 0.023 | 0.062 |
| | 4-pyridin-2- | | | | |
| | ylpiperazine | | | | |
| 314 | 1-(3,5-dichloropyridin-2-yl)-4-[4-fluoro- | 455.0 | M + H | 1.843 | |
| | 3-(pyridin-2- | | | | |
| | ylethynyl)benzoyl]piperazine | | | | |
| 315 | 1-(3-chloropyridin-2-yl)-4-[4-fluoro-3- | 421.1 | M + H | 0.270 | |
| | (pyridin-2-ylethynyl) benzoyl]piperazine | | | | |
| 316 | 3-{4-[4-fluoro-3-(pyridin-2-ylethynyl)benzoyl]piperazin- | 427.1 | M + H | 0.002 | 0.024 |
| | 1-yl}-1,2- | | | | |
| | benzisoxazole | | | | |
| 317 | 2-{4-[4-fluoro-3-(pyridin-2-ylethynyl)benzoyl]piperazin- | 418.1 | M + H | 0.020 | 0.030 |
| | 1-yl}-4-methoxy | | | | |
| | pyrimidine | | | | |

### Example 13

### 1-[4-Ethoxy-3-(pyridin-2-ylethynyl)benzoyl]-4-pyridin-2-ylpiperazin (Compound 318)

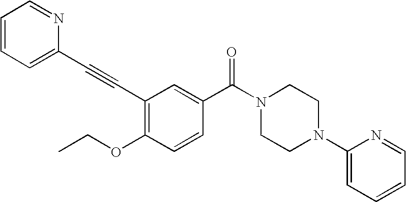

### Step 1: Methyl 4-ethoxy-3-iodobenzoate

Methyl 4-hydroxy-3-iodobenzoate (2.78 g, 10 mmol) was dissolved in 20 mL of DMF and treated with Cs₂CO₃ (6.5 g, 20 mmol) and ethyliodide (1.0 mL, 12 mmol). The resulting suspension was stirred at room temperature overnight. The reaction mixture was subsequently diluted with EtOAc and washed with water (×2) and brine. The organic layer was dried (MgSO₄), filtered, and concentrated at reduced pressure to yield 3.0 g of a white solid. The crude material was used in the next step without additional purification.

### Step 2: Methyl 4-ethoxy-3-(pyridin-2-ylethynyl)benzoate

Crude methyl 4-ethoxy-3-iodobenzoate (10 mmol), 2-ethylnylpyridine (1.6 mL, 15 mmol), and triethylamine (3.1 mL, 22 mmol) are dissolved in 50 mL of toluene and purged with nitrogen. Then CuI (390 mg, 2 mmol) and Pd(Ph₃P)₂Cl₂ (1.45 g, 2 mmol) are added and the resulting suspension is stirred at 100° C. for 6 hours. The reaction is concentrated at reduced pressure and purified by flash chromatography on silica (40:1 CH₂Cl₂/EtOAc) to yield 1.25 g (44% for 2 steps) of the product as a white solid.

### Step 3: 4-Ethoxy-3-(pyridin-2-ylethynyl)benzoic acid

Methyl 4-ethoxy-3-(pyridin-2-ylethynyl)benzoate (1.1 g, 3.9 mmol) was dissolved in a mixture of THF (75 mL), MeOH (25 mL), and H₂O (25 mL) and treated with lithium hydroxide monohydrate (420 mg, 10 mmol). The reaction was stirred at room temperature overnight and then concentrated at reduced pressure. The remaining residue was diluted with 50 mL of H₂O and acidified to pH 4.0 with 1N HCl. The resulting precipitate was collected by suction filtration. The collected precipitate was dried in a vacuum oven at 50° C. for 3 hours to yield 857 mg (82%) of the carboxylic acid as an off-white solid. No additional purification of the carboxylic acid was required.

### Step 4: 1-[4-Ethoxy-3-(pyridin-2-ylethynyl)benzoyl]-4-pyridin-2-ylpiperazin (Compound 318)

4-Ethoxy-3-(pyridin-2-ylethynyl)benzoic acid (53 mg, 0.2 mmol), 1-(pyridin-2-yl)piperazine (38 uL, 0.25 mmol), and triethylamine (139 uL, 1.0 mmol) were dissolved in 3 mL of CH₂Cl₂ and treated with PyBOP (130 mg, 0.25 mmol). The reaction was stirred at room temperature overnight and directly purified by flash chromatography on silica gel (CH₂Cl₂/EtOAc) to yield 59 mg (92%) of the product as a tan solid.

Compounds 318-322, shown in Table 13 below, were prepared using the procedure of Example 13 described above.

### UNLESS NOTED OTHERWISE THE FOLLOWING VALUES REFER TO FORMULA I

### WHEREIN R₁, R₄, R₄ₐ, R₅, R₅ₐ═H; X₂═CH; Z=CO

TABLE 13

| Cmpd | Name | R₂ | X₁ | R₆ |
|---|---|---|---|---|
| 318 | 1-[4-ethoxy-3-(pyridin-2- ylethynyl) benzoyl]-4- pyridin-2-ylpiperazine | 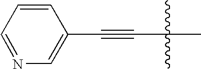 | COC₂H₅ | 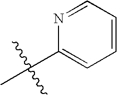 |
| 319 | 1-(3,5-dichloro pyridin-2- yl)-4-[4-ethoxy-3-(pyridin-2- ylethynyl) benzoyl] piperazine | 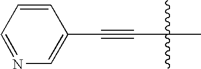 | COC₂H₅ | 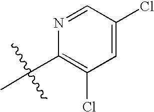 |
| 320 | 2-{4-[4-ethoxy-3-(pyridin-2- ylethynyl) benzoyl] piperazin-1-yl}-4-methoxy pyrimidine | 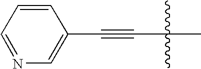 | COC₂H₅ | 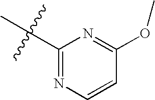 |
| 321 | 1-(3-chloropyridin-2-yl)-4- [4-ethoxy-3-(pyridin-2- ylethynyl) benzoyl] piperazine | 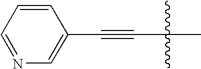 | COC₂H₅ | 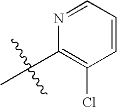 |
| 322 | 3-{4-[4-ethoxy-3-(pyridin-2- ylethynyl) benzoyl] piperazin-1-yl}-1,2- benzisoxazole | 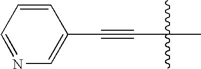 | COC₂H₅ | 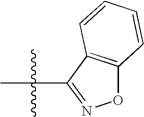 |

TABLE 13A

| | | | | Biological | |
|---|---|---|---|---|---|
| | | | | ctivity | |
| | | LCMS data | | Median Ki | IC50 |
| Cmpd | Name | Mass | Ion | (μM) | (uM) |
| 318 | 1-[4-ethoxy-3-(pyridin-2-ylethynyl)benzoyl]- | 413.1 | M + H | 0.018 | 0.019 |
| | 4-pyridin-2-ylpiperazine | | | | |
| 319 | 1-(3,5-dichloropyridin-2-yl)-4-[4- | 481.1 | M + H | 0.652 | |
| | ethoxy-3-(pyridin-2-ylethynyl)benzoyl]piperazine | | | | |
| 320 | 2-{4-[4-ethoxy-3-(pyridin-2-ylethynyl)benzoyl]piperazin- | 444.2 | M + H | 0.020 | 0.031 |
| | 1-yl}-4- | | | | |
| | methoxypyrimidine | | | | |
| 321 | 1-(3-chloropyridin-2-yl)-4-[4-ethoxy-3- | 447.1 | M + H | 0.149 | |
| | (pyridin-2-ylethynyl)benzoyl]piperazine | | | | |
| 322 | 3-{4-[4-ethoxy-3-(pyridin-2-ylethynyl)benzoyl]piperazin- | 453.1 | M + H | 0.006 | 0.035 |
| | 1-yl}-1,2- | | | | |
| | benzisoxazole | | | | |

### Example 14

### 1-{[4-(Cyclopropylmethoxy)-3-(pyridin-2-ylethynyl)phenyl]carbonyl}-4-pyridin-2-ylpiperazin (Compound 323)

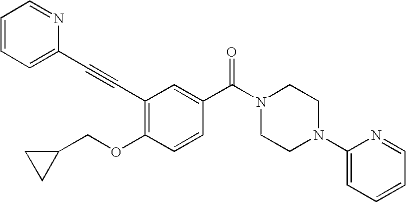

### Step 1: Methyl 4-(cyclopropylmethoxy)-3-iodobenzoate

Methyl 4-hydroxy-3-iodobenzoate (2.78 g, 10 mmol) was dissolved in 20 mL of DMF and treated with Cs₂CO₃ (6.5 g, 20 mmol) and cyclopropylmethyl bromide (1.25 mL, 12 mmol). The resulting suspension was stirred at room temperature overnight. The reaction mixture was subsequently diluted with EtOAc and washed with water (×2) and brine. The organic layer was dried (MgSO₄), filtered, and concentrated at reduced pressure to yield 3.3 g of a pale yellow oil. The crude material was used in the next step without additional purification.

### Step 2: Methyl 4-(cyclopropylmethoxy)-3-(pyridin-2-ylethynyl)benzoate

Crude methyl 4-(cyclopropylmethoxy)-3-iodobenzoate (10 mmol), 2-ethylnylpyridine (1.6 mL, 15 mmol), and triethylamine (3.1 mL, 22 mmol) were dissolved in 50 mL of toluene and purged with nitrogen. Then CuI (390 mg, 2 mmol) and Pd(Ph₃P)₂Cl₂ (1.45 g, 2 mmol) were added and the resulting suspension was stirred at 100° C. for 6 hours. The reaction was concentrated at reduced pressure and purified by flash chromatography on silica (CH₂Cl₂/EtOAc) to yield 1.52 g (50% for 2 steps) of the product as an oil.

### Step 3: 4-(Cyclopropylmethoxy)-3-(pyridin-2-ylethynyl)benzoic acid

Methyl 4-(cyclopropylmethoxy)-3-(pyridin-2-ylethynyl)benzoate (1.5 g, 4.9 mmol) was dissolved in a mixture of THF (75 mL), MeOH (25 mL), and H₂O (25 mL) and treated with lithium hydroxide monohydrate (420 mg, 10 mmol). The reaction was stirred at room temperature overnight and then concentrated at reduced pressure. The remaining residue was diluted with 50 mL of H₂O and acidified to pH 4.0 with 1N HCl. The resulting precipitate was collected by suction filtration. The collected precipitate was dried in a vacuum oven at 50° C. for 3 hours to yield 1.31 g (91%) of the carboxylic acid as a pale yellow solid. No additional purification of the carboxylic acid was required.

### Step 4: 1-{[4-(Cyclopropylmethoxy)-3-(pyridin-2-ylethynyl)phenyl]carbonyl}-4-pyridin-2-ylpiperazin (Compound 323)

4-(Cyclopropylmethoxy)-3-(pyridin-2-ylethynyl)benzoic acid (59 mg, 0.2 mmol), 1-(pyridin-2-yl)piperazine (61 uL, 0.4 mmol), and triethylamine (84 uL, 0.6 mmol) were dissolved in 4 mL of CH₂Cl₂ and treated with HOBt (40 mg, 0.3 mmol) and EDC (58 mg, 0.3 mmol). The reaction was stirred at room temperature overnight and directly purified by flash chromatography on silica gel (CH₂Cl₂/EtOAc) to yield 79 mg (90%) of the product as a white solid.

Compounds 323-325, shown in Table 14, were prepared using the procedure of Example 13 described above.

### UNLESS NOTED OTHERWISE THE FOLLOWING VALUES REFER TO FORMULA I

### WHEREIN R₁, R₄, R₄ₐ, R₅, R₅ₐ═H; X₂═CH; Z=CO

TABLE 14

| Cmpd | Name | R₂ | X₁ | R₆ |
|---|---|---|---|---|
| 323 | 1-{[4-(cyclopropyl methoxy)-3-(pyridin- 2-ylethynyl)phenyl] carbonyl}-4-pyridin- 2-ylpiperazine | 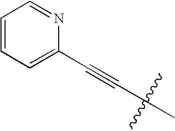 | 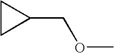 | 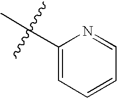 |
| 324 | 3-(4-{[4-(cyclopropyl methoxy)-3-(pyridin- 2-ylethynyl)phenyl] carbonyl}piperazin- 1-yl)-1,2- benzisoxazole | 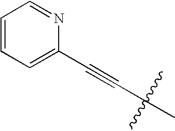 | 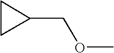 | 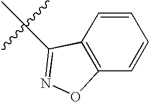 |
| 325 | 2-(4-{[4-(cyclopropyl methoxy)-3-(pyridin- 2-ylethynyl)phenyl] carbonyl}piperazin- 1-yl)pyrimidine | 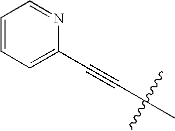 | 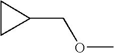 | 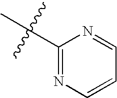 |

TABLE 14A

| | | | | Biological Activity | |
|---|---|---|---|---|---|
| | | LCMS data | | Median | |
| Cmpd | Name | Mass | Ion | Ki (μM) | IC50 (uM) |
| 323 | 1-{[4-(cyclopropylmethoxy)-3-(pyridin-2- | 439.2 | M + H | 0.198 | |
| | ylethynyl)phenyl]carbonyl}-4-pyridin-2- | | | | |
| | ylpiperazine | | | | |
| 324 | 3-(4-{[4-(cyclopropylmethoxy)-3-(pyridin-2- | 479.1 | M + H | 0.005 | 0.078 |
| | ylethynyl)phenyl]carbonyl}piperazin-1-yl)- | | | | |
| | 1,2-benzisoxazole | | | | |
| 325 | 2-(4-{[4-(cyclopropylmethoxy)-3-(pyridin-2- | 440.1 | M + H | 1.343 | |
| | ylethynyl)phenyl]carbonyl}piperazin-1- | | | | |
| | yl)pyrimidine | | | | |

### Example 15

### 1-(4-Chlorophenyl)-4-{[4-methoxy-3-(pyridin-2-ylethynyl)phenyl]carbonyl}piperazin-2-one (Compound 326)

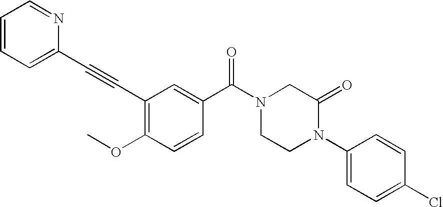

### Step 1: Methyl 4-methoxy-3-(pyridin-2-ylethynyl)benzoate

Methyl 3-iodo-4-methoxybenzoate (6.0 g, 20.4 mmol), 2-ethylnylpyridine (3.14 mL, 31.1 mmol), and triethylamine (6.2 mL, 44.7 mmol) were dissolved in 100 mL of toluene and purged with nitrogen. Then CuI (0.78 g, 3.9 mmol) and Pd(Ph₃P)₂Cl₂ (2.9 g, 4.1 mmol) were added and the resulting suspension was stirred at 100° C. for 6 hours. The reaction was concentrated at reduced pressure and purified by flash chromatography on silica (20:1 CH₂Cl₂/EtOAc) to yield 5.3 g (96%) of product as a brown solid.

### Step 2: 4-Methoxy-3-(pyridin-2-ylethynyl)benzoic acid

Methyl 4-methoxy-3-(pyridin-2-ylethynyl)benzoate (5.3 g, 20 mmol) was dissolved in a mixture of THF (150 mL), MeOH (20 mL), and H₂O (40 mL) and treated with lithium hydroxide monohydrate (1.68 g, 40 mmol). The reaction was stirred at room temperature overnight and then concentrated at reduced pressure to an approximate volume of 40 mL. The remaining solution was diluted with an additional 50 mL of H₂O, washed with Et₂O (×2), and acidified to pH 4.0. The resulting precipitate was collected by suction filtration. The filtrate was saturated with solid NaCl and extracted with EtOAc (2×100 mL). The organic extracts were concentrated to yield a solid residue that was added to the collected precipitate and the combined solids were dried in a vacuum oven at 50° C. for 3 hours to yield 4.65 g (93%) of the carboxylic acid as a tan solid. No additional purification of the carboxylic acid was required.

### Step 3 1-(4-Chlorophenyl)-4-{[4-methoxy-3-(pyridin-2-ylethynyl)phenyl]carbonyl}piperazin-2-one (Compound 326)

4-Methoxy-3-(pyridin-2-ylethynyl)benzoic acid (51 mg, 0.2 mmol), 1-(4-chlorophenyl)piperazin-2-one (74 mg, 0.3 mmol), and triethylamine (105 uL, 0.75 mmol) were dissolved in 3 mL of CH₂Cl₂ and treated with HOBt (34 mg, 0.25 mmol) and EDC (48 mg, 0.25 mmol). The reaction was stirred at room temperature overnight and directly purified by flash chromatography on silica gel (CH₂Cl₂/EtOAc) to yield 85 mg (95%) of the product as a white solid.

Compounds 326-330, shown in Table 15 below, were prepared using the procedure of Example 15 described above.

### UNLESS NOTED OTHERWISE THE FOLLOWING VALUES REFER TO FORMULA I

### WHEREIN R₁, R₄, R₄ₐ, R₅, R₅ₐ═H; X₂═CH; Z=CO

TABLE 15

| Cmpd | Name | R₂ | X₁ | R₅ | R₆ |
|---|---|---|---|---|---|
| 326 | 1-(4-chlorophenyl)- 4-{[4-methoxy-3- (pyridin-2-yl ethynyl)phenyl]car bonyl}piperazin-2- one | 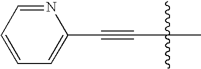 | COME | 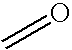 | 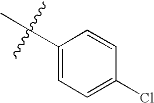 |
| 327 | 1-(3-chlorophenyl)- 4-{[4-methoxy-3- (pyridin-2-yl ethynyl)phenyl]car bonyl}piperazin-2- one | 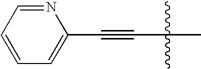 | COMe | 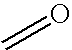 | 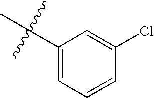 |
| 328 | 1-(2-chlorophenyl)- 4-{[4-methoxy-3- pyridin-2-yl ethynyl)phenyl]car bonyl}piperazin-2- one | 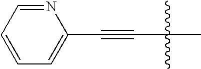 | COMe | 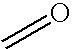 | 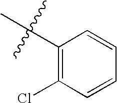 |
| 329 | 4-{[4-methoxy-3- (pyridin-2-yl ethynyl)phenyl]car bonyl}-1-phenyl piperazine-2-one | 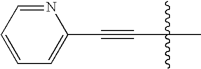 | COMe | 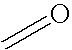 | 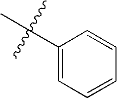 |
| 330 | 4-{[4-methoxy-3- (pyridin-2- ylethynyl)phenyl] carbonyl}-1- pyridin-2- ylpiperazin-2- one | 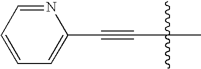 | COMe | 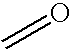 | 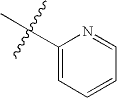 |

TABLE 15A

| | | | | Biological Activity | |
|---|---|---|---|---|---|
| | | LCMS data | | Median | |
| Cmpd | Name | Mass | Ion | Ki (μM) | IC50 (uM) |
| 326 | 1-(4-chlorophenyl)-4-{[4-methoxy-3- | 446.1 | M + H | 0.072 | 0.029 |
| | (pyridin-2-ylethynyl)phenyl]carbonyl}piperazin- | | | | |
| | 2-one | | | | |
| 327 | 1-(3-chlorophenyl)-4-{[4-methoxy-3- | 446.1 | M + H | 0.040 | 0.049 |
| | (pyridin-2-ylethynyl)phenyl]carbonyl}piperazin- | | | | |
| | 2-one | | | | |
| 328 | 1-(2-chlorophenyl)-4-{[4-methoxy-3- | 446.1 | M + H | 0.096 | 0.050 |
| | (pyridin-2-ylethynyl)phenyl]carbonyl}piperazin- | | | | |
| | 2-one | | | | |
| 329 | 4-{[4-methoxy-3-(pyridin-2-ylethynyl)phenyl]carbonyl}- | 412.1 | M + H | 0.084 | 0.118 |
| | 1-phenylpiperazin-2- | | | | |
| | one | | | | |
| 330 | 4-{[4-methoxy-3-(pyridin-2-ylethynyl)phenyl]carbonyl}- | 413.1 | M + H | 0.122 | |
| | 1-pyridin-2-ylpiperazin- | | | | |
| | 2-one | | | | |

### Example 16

### 1-Benzyl-4-[4-methoxy-3-(pyridin-2-ylethynyl)benzoyl]piperazin-2-one (Compound 331)

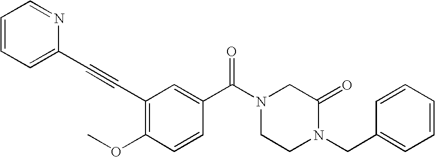

### Step 1: 4-(4-Methoxy-3-(pyridin-2-ylethynyl)benzoyl)piperazin-2-one

4-Methoxy-3-(pyridin-2-ylethynyl)benzoic acid (760 mg, 3.0 mmol), piperazin-2-one (455 mg, 4.5 mmol), and triethylamine (0.7 mL, 5 mmol) were dissolved in 30 mL of CH₂Cl₂ and treated with HOBt (608 mg, 4.5 mmol) and EDC (864 mg, 4.5 mmol). The reaction was stirred at room temperature overnight. The reaction was diluted with EtOAc and washed with water and brine. The organic layer was dried (Na₂SO₄), filtered, and purified by flash chromatography on silica gel (CH₂Cl₂/MeOH) to yield 469 mg (47%) of the product as a tan solid.

### Step 2: 1-Benzyl-4-[4-methoxy-3-(pyridin-2-ylethynyl)benzoyl]piperazin-2-one (Compound 331)

4-(4-Methoxy-3-(pyridin-2-ylethynyl)benzoyl)piperazin-2-one (50 mg, 0.15 mmol) was dissolved in 3 mL of DMF, cooled to −50° C., and treated with 400 uL of 0.5 M KHMDS in toluene (0.2 mmol). The reaction was stirred at −50° C. for 2 min. and treated with BnBr (42 uL, 0.35 mmol). The cold bath was removed and the reaction was warmed to room temperature. Upon reaching room temperature, the reaction was quenched with water, diluted with EtOAc, and washed with water and brine. The organic layer was dried (Na₂SO₄), filtered, and purified by flash chromatography on silica gel (CH₂Cl₂/EtOAc) to yield 30 mg (47%) of the product as an off white solid.

Compounds 331-334, shown in Table 16 below, were prepared using the procedure of Example 16 described above.

### UNLESS NOTED OTHERWISE THE FOLLOWING VALUES REFER TO FORMULA I

### WHEREIN R₁, R₄, R₄ₐ, R₅, R₅ₐ═H; X₂═CH; Z=CO

TABLE 16

| Cmpd | Name | R₂ | X₁ | R₅ | R₆ |
|---|---|---|---|---|---|
| 331 | 1-benzyl-4-[4- methoxy-3- (pyridin-2-yl ethynyl)benzoyl] piperazin-2-one | 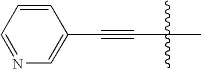 | COMe | 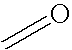 | 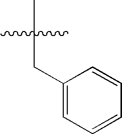 |
| 332 | 1-(2-chlorobenzyl)- 4-[4-methoxy-3- (pyridin-2-yl ethynyl)benzoyl] piperazin-2-one | 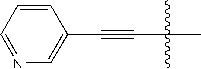 | COMe | 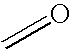 | 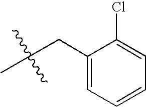 |
| 333 | 1-(3-chlorobenzyl)- 4-[4-methoxy-3- (pyridin-2-yl ethynyl)benzoyl] piperazin-2-one | 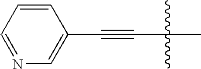 | COMe | 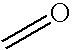 | 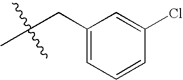 |
| 334 | 1-(4-chlorobenzyl)- 4-[4-methoxy-3- (pyridin-2-yl ethynyl)benzoyl] piperazin-2-one | 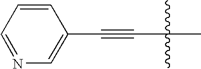 | COMe | 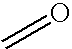 | 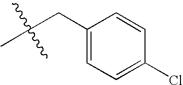 |

TABLE 16A

| | | | | Biological Activity | |
|---|---|---|---|---|---|
| | | LCMS data | | Median | |
| Cmpd | Name | Mass | Ion | Ki (μM) | IC50 (uM) |
| 331 | 1-benzyl-4-[4-methoxy-3-(pyridin-2- | 426.5 | M + H | 0.061 | 0.051 |
| | ylethynyl)benzoyl] piperazin-2-one | | | | |
| 332 | 1-(2-chlorobenzyl)-4-[4-methoxy-3- | 460.9 | M + H | 0.032 | 0.051 |
| | (pyridin-2-ylethynyl)benzoyl]piperazin-2- | | | | |
| | one | | | | |
| 333 | 1-(3-chlorobenzyl)-4-[4-methoxy-3- | 460.9 | M + H | 0.034 | 0.053 |
| | (pyridin-2-ylethynyl)benzoyl]piperazin-2- | | | | |
| | one | | | | |
| 334 | 1-(4-chlorobenzyl)-4-[4-methoxy-3- | 460.9 | M + H | 0.054 | 0.096 |
| | (pyridin-2-ylethynyl)benzoyl]piperazin-2- | | | | |
| | one | | | | |

### Example 17

### 1-Pyridin-2-yl-4-{[3-(pyridin-2-ylethynyl)-4-(trifluoromethoxy)phenyl]carbonyl}piperazine (Compound 340)

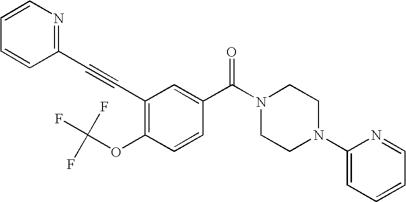

### Step 1: 3-Bromo-4-(trifluoromethoxy)benzoic acid

A solution of hydrogen peroxide (30% in water, 115 mL) in 15% aqueous NaOH was added slowly to a solution of 3-bromo-4-(trifluoromethoxy)benzaldehyde (25 g, 93 mmol) in methanol (115 mL) at 0° C. After the addition the reaction mixture was warmed up to room temperature and stirred for 4 hours. The reaction was monitored by TLC (10% MeOH in CH₂Cl₂). After the reaction was complete the reaction mixture was acidified with 5 N HCl to pH=1. The white solid formed was isolated by filtration, washed with water (2×), and then dried at 50° C. overnight to yield the title compound as a white solid (24.1 g, 91% yield).

### Step 2: Methyl 3-bromo-4-(trifluoromethoxy)benzoate

HCl (concentrated, 20 mL) was added to a solution of 3-bromo-4-(trifluoromethoxy)benzoic acid (30 g, 105 mmol) in methanol (160 mL). The mixture was heated at 70° C. for 20 h. After the reaction is complete the reaction mixture was concentrated to give a semi-solid. This solid was stirred in hexane (250 mL) for 2 h. Unreacted solid was removed by filtration. The filtrate was evaporated to yield the title compound as an oil (28.1 g, 89% yield).

### Step 3: Methyl 4-(trifluoromethoxy)-3-(pyridin-2-ylethynyl)benzoate

The title compound was prepared from methyl 3-bromo-4-(trifluoromethoxy)benzoate (step 2) in substantially the same manner as described in Example 3, step 3.

### Step 4: 3-(Pyridin-2-ylethynyl)-4-(trifluoromethoxy)benzoic acid

The title compound was prepared from methyl 4-(trifluoromethoxy)-3-(pyridin-2-ylethynyl)benzoate (step 3) in substantially the same manner as described in Example 3, step 4.

### Step 5: 1-Pyridin-2-yl-4-{[3-(pyridin-2-ylethynyl)-4-(trifluoro methoxy)phenyl]carbonyl}piperazine

The title compound was prepared from 3-(pyridin-2-ylethynyl)-4-(trifluoro methoxy)benzoic acid (step 4) and 1-(pyridin-2-yl)piperazine in substantially the same manner as described in Example 3, step 5.

Compounds 335-340 were synthesized according to Example 17.

### UNLESS NOTED OTHERWISE THE FOLLOWING VALUES REFER TO FORMULA I

### WHEREIN R₁, R₄, R₄ₐ, R₅, R₅ₐ═H; X₂═CH; Z=CO

TABLE 17

| | | | | |
|---|---|---|---|---|
| Cmpd | Name | R₂ | X₁ | R₆ |

| | | | | |
|---|---|---|---|---|
| 335 | 2-{4-[3-(pyridin-2-yl ethynyl)-4-(trifluoro methoxy)benzoyl] piperazin-1-yl}pyrazine | 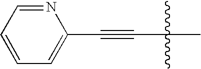 | COCF₃ | 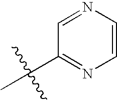 |
| 336 | 1-(3-chloropyridin-2- yl)-4-[3-(pyridin-2-yl ethynyl)-4-(trifluoro methoxy)benzoyl] piperazine | 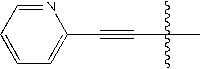 | COCF₃ | 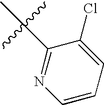 |
| 337 | 1-[3-(pyridin-2- ylethynyl)-4-(trifluoro methoxy)benzoyl]-4- [3-(trifluoromethyl) phenyl]piperazine | 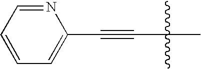 | COCF₃ | 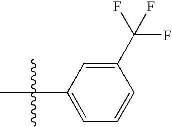 |
| 338 | 3-{4-[3-(pyridin-2- ylethynyl)-4-(trifluoro methoxy)benzoyl] piperazin-1-yl}-1,2- benzisoxazole | 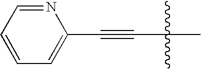 | COCF₃ | 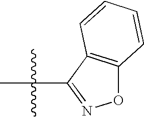 |
| 339 | 5-bromo-4-methoxy- 2-{4-[3-(pyridin-2- ylethynyl)-4-(trifluoro methoxy)benzoyl] piperazin-1-yl} pyrimidine | 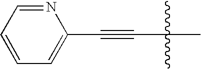 | COCF₃ | 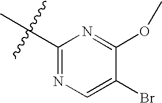 |
| 340 | 1-pyridin-2-yl-4-{[3- (pyridin-2 [symbol] ylethynyl)- 4-(trifluoromethoxy) phenyl]carbonyl} piperazine | 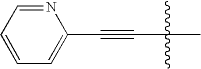 | COCF₃ | 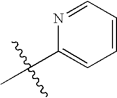 |

TABLE 17A

| | | | | Biological Activity | |
|---|---|---|---|---|---|
| | | LCMS data | | Median | |
| Cmpd | Name | Mass | Ion | Ki (μM) | IC50 (uM) |
| 335 | 2-{4-[3-(pyridin-2-ylethynyl)-4-(trifluoromethoxy)benzoyl]piperazin- | 454.1 | M + H | 0.176 | 0.273 |
| | 1-yl}pyrazine | | | | |
| 336 | 1-(3-chloropyridin-2-yl)-4-[3-(pyridin-2- | 487.0 | M + H | 1.066 | |
| | ylethynyl)-4-(trifluoromethoxy)benzoyl]piperazine | | | | |
| 337 | 1-[3-(pyridin-2-ylethynyl)-4-(trifluoromethoxy)benzoyl]- | 520.1 | M + H | 1.569 | |
| | 4-[3-(trifluoromethyl)phenyl]piperazine | | | | |
| 338 | 3-{4-[3-(pyridin-2-ylethynyl)-4-(trifluoromethoxy)benzoyl]piperazin- | 493.1 | M + H | 0.001 | 0.016 |
| | 1-yl}-1,2-benzisoxazole | | | | |
| 339 | 5-bromo-4-methoxy-2-{4-[3-(pyridin-2- | 562.0 | M + H | 0.702 | |
| | ylethynyl)-4-(trifluoromethoxy)benzoyl]piperazin- | | | | |
| | 1-yl}pyrimidine | | | | |
| 340 | 1-pyridin-2-yl-4-{[3-(pyridine-2-ylethynyl)-4- | 453.2 | M + H | 0.002 | 0.007 |
| | (trifluoromethoxy)phenyl]carbonyl}piperazine | | | | |

### Example 18

### 2-{4-[4-Methoxy-3-(pyridin-2-ylethynyl)benzoyl]piperazin-1-yl}pyrimidine (Compound 353)

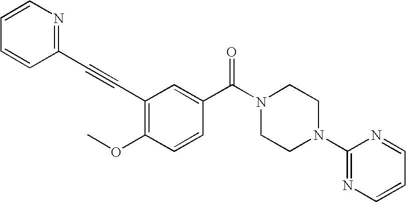

### Step 1: Methyl 4-methoxy-3-((trimethylsilyl)ethynyl)benzoate

The title compound was prepared from methyl 3-bromo-4-methoxybenzoate and ethynyltrimethylsilane in substantially the same manner as described in Example 3, step 3.

### Step 2: Methyl 3-ethynyl-4-methoxybenzoate

A mixture of methyl 4-methoxy-3-((trimethylsilyl)ethynyl)benzoate (4.2 g, 16.0 mmol) and potassium carbonate (1.3 g, 9.6 mmol) in a mixed solvent of methanol and tetrahydrofuran (1:1; 20 mL) was stirred at room temperature for 2 h. After the reaction was complete the reaction mixture was dried with anhydrous Na₂SO₄, filtered and evaporated to yield the title compound as an oil (3.7 g, 64% yield).

### Step 3: 3-Ethynyl-4-methoxybenzoic acid

The title compound was prepared from methyl 3-ethynyl-4-methoxybenzoate (step 2) in substantially the same manner as described in Example 3, step 4.

### Step 4: (3-Ethynyl-4-methoxyphenyl)(4-(pyrimidin-2-yl)piperazin-1-yl)methanone

The title compound was prepared from 3-Ethynyl-4-methoxybenzoic acid (step 3) and 2-(piperazin-1-yl)pyrimidine in substantially the same manner as described in Example 3, step 5.

### Step 5: 2-{4-[4-Methoxy-3-(pyridin-2-ylethynyl)benzoyl]piperazin-1-yl}pyrimidine

The title compound was prepared from methyl (3-ethynyl-4-methoxyphenyl)(4-(pyrimidin-2-yl)piperazin-1-yl)methanone (step 4) in substantially the same manner as described in Example 3, step 3.

Compounds 341-364 were synthesized according to Example 18.

### UNLESS NOTED OTHERWISE THE FOLLOWING VALUES REFER TO FORMULA I

### WHEREIN R₁, R₄, R₄ₐ, R₅, R₅ₐ═H; X₁═COMe, X₂═CH; Z=CO

TABLE 18

| Cmpd | Name | R₂ | Noted Values | R₆ |
|---|---|---|---|---|
| 341 | 1-(3-chloropyridin-2-yl)-4- [4-methoxy-3-(phenyl ethynyl) benzoyl] piperazine | 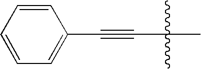 | COMe | 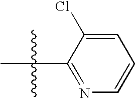 |
| 342 | 1-(3-chloropyridin-2-yl)-4- {4-methoxy-3-[(2-nitro phenyl)ethynyl]benzoyl} piperazine | 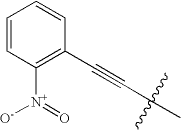 | COMe | 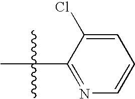 |
| 343 | 1-(3-{[3-(benzyloxy)phenyl] ethynyl}-4-methoxy benzoyl)-4-(3-chloropyridin- 2-yl)piperazine | 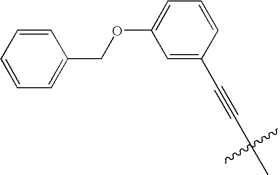 | COMe | 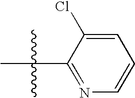 |
| 344 | 1-(3-chloropyridin-2-yl)-4- (4-methoxy-3-{[3-(trifluoro methoxy)phenyl]ethynyl} benzoyl)piperazine | 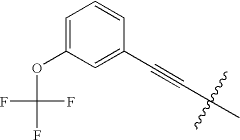 | COMe | 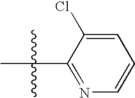 |
| 345 | 1-(3-chloropyridin-2-yl)-4- {4-methoxy-3-[(3- nitrophenyl)ethynyl]benzoyl} piperazine | 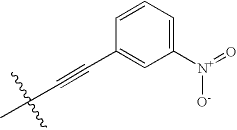 | COMe | 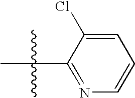 |
| 346 | 1-(3-{[4-(benzyloxy)phenyl] ethynyl}-4-methoxy benzoyl)-4-(3-chloropyridin- 2-yl)piperazine | 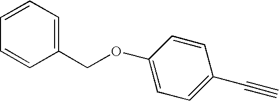 | COMe | 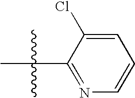 |
| 347 | 1-{4-[(5-{[4-(3- chloropyridin- 2-yl)piperazin-1-yl] carbonyl}-2-methoxy phenyl)ethynyl]phenyl} ethanone | 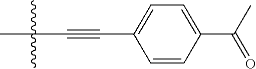 | COMe | 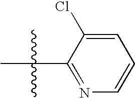 |
| 348 | 1-(3-chloropyridin-2-yl)-4- (4-methoxy-3-{[4-(trifluoro methoxy)phenyl]ethynyl} benzoyl)piperazine | 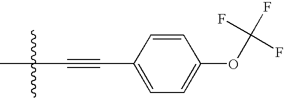 | COMe | 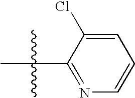 |
| 349 | 1-(3-chloropyridin-2-yl)-4- (4-methoxy-3-{[4-(trifluoro methyl)phenyl]ethynyl} benzoyl)piperazine | 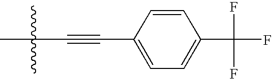 | COMe | 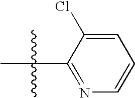 |
| 350 | 1-(3-chloropyridin-2-yl)-4- {4-methoxy-3-[(4- nitrophenyl)ethynyl]benzoyl} piperazine | 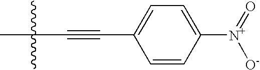 | COMe | 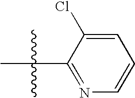 |
| 351 | 1-(3-chloropyridin-2-yl)-4- {3-[(2- fluorophenyl)ethynyl]-4- methoxybenzoyl}piperazine | 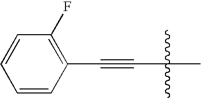 | COMe | 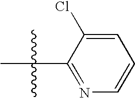 |
| 352 | 1-{3-[(4- chlorophenyl)ethynyl]-4- methoxybenzoyl}-4-(3- chloropyridin-2- yl)piperazine | 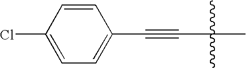 | COMe | 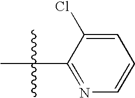 |
| 353 | 2-{4-[4-methoxy-3-(pyridin- 2- ylethynyl)benzoyl]piperazin- 1-yl}pyrimidine | 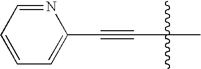 | COMe | 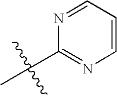 |
| 354 | 2-{4-[4-methoxy-3-(pyridin- 3- ylethynyl)benzoyl]piperazin- 1-yl}pyrimidine | 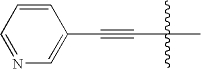 | COMe | 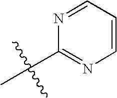 |
| 355 | 2-{4-[4-methoxy-3-(pyridin- 4- ylethynyl)benzoyl]piperazin- 1-yl}pyrimidine | 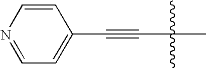 | COMe | 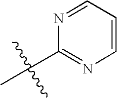 |
| 256 | 2-[4-(4-methoxy-3-{[3- (trifluoromethoxy)phenyl] ethynyl}benzoyl)piperazin-1- yl]pyrimidine | 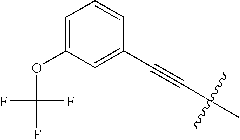 | COMe | 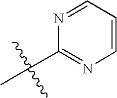 |
| 357 | 2-(4-{4-methoxy-3-[(3- nitrophenyl)ethynyl]benzoyl} piperazin-1-yl)pyrimidine | 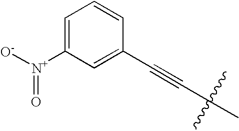 | COME | 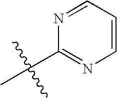 |
| 358 | 2-[4-(3-{[4- (benzyloxy)phenyl]ethynyl}- 4- methoxybenzoyl)piperazin- 1-yl]pyrimidine | 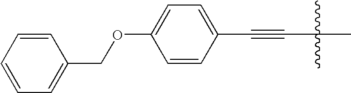 | COMe | 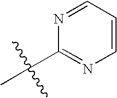 |
| 359 | 2-(4-{3-[(2-fluoro phenyl)ethynyl]-4- methoxybenzoyl}piperazin- 1-yl) pyrimidine | 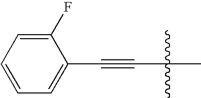 | COMe | 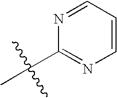 |
| 360 | 2-(4-{3-[(2-chloro phenyl)ethynyl]-4- methoxybenzoyl}piperazin- 1-yl) pyrimidine | 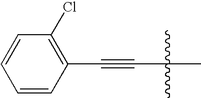 | COMe | 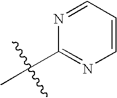 |
| 361 | 3-({2-methoxy-5-[(4- pyrimidin-2-ylpiperazin-1- yl)carbonyl]phenyl}ethynyl) benzo nitrile | 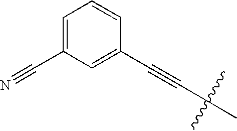 | COMe | 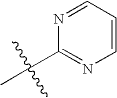 |
| 362 | 2-(4-{3-[(4- fluorophenyl)ethynyl]-4- methoxy benzoyl}piperazin-1- yl)pyrimidine | 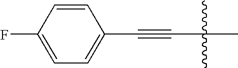 | COMe | 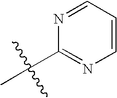 |
| 363 | 2-(4-{3-[(4-chloro phenyl)ethynyl]-4- methoxybenzoyl}piperazin- 1-yl) pyrimidine | 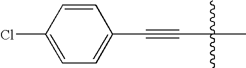 | COMe | 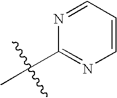 |
| 364 | 2-[4-(3-{[3- (difluoromethoxy)phenyl] ethynyl}-4- methoxybenzoyl)piperazin- 1-yl] pyrimidine | 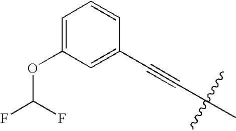 | COMe | 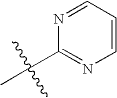 |

TABLE 18A

| | | | | Biological Activity | |
|---|---|---|---|---|---|
| | | LCMS data | | Median Ki | |
| Cmpd | Name | Mass | Ion | (μM) | IC50 (uM) |
| 341 | 1-(3-chloropyridin-2-yl)-4-[4-methoxy-3- | 432.1 | M + H | 1.130 | |
| | (phenylethynyl) benzoyl]piperazine | | | | |
| 342 | 1-(3-chloropyridin-2-yl)-4-{4-methoxy-3- | 477.0 | M + H | IC50 > 10 | |
| | [(2-nitrophenyl)ethynyl]benzoyl}piperazine | | | Um | |
| 343 | 1-(3-{[3-(benzyloxy)phenyl] ethynyl}-4- | 538.1 | M + H | IC50 > 10 | |
| | methoxybenzoyl)-4-(3-chloropyridin-2-yl)piperazine | | | uM | |
| 344 | 1-(3-chloropyridin-2-yl)-4-(4-methoxy-3- | 516.0 | M + H | IC50 > 10 | |
| | {[3-(trifluoromethoxy)phenyl]ethynyl}benzoyl)piperazine | | | uM | |
| 345 | 1-(3-chloropyridin-2-yl)-4-{4-methoxy-3- | 447.0 | M + H | 3.207 | |
| | [(3-nitrophenyl)ethynyl]benzoyl}piperazine | | | | |
| 346 | 1-(3-{[4-(benzyloxy)phenyl] ethynyl}-4- | 538.0 | M + H | IC50 > 10 | |
| | methoxybenzoyl)-4-(3-chloropyridin-2-yl)piperazine | | | Um | |
| 347 | 1-{4-[(5-{[4-(3-chloropyridin-2-yl)piperazin- | 474.1 | M + H | IC50 > 10 | |
| | 1-yl]carbonyl}-2-methoxyphenyl)ethynyl]phenyl}ethanone | | | uM | |
| 348 | 1-(3-chloropyridin-2-yl)-4-(4-methoxy-3- | 516.0 | M + H | IC50 > 10 | |
| | {[4-(trifluoro methoxy)phenyl]ethynyl}benzoyl)piperazine | | | uM | |
| 349 | 1-(3-chloropyridin-2-yl)-4-(4-methoxy-3- | 500.0 | M + H | IC50 > 10 | |
| | {[4-(trifluoromethyl)phenyl]ethynyl}benzoyl)piperazine | | | uM | |
| 350 | 1-(3-chloropyridin-2-yl)-4-{4-methoxy-3- | 477.0 | M + H | IC50 > 10 | |
| | [(4-nitrophenyl)ethynyl]benzoyl}piperazine | | | uM | |
| 351 | 1-(3-chloropyridin-2-yl)-4-{3-[(2- | 450.0 | M + H | 4.301 | |
| | fluorophenyl)ethynyl]-4- | | | | |
| | methoxybenzoyl}piperazine | | | | |
| 352 | 1-{3-[(4-chlorophenyl) ethynyl]-4- | 466.0 | M + H | 3.192 | |
| | methoxybenzoyl}-4-(3-chloropyridin-2-yl)piperazine | | | | |
| 353 | 2-{4-[4-methoxy-3-(pyridin-2-ylethynyl)benzoyl]piperazin- | 400.1 | M + H | 0.031 | 0.072 |
| | 1-yl}pyrimidine | | | | |
| 354 | 2-{4-[4-methoxy-3-(pyridin-3-ylethynyl)benzoyl]piperazin- | 400.1 | M + H | 4.972 | |
| | 1-yl}pyrimidine | | | | |
| 355 | 2-{4-[4-methoxy-3-(pyridin-4-ylethynyl)benzoyl]piperazin- | 400.1 | M + H | 1.568 | |
| | 1-yl}pyrimidine | | | | |
| 356 | 2-[4-(4-methoxy-3-{[3-(trifluoromethoxy)phenyl]ethynyl}benzoyl)piperazin- | 483.1 | M + H | 0.290 | |
| | 1-yl]pyrimidine | | | | |
| 357 | 2-(4-{4-methoxy-3-[(3-nitrophenyl)ethynyl]benzoyl}piperazin- | 444.1 | M + H | 0.239 | |
| | 1-yl)pyrimidine | | | | |
| 358 | 2-[4-(3-{[4-(benzyloxy)phenyl] ethynyl}-4- | 505.1 | M + H | IC50 > 10 | |
| | methoxybenzoyl) piperazin-1-yl]pyrimidine | | | uM | |
| 359 | 2-(4-{3-[(2-fluorophenyl) ethynyl]-4- | 417.1 | M + H | 0.287 | |
| | methoxybenzoyl} piperazin-1-yl)pyrimidine | | | | |
| 360 | 2-(4-{3-[(2-chlorophenyl) ethynyl]-4- | 433.0 | M + H | 4.498 | |
| | methoxybenzoyl} piperazin-1-yl)pyrimidine | | | | |
| 361 | 3-({2-methoxy-5-[(4-pyrimidin-2- | 424.1 | M + H | 0.064 | 0.056 |
| | ylpiperazin-1-yl)carbonyl] phenyl}ethynyl)benzonitrile | | | | |
| 362 | 2-(4-{3-[(4-fluorophenyl) ethynyl]-4- | 417.1 | M + H | 0.068 | 1.450 |
| | methoxybenzoyl} piperazin-1-yl)pyrimidine | | | | |
| 363 | 2-(4-{3-[(4-chlorophenyl) ethynyl]-4- | 433.0 | M + H | 0.891 | |
| | methoxybenzoyl} piperazin-1-yl)pyrimidine | | | | |
| 364 | 2-[4-(3-{[3-(difluoromethoxy) phenyl]ethynyl}- | 465.1 | M + H | 0.230 | |
| | 4-methoxy benzoyl)piperazin-1- | | | | |
| | yl] pyrimidine | | | | |

### Example 19

### 3-(4-{[3-(pyridin-2-ylethynyl)-4-(trifluoromethyl)phenyl]carbonyl}piperazin-1-yl)-1,2-benzisoxazole (Compound 374)

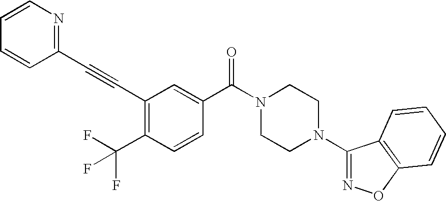

### Step 1: Methyl 3-amino-4-(trifluoromethyl)benzoate

A solution of 3-amino-4-(trifluoromethyl)benzoic acid (10 g, 48.8 mmol) and concentrated HCl (36%, 5.5 mL) in methanol (42 mL) was heated at 70° C. for 10 hours. After the reaction is complete, the reaction mixture was cooled down and concentrated in vacuo to afford methyl 3-amino-4-(trifluoromethyl)benzoate, HCl salt as a white solid (8.9 g, 34.8 mmol; 71% yield).

### Step 2: Methyl 3-iodo-4-(trifluoromethyl)benzoate

A solution of sodium nitrite (1.34 g 19.3 mmol) in water (7.0 mL) was added dropwise to a rapidly stirred suspension of methyl 3-amino-4-(trifluoromethyl)benzoate, HCl salt (4.5 g, 17.5 mmol) from step 1 in 6 N aqueous HCl (11 mL) at −5 to 0° C. over a period of five min. After the reaction was stirred at −5° C. for 30 min., a solution of potassium iodide (2.9 g, 17.5 mmol) in water (6.0 mL) and a small crystal of iodine were added slowly to the diazonium chloride formed in the reaction suspension. The resulting dark red solution was allowed to warm to room temperature and heated at 90° C. for one hour. The reaction mixture was extracted with ethyl acetate. The collected ethyl acetate extracts were washed with water. Separation and evaporation afforded methyl 3-iodo-4-(trifluoromethyl)benzoate as a dark brown solid (5.2 g, 15.8 mmol; 90% yield).

### Step 3: Methyl 3-(pyridin-2-ylethynyl)-4-(trifluoromethyl)benzoate

A mixture of methyl 3-iodo-4-(trifluoromethyl)benzoate (3 g, 9.1 mmol) from step 2,2-ethylnylpyridine (1.42 mL, 13.6 mmol), dichlorobistriphenylphosphine palladium(II) (1.28 g, 1.8 mmol), copper iodide (0.36 g, 1.82 mmol) and triethylamine (2.6 mL, 18.2 mmol) in toluene (46 mL) was stirred at 100° C. for six hours. The reaction mixture was monitored by LC-MS. After the reaction was complete, the reaction mixture was then allowed to cool down to room temperature. The reaction mixture was concentrated to yield a semi-solid residue. This residue was dissolved in ethyl acetate and the un-dissolved dark solid was removed by filtration. The ethyl acetate filtrate was washed with water and brine, dried over magnesium sulfate, filtered, and concentrated in vacuo to provide a brown crude solid. This material was purified by flash chromatography on SiO₂ (gradient elution using 0-3% MeOH in CH₂Cl₂) to yield the title compound as a brown solid (1.5 g, 4.9 mmol; 54% yield).

### Step 4: 3-(pyridin-2-ylethynyl)-4-(trifluoromethyl)benzoic acid

A 1.0 N solution of aqueous sodium hydroxide (7.3 mL, 7.3 mmol) was added to a solution of methyl 3-(pyridin-2-ylethynyl)-4-(trifluoromethyl)benzoate (1.1 g, 3.7 mmol) from step 3 in a mixed solvent of methanol and tetrahydrofuran (1:1; 20 mL) with stirring at room temperature. The reaction was complete in six hours. The reaction was acidified with 2.0 N aqueous HCl (3.7 mL, 7.3 mmol) to pH=1. The suspended mixture was filtered and evaporated to afford a light brown solid (1.5 g, 3.7 mmol; 100% yield) as a di-sodium chloride salt, which was used for the next reaction without any further purification.

### Step 5: 3-(4-{[3-(pyridin-2-ylethynyl)-4-(trifluoromethyl)phenyl]carbonyl}piperazin-1-yl)-1,2-benzisoxazole

Triethylamine (1.1 mL, 8.1 mmol) was added to a mixture of 3-(pyridin-2-ylethynyl)-4-(trifluoromethyl)benzoic acid (di-sodium chloride salt, 1.1 g, 2.7 mmol) from step 4, 1-[3-(dimethylamino)propyl]-3-ethylcarbodiimide hydrochloride (0.62 g, 3.2 mmol), 1-hydroxy-7-azabenzotriazole (0.44 mg, 3.2 mmol) and 3-(piperazin-1-yl)benzo[d]isoxazole (0.62 g, 3.0 mmol) in dichloromethane (20 mL) with stirring at room temperature under an atmosphere of nitrogen. The reaction mixture was stirred at room temperature overnight. The reaction was quenched with a small amount of water. Solvents were removed and the residue was purified by flash chromatography on SiO₂ (gradient elution using 40-60% EtOAc in hexane) to yield the title compound as a white solid (0.87 g, 67% yield).

Compounds 365-381 were synthesized according to Example 19.

### UNLESS NOTED OTHERWISE THE FOLLOWING VALUES REFER TO FORMULA I

### WHEREIN R₁, R₄, R₄ₐ, R₅, R₅ₐ═H; X₂═CH; Z=CO

TABLE 19

| Cmpd | Name | R₂ | X₁ | R₆ |
|---|---|---|---|---|
| 365 | 5-bromo-4-methoxy- 2-(4-{[3-(pyridin-2-yl ethynyl)-4-(trifluoro methyl)phenyl]carbonyl} piperazin-1-yl) pyrimidine | 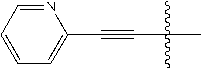 | CCF₃ | 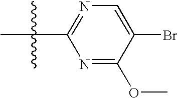 |
| 366 | 3-(4-{[3-(pyridin-2- ylethynyl)-4-(trifluoro methyl)phenyl]carbonyl} piperazin-1-yl) pyrazine-2- carbonitrile | 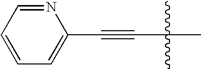 | CCF₃ | 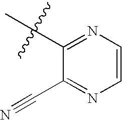 |
| 367 | 1-(4-methylpyridin-2- yl)-4-{[3-(pyridin-2- ylethynyl)-4- (trifluoromethyl)phenyl] carbonyl}piperazine | 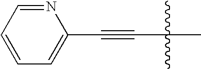 | CCF₃ | 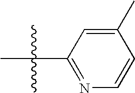 |
| 368 | 1-(3,5-dichloropyridin- 2-yl)-4-{[3-(pyridin-2- ylethynyl)-4-(trifluoro methyl)phenyl] carbonyl}piperazine | 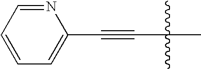 | CCF₃ | 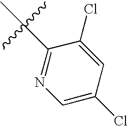 |
| 369 | 1-pyridin-2-yl-4-{[3- (pyridin-2-ylethynyl)- 4-(trifluoromethyl) phenyl]carbonyl} piperazine | 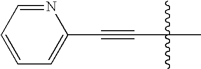 | CCF₃ | 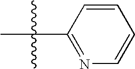 |
| 370 | 2-(4-{[3-(pyridin-2- ylethynyl)-4-(trifluoro methyl)phenyl] carbonyl}piperazin-2-yl) pyrimidine | 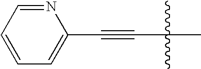 | CCF₃ | 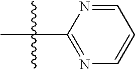 |
| 371 | 1-(3-chloropyridin- 2-yl)-4-{[3-(pyridin-2- ylethynyl)-4-(trifluoro methyl)phenyl] carbonyl}piperazine | 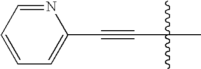 | CCF₃ | 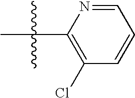 |
| 372 | 1-(5-methylpyridin- 2-yl)-4-{[3-(pyridin-2- ylethynyl)-4-(trifluoro methyl)phenyl] carbonyl}piperazine | 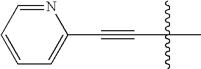 | CCF₃ | 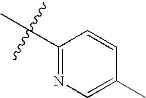 |
| 373 | 4-methoxy-2-(4-{[3- (pyridin-2-ylethynyl)- 4-(trifluoromethyl) phenyl]carbonyl} piperazin-1-yl) pyrimidine | 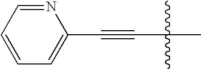 | CCF₃ | 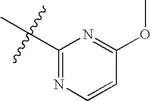 |
| 374 | 3-(4-{[3-(pyridin-2- ylethynyl)-4-(trifluoro methyl)phenyl] carbonyl}piperazin-1-yl)- 1,2-benzisoxazole | 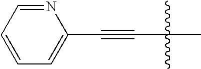 | CCF₃ | 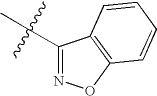 |
| 375 | 1-(furan-2-ylcarbonyl)- 4-{[3-(pyridin-2-yl ethynyl)-4-(trifluoro methyl)phenyl] carbonyl}piperazine | 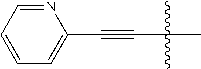 | CCF₃ | 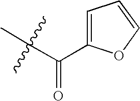 |
| 376 | 1-(3-fluorobenzyl)-4- {[3-(pyridin-2-yl ethynyl)-4-(trifluoro methyl)phenyl] carbonyl}piperazine | 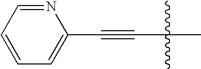 | CCF₃ | 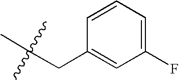 |
| 377 | 2-(4-{[3-(pyridin-2- ylethynyl)-4-(trifluoro methyl)phenyl]carbonyl} piperazin-1-yl)-1,3- benzothiazole | 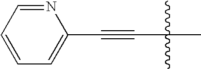 | CCF₃ | 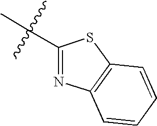 |
| 378 | 1-{[3-(pyridin-2- ylethynyl)-4-(trifluoro methyl)phenyl]carbonyl}- 4-(1,3-thiazol-2- yl)piperazine | 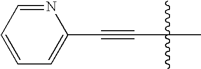 | CCF₃ | 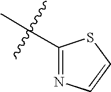 |
| 379 | 1-{[3-(pyridin-2- ylethynyl)-4-(trifluoro methyl)phenyl] carbonyl}- 4-[5-(trifluoro methyl) pyridin-2-yl] piperazine | 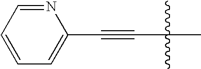 | CCF₃ | 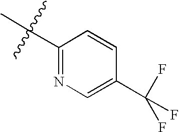 |
| 380 | 1-(6-methylpyridin-2- yl)-4-{[3-(pyridine-2- ylethynyl)-4-(trifluoro methyl)phenyl] carbonyl} piperazine | 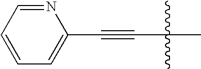 | CCF₃ | 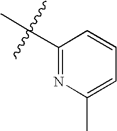 |
| 381 | 2-(4-{[3-(pyridin-2- ylethynyl)-4-(trifluoro methyl)phenyl] carbonyl} piperazin-1-yl) pyrazine | 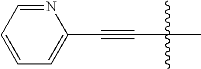 | CCF₃ | 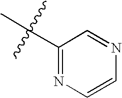 |

TABLE 19A

| | | | | Biological Activity | |
|---|---|---|---|---|---|
| | | LCMS data | | Median Ki | |
| Cmpd | Name | Mass | Ion | (μM) | IC50 (uM) |
| 365 | 5-bromo-4-methoxy-2-(4-{[3-(pyridin-2- | 546.0 | M + H | IC50 > 10 uM | |
| | ylethynyl)-4- | | | | |
| | (trifluoromethyl)phenyl]carbonyl}piperazin- | | | | |
| | 1-yl)pyrimidine | | | | |
| 366 | 3-(4-{[3-(pyridin-2-ylethynyl)-4- | 463.1 | M + H | 0.459 | |
| | (trifluoromethyl)phenyl]carbonyl}piperazin- | | | | |
| | 1-yl) pyrazine-2-carbonitrile | | | | |
| 367 | 1-(4-methylpyridin-2-yl)-4-{[3-(pyridin- | 462.0 | M + H | 0.008 | 0.016 |
| | 2-ylethynyl)-4- | | | | |
| | (trifluoromethyl)phenyl]carbonyl}piperazine | | | | |
| 368 | 1-(3,5-dichloropyridin-2-yl)-4-{[3- | 505.0 | M + H | 2.924 | |
| | (pyridin-2-ylethynyl)-4- | | | | |
| | (trifluoromethyl)phenyl]carbonyl}piperazine | | | | |
| 369 | 1-pyridin-2-yl-4-{[3-(pyridin-2- | 437.1 | M + H | 0.031 | 0.030 |
| | ylethynyl)-4- | | | | |
| | (trifluoromethyl)phenyl]carbonyl}piperazine | | | | |
| 370 | 2-(4-{[3-(pyridin-2-ylethynyl)-4- | 438.1 | M + H | 0.164 | |
| | (trifluoromethyl)phenyl]carbonyl}piperazin- | | | | |
| | 1-yl)pyrimidine | | | | |
| 371 | 1-(3-chloropyridin-2-yl)-4-{[3-(pyridin- | 471.1 | M + H | 1.001 | |
| | 2-ylethynyl)-4- | | | | |
| | (trifluoromethyl)phenyl]carbonyl}piperazine | | | | |
| 372 | 1-(5-methylpyridin-2-yl)-4-{[3-(pyridin- | 451.1 | M + H | 0.292 | |
| | 2-ylethynyl)-4- | | | | |
| | (trifluoromethyl)phenyl]carbonyl}piperazine | | | | |
| 373 | 4-methoxy-2-(4-{[3-(pyridin-2- | 468.1 | M + H | 0.040 | 0.034 |
| | ylethynyl)-4-(trifluoromethyl)phenyl]carbonyl}piperazin- | | | | |
| | 1-yl)pyrimidine | | | | |
| 374 | 3-(4-{[3-(pyridin-2-ylethynyl)-4- | 477.1 | M + H | 0.004 | 0.053 |
| | (trifluoromethyl)phenyl]carbonyl}piperazin- | | | | |
| | 1-yl)-1,2-benzisoxazole | | | | |
| 375 | 1-(furan-2-ylcarbonyl)-4-{[3-(pyridin-2- | 454.1 | M + H | 0.029 | 0.040 |
| | ylethynyl)-4- | | | | |
| | (trifluoromethyl)phenyl]carbonyl}piperazine | | | | |
| 376 | 1-(3-fluorobenzyl)-4-{[3-(pyridin-2- | 468.1 | M + H | 0.811 | |
| | ylethynyl)-4- | | | | |
| | (trifluoromethyl)phenyl]carbonyl}piperazine | | | | |
| 377 | 2-(4-{[3-(pyridin-2-ylethynyl)-4- | 493.1 | M + H | IC50 > 10 uM | |
| | (trifluoromethyl)phenyl]carbonyl}piperazin- | | | | |
| | 1-yl)-1,3-benzothiazole | | | | |
| 378 | 1-{[3-(pyridin-2-ylethynyl)-4- | 443.1 | M + H | 0.049 | 0.043 |
| | (trifluoromethyl)phenyl]carbonyl}-4- | | | | |
| | (1,3-thiazol-2-yl)piperazine | | | | |
| 379 | 1-{[3-(pyridin-2-ylethynyl)-4- | 505.1 | M + H | 3.126 | |
| | (trifluoromethyl)phenyl]carbonyl}-4-[5- | | | | |
| | (trifluoromethyl) pyridin-2-yl]piperazine | | | | |
| 380 | 1-(6-methylpyridin-2-yl)-4-{[3-(pyridin- | 451.1 | M + H | 0.352 | |
| | 2-ylethynyl)-4- | | | | |
| | (trifluoromethyl)phenyl]carbonyl}piperazine | | | | |
| 381 | 2-(4-{[3-(pyridin-2-ylethynyl)-4- | 438.1 | M + H | 0.162 | |
| | (trifluoromethyl)phenyl]carbonyl}piperazin- | | | | |
| | 1-yl)pyrazine | | | | |

### Example 20

### 1-{[4-(Difluoromethoxy)-3-(pyridin-2-ylethynyl)phenyl]carbonyl}-4-pyridin-2-ylpiperazin (Compound 385)

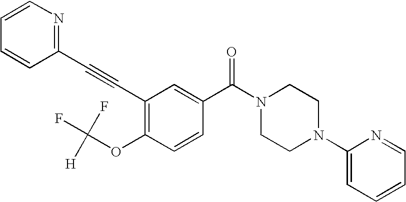

### Step 1: Methyl 4-(difluoromethoxy)-3-iodobenzoate

A cold solution of difluoroiodomethane (5.0 g, 28.0 mmol) in DMF (15 mL) was added to a stirred suspension of potassium carbonate (5.2 g, 37.4 mmol) and methyl 4-hydroxy-3-iodobenzoate (5.4 g, 97%, 18.7 mmol) in DMF (65 mL) at 0° C. under an atmosphere of nitrogen. After the reaction was stirred at 0° C. for 30 min., the reaction mixture was stirred at room temperature for 2.5 hours. After the reaction was complete, solid material was removed by filtration and the filtrate was concentrated to yield a semi-solid residue. This residue was purified by flash chromatography on SiO₂ (gradient elution using EtOAc/hexane 15/85) to yield the title compound as a white solid (5.0 g, 81% yield).

### Step 2: Methyl 4-(difluoromethoxy)-3-(pyridin-2-ylethynyl)benzoate

A mixture of methyl 4-(difluoromethoxy)-3-iodobenzoate (2 g, 6.1 mmol) from step 1,2-ethylnylpyridine (0.94 mL, 9.2 mmol), dichlorobistriphenylphosphine palladium(II) (0.86 g, 1.2 mmol), copper iodide (0.23 g, 1.2 mmol) and triethylamine (1.7 mL, 12.2 mmol) in toluene (30 mL) was stirred at 100° C. under an atmosphere of nitrogen for six hours. After the reaction was complete, the reaction mixture was concentrated to yield a semi-solid residue. This residue was purified by flash chromatography on SiO₂ (gradient elution using EtOAc/hexane 20/80) to yield the title compound as a white solid (1.47 g, 80% yield).

### Step 3: 4-(difluoromethoxy)-3-(pyridin-2-ylethynyl)benzoic acid

A 1.0 N solution of aqueous sodium hydroxide (9.6 mL, 9.6 mmol) was added to a solution of methyl 4-(difluoromethoxy)-3-(pyridin-2-ylethynyl)benzoate (1.5 g, 4.8 mmol) from step 2 in a mixed solvent of methanol and tetrahydrofuran (1:1; 26 mL) with stirring at room temperature. The reaction was complete in three hours. The reaction was acidified with 2.0 N aqueous HCl (5.0 mL, 10.0 mmol) to pH=1. The suspended mixture was evaporated to afford a grey solid (1.84 g, 95% yield) containing two equivalents of sodium chloride, which was used for the next reaction without any further purification.

### Step 4: 1-{[4-(difluoromethoxy)-3-(pyridin-2-ylethynyl)phenyl]carbonyl}-4-pyridin-2-ylpiperazin

Triethylamine (0.48 mL, 3.5 mmol) was added to a mixture of 4-(difluoromethoxy)-3-(pyridin-2-ylethynyl)benzoic acid containing two equivalents of sodium chloride (700 mg, 1.72 mmol), 1-[3-(dimethylamino)propyl]-3-ethylcarbodiimide hydrochloride (0.43 g, 2.24 mmol), 1-hydroxy-7-azabenzotriazole (0.31 g, 2.24 mmol) and 2-(piperazin-1-yl)pyrazine (0.31 mL, 2.1 mmol) in dichloromethane (26 mL) with stirring at room temperature under an atmosphere of nitrogen. The reaction mixture was stirred at room temperature overnight. The reaction was quenched with small amount of water. The solvents were removed and the residue was purified by flash chromatography on SiO₂ (column diam: 60 mm; fraction size: 100 mL; gradient elution using 0-8% methanol in dichloromethane). Fractions 30-33 were combined and evaporated to give an oil, which was dissolved in methanol (20 mL). Aqueous HCl (2.0 N, 1.8 mL) was added to this methanol solution. The mixture was then stirred at room temperature for 20 min. Evaporation yielded a semi-solid, which was triturated with dichloromethane (3×) and dried in vacuo at 50° C. for 7 hours to afford the di-HCl product as a light green solid (0.81 g, 93% yield).

Compounds 382-385 were synthesized according to Example 20.

### UNLESS NOTED OTHERWISE THE FOLLOWING VALUES REFER TO FORMULA I

### WHEREIN R₁, R₄, R₄ₐ, R₅, R₅ₐ═H; X₂═CH; Z=CO

TABLE 20

| Cmpd | Name | R₂ | X₁ | R₆ |
|---|---|---|---|---|
| 382 | 5-bromo-2-(4-{[4- (difluoromethoxy)-3-(pyridin- 2-ylethynyl) phenyl]carbonyl}piperazin-1- yl)-4-methoxy pyrimidine | 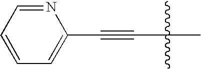 | COCHF₂ | 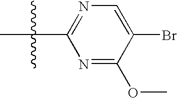 |
| 383 | 3-(4-{[4-(difluoro methoxy)-3- (pyridin-2- ylethynyl)phenyl]carbonyl} piperazin-1-yl)-1,2- benzisothiazole | 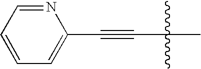 | COCHF₂ | 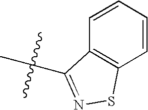 |
| 384 | 3-(4-{[4-(difluoro methoxy)-3- (pyridin-2- ylethynyl)phenyl]carbonyl} piperazin-1-yl)-1,2- benzisothiazole | 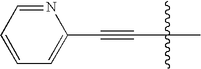 | COCHF₂ | 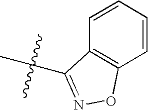 |
| 385 | 1-{[4-(difluoromethoxy)-3- (pyridin-2-ylethynyl) phenyl]carbonyl}-4-pyridin-2- ylpiperazine | 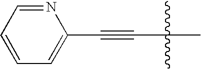 | COCHF₂ | 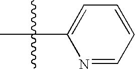 |

TABLE 20A

| | | | | Biological Activity | |
|---|---|---|---|---|---|
| | | LCMS data | | Median | |
| Cmpd | Name | Mass | Ion | Ki (μM) | IC50 (uM) |
| 382 | 5-bromo-2-(4-{[4-(difluoro methoxy)-3- | 544.0 | M + H | 0.035 | 0.104 |
| | (pyridin-2-yl ethynyl)phenyl]carbonyl}piperazin- | | | | |
| | 1-yl)-4-methoxy pyrimidine | | | | |
| 383 | 3-(4-{[4-(difluoromethoxy)-3-(pyridin-2- | 491.1 | M + H | 0.001 | 0.041 |
| | ylethynyl)phenyl] carbonyl}piperazin-1-yl)- | | | | |
| | 1,2-benzisothiazole | | | | |
| 384 | 3-(4-{[4-(difluoromethoxy)-3-(pyridin-2- | 475.1 | M + H | 0.001 | 0.016 |
| | ylethynyl)phenyl] carbonyl}piperazin-1-yl)- | | | | |
| | 1,2-benzisoxazole | | | | |
| 385 | 1-{[4-(difluoromethoxy)-3-(pyridin-2- | 453.0 | M + H | 0.007 | 0.013 |
| | ylethynyl)phenyl] carbonyl}-4-pyridin-2- | | | | |
| | ylpiperazine | | | | |

### Example 21

### 1-[4-chloro-3-(pyridin-2-ylethynyl)benzoyl]-4-pyridin-2-ylpiperazin (Compound 386)

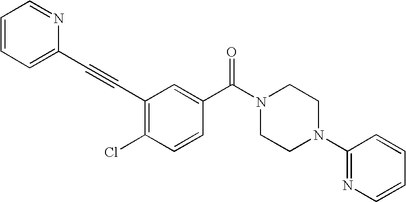

### Step 1: methyl 4-chloro-3-(pyridin-2-ylethynyl)benzoate

Methyl 3-bromo-4-chlorobenzoate (1.758 g, 7.089 mmol), 2-ethynyl pyridine (1.40 mL, 13.9 mmol), and triethylamine (2.20 mL, 15.8 mmol) were dissolved in 34 mL dry toluene. Nitrogen gas was bubbled through the mixture for 10 minutes, and then dichlorobis(triphenylphosphine)-palladium(II) (1.00 g, 1.42 mmol) and copper(I) iodide (0.268 g, 1.41 mmol) were added to the mixture. Nitrogen was bubbled through the mixture for another 5 minutes, and then the mixture was then heated to 100° C. for 6 hours. The mixture was cooled, and then filtered through a pad of Celite. The Celite pad was washed with ethyl acetate (2×) and then ˜5% methanol/methylene chloride (2×). The combined filtrate was evaporated and the residue was chromatographed on silica gel using a gradient elution of ethyl acetate in methylene chloride. Methyl 4-chloro-3-(pyridin-2-ylethynyl)benzoate is obtained (0.843 g, 3.11 mmol; 44% yield) as a light brown-gray solid.

### Step 2: 4-chloro-3-(pyridin-2-ylethynyl)benzoic acid

Methyl 4-chloro-3-(pyridin-2-ylethynyl)benzoate (0.413 g, 1.52 mmol) was dissolved in 6 mL of methanol. Aqueous 2N NaOH (1.52 mL, 3.05 mmol) was added, and the mixture was stirred 24 hours at room temperature. Aqueous 2N HCl (1.52 mL, 3.05 mmol) was added, and the mixture was stirred 5 minutes at room temperature. The mixture was evaporated to dryness to afford 4-chloro-3-(pyridin-2-ylethynyl)benzoic acid (0.580 g) as a light gray solid containing 2 equivalents of sodium chloride. This material was used as is for subsequent reactions.

### Step 3: 1-[4-chloro-3-(pyridin-2-ylethynyl)benzoyl]-4-pyridin-2-yl piperazine

4-Chloro-3-(pyridin-2-ylethynyl)benzoic acid containing 2 equivalents of sodium chloride (0.040 g, 0.107 mmol) was dissolved in 0.8 mL dimethylformamide. N-(3-dimethylaminopropyl)-N′-ethyl-carbodiimide hydrochloride (EDCl, 0.027 g, 0.141 mmol) was added, followed by 1-hydroxy-7-azabenzotriazole (HOAt, 0.019 g, 0.140 mmol) and then 1-(2-pyridyl)-piperazine (0.016 mL, 0.110 mmol). Triethylamine (0.045 mL, 0.323 mmol) was added, and the mixture was stirred overnight at room temperature. The mixture was then partitioned between ethyl acetate and water, and the aqueous layer was extracted with ethyl acetate. The combined organic phase was pumped dry, and was purified by prep HPLC using a Gilson reversed-phase HPLC with TFA modified water and acetonitrile as eluant. The solid obtained from the fractions containing the desired product was taken up in 0.7 mL methanol, and 2N HCl (0.050 mL, 0.100 mmol) was added. The mixture was stirred at room temperature for 5 minutes, and was then pumped dry to afford the HCl salt of 1-[4-chloro-3-(pyridin-2-ylethynyl)benzoyl]-4-pyridin-2-ylpiperazin (0.026 g, 0.055 mmol; 51% yield) as a light greenish-white solid.

Compounds 386-396, shown in Table 21 below, were prepared using the procedure of Example 21 described above.

### UNLESS NOTED OTHERWISE THE FOLLOWING VALUES REFER TO FORMULA I

### WHEREIN R₁, R₄, R₄ₐ, R₅, R₅ₐ═H; X₂═CH; Z=CO

TABLE 21

| Cmpd | Name | R₂ | X₁ | R₆ |
|---|---|---|---|---|
| 386 | 1-[4-chloro-3-(pyridin- 2-ylethynyl)benzoyl]- 4-pyiidin-2-yl piperazine | 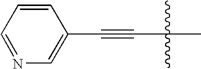 | CCl | 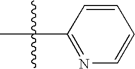 |
| 387 | 2-{4-[4-chloro-3- (pyridin-2-ylethynyl) benzoyl]piperazin-1- yl}pyrimidine | 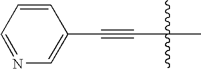 | CCl | 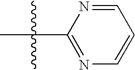 |
| 388 | 2-{4-[4-chloro-3- (pyridin-2-ylethynyl) benzoyl]piperazin-1- yl}pyrazine | 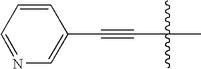 | CCl | 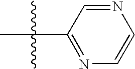 |
| 389 | 2-{4-(4-chloro-3- (pyridin-2-ylethynyl) benzoyl]piperazin-1- yl}nicotinonitrile | 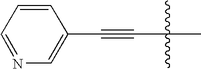 | CCl | 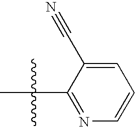 |
| 390 | 1-[4-chloro-3-(pyridin- 2-ylethynyl)benzoyl]- 4-(1,3-thiazol-2-yl) piperazine | 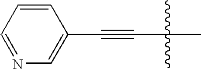 | CCl | 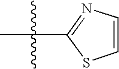 |
| 391 | 1-[4-chloro-3-(pyridin- 2-ylethynyl)benzoyl]- 4-pyridin-4-yl piperazine | 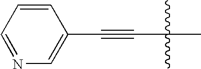 | CCl | 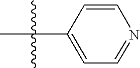 |
| 392 | 1-[4-chloro-3-(pyridin- 2-ylethynyl)benzoyl]- 4-[3- (trifluoromethyl) phenyl]piperazine | 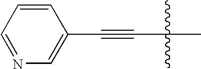 | CCl | 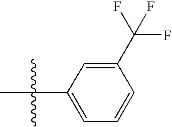 |
| 393 | 3-(4-{[4-chloro-3- (pyridin-2-ylethynyl) phenyl]carbonyl} piperazin-1- yl)phenol | 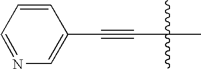 | CCl | 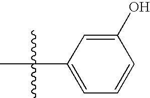 |
| 394 | 1-(3-chloropyridin-2- yl)-4-{[4-chloro-3- (pyridin-2-ylethynyl) phenyl]carbonyl} piperazine | 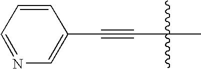 | CCl | 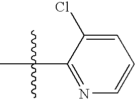 |
| 395 | 1-{[4-chloro-3- (pyridin-2-ylethynyl) phenyl]carbonyl}-4- (3-methoxyphenyl) piperazine | 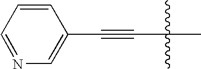 | CCl | 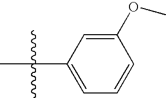 |
| 396 | 3-(4-{[4-chloro-3- pyridin-2-ylethynyl) phenyl]carbonyl} piperazin-1- yl)pyrazine-2- carbonitrile | 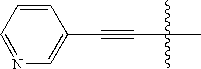 | CCl | 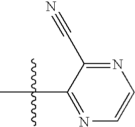 |

TABLE 21A

| | | | | Biological | |
|---|---|---|---|---|---|
| | | | | Activity | |
| | | LCMS data | | Median Ki | IC50 |
| Cmpd | Name | Mass | Ion | (μM) | (uM) |
| 386 | 1-[4-chloro-3-(pyridin-2-ylethynyl)benzoyl]- | 403.1 | M + H | 0.003 | 0.039 |
| | 4-pyridin-2-ylpiperazine | | | | |
| 387 | 2-{4-[4-chloro-3-(pyridin-2- | 404.1 | M + H | 0.010 | 0.131 |
| | ylethynyl)benzoyl]piperazin-1-yl}pyrimidine | | | | |
| 388 | 2-{4-[4-chloro-3-(pyridin-2- | 404.1 | M + H | 0.013 | 0.094 |
| | ylethynyl)benzoyl]piperazin-1-yl}pyrazine | | | | |
| 389 | 2-{4-[4-chloro-3-(pyridin-2- | 428.1 | M + H | 0.063 | 0.239 |
| | ylethynyl)benzoyl]piperazin-1- | | | | |
| | yl}nicotinonitrile | | | | |
| 390 | 1-[4-chloro-3-(pyridin-2-ylethynyl)benzoyl]- | 409.1 | M + H | 0.007 | 0.039 |
| | 4-(1,3-thiazol-2-yl)piperazine | | | | |
| 391 | 1-[4-chloro-3-(pyridin-2-ylethynyl)benzoyl]- | 403.1 | M + H | 0.079 | 1.494 |
| | 4-pyridin-4-ylpiperazine | | | | |
| 392 | 1-[4-chloro-3-(pyridin-2-ylethynyl)benzoyl]- | 470.1 | M + H | 0.120 | |
| | 4-[3-(trifluoromethyl)phenyl]piperazine | | | | |
| 393 | 3-(4-{[4-chloro-3-(pyridin-2- | 418.1 | M + H | 0.012 | 0.144 |
| | ylethynyl)phenyl]carbonyl}piperazin-1- | | | | |
| | yl)phenol | | | | |
| 394 | 1-(3-chloropyridin-2-yl)-4-{[4-chloro-3- | 437.1 | M + H | 0.021 | 0.145 |
| | (pyridin-2- | | | | |
| | ylethynyl)phenyl]carbonyl}piperazine | | | | |
| 395 | 1-{[4-chloro-3-(pyridin-2- | 432.1 | M + H | 0.019 | 0.113 |
| | ylethynyl)phenyl]carbonyl}-4-(3- | | | | |
| | methoxyphenyl)piperazine | | | | |
| 396 | 3-(4-{[4-chloro-3-(pyridin-2- | 429.1 | M + H | 0.017 | 0.047 |
| | ylethynyl)phenyl]carbonyl}piperazin-1- | | | | |
| | yl)pyrazine-2-carbonitrile | | | | |

Variations, modifications, and other implementations of what is described herein will occur to those of ordinary skill in the art without departing from the spirit and the essential characteristics of the present teachings. Accordingly, the invention is intended to include all such modifications and implementations, and their equivalents.

Each reference cited in the present application, including books, patents, published applications, journal articles and other publications, is incorporated herein by reference in its entirety.

What is claimed is:

1. A compound of Formula I: 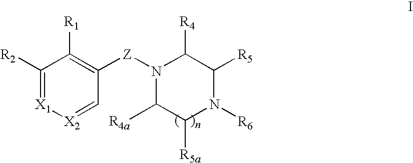
wherein:
R₁ is each independently selected from H, C₁₋₆ alkyl, halogen, OH, and OC₁₋₆ alkyl;
R₂ is selected from -(L₁)ₐ-(Y)_{c}-(L₂)_{b}-Q₃, -L₃-Q₄ and -L₄-Q₅;
L₃ is C₂₋₁₂ alkynyl optionally substituted with 1-3 substituents selected from OH and halogen;
L₁ and L₂ are each independently C₁₋₃ alkyl;
L₄ is C₂₋₁₂ alkenyl optionally substituted with 1-3 substituents selected from OH and halogen;
n is 1 or 2
R₄, R₄ₐ, R₅, and R₅ₐ are each independently selected from H, and C₁₋₆ alkyl or R₄ and one of R₅ₐ together can form a bridging methylene; or R₅ can be together with the carbon to which it is attached —C(═O);
R₆ is selected from H, CH₃, -(L₅)-(3- to 14-membered heterocycle), -(L₅)-(5 to 14 membered heteroaromatic), (L₅)-(3- to 10-membered cycloalkyl), (L₅)-(C₆₋₁₄ aryl) and -(L₅)-C₁₋₆ alkyl each of which except H can be optionally substituted with 1 to 3 substituents independently selected from H, C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl, CN, (5- to 14-membered heteroaromatic), NR₁R₁, SO₂C₁₋₆ alkyl, SO₂, SO₂NR₁R₁, C₁₋₆ alkylaryl, COC₁₋₆ alkyl, and (3- to 14-membered heterocycle) optionally substituted with NO₂.
L₅ is selected from a bond, C₁₋₃ alkyl, —C(═O)—, SO₂, (3- to 6-membered heterocycle) and (5- to 14-membered heteroaromatic);
X₁, X₂ are independently CR₃ or N;
each R₃ is independently selected from H, C₁₋₆ alkyl, halogen, OH, OC₁₋₆ alkyl, SO₂, (3- to 14-membered heterocyclic) and (5- to 14-membered heteroaromatic), wherein each of C₁₋₆ alkyl or OC₁₋₆ alkyl can be optionally substituted with 1 to 3 substituents independently selected from halogen, OH, OC₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl), cycloalkyl, NR₁R₁, and CN;
Z is CO;
Y is CR₇R₈, NR₉, O, or S;
R₇, R₈, R₉ are independently H, C₁₋₆ alkyl, halogen, OH, or OC₁₋₆ alkyl
a, b and c are independently 0 or 1;
Q₃ is selected from C₆₋₁₄ aryl, (5- to 14-membered heterocyclic), (5- to 14-membered heteroaromatic), and (4- to 9-membered carbocyclic); each of which can be optionally substituted with 1 to 3 substituents independently selected from C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl), OC₁₋₃ haloalkyl, OC₁₋₆ alkylaryl and CN;
Q₄ is selected from H, C₆₋₁₄ aryl, (5- to 14-membered heterocyclic), (5- to 14-membered heteroaromatic), and (4- to 9-membered carbocyclic); each of which except H can be optionally substituted with 1 to 3 substituents independently selected from C₁₋₆ alkyl, halogen, OH, OC₁₋₆ alkyl, —C(═O)O—(C₁₋₆ alkyl), —C(═O)C₁₋₆ alkyl, NO₂, C₁₋₃ haloalkyl, —S—C₁₋₆ alkyl —NH₂, —NH—(C₁₋₆ alkyl), —N(C₁₋₆ alkyl)(C₁₋₆ alkyl), OC₁₋₃ haloalkyl, OC₁₋₆ alkylaryl and CN; and
Q₅ is selected from C₆₋₁₄ aryl, (5- to 14-membered heterocyclic), (5- to 14-membered heteroaromatic), and (4- to 9-membered carbocyclic); each of which can be optionally substituted with 1 to 3 substituents independently selected from C₁₋₆ alkyl, halogen, OH, OC₁₋₆ alkyl, —C(═O)O—(C₁₋₆ alkyl), NO₂, C₁₋₃ haloalkyl, —S—C₁₋₁₆ alkyl —NH₂, —NH—(C₁₋₆ alkyl), —N(C₁₋₆ alkyl)(C₁₋₆ alkyl), OC₁₋₃ haloalkyl, OC₁₋₆ alkylaryl and CN.

2. A compound of Formula I: 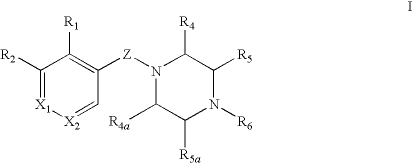
wherein:
R₁ is H, C₁₋₆ alkyl, halogen, OH, or OC₁₋₆ alkyl;
R₂ is -(L₁)ₐ-(Y)_{c}-(L₂)_{b}-Q₃, -L₃-Q₄ or -L₄-Q₅;
L₃ is C₂₋₁₂ alkynyl optionally substituted with 1-3 substituents selected from OH and halogen;
L₁ and L₂ are each independently C₁₋₃ alkyl;
L₄ is C₂₋₁₂ alkenyl optionally substituted with 1-3 substituents selected from OH and halogen;
R₄, R₄ₐ, R₅, and R₅ₐ are each independently H or C₁₋₆ alkyl;
R₆ is selected from H, CH₃, -(L₅)-2-pyridyl, -(L₅)-4-pyridyl, -(L₅)-pyrazinyl, -(L₅)-phenyl, -(L₅)-(3-14 membered heterocyclic), -(L₅)-(5 to 14 membered heteroaromatic), and (L₅)-(C₆₋₁₄ aryl) each of which except H can be optionally substituted with 1 to 3 substituents independently selected from H, C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl, COC₁₋₆ alkyl and CN;
X₁, X₂ are independently CR₃ or N;
L₅ is selected from a bond, C₁₋₃ alkyl, —C(═O)—, SO₂, (3- to 6-membered heterocycle) and (5- to 14-membered heteroaromatic).
each R₃ is independently selected from H, C₁₋₆ alkyl, halogen, OH, OC₁₋₆ alkyl, SO₂, (3- to 14-membered heterocyclic) and (5- to 14-membered heteroaromatic), wherein each of C₁₋₆ alkyl or OC₁₋₆ alkyl can be optionally substituted with 1 to 3 substituents independently selected from halogen, OH, OC₁₋₆ alkyl, —C(═O)O—(C₁₋₆ alkyl), NO₂, C₁₋₃ haloalkyl, —S—C₁₋₁₆ alkyl —NH₂, —NH—(C₁₋₆ alkyl), —N(C₁₋₆ alkyl)(C₁₋₆ alkyl), cycloalkyl, NR₁R₁, and CN;
Z is CO;
Y is selected from CR₇R₈, NR₉, O, or S;
R₇, R₈, R₉ are independently selected from H, C₁₋₆ alkyl, halogen, OH, and OC₁₋₆ alkyl;
a, b and c are independently 0 or 1; and
Q₃ is selected from C₆₋₁₄ aryl, (5- to 14-membered heterocyclic), (5- to 14-membered heteroaromatic), or (4- to 9-membered carbocyclic); each of which can be optionally substituted with 1 to 3 substituents independently selected from C₁₋₆ alkyl, halogen, OH, OC₁₋₆ alkyl, —C(═O)O—(C₁₋₆ alkyl), NO₂, C₁₋₃ haloalkyl, —S—C₁₋₁₆ alkyl —NH₂, —NH—(C₁₋₆ alkyl), —N(C₁₋₆ alkyl)(C₁₋₆ alkyl), OC₁₋₃ haloalkyl, OC₁₋₆ alkylaryl and CN;
Q₄ is selected from H, C₆₋₁₄ aryl, 5 to 14 membered heterocyclic, 5 to 14 membered heteroaromatic, or 4 to 9 membered carbocyclic; each of which except H can be optionally substituted with 1 to 3 substituents independently selected from C₁₋₆ alkyl, halogen, OH, OC₁₋₆ alkyl, —C(═O)O—(C₁₋₆ alkyl), —C(═O)C₁₋₁₆ alkyl, NO₂, C₁₋₃ haloalkyl, —S—C₁₋₆ alkyl —NH₂, —NH—(C₁₋₆ alkyl), —N(C₁₋₆ alkyl)(C₁₋₆ alkyl), OC₁₋₃haloalkyl, OC₁₋₆alkylaryl and CN;
Q₅ is selected from C₆₋₁₄ aryl, (5 to 14 membered heterocyclic), (5 to 14 membered heteroaromatic), and (4 to 9 membered carbocyclic); each of which can be optionally substituted with 1 to 3 substituents independently selected from C₁₋₆ alkyl, halogen, OH, OC₁₋₆ alkyl, —C(═O)O—(C₁₋₆ alkyl), NO₂, C₁₋₃ haloalkyl, —S—C₁₋₁₆ alkyl —NH₂, —NH—(C₁₋₆ alkyl), —N(C₁₋₆ alkyl)(C₁₋₆ alkyl), OC₁₋₃haloalkyl, OC₁₋₆alkylaryl and CN.

3. The compound of claim 1 or 2, wherein R₂ is -L₃-Q₄.

4. The compound of claim 1 or 2, wherein R₆ is selected from -(L₅)-2-pyridyl, -(L₅)-4-pyridyl, -(L₅)-pyrazinyl, and -(L₅)-phenyl.

5. The compound of claim 1 or 2 wherein X₁ is COCHF₂ or COCF₃.

6. The compound of claim 1 or 2, wherein R₃ is selected from H, OCHF₂, OCF₃, ethoxy, cyclopropylmethyloxy, and CF₃.

7. The compound of claim 1 or 2, wherein R₁, R₄, R₄ₐ, R₅, and R₅ₐ, are each H.

8. The compound of claim 1 or 2, wherein the 3-14 membered heterocycle of R₆ is selected from aziridinyl, azetidinyl, 1,4-dioxanyl, hexahydroazepinyl, piperazinyl, piperidinyl, piperazinyl, pyrrolidinyl, morpholinyl, thiomorpholinyl, dihydrobenzimidazolyl, dihydrobenzofuranyl, dihydrobenzothienyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, dihydro-1,4-dioxanyl, tetrahydrofuranyl, tetrahydrothienyl, tetrahydroquinolinyl, and tetrahydroisoquinolinyl.

9. The compound of claim 1 or 2, wherein the 5 to 14 membered heteroaromatic of R₆ is selected from furyl, thienyl, pyridyl, pyrrolyl, oxazolyl, thiazolyl, isothiazolyl, imidazolyl, pyrazolyl, isoxazolyl, triazolyl, oxadiazolyl, pyrimidinyl, pyrazinyl, indolyl, benzimidazolyl, benzothiophenyl, quinolinyl, isoquinolinyl, quinoxalinyl, and benzothiazolyl

10. The compound of claim 1 or 2, wherein L₅ is selected from a bond, SO₂ and —C(═O)—.

11. The compound of claim 1 or 2, wherein X₁ is CR₃; X₂ is CH, R₆ is -(L₅)-2-pyridyl optionally substituted with halogen or C₁₋₆alkyl, wherein L₅ is a bond, R₄ₐ and R₅ form a bridging methylene, R₂ is -L₃-Q₄, L₃ is C₂ alkynyl, and Q₄ is 2-pyridyl or phenyl optionally substituted with 1 to 3 substituents independently selected from C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl) and CN.

12. The compound of claim 1 or 2, wherein R₃ is OC₁₋₆ alkyl.

13. The compound of claim 1 or 2, wherein R₂ is -L₃-Q₄ and L₃ is C₂₋₃ alkynyl.

14. The compound of claim 1 or 2, wherein Q₄ is selected from C₆₋₁₄ aryl, 5- to 14-membered heterocycle and 5- to 14-membered heteroaromatic optionally substituted with 1 to 3 substituents independently selected from C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl) and CN.

15. The compound of claim 14, wherein Q₄ is pyridinyl optionally substituted with 1 to 3 substituents independently selected from C₁₋₆ alkyl, halogen, OH, OC₁₋₆ alkyl, —C(═O)O—(C₁₋₆ alkyl), —C(═O)C₁₋₆ alkyl, NO₂, C₁₋₃ haloalkyl, —S—C₁₋₆ alkyl —NH₂, —NH—(C₁₋₆ alkyl), —N(C₁₋₆ alkyl)(C₁₋₆ alkyl), OC₁₋₃haloalkyl, OC₁₋₆alkylaryl and CN

16. The compound of claim 1 or 2, wherein R₆ is (5- to 14-membered heteroaromatic) optionally substituted with 1 to 3 substituents independently selected from C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl and CN.

17. The compound of claim 1 or 2, wherein:
R₂ is selected from methyl-5-phenylethynyl-pyridine and methyl-2-phenylethynyl-pyridine; and
R₆ is selected from phenyl, 2-pyridyl, methyl-pyrimidine, methyl-nicotinonitrile, methyl-5-trifluoromethyl-pyridine, 2,4-dimethyl-pyridine, 2,6-dimethylpyridine, methyl-6-trifluoromethyl-pyridine, methyl-pyrazine, methyl-3-trifluoromethyl-pyridine, methyl-nicotinonitrile, methyl-pyrimidine, and 4-pyridyl.

18. The compound of claim 1, wherein the compound is selected from 1-{[5-(phenyl ethynyl)pyridin-3-yl]carbonyl}-4-pyridin-2-ylpiperazin; 2-{4-[2-(phenylethynyl)isonicotinoyl]piperazin-1-yl}pyrimidine; 1-[2-(phenylethynyl)isonicotinoyl]-4-pyridin-2-ylpiperazin; 6-{4-[2-(phenylethynyl)isonicotinoyl]piperazin-1-yl}nicotinonitrile; 1-[2-(phenylethynyl)isonicotinoyl]-4-[5-(trifluoromethyl)pyridin-2-yl]piperazine; 1-(4-methylpyridin-2-yl)-4-[2-(phenylethynyl)isonicotinoyl]piperazine; 1-(6-methylpyridin-2-yl)-4-[2-(phenylethynyl)isonicotinoyl]piperazine; 1-[2-(phenylethynyl)isonicotinoyl]-4-[6-(trifluoromethyl)pyridin-2-yl]piperazine; 2-{4-[2-(phenylethynyl)isonicotinoyl]piperazin-1-yl}pyrazine; 2-(4-{[5-(phenylethynyl)pyridin-3-yl]carbonyl}piperazin-1-yl)pyrimidine; 6-(4-{[5-(phenylethynyl)pyridin-3-yl]carbonyl}piperazin-1-yl)nicotinonitrile; 1-{[5-(phenylethynyl)pyridin-3-yl]carbonyl}-4-[5-(trifluoromethyl)pyridin-2-yl]piperazine; 1-(4-methylpyridin-2-yl)-4-{[5-(phenylethynyl)pyridin-3-yl]carbonyl}piperazine; 1-{[5-(phenylethynyl)pyridin-3-yl]carbonyl}-4-[3-(trifluoromethyl)pyridin-2-yl]piperazine; 2-(4-{[5-(phenylethynyl)pyridin-3-yl]carbonyl}piperazin-1-yl)nicotinonitrile; 4,6-dimethyl-2-(4-{[5-(phenylethynyl)pyridin-3-yl]carbonyl}piperazin-1-yl)pyrimidine; 2-(4-{[5-(phenylethynyl)pyridin-3-yl]carbonyl}piperazin-1-yl)pyrazine; 1-{[5-(phenylethynyl)pyridin-3-yl]carbonyl}-4-pyridin-4-ylpiperazin; 2-{4-[2-(phenylethynyl)isonicotinoyl]piperazin-1-yl}nicotinonitrile; 1-(6-methylpyridin-2-yl)-4-{[5-(phenylethynyl)pyridin-3-yl]carbonyl}piperazine; 1-[4-methoxy-3-(pyridin-2-ylethynyl)benzoyl]-4-pyridin-2-ylpiperazin or 1-[2-(phenylethynyl)isonicotinoyl]-4-pyridin-4-ylpiperazin.

19. The compound of claim 1, wherein the compound is 1-[4-methoxy-3-(pyridin-2-ylethynyl)benzoyl]-4-[5-(trifluoromethyl)pyridin-2-yl]piperazine; 1-[4-methoxy-3-(pyridin-2-ylethynyl)benzoyl]-4-[3-(trifluoromethyl)pyridin-2-yl]piperazine; 1-(3,5-dichloropyridin-2-yl)-4-[4-methoxy-3-(pyridin-2-ylethynyl)benzoyl]piperazine; 1-(3-chloropyridin-2-yl)-4-[4-methoxy-3-(pyridin-2-ylethynyl)benzoyl]piperazine; 1-[4-methoxy-3-(pyridin-2-ylethynyl)benzoyl]-4-[3-(trifluoromethyl)phenyl]piperazine; 1-(5-chloropyridin-2-yl)-4-[4-methoxy-3-(pyridin-2-ylethynyl)benzoyl]piperazine; 1-(3-chlorophenyl)-4-[4-methoxy-3-(pyridin-2-ylethynyl)benzoyl]piperazine; 1-[3-chloro-5-(trifluoromethyl)pyridin-2-yl]-4-[4-methoxy-3-(pyridin-2-ylethynyl)benzoyl]piperazine; 1-[4-methoxy-3-(pyridin-2-ylethynyl)benzoyl]-4-(4-methylpyridin-2-yl)piperazine; 2-{4-[4-methoxy-3-(pyridin-2-ylethynyl)benzoyl]piperazin-1-yl}-4,6-dimethylpyrimidine; 3-{4-[4-methoxy-3-(pyridin-2-ylethynyl)benzoyl]piperazin-1-yl}pyrazine-2-carbonitrile; 2-{4-[4-methoxy-3-(pyridin-2-ylethynyl)benzoyl]piperazin-1-yl}-4-(trifluoromethyl)pyrimidine; 3-{4-[4-methoxy-3-(pyridin-2-ylethynyl)benzoyl]piperazin-1-yl}phenol; 1-[4-methoxy-3-(pyridin-2-ylethynyl)benzoyl]-4-(3-methylphenyl)piperazine; 5-bromo-4-methoxy-2-{4-[4-methoxy-3-(pyridin-2-ylethynyl)benzoyl]piperazin-1-yl}pyrimidine; 1-[4-methoxy-3-(pyridin-2-ylethynyl)benzoyl]-4-(6-methylpyridin-2-yl)piperazine; (1R,4S)-2-(4-chlorophenyl)-5-[4-methoxy-3-(pyridin-2-ylethynyl)benzoyl]-2,5-diazabicyclo[2.2.1]heptane; 4-methoxy-2-{4-[4-methoxy-3-(pyridin-2-ylethynyl)benzoyl]piperazin-1-yl}pyrimidine; 3-{4-[4-methoxy-3-(pyridin-2-ylethynyl)benzoyl]piperazin-1-yl}-1,2-benzisothiazole; 6-{4-[4-methoxy-3-(pyridin-2-ylethynyl)benzoyl]-1,4-diazepane-1-yl}nicotinonitrile or 1-[4-methoxy-3-(pyridin-2-ylethynyl)benzoyl]-4-(5-methylpyridin-2-yl)piperazine; 2-{4-[4-methoxy-3-(pyridin-2-ylethynyl)benzoyl]piperazin-1-yl}-6-methylpyrazine; 1-[4-methoxy-3-(pyridin-2-ylethynyl)benzoyl]-4-pyridin-2-yl-1,4-diazepane; 1-[4-methoxy-3-(pyridin-2-ylethynyl)benzoyl]-4-[5-(trifluoromethyl)-1,3,4-thiadiazol-2-yl]piperazine; (1R,4S)-2-(3-fluorophenyl)-5-[4-methoxy-3-(pyridin-2-ylethynyl)benzoyl]-2,5-diazabicyclo[2.2.1]heptane; 1-[4-methoxy-3-(pyridin-2-ylethynyl)benzoyl]-4-(3-methylpyridin-2-yl)piperazine; 1-[4-methoxy-3-(pyridin-2-ylethynyl)benzoyl]-4-[3-(trifluoromethyl)pyridin-2-yl]-1,4-diazepane; 1-[4-methoxy-3-(pyridin-2-ylethynyl)benzoyl]-4-[5-(trifluoromethyl)pyridin-2-yl]-1,4-diazepane; 1-[4-methoxy-3-(pyridin-2-ylethynyl)benzoyl]-4-(6-methylpyridin-2-yl)-1,4-diazepane; 1-[3-chloro-5-(trifluoromethyl)pyridin-2-yl]-4-[4-methoxy-3-(pyridin-2-ylethynyl)benzoyl]-1,4-diazepane; 1-(6-methoxypyridin-2-yl)-4-[4-methoxy-3-(pyridin-2-ylethynyl)benzoyl]piperazine; 2-{4-[4-methoxy-3-(pyridin-2-ylethynyl)benzoyl]-1,4-diazepane-1-yl}nicotinonitrile; 1-[4-methoxy-3-(pyridin-2-ylethynyl)benzoyl]-4-(5-nitropyridin-2-yl)-1,4-diazepane; 1-(2-chlorophenyl)-4-[4-methoxy-3-(pyridin-2-ylethynyl)benzoyl]piperazine; 1-(4-chlorophenyl)-4-[4-methoxy-3-(pyridin-2-ylethynyl)benzoyl]piperazine; 1-(3,4-dichlorophenyl)-4-[4-methoxy-3-(pyridin-2-ylethynyl)benzoyl]piperazine; 1-(2,3-dimethylphenyl)-4-[4-methoxy-3-(pyridin-2-ylethynyl)benzoyl]piperazine; 2-isopropyl-4-{4-[4-methoxy-3-(pyridin-2-ylethynyl)benzoyl]piperazin-1-yl}-6-methylpyrimidine; 1-[4-methoxy-3-(pyridin-2-ylethynyl)benzoyl]-4-(2-methylphenyl)piperazine; 1-[4-methoxy-3-(pyridin-2-ylethynyl)benzoyl]-4-(4-methylphenyl)piperazine; or 1-(3-fluorophenyl)-4-[4-methoxy-3-(pyridin-2-ylethynyl)benzoyl]piperazine, 1-[4-methoxy-3-(pyridin-2-ylethynyl)benzoyl]-4-(phenylsulfonyl)piperazine; 1-[(5-chloro-2-thienyl)sulfonyl]-4-[4-methoxy-3-(pyridin-2-ylethynyl)benzoyl]piperazine; (1R,4S)-2-[4-methoxy-3-(pyridin-2-ylethynyl)benzoyl]-5-(4-methylphenyl)-2,5-diazabicyclo[2.2.1]heptane; (1S,4R)-2-(4-fluorophenyl)-5-[4-methoxy-3-(pyridin-2-ylethynyl)benzoyl]-2,5-diazabicyclo[2.2.1]heptane; 1-[4-methoxy-3-(pyridin-2-ylethynyl)benzoyl]-4-[4-(trifluoromethyl)phenyl]piperazine; 1-[4-methoxy-3-(pyridin-2-ylethynyl)benzoyl]-4-(5-nitropyridin-2-yl)piperazine; 1-(2-methoxyphenyl)-4-[4-methoxy-3-(pyridin-2-ylethynyl)benzoyl]piperazine; 1-(4-fluorophenyl)-4-[4-methoxy-3-(pyridin-2-ylethynyl)benzoyl]piperazine; 1-[4-methoxy-3-(pyridin-2-ylethynyl)benzoyl]-4-(4-nitrophenyl)piperazine; 1-(4-methoxyphenyl)-4-[4-methoxy-3-(pyridin-2-ylethynyl)benzoyl]piperazine; 1-(benzylsulfonyl)-4-[4-methoxy-3-(pyridin-2-ylethynyl)benzoyl]piperazine; 1-(2,3-dihydro-1,4-benzodioxin-6-ylsulfonyl)-4-[4-methoxy-3-(pyridin-2-ylethynyl)benzoyl]piperazine; 1-[4-methoxy-3-(pyridin-2-ylethynyl)benzoyl]-4-pyridin-4-ylpiperazin; 1-(4-{4-[4-methoxy-3-(pyridin-2-ylethynyl)benzoyl]piperazin-1-yl}phenyl)ethanone; 1-[4-methoxy-3-(pyridin-2-ylethynyl)benzoyl]-4-[4-(methylsulfonyl)phenyl]piperazine; 1-[(3,4-dichlorophenyl)sulfonyl]-4-[4-methoxy-3-(pyridin-2-ylethynyl)benzoyl]piperazine; 1-[4-fluoro-2-(methylsulfonyl)phenyl]-4-[4-methoxy-3-(pyridin-2-ylethynyl)benzoyl]piperazine; 1-(3-methoxyphenyl)-4-[4-methoxy-3-(pyridin-2-ylethynyl)benzoyl]piperazine; 1-(2,5-dimethylphenyl)-4-[4-methoxy-3-(pyridin-2-ylethynyl)benzoyl]piperazine; 1-[(4-chlorophenyl)sulfonyl]-4-[4-methoxy-3-(pyridin-2-ylethynyl)benzoyl]piperazine; 1-benzoyl-4-[4-methoxy-3-(pyridin-2-ylethynyl)benzoyl]piperazine; 1-(ethylsulfonyl)-4-[4-methoxy-3-(pyridin-2-ylethynyl)benzoyl]piperazine; 1-[4-methoxy-3-(pyridin-2-ylethynyl)benzoyl]-4-[2-(trifluoromethyl)phenyl]piperazine; 1-[4-methoxy-3-(pyridin-2-ylethynyl)benzoyl]-4-(1,3-thiazol-2-yl)piperazine; 1-(cyclopropylcarbonyl)-4-[4-methoxy-3-(pyridin-2-ylethynyl)benzoyl]piperazine; 1-[4-methoxy-3-(pyridin-2-ylethynyl)benzoyl]-4-(tetrahydrofuran-2-ylcarbonyl)piperazine; 1-[4-methoxy-3-(pyridin-2-ylethynyl)benzoyl]-2-methyl-4-phenylpiperazine; 3-{4-[4-methoxy-3-(pyridin-2-ylethynyl)benzoyl]piperazin-1-yl}-1,2-benzisoxazole; 6-{4-[4-methoxy-3-(pyridin-2-ylethynyl)benzoyl]piperazin-1-yl}nicotinonitrile; 1-[4-methoxy-3-(pyridin-2-ylethynyl)benzoyl]-4-[(4-methylphenyl)sulfonyl]piperazine; 5-{4-[4-methoxy-3-(pyridin-2-ylethynyl)benzoyl]piperazin-1-yl}-4-nitrothiophene-2-sulfonamide; 1-(6-chloropyridin-2-yl)-4-[4-methoxy-3-(pyridin-2-ylethynyl)benzoyl]piperazine; 2-{4-[4-methoxy-3-(pyridin-2-ylethynyl)benzoyl]piperazin-1-yl}-1,3-benzothiazole; 2-{4-[4-methoxy-3-(pyridin-2-ylethynyl)benzoyl]piperazin-1-yl}-1,3-benzoxazole; 1-(2-furoyl)-4-[4-methoxy-3-(pyridin-2-ylethynyl)benzoyl]piperazine; 1-(1,3-benzodioxol-5-ylmethyl)-4-[4-methoxy-3-(pyridin-2-ylethynyl)benzoyl]piperazine; 1-(1,3-benzodioxol-5-ylmethyl)-4-[4-methoxy-3-(pyridin-2-ylethynyl)benzoyl]piperazine; 1-(1,3-benzodioxol-5-ylmethyl)-4-[4-methoxy-3-(pyridin-2-ylethynyl)benzoyl]piperazine; 7-bromo-3-{4-[4-methoxy-3-(pyridin-2-ylethynyl)benzoyl]piperazin-1-yl}isoquinoline; 5-bromo-2-{4-[4-methoxy-3-(pyridin-2-ylethynyl)benzoyl]piperazin-1-yl}pyrimidine; 1-(2-methoxybenzoyl)-4-[4-methoxy-3-(pyridin-2-ylethynyl)benzoyl]piperazine; 1-(3-methoxybenzoyl)-4-[4-methoxy-3-(pyridin-2-ylethynyl)benzoyl]piperazine; 1-(4-methoxybenzoyl)-4-[4-methoxy-3-(pyridin-2-ylethynyl)benzoyl]piperazine; 1-(2-fluorobenzyl)-4-[4-methoxy-3-(pyridin-2-ylethynyl)benzoyl]piperazine; 1-(3-fluorobenzyl)-4-[4-methoxy-3-(pyridin-2-ylethynyl)benzoyl]piperazine; 1-(4-fluorobenzyl)-4-[4-methoxy-3-(pyridin-2-ylethynyl)benzoyl]piperazine; 1-[(5-bromo-2-thienyl)sulfonyl]-4-[4-methoxy-3-(pyridin-2-ylethynyl)benzoyl]piperazine; 1-[(3,5-dimethylisoxazol-4-yl)sulfonyl]-4-[4-methoxy-3-(pyridin-2-ylethynyl)benzoyl]piperazine; 1-(3,5-dichlorophenyl)-4-[4-methoxy-3-(pyridin-2-ylethynyl)benzoyl]piperazine; 1-[4-methoxy-3-(pyridin-2-ylethynyl)benzoyl]-4-{[3-methoxy-4-(1H-tetrazol-1-yl)phenyl]sulfonyl}piperazine; 5-({4-[4-methoxy-3-(pyridin-2-ylethynyl)benzoyl]piperazin-1-yl}sulfonyl)-N,N-dimethylnaphthalene-1-amine; 1-(3-chlorobenzyl)-4-[4-methoxy-3-(pyridin-2-ylethynyl)benzoyl]piperazine; 1-(4-chlorobenzyl)-4-[4-methoxy-3-(pyridin-2-ylethynyl)benzoyl]piperazine; 1-[4-methoxy-3-(pyridin-2-ylethynyl)benzoyl]-4-(5-nitro-1,3,4-thiadiazol-2-yl)piperazine; 1-(2,6-dichlorobenzyl)-4-[4-methoxy-3-(pyridin-2-ylethynyl)benzoyl]piperazine; 1-[(2-chlorophenyl)sulfonyl]-4-[4-methoxy-3-(pyridin-2-ylethynyl)benzoyl]piperazine; 1-[(3-chlorophenyl)sulfonyl]-4-[4-methoxy-3-(pyridin-2-ylethynyl)benzoyl]piperazine; 1-(2,4-dichlorobenzyl)-4-[4-methoxy-3-(pyridin-2-ylethynyl)benzoyl]piperazine; 1-[4-methoxy-3-(pyridin-2-ylethynyl)benzoyl]-4-(3-phenyl-1,2,4-thiadiazol-5-yl)piperazine; 2-{4-[4-methoxy-3-(pyridin-2-ylethynyl)benzoyl]piperazin-1-yl}-6-nitro-1,3-benzothiazole; 1-[4-methoxy-3-(pyridin-2-ylethynyl)benzoyl]-4-[5-(1-methyl-5-nitro-1H-imidazol-2-yl)-1,3,4-thiadiazol-2-yl]piperazine; The compound of claim 1, wherein the compound is 1-[4-methoxy-3-(pyridin-2-ylethynyl)benzoyl]-4-{[4-(1H-tetrazol-1-yl)phenyl]sulfonyl}piperazine; 1-(4-bromo-2-fluorobenzyl)-4-[4-methoxy-3-(pyridin-2-ylethynyl)benzoyl]piperazine; tert-butyl 4-[4-methoxy-3-(pyridin-2-ylethynyl)benzoyl]piperazine-1-carboxylate; 1-[4-methoxy-3-(pyridin-2-ylethynyl)benzoyl]-4-(2-naphthylsulfonyl)piperazine; 1-(3,4-dichlorobenzyl)-4-[4-methoxy-3-(pyridin-2-ylethynyl)benzoyl]piperazine; 1-(2-chloro-6-fluorobenzyl)-4-[4-methoxy-3-(pyridin-2-ylethynyl)benzoyl]piperazine; 1-(1-benzothiophene-2-yl)-4-[4-methoxy-3-(pyridin-2-ylethynyl)benzoyl]piperazine; 1-[4-methoxy-3-(pyridin-2-ylethynyl)benzoyl]-4-(5-phenyl-4H-1,2,4-triazol-3-yl)piperazine; 1-[4-methoxy-3-(pyridin-2-ylethynyl)benzoyl]-4-phenylpiperazine; 4-amino-2-{4-[4-methoxy-3-(pyridin-2-ylethynyl)benzoyl]piperazin-1-yl}pyrimidine-5-carbonitrile; 4-chloro-6-{4-[4-methoxy-3-(pyridin-2-ylethynyl)benzoyl]piperazin-1-yl}-2-(methylthio)pyrimidine; 2-chloro-5-fluoro-4-{4-[4-methoxy-3-(pyridin-2-ylethynyl)benzoyl]piperazin-1-yl}pyrimidine; 4-{4-[4-methoxy-3-(pyridin-2-ylethynyl)benzoyl]piperazin-1-yl}-2-(methylthio)pyrimidine; 4-chloro-6-{4-[4-methoxy-3-(pyridin-2-ylethynyl)benzoyl]piperazin-1-yl}-2-methylpyrimidine; 5-fluoro-2-{4-[4-methoxy-3-(pyridin-2-ylethynyl)benzoyl]piperazin-1-yl}pyrimidine; 5-fluoro-2-{4-[4-methoxy-3-(pyridin-2-ylethynyl)benzoyl]piperazin-1-yl}pyrimidine; 5-fluoro-2-{4-[4-methoxy-3-(pyridin-2-ylethynyl)benzoyl]piperazin-1-yl}pyrimidin-4-amine; 3-methoxy-6-{4-[4-methoxy-3-(pyridin-2-ylethynyl)benzoyl]piperazin-1-yl}pyridazine; 6-chloro-3-{4-[4-methoxy-3-(pyridin-2-ylethynyl)benzoyl]piperazin-1-yl}-4-methylpyridazine; 2-chloro-3-{4-[4-methoxy-3-(pyridin-2-ylethynyl)benzoyl]piperazin-1-yl}pyrazine; 2,4-dimethoxy-6-{4-[4-methoxy-3-(pyridin-2-ylethynyl)benzoyl]piperazin-1-yl}-1,3,5-triazine; 1-chloro-4-{4-[4-methoxy-3-(pyridin-2-ylethynyl)benzoyl]piperazin-1-yl}phthalazine; 3-{4-[4-methyl-3-(pyridin-2-ylethynyl)benzoyl]piperazin-1-yl}-1,2-benzisoxazole; 4-methoxy-2-{4-[4-methyl-3-(pyridin-2-ylethynyl)benzoyl]piperazin-1-yl}pyrimidine; 2-{4-[4-methyl-3-(pyridin-2-ylethynyl)benzoyl]piperazin-1-yl}pyrimidine; 1-(3,5-dichloropyridin-2-yl)-4-[4-methyl-3-(pyridin-2-ylethynyl)benzoyl]piperazine; 1-(3-chloropyridin-2-yl)-4-[4-methyl-3-(pyridin-2-ylethynyl)benzoyl]piperazine; 2-{4-[4-fluoro-3-(pyridin-2-ylethynyl)benzoyl]piperazin-1-yl}pyrimidine; 1-[4-fluoro-3-(pyridin-2-ylethynyl)benzoyl]-4-pyridin-2-ylpiperazin; 1-(3,5-dichloropyridin-2-yl)-4-[4-fluoro-3-(pyridin-2-ylethynyl)benzoyl]piperazine; 1-(3-chloropyridin-2-yl)-4-[4-fluoro-3-(pyridin-2-ylethynyl)benzoyl]piperazine; 3-{4-[4-fluoro-3-(pyridin-2-ylethynyl)benzoyl]piperazin-1-yl}-1,2-benzisoxazole; 2-{4-[4-fluoro-3-(pyridin-2-ylethynyl)benzoyl]piperazin-1-yl}-4-methoxypyrimidine; 1-[4-ethoxy-3-(pyridin-2-ylethynyl)benzoyl]-4-pyridin-2-ylpiperazin; 1-(3,5-dichloropyridin-2-yl)-4-[4-ethoxy-3-(pyridin-2-ylethynyl)benzoyl]piperazine; 2-{4-[4-ethoxy-3-(pyridin-2-ylethynyl)benzoyl]piperazin-1-yl}-4-methoxypyrimidine; 1-(3-chloropyridin-2-yl)-4-[4-ethoxy-3-(pyridin-2-ylethynyl)benzoyl]piperazine; 3-{4-[4-ethoxy-3-(pyridin-2-ylethynyl)benzoyl]piperazin-1-yl}-1,2-benzisoxazole; 1-{[4-(cyclopropylmethoxy)-3-(pyridin-2-ylethynyl)phenyl]carbonyl}-4-pyridin-2-ylpiperazin; 3-(4-{[4-(cyclopropylmethoxy)-3-(pyridin-2-ylethynyl)phenyl]carbonyl}piperazin-1-yl)-1,2-benzisoxazole; 2-(4-{[4-(cyclopropylmethoxy)-3-(pyridin-2-ylethynyl)phenyl]carbonyl}piperazin-1-yl)pyrimidine; 1-(4-chlorophenyl)-4-{[4-methoxy-3-(pyridin-2-ylethynyl)phenyl]carbonyl}piperazin-2-one; 1-(3-chlorophenyl)-4-{[4-methoxy-3-(pyridin-2-ylethynyl)phenyl]carbonyl}piperazin-2-one; 1-(2-chlorophenyl)-4-{[4-methoxy-3-(pyridin-2-ylethynyl)phenyl]carbonyl}piperazin-2-one; 4-{[4-methoxy-3-(pyridin-2-ylethynyl)phenyl]carbonyl}-1-phenylpiperazin-2-one; 4-{[4-methoxy-3-(pyridin-2-ylethynyl)phenyl]carbonyl}-1-pyridin-2-ylpiperazin-2-one; 1-benzyl-4-[4-methoxy-3-(pyridin-2-ylethynyl)benzoyl]piperazin-2-one; 1-(2-chlorobenzyl)-4-[4-methoxy-3-(pyridin-2-ylethynyl)benzoyl]piperazin-2-one; 1-(3-chlorobenzyl)-4-[4-methoxy-3-(pyridin-2-ylethynyl)benzoyl]piperazin-2-one; 1-(4-chlorobenzyl)-4-[4-methoxy-3-(pyridin-2-ylethynyl)benzoyl]piperazin-2-one; 2-{4-[3-(pyridin-2-ylethynyl)-4-(trifluoromethoxy)benzoyl]piperazin-1-yl}pyrazine; 1-(3-chloropyridin-2-yl)-4-[3-(pyridin-2-ylethynyl)-4-(trifluoromethoxy)benzoyl]piperazine; 1-[3-(pyridin-2-ylethynyl)-4-(trifluoromethoxy)benzoyl]-4-[3-(trifluoromethyl)phenyl]piperazine; 3-{4-[3-(pyridin-2-ylethynyl)-4-(trifluoromethoxy)benzoyl]piperazin-1-yl}-1,2-benzisoxazole; 5-bromo-4-methoxy-2-{4-[3-(pyridin-2-ylethynyl)-4-(trifluoromethoxy)benzoyl]piperazin-1-yl}pyrimidine; 1-pyridin-2-yl-4-{[3-(pyridin-2-ylethynyl)-4-(trifluoromethoxy)phenyl]carbonyl}piperazine; 1-(3-chloropyridin-2-yl)-4-[4-methoxy-3-(phenylethynyl)benzoyl]piperazine; 1-(3-chloropyridin-2-yl)-4-{4-methoxy-3-[(2-nitrophenyl)ethynyl]benzoyl}piperazine or 1-(3-{[3-(benzyloxy)phenyl]ethynyl}-4-methoxybenzoyl)-4-(3-chloropyridin-2-yl)piperazine, 1-(3-chloropyridin-2-yl)-4-(4-methoxy-3-{[3-(trifluoromethoxy)phenyl]ethynyl}benzoyl)piperazine; 1-(3-chloropyridin-2-yl)-4-{4-methoxy-3-[(3-nitrophenyl)ethynyl]benzoyl}piperazine; 1-(3-{[4-(benzyloxy)phenyl]ethynyl}-4-methoxybenzoyl)-4-(3-chloropyridin-2-yl)piperazine; 1-{4-[(5-{[4-(3-chloropyridin-2-yl)piperazin-1-yl]carbonyl}-2-methoxyphenyl)ethynyl]phenyl}ethanone; 1-(3-chloropyridin-2-yl)-4-(4-methoxy-3-{[4-(trifluoromethane)phenyl]ethynyl}benzoyl)piperazine; 1-(3-chloropyridin-2-yl)-4-{4-methoxy-3-[(4-nitrophenyl)ethynyl]benzoyl}piperazine; 1-(3-chloropyridin-2-yl)-4-{3-[(2-fluorophenyl)ethynyl]-4-methoxybenzoyl}piperazine; 1-{3-[(4-chlorophenyl)ethynyl]-4-methoxybenzoyl}-4-(3-chloropyridin-2-yl)piperazine; 2-{4-[4-methoxy-3-(pyridin-2-ylethynyl)benzoyl]piperazin-1-yl}pyrimidine; 2-{4-[4-methoxy-3-(pyridin-3-ylethynyl)benzoyl]piperazin-1-yl}pyrimidine; 2-{4-[4-methoxy-3-(pyridin-4-ylethynyl)benzoyl]piperazin-1-yl}pyrimidine; 2-[4-(4-methoxy-3-{[3-(trifluoromethoxy)phenyl]ethynyl}benzoyl)piperazin-1-yl]pyrimidine; 2-(4-{4-methoxy-3-[(3-nitrophenyl)ethynyl]benzoyl}piperazin-1-yl)pyrimidine; 2-[4-(3-{[4-(benzyloxy)phenyl]ethynyl}-4-methoxybenzoyl)piperazin-1-yl]pyrimidine; 2-(4-{3-[(2-fluorophenyl)ethynyl]-4-methoxybenzoyl}piperazin-1-yl)pyrimidine; 2-(4-{3-[(2-chlorophenyl)ethynyl]-4-methoxybenzoyl}piperazin-1-yl)pyrimidine; 3-({2-methoxy-5-[(4-pyrimidin-2-ylpiperazin-1-yl)carbonyl]phenyl}ethynyl)benzonitrile; 2-(4-{3-[(4-fluorophenyl)ethynyl]-4-methoxybenzoyl}piperazin-1-yl)pyrimidine; 2-(4-{3-[(4-chlorophenyl)ethynyl]-4-methoxybenzoyl}piperazin-1-yl)pyrimidine; 2-[4-(3-{[3-(difluoromethoxy)phenyl]ethynyl}-4-methoxybenzoyl)piperazin-1-yl]pyrimidine, 5-bromo-4-methoxy-2-(4-{[3-(pyridin-2-ylethynyl)-4-(trifluoromethyl)phenyl]carbonyl}piperazin-1-yl)pyrimidine, 3-(4-{[3-(pyridin-2-ylethynyl)-4-(trifluoromethyl)phenyl]carbonyl}piperazin-1-yl)pyrazine-2-carbonitrile; 1-(4-methylpyridin-2-yl)-4-{[3-(pyridin-2-ylethynyl)-4-(trifluoromethyl)phenyl]carbonyl}piperazine; 1-(3,5-dichloropyridin-2-yl)-4-{[3-(pyridin-2-ylethynyl)-4-(trifluoromethyl)phenyl]carbonyl}piperazine; 1-pyridin-2-yl-4-{[3-(pyridin-2-ylethynyl)-4-(trifluoromethyl)phenyl]carbonyl}piperazine; 2-(4-{[3-(pyridin-2-ylethynyl)-4-(trifluoromethyl)phenyl]carbonyl}piperazin-1-yl)pyrimidine; 1-(3-chloropyridin-2-yl)-4-{[3-(pyridin-2-ylethynyl)-4-(trifluoromethyl)phenyl]carbonyl}piperazine; 1-(5-methylpyridin-2-yl)-4-{[3-(pyridin-2-ylethynyl)-4-(trifluoromethyl)phenyl]carbonyl}piperazine; 4-methoxy-2-(4-{[3-(pyridin-2-ylethynyl)-4-(trifluoro methyl)phenyl]carbonyl}piperazin-1-yl)pyrimidine; 3-(4-{[3-(pyridin-2-ylethynyl)-4-(trifluoromethyl)phenyl]carbonyl}piperazin-1-yl)-1,2-benzisoxazole; 1-(furan-2-ylcarbonyl)-4-{[3-(pyridin-2-ylethynyl)-4-(trifluoromethyl)phenyl]carbonyl}piperazine; 1-(3-fluorobenzyl)-4-{[3-(pyridin-2-ylethynyl)-4-(trifluoromethyl)phenyl]carbonyl}piperazine; 2-(4-{[3-(pyridin-2-ylethynyl)-4-(trifluoromethyl)phenyl]carbonyl}piperazin-1-yl)-1,3-benzothiazole; 1-{[3-(pyridin-2-ylethynyl)-4-(trifluoromethyl)phenyl]carbonyl}-4-(1,3-thiazol-2-yl)piperazine; 1-{[3-(pyridin-2-ylethynyl)-4-(trifluoromethyl)phenyl]carbonyl}-4-[5-(trifluoromethyl)pyridin-2-yl]piperazine; 1-(6-methylpyridin-2-yl)-4-{[3-(pyridin-2-ylethynyl)-4-(trifluoromethyl)phenyl]carbonyl}piperazine; 2-(4-{[3-(pyridin-2-ylethynyl)-4-(trifluoromethyl)phenyl]carbonyl}piperazin-1-yl)pyrazine; 5-bromo-2-(4-{[4-(difluoromethoxy)-3-(pyridin-2-ylethynyl)phenyl]carbonyl}piperazin-1-yl)-4-methoxypyrimidine; or 3-(4-{[4-(difluoromethoxy)-3-(pyridin-2-ylethynyl)phenyl]carbonyl}piperazin-1-yl)-1,2-benzisothiazole; 3-(4-{[4-(difluoromethoxy)-3-(pyridin-2-ylethynyl)phenyl]carbonyl}piperazin-1-yl)-1,2-benzisoxazole; 1-{[4-(difluoromethoxy)-3-(pyridin-2-ylethynyl)phenyl]carbonyl}-4-pyridin-2-ylpiperazin; 1-[4-chloro-3-(pyridin-2-ylethynyl)benzoyl]-4-pyridin-2-ylpiperazin; 2-{4-[4-chloro-3-(pyridin-2-ylethynyl)benzoyl]piperazin-1-yl}pyrimidine; 2-{4-[4-chloro-3-(pyridin-2-ylethynyl)benzoyl]piperazin-1-yl}pyrazine; 2-{4-[4-chloro-3-(pyridin-2-ylethynyl)benzoyl]piperazin-1-yl}nicotinonitrile; 1-[4-chloro-3-(pyridin-2-ylethynyl)benzoyl]-4-(1,3-thiazol-2-yl)piperazine; 1-[4-chloro-3-(pyridin-2-ylethynyl)benzoyl]-4-pyridin-4-ylpiperazin; 1-[4-chloro-3-(pyridin-2-ylethynyl)benzoyl]-4-[3-(trifluoromethyl)phenyl]piperazine; 1-[4-chloro-3-(pyridin-2-ylethynyl)benzoyl]-4-[3-(trifluoromethyl)phenyl]piperazine; 3-(4-{[4-chloro-3-(pyridin-2-ylethynyl)phenyl]carbonyl}piperazin-1-yl)phenol; 1-(3-chloropyridin-2-yl)-4-{[4-chloro-3-(pyridin-2-ylethynyl)phenyl]carbonyl}piperazine; 1-(4-chloropyridin-2-yl)-4-{[4-chloro-3-(pyridin-2-ylethynyl)phenyl]carbonyl}piperazine or 3-(4-{[4-chloro-3-(pyridin-2-ylethynyl)phenyl]carbonyl}piperazin-1-yl)pyrazine-2-carbonitrile

20. The compound of claim 21, wherein the compound is selected from 1-pyridin-2-yl-4-{[3-(pyridin-2-ylethynyl)-4-(trifluoromethoxy)phenyl]carbonyl}piperazine and 1-{[4-(difluoromethoxy)-3-(pyridin-2-ylethynyl)phenyl]carbonyl}-4-pyridin-2-ylpiperazin.

21. The compound of claim 1 or 2, wherein Y is O, and Q₃ and Q₅ are each phenyl optionally substituted with 1 to 3 substituents independently selected from C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl) and CN.

22. The compound of claim 1 or 2, wherein R₂ is selected from —CH═CH—, —CH₂—O— and —O—CH₂—; Y is O; and Q₃ and Q₅ are each phenyl optionally substituted with 1 to 3 substituents independently selected from C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl) and CN.

23. The compound of claim 1 or 2, wherein the compound is 3-({3-[(4-pyridin-2-ylpiperazin-1-yl)methyl]phenyl}ethynyl)phenol; 1-[3-(cyclohex-1-en-1-ylethynyl)benzyl]-4-pyridin-2-ylpiperazin; 1-[3-(3-phenylprop-1-yn-1-yl)benzyl]-4-pyridin-2-ylpiperazin; 3-({3-[(4-pyridin-2-yl piperazin-1-yl)methyl]phenyl}ethynyl)aniline; and 1-{3-[(3-methoxyphenyl)ethynyl]benzyl}-4-pyridin-2-ylpiperazin.

24. The compound of claim 1 or 2, wherein the compound is 3-({3-[(4-pyridin-2-ylpiperazin-1-yl)sulfonyl]phenyl}ethynyl)phenol; 1-{[3-(cyclohex-1-en-1-ylethynyl)phenyl]sulfonyl}-4-pyridin-2-ylpiperazin; 1-{[3-(3-phenylprop-1-yn-1-yl)phenyl]sulfonyl}-4-pyridin-2-ylpiperazin; 1-({3-[(3-methoxyphenyl)ethynyl]phenyl}sulfonyl)-4-pyridin-2-ylpiperazin; or 1-{[3-(phenylethynyl)phenyl]sulfonyl}-4-pyridin-2-yl-piperazine.

25. The compound of claim 1 or 2, wherein:
R₂ is -L₃-Q₄;
Q₄ is 5 to 14 membered heteroaromatic optionally substituted with 1 to 3 substituents independently selected from C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl) and CN; and
R₆ is -(L₅)-(5 to 14 membered heteroaromatic) optionally substituted with 1 to 3 substituents independently selected from H, C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl and CN.

26. The compound of claim 1 or 2, wherein Q₄ is pyridyl optionally substituted with 1 to 3 substituents independently selected from C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl) and CN.

27. The compound of claim 1 or 2, wherein:
Q₄ is pyrid-2-yl; and
R₆ is -(L₅)-(pyrid-2-yl) optionally substituted with 1 to 3 substituents independently selected from H, C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl and CN.

28. The compound of claim 1 or 2, wherein X₁ is CR₃ and X₂ is CH.

29. A method of treating a patient suffering from a chronic condition selected from schizophrenia, paranoia, depression, manic-depressive illness, anxiety, panic disorders, social anxiety, obsessive compulsive disorders, generalized anxiety disorders, phobias, post-traumatic stress disorder, bipolar disorder, Asperger's syndrome, pervasive developmental disorders, gastrointestinal disorders such as gastroesophageal reflux disease, dyspepsia, irritable bowel syndrome, functional bloating, functional diarrhea, chronic constipation, functional disturbances of the biliary tract, migraine, chronic pain, fibromyalgia, neuropathic pain, post-herpatic neuropathic pain, addiction, Parkinson's disease, senile dementia, levadopa-induced dyskinesia, Alzheimer's disease, Huntington's chorea, amyotrophic lateral sclerosis, multiple sclerosis, Down Syndrome, fragile-X syndrome, autistic spectrum disorders, attention deficit hyperactivity disorder, stroke, ischemic injury, epilepsy, hypoglycemia and obesity comprising providing a therapeutically effective amount of compound of Formula I: 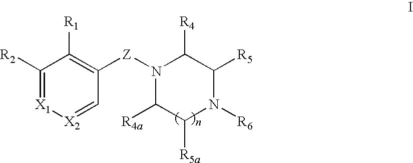
wherein:
R₁ is each independently selected from H, C₁₋₆ alkyl, halogen, OH, and OC₁₋₆ alkyl;
R₂ is selected from -(L₁)ₐ-(Y)_{c}-(L₂)_{b}-Q₃, -L₃-Q₄ and -L₄-Q₅;
L₃ is C₂₋₁₂ alkynyl optionally substituted with 1-3 substituents selected from OH and halogen;
L₁ and L₂ are each independently C₁₋₃ alkyl;
L₄ is C₂₋₁₂ alkenyl optionally substituted with 1-3 substituents selected from OH and halogen;
n is 1 or 2
R₄, R₄ₐ, R₅, and R₅ₐ are each independently selected from H, (═O) and C₁₋₆ alkyl; or R₄ and one of R₅ₐ together can form a bridging methylene; or R₅ can be together with the carbon to which it is attached —C(═O)
R₆ is selected from H, CH₃, -(L₅)-(3- to 14-membered heterocycle), -(L₅)-(5 to 14 membered heteroaromatic), (L₅)-(3- to 10-membered cycloalkyl), (L₅)-(C₆₋₁₄ aryl) and -(L₅)-C₁₋₆ alkyl each of which except H can be optionally substituted with 1 to 3 substituents independently selected from H, C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl, CN, (5- to 14-membered heteroaromatic), NR₁R₁, SO₂C₁₋₆ alkyl, SO₂, SO₂NR₁R₁, C₁₋₆ alkylaryl, COC₁₋₆ alkyl, and (3- to 14-membered heterocycle) optionally substituted with NO₂.
L₅ is selected from a bond, C₁₋₃ alkyl, —C(═O)—, SO₂, (3- to 6-membered heterocycle) and (5- to 14-membered heteroaromatic).
X₁, X₂ are independently CR₃ or N;
each R₃ is independently selected from H, C₁₋₆ alkyl, halogen, OH, OC₁₋₆ alkyl, SO₂, (3- to 14-membered heterocycle) and (5- to 14-membered heteroaromatic), wherein each of C₁₋₆ alkyl or OC₁₋₆ alkyl can be optionally substituted with 1 to 3 substituents independently selected from halogen, OH, OC₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl), cycloalkyl, NR₁R₁, and CN;
Z is CO;
Y is selected from CR₇R₈, NR₉, O, and S;
R₇, R₈, R₉ are independently selected from H, C₁₋₆ alkyl, halogen, OH, and OC₁₋₆ alkyl
a, b and c are independently 0 or 1; and
Q₃ is selected from C₆₋₁₄ aryl, (5- to 14-membered heterocyclic), (5- to 14-membered heteroaromatic), and (4- to 9-membered carbocyclic); each of which can be optionally substituted with 1 to 3 substituents independently selected from C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl), OC₁₋₃ haloalkyl, OC₁₋₆ alkylaryl and CN;
Q₄ is selected from H, C₆₋₁₄ aryl, (5- to 14-membered heterocyclic), (5- to 14-membered heteroaromatic), and (4- to 9-membered carbocyclic); each of which except H can be optionally substituted with 1 to 3 substituents independently selected from C₁₋₆ alkyl, halogen, OH, OC₁₋₆ alkyl, —C(═O)O—(C₁₋₆ alkyl), —C(═O)C₁₋₆ alkyl, NO₂, C₁₋₃ haloalkyl, —S—C₁₋₆ alkyl —NH₂, —NH—(C₁₋₆ alkyl), —N(C₁₋₆ alkyl)(C₁₋₆ alkyl), OC₁₋₃ haloalkyl, OC₁₋₆ alkylaryl and CN;
Q₅ is selected from C₆₋₁₄ aryl, (5- to 14-membered heterocyclic), (5- to 14-membered heteroaromatic), and (4- to 9-membered carbocyclic); each of which can be optionally substituted with 1 to 3 substituents independently selected from C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl), OC₁₋₃ haloalkyl, OC₁₋₆ alkylaryl and CN.

30. A method of treating a patient suffering from a chronic condition selected from schizophrenia, paranoia, depression, manic-depressive illness, anxiety, panic disorders, social anxiety, obsessive compulsive disorders, generalized anxiety disorders, phobias, post-traumatic stress disorder, bipolar disorder, Asperger's syndrome, pervasive developmental disorders, gastrointestinal disorders such as gastroesophageal reflux disease, dyspepsia, irritable bowel syndrome, functional bloating, functional diarrhea, chronic constipation, functional disturbances of the biliary tract, migraine, chronic pain, fibromyalgia, neuropathic pain, post-herpatic neuropathic pain, addiction, Parkinson's disease, senile dementia, levadopa-induced dyskinesia, Alzheimer's disease, Huntington's chorea, amyotrophic lateral sclerosis, multiple sclerosis, Down Syndrome, fragile-X syndrome, autistic spectrum disorders, attention deficit hyperactivity disorder, stroke, ischemic injury, epilepsy, hypoglycemia and obesity comprising providing a therapeutically effective amount of compound of Formula I: 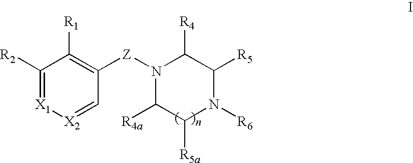
wherein:
R₁ is H, C₁₋₆ alkyl, halogen, OH, or OC₁₋₆ alkyl;
R₂ is -(L₁)ₐ-(Y)_{c}-(L₂)_{b}-Q₃, -L₃-Q₄ or -L₄-Q₅;
L₃ is C₂₋₁₂ alkynyl optionally substituted with 1-3 substituents selected from OH and halogen;
L₁ and L₂ are each independently C₁₋₃ alkyl;
L₄ is C₂₋₁₂ alkenyl optionally substituted with 1-3 substituents selected from OH and halogen;
R₄, R₄ₐ, R₅, and R₅ₐ are each independently H or C₁₋₆ alkyl;
R₆ is selected from H, CH₃, -(L₅)-2-pyridyl, -(L₅)-4-pyridyl, -(L₅)-pyrazinyl, -(L₅)-phenyl, -(L₅)-(3-14 membered heterocyclic), -(L₅)-(5 to 14 membered heteroaromatic) and (L₅)-(C₆₋₁₄ aryl), each of which except H can be optionally substituted with 1 to 3 substituents independently selected from H, C₁₋₆ alkyl, halogen, OH, OC₁₋₆ alkyl, —C(═O)O—(C₁₋₆ alkyl), NO₂, C₁₋₃ haloalkyl, —S—C₁₋₁₆ alkyl, COC₁₋₆ alkyl and CN;
X₁, X₂ are independently CR₃ or N;
L₅ is selected from a bond, C₁₋₃ alkyl, —C(═O)—, SO₂, (3- to 6-membered heterocycle) and (5- to 14-membered heteroaromatic).
X₁, X₂ are independently CR₃ or N;
each R₃ is independently selected from H, C₁₋₆ alkyl, halogen, OH, OC₁₋₆ alkyl, SO₂, (3- to 14-membered heterocyclic) and (5- to 14-membered heteroaromatic), wherein each of C₁₋₁₆ alkyl or OC₁₋₆ alkyl can be optionally substituted with 1 to 3 substituents independently selected from halogen, OH, OC₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl), cycloalkyl, NR₁R₁, and CN;
Z is CO;
Y is selected from CR₇R₈, NR₉, O, and S;
R₇, R₈, R₉ are independently selected from H, C₁₋₆ alkyl, halogen, OH, and OC₁₋₆ alkyl;
a, b and c are independently 0 or 1; and
Q₃ is selected from C₆₋₁₄ aryl, (5- to 14-membered heterocyclic), (5- to 14-membered heteroaromatic) and (4- to 9-membered carbocyclic); each of which can be optionally substituted with 1 to 3 substituents independently selected from C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl), OC₁₋₃ haloalkyl, OC₁₋₆ alkylaryl and CN;
Q₄ is selected from H, C₆₋₁₄ aryl, (5- to 14-membered heterocyclic), (5- to 14-membered heteroaromatic), and (4- to 9-membered carbocyclic); each of which except H can be optionally substituted with 1 to 3 substituents independently selected from C₁₋₆ alkyl, halogen, OH, OC₁₋₆ alkyl, —C(═O)O—(C₁₋₆ alkyl), —C(═O)C₁₋₆ alkyl, NO₂, C₁₋₃ haloalkyl, —S—CO₁₆ alkyl —NH₂, —NH—(C₁₋₆ alkyl), —N(C₁₋₆ alkyl)(C₁₋₆ alkyl), OC₁₋₃ haloalkyl, OC₁₋₆ alkylaryl and CN;
Q₅ is selected from C₆₋₁₄ aryl, (5- to 14-membered heterocyclic), 5- to 14-membered heteroaromatic), and (4- to 9-membered carbocyclic); each of which can be optionally substituted with 1 to 3 substituents independently selected from C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl), OC₁₋₃ haloalkyl, OC₁₋₆ alkylaryl and CN.

31. The method of treatment of claim 29 or 30, wherein the patient is a human.

32. A pharmaceutical composition comprising a pharmaceutically acceptable carrier and a compound according to claim 1 or 2.

33. A synthetic process for preparing a compound of Formula IV: 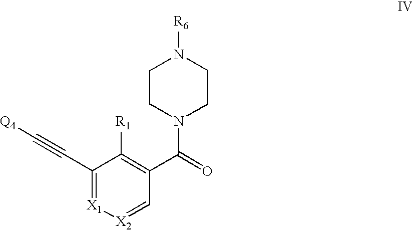
comprising:
reacting a compound of Formula III: 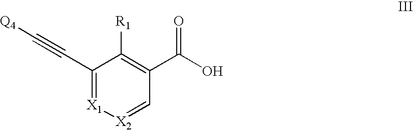
with an N-substituted piperazine of Formula IIIa: 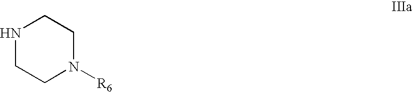
for a time and under conditions effective to form the compound of Formula IV;
wherein:
X₁ and X₂ are each independently CR₃ or N;
R₁ is H, C₁₋₆alkyl, halogen, OH, or OC₁₋₆alkyl;
each R₃ is independently selected from H, C₁₋₆ alkyl, halogen, OH, OC₁₋₆ alkyl, SO₂, (3- to 14-membered heterocyclic) and (5- to 14-membered heteroaromatic), wherein each of C₁₋₆ alkyl or OC₁₋₆ alkyl can be optionally substituted with 1 to 3 substituents independently selected from halogen, OH, OC₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl), cycloalkyl, NR₁R₁, and CN;
R₆ is selected from H, CH₃, -(L₅)-(3- to 14-membered heterocycle), -(L₅)-(5 to 14 membered heteroaromatic), (L₅)-(3- to 10-membered cycloalkyl), (L₅)-(C₆₋₁₄ aryl) and -(L₅)-C₁₋₆ alkyl each of which except H can be optionally substituted with 1 to 3 substituents independently selected from H, C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl, CN, (5- to 14-membered heteroaromatic), NR₁R₁, SO₂C₁₋₆ alkyl, SO₂, SO₂NR₁R₁, C₁₋₆alkylaryl, COC₁₋₆ alkyl, and (3- to 14-membered heterocycle) optionally substituted with NO₂; and
Q₄ is selected from H, C₆₋₁₄ aryl, (5- to 14-membered heterocyclic), (5- to 14-membered heteroaromatic), and (4- to 9-membered carbocyclic); each of which except H can be optionally substituted with 1 to 3 substituents independently selected from C₁₋₆ alkyl, halogen, OH, OC₁₋₆ alkyl, —C(═O)O—(C₁₋₆ alkyl), —C(═O)C₁₋₆ alkyl, NO₂, C₁₋₃ haloalkyl, —S—C₁₋₆ alkyl —NH₂, —NH—(C₁₋₆ alkyl), —N(C₁₋₆ alkyl)(C₁₋₆ alkyl), OC₁₋₃ haloalkyl, OC₁₋₆ alkylaryl and CN.

34. A synthetic process for preparing a compound of Formula IV: 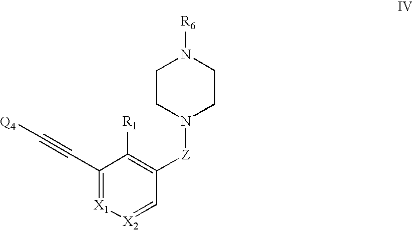
comprising:
reacting a compound of Formula VI: 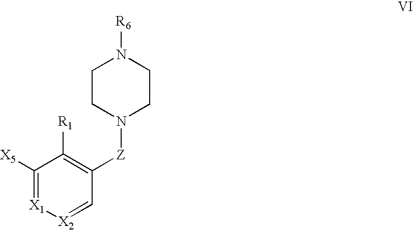
where X₅ is halogen, with an acetylene of Formula Q₄-CCH; in the presence of a palladium triphenylphosphine-containing catalyst for a time and under conditions effective to form the compounds of Formula IV;
wherein:
X₁ and X₂ are each independently CR₃ or N;
R₁ is H, C₁₋₆alkyl, halogen, OH, or OC₁₋₆alkyl;
each R₃ is independently selected from H, C₁₋₆ alkyl, halogen, OH, OC₁₋₆ alkyl, SO₂, (3- to 14-membered heterocyclic) and (5- to 14-membered heteroaromatic), wherein each of C₁₋₆ alkyl or OC₁₋₆ alkyl can be optionally substituted with 1 to 3 substituents independently selected from halogen, OH, OC₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl), cycloalkyl, NR₁R₁, and CN;
R₆, is selected from H, CH₃, -(L₅)-2-pyridyl, -(L₅)-4-pyridyl, -(L₅)-pyrazinyl, -(L₅)-phenyl, -(L₅)-(3-14 membered heterocyclic), and -(L₅)-(5- to 14-membered heteroaromatic), each of which except H can be optionally substituted with 1 to 3 substituents independently selected from H, C₁₋₆ alkyl, halogen, OH, OC₁₋₆ alkyl, —C(═O)O—(C₁₋₆ alkyl), NO₂, C₁₋₃ haloalkyl, —S—C₁₋₆ alkyl, COC₁₋₆ alkyl and CN;
Z is CO;
L₅ is a bond or C₁₋₃ alkyl; and
Q₄ is selected from H, C₆₋₁₄ aryl, (−5 to 14-membered heterocyclic), (5- to 14-membered heteroaromatic), and (4- to 9-membered carbocyclic); each of which except H can be optionally substituted with 1 to 3 substituents independently selected from C₁₋₆ alkyl, halogen, OH, OC₁₋₆ alkyl, —C(═O)O—(C₁₋₆ alkyl), —C(═O)C₁₋₆ alkyl, NO₂, C₁₋₃ haloalkyl, —S—C₁₋₆ alkyl —NH₂, —NH—(C₁₋₆ alkyl), —N(C₁₋₆ alkyl)(C₁₋₆ alkyl), OC₁₋₃ haloalkyl, OC₁₋₆alkylaryl and CN.

35. The process of claim 34, wherein X₅ is bromine, and the palladium triphenylphosphine-containing catalyst is Pd (PPh₃)₂Cl₂.

36. A synthetic process for preparing a compound of Formula IX: 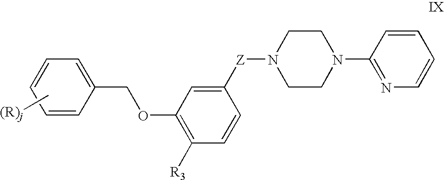
wherein:
each R₃ is independently selected from H, C₁₋₆ alkyl, halogen, OH, OC₁₋₆ alkyl, SO₂, (3- to 14-membered heterocycle) and (5- to 14-membered heteroaromatic), wherein each of C₁₋₆ alkyl or OC₁₋₆ alkyl can be optionally substituted with 1 to 3 substituents independently selected from halogen, OH, OC₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl), cycloalkyl, NR₁R₁, and CN;
Z is CO;
each R is independently selected from C₁₋₆ alkyl, halogen, OH, C₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl) and CN; and
j is 0, 1, 2, or 3
comprising: reacting a compound of Formula VIII: 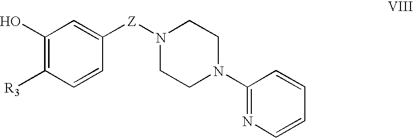
with a benzyl halide derivative of formula VIIIa: 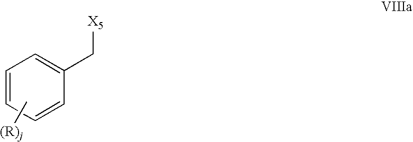
where X₅ is halogen, for a time and under conditions effective to form the compound of Formula IX.

37. The process of claim 36, wherein X₅ is bromine.

38. A synthetic process for preparing a compound of Formula XI: 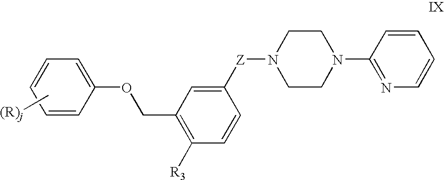
wherein:
Z is CO;
each R₃ is independently selected from H, C₁₋₆ alkyl, halogen, OH, OC₁₋₆ alkyl, SO₂, (3- to 14-membered heterocycle) and (5- to 14-membered heteroaromatic), wherein each of C₁₋₆ alkyl or OC₁₋₆ alkyl can be optionally substituted with 1 to 3 substituents independently selected from halogen, OH, OC₁₋₆ alkyl, —C(═O)O—(C₁₋₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl), cycloalkyl, NR₁R₁, and CN; and
j is 0, 1, 2, or 3;
comprising: reacting a compound of Formula X, 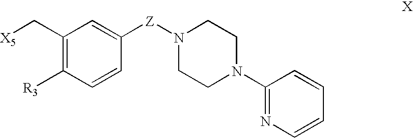
wherein X₅ is halogen, with a phenol derivative of Formula Xa: 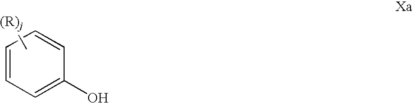
for a time and under conditions effective to form the compound of Formula XI.

39. A synthetic process for preparing a compound of Formula XIII: 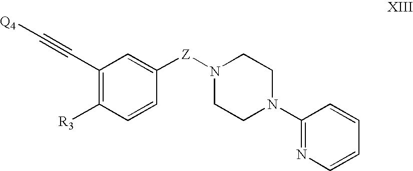
wherein:
Q₄ is selected from H, C₆₋₁₄ aryl, (5- to 14-membered heterocyclic), (5- to 14-membered heteroaromatic), and (4- to 9-membered carbocyclic); each of which except H can be optionally substituted with 1 to 3 substituents independently selected from C₁₋₆ alkyl, halogen, OH, OC₁₋₆ alkyl, —C(═O)O—(C₁₋₆ alkyl), —C(═O)C₁₋₆ alkyl, NO₂, C₁₋₃ haloalkyl, —S—C₁₋₆ alkyl —NH₂, —NH—(C₁₋₆ alkyl), —N(C₁₋₁₆ alkyl)(C₁₋₆ alkyl), OC₁₋₃ haloalkyl, OC₁₋₆ alkylaryl and CN; and
each R₃ is independently selected from H, C₁₋₆ alkyl, halogen, OH, OC₁₋₆ alkyl, SO₂, (3- to 14-membered heterocycle) and (5- to 14-membered heteroaromatic), wherein each of C₁₋₆ alkyl or OC₁₋₆ alkyl can be optionally substituted with 1 to 3 substituents independently selected from halogen, OH, OC₁₋₆ alkyl, —C(═O)O—(C₁₋₁₆ alkyl), NO₂, C₁₋₃ haloalkyl, —S—C₁₋₆ alkyl —NH₂, —NH—(C₁₋₆ alkyl), —N(C₁₋₆ alkyl)(C₁₋₆ alkyl), cycloalkyl, NR₁R₁, and CN;
Z is CO;
comprising: reacting a compound of Formula XII: 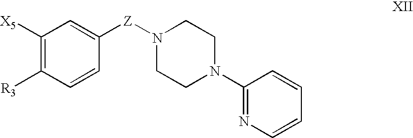
wherein X₅ is halogen, with an acetylene of Formula Q₄-CCH, in the presence of a palladium triphenylphosphine-containing catalyst for a time and under conditions effective to form the compounds of Formula XIII.
